SEQUENCE LISTING (110 sequences)

SEQ ID NO: 1 - DNA, 300 bp, Figwort mosaic virus
atttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttga

SEQ ID NO: 2 - DNA, 300 bp, Figwort mosaic virus
gcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagta

SEQ ID NO: 3 - DNA, 301 bp, Figwort mosaic virus
ctgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaaga

SEQ ID NO: 4 - DNA, 1983 bp, Solanum sp.
ttcaaatttcatttgtgtcatataaattgagacatataattgtcggcacatgctcatgtatccaaacaaggataatttgatcatctattcttatatatttgaaaattacgataataatactttaaatcacaataattaacaagttaaaatatttaaaagtcatataaaaaattaattgactctcaaaattctgtaagtactataaattaaaataaataacaacttaagaatttcaaagtcataaaaaatttggtggctctctaaaatatatcaatgtcacataaaaagtaacatatattattcagaaattacgtaaaagataccacaaattacaataattaacaacttgaaatatttaaaatacataaaaataattaattttagaaattccaggcgtgccacataaattgggacaacgaaataatatatactattattttaaaattatgtaaaaaaataattctaaatcatgataattaataacttaaaatattattaaaaatcatataaaaatttaaataattgctcaggtttcagccgtattacataaattaggataaaaaataatatatattgggccccgtgctggcacgggggcccgtatctagtttatataataaatatcgtttctagtctatctcttctgatgctaaataaagtctgtgattatcttttaattttttctactcagcatggggtgccgtatctagtttatataataaatatcgtttctagtctatctcttctgatgctaaataaagtcagtgattattttttaattttttctactaggtaatgtaaaattcttatgttaaccaaataaattgagacaaattaattcagttaaccagagttaagagtaaagtactattgcaagaaaatatcaaaggcaaaagaaaagatcatgaaagaaaatatcaaagaaaaagaagaggttacaatcaaactcccataaaactccaaaaataaacattcaaattgcaaaaacatccaatcaaattgctctacttcacggggcccacgccggctgcatctcaaactttcccacgtgacatcccataacaaatcaccaccgtaacccttctcaaaactcgacacctcactctttttctctatattacaataaaaaatatacgtgtcctttacgttatttcactaccactttccactctccaatccccatactctctgctccaatcttcattttgcttcgtgaattcatcttcatcgaatttctcgacgcttcttcgctaatttcctcgttacttcactagaaatcgacgtttctagctgaacttgaggtaaatttctagtgattatactgtacatttcgcataatttaggatcgtatttgatgatatgttttacgcttgattgatcgagaacttaaagcttttttgatctgaaatttgttttttggcatactcgagttgagatcctggttaaatcagtgttatttcgattgaattttagaaaaatttggtgttaattttcagtattttcatggtttaatgtgtataaacaagcttaatttttcaaattcaggctcgtttaaccttttaattacagcatatttctggaaaaaagtttggtgatttctctagatgttttattcgagaaaaaaacaaaaacgaaaaaaggggaaatgctgttctgtatgtacaaaaagtgattgatcagcttttggtcaccgacatacatttgattagtacatacacgagtcatacgagtatatttccgtgtgcactttattgttttgaaggaattctggatttggttgattcctttttaaaacttctaagttttttttgttgcattttactctaattaagtcttctctgtgaactgacaaatactcaccaggaacacattacaaccttcatttgattatccgcgaacgatccattgcttttgtgtatattgcttttgtattgactgattttgtattgtattagcagtgaattaagccagtgggaggatatg

SEQ ID NO: 5 - DNA, 342 bp, Solanum sp.
aaaattcttatgttaaccaaataaattgagacaaattaattcagttaaccagagttaagagtaaagtactattgcaagaaaatatcaaaggcaaaagaaaagatcatgaaagaaaatatcaaagaaaaagaagaggttacaatcaaactcccataaaactccaaaaataaacattcaaattgcaaaaacatccaatcaaattgctctacttcacggggcccacgccggctgcatctcaaactttcccacgtgacatcccataacaaatcaccaccgtaacccttctcaaaactcgacacctcactctttttctctatattacaataaaaaatatacgtgtcc

SEQ ID NO: 6 - DNA, 151 bp, Solanum sp.
cattcaaattgcaaaaacatccaatcaaattgctctacttcacggggcccacgccggctgcatctcaaactttcccacgtgacatcccataacaaatcaccaccgtaacccttctcaaaactcgacacctcactctttttctctatattac

SEQ ID NO: 7 - DNA, 1071 bp, Solanum sp.
taatataacataccatgggtggagctagaagtctgattacaaatttcgtcaaattcaacaatatttgcttaaataatatatttgtatagtaattttttttacaaaatatatacaaatttaggtcaaggattcagttattaaccctttaaaatcgtgtcataaaattcaatgttaaaattctgactttccccgtgcttaacattacttatcaaatttatgtttctgtgtagaaaagtactagtactactctttgactcgtctagacgtctactatagatctccttagattaaaaactccagttttaatattttcctcacaattattattcttaatctaccacctaccggagtcacaaatatattaaatgaaaatattctatctattaatttatgatctacctattgataatttgtaatctagtcaaaatgatggcaaaaaaaatataatatctagactgaagttcttagtcaatagcgtaaatgaaagaaaaaaaaaaaagctcaagaagaaacatgatatctttgttgctctgattcgtaaaaaaaaaaacatagtaacttcataaaatatcttatcctttggacagagcgatgaaaaaaatatattactagtaatactgagattagttacctgagactatttcctatcttctgttttgatttgatttattaaggaaaattatgtttcaacggccatgcttatccatgcattattaatgatcaatatattactaaatgctattactataggttgcttatatgttctgtaatactgaatatgatgtataactaatacatacattaaattctctaataaatctatcaacagaagcctaagagattaacaaatactactattatccagactaagttatttttctgtttactacagatccttccaagaacaaaaacttaataattgtatggctgctatacataattccccacctaccgcttcctggaataattgatatggaagccgcctctaaaattgaataattatactgttttacatattatataaagcaaggtatagcccaatgaattttcattcaaaagctagcaataatg

SEQ ID NO: 8 - DNA, 200 bp, Solanum sp.
aagttatttttctgtttactacagatccttccaagaacaaaaacttaataattgtatggctgctatacataattccccacctaccgcttcctggaataattgatatggaagccgcctctaaaattgaataattatactgttttacatattatataaagcaaggtatagcccaatgaattttcattcaaaagctagcaata

SEQ ID NO: 9 - DNA, 460 bp, Solanum sp.
ctagtaatactgagattagttacctgagactatttcctatcttctgttttgatttgatttattaaggaaaattatgtttcaacggccatgcttatccatgcattattaatgatcaatatattactaaatgctattactataggttgcttatatgttctgtaatactgaatatgatgtataactaatacatacattaaattctctaataaatctatcaacagaagcctaagagattaacaaatactactattatccagactaagttatttttctgtttactacagatccttccaagaacaaaaacttaataattgtatggctgctatacataattccccacctaccgcttcctggaataattgatatggaagccgcctctaaaattgaataattatactgttttacatattatataaagcaaggtatagcccaatgaattttcattcaaaagctagcaata

SEQ ID NO: 10 - DNA, 1776 bp, Brassica sp.
caccggctgcagatatttttttaagttttcttctcacatgggagaagaagaagccaagcacgatcctccatcctcaactttatagcatttttttcttttctttccggctaccactaacttctacagttctacttgtgagtcggcaaggacgtttcctcatattaaagtaaagacatcaaataccataatcttaatgctaattaacgtaacggatgagttctataacataacccaaactagtctttgtgaacattaggattgggtaaaccaatatttacattttaaaaacaaaatacaaaaagaaacgtgataaactttataaaagcaattatatgatcacggcatctttttcacttttccgtaaatatatataagtggtgtaaatatcagatatttggagtagaaaaaaaaaaaaagaaaaaagaaatatgaagagaggaaataatggaggggcccacttgtaaaaaagaaagaaaagagatgtcactcaatcgtctcacacgggcccccgtcaatttaaacggcctgccttctgcccaatcgcatcttaccagaaccagagagattcattaccaaagagatagagagagagagaaagagaggagacagagagagagtttgaggaggagcttcttcgtagggttcatcgttattaacgttaaatcttcatccccccctacgtcagccagctcaaggtccctttcttcttccatttcttctcatttttacgttgttttcaatcttggtctgttcttttcttatcgcttttctattctatctatcatttttgcatttcagtcgatttaattctagatctgttaatatttattgcattaaactatagatctggtcttgattctctgttttcatgtgtgaaatcttgatgctgtctttaccattaatctgattatattgtctataccgtggagaatatgaaatgttgcattttcatttgtccgaatacaaactgtttgactttcaatcttttttaatgatttattttgatgggttggtggagttgaaaaatcaccatagcagtctcacgtcctggtcttagaaatatccttcctattcaaagttatatatatttgtttacttgtcttagatctggatctgagacatgtaagtacctatttgttgaatctttgggtaaaaaacttatgtctctgggtaaaatttgcttggagatttgaccgattcctattggctcttgattctgtagttacctaatacatgaaaaagtttcatttggcctatgctcacttcatgcttacaaacttttctttgcaaattaattggattagatgctccttcatagattcagatgcaatagatttgcatgaagaaaataataggattcatgacagtaaaaaagattgtatttttgtttgtttgtttatgtttaaaagtctatatgttgacaatagagttgctctcaactgtttcatttagctttttgtttttgtcaagttgcttattcttagagacattgtgattatgacttgtcttctctaacgtagtttagtaataaaagacgaaagaaattgatatccacaagaaagagatgtaagctgtaacgtatcaaatctcattaataactagtagtattctcaacgctatcgtttatttctttctttggtttgccactatatgccgcttctctcctcttttgtcccacgtactatccatttttttgaaactttaataacgtaacactgaatattaatttgttggtttttttaactttgagtctttgcttttggtttatgcagaaac

SEQ ID NO: 11 - DNA, 515 bp, Brassica sp.
tgggagaagaagaagccaagcacgatcctccatcctcaactttatagcatttttttcttttctttccggctaccactaacttctacagttctacttgtgagtcggcaaggacgtttcctcatattaaagtaaagacatcaaataccataatcttaatgctaattaacgtaacggatgagttctataacataacccaaactagtctttgtgaacattaggattgggtaaaccaatatttacattttaaaaacaaaatacaaaaagaaacgtgataaactttataaaagcaattatatgatcacggcatctttttcacttttccgtaaatatatataagtggtgtaaatatcagatatttggagtagaaaaaaaaaaaaagaaaaaagaaatatgaagagaggaaataatggaggggcccacttgtaaaaaagaaagaaaagagatgtcactcaatcgtctcacacgggcccccgtcaatttaaacggcctgccttctgcccaatcgcatcttacca

SEQ ID NO: 12 - DNA, 1024 bp, Brassica sp.
aagctttcttcatcggtgattgattcctttaaagacttatgtttcttatcttgcttctgaggcaagtattcagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatggttgttagagttgggttgaatgagatatacgttcaagtgaatggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcatta

SEQ ID NO: 13 - DNA, 1250 bp, Brassica sp.
tgattctattgactgcagaatatttgataatacagttttttgtgtaacttacttaaatgttttgaactacacgttttgaaaagttaacctgttggttaaatggttagctatgactctcgcaacaaacccaacccttaagatgatgatggtttaacatttgacaacatagttaagactgtgtctatataatagtcaacaaattcagattgtagtattatggagtcaacatatttcgagatcaaaaacattcaaaacgtaaatctatcgacgtctcacatagttttgttatgaagctgatgaaaaaagttggaagacatagttttgcaaacatcatttgttgctaacgtataaacgttggtttgattaaatgtaataggataaggatatccgtttgttcatataattgagttaaattatattttggttattataatatgttaagttgaaaataaataggtccaacaaccttgtttaaatagattttttaggagtgattcccttttaatagtatagattatactctcttcctaatcgaccttccgtggggtaaagtggtcaattatattctttatggatgagcttgattgagaatgggtttatgggttatgacaagggcatgtacaaatgtcactgcctcttgacatgcaaccgaacagttggcgactcaagtcgcagaagatacaacggaccaaaccctccgagtgtcgccgcgtctgttatgtgtcacctttttgtctcctttccttaaaaattggtaactcatttttcaaaaaaagaagaggatagttttggctgtatctcctaaactattcgatcacaacgccagatattttaatactggatactagtgatgtaatttgatttgttaattgtcaaaaagtagattctcctatctcgtttttagttcaattattatatggttaaatgaatttaagtcgattagaaatgattagttaatcaaccagagttgctctataagtctatactgataacatgaaccattttctaaaaatgagatagatacatttgaattttgtcgtggtttggagtatgcggagatagtcgtacgcgcatgaacatcatgagacacttgcttcagctcacagagtgacgtgtaaagaccatagacccacgacttcatgcaaacccattcctacgtggcacaaaccttcatgctcactccacatatataaactcctaccaagtctccatgtttcttcatccatctatcacaaaaacacacaaacaat

SEQ ID NO: 14 - DNA, 143 bp, Gossypium sp.
gtcgactcgatcacggcacgtggatgagagagaaaatgagaaacaagtggtggagtaaaatgacgaaaataggtccctattccaaggagggaaagcttaaaacaaaaaagcttaaatacaggcgccccccttgaacacagaaa

SEQ ID NO: 15 - DNA, 660 bp, Sesamum sp.
catatgtgaaatgtaatggaaaatgcgacaagaattgcaatagagaaaatccaatttgcagagattacatgaaaagaatttgtacaaatagcatatatatgttaaaatgaaatgggacatgccacattatgtggaataaaaaagacaatttgcttggaattaattatagaataaatgtgttacatttaatatgtgattaatcactttttttgaattgtacatctatcacatgacaagttcattatatttgacatataatttgtttatgtctagtcaagcctaattaaatttctcggaaagcacaaaatttttttgtcctaaccaggtttgaacaaccaaacaaatcacaaagcaggtgtatcgcacttgcgatgtgatcggtcactttttctaaattgtacatcattcacacgacaactgtattgtgctccaagttcaattgagtgcggttggagctataatttccttgaacacacaatgtggaatgtgcacactccatgtgggccaatgagcggatgacacgtggcgggcaacttacctcgttacgttgaggcatgcatgaaagggggatctcttgaggtggaggggtgggggcgggggttgggggggggcccctcctcagacaggtctatatttatgagacctcgtaaggcagaacgc

SEQ ID NO: 16 - DNA, 527 bp, Glycine max
tgttttgtttttggttatgggattaattttttaattacgaagaagcttttagagcatcacccgaatctaattcgttttggcttttgtgatcttgatgtaaatctatactaacttggtttgggcaagagaaattggtccttgctcaagtccattctaggacgaaaataaaaatataacagggtatagcagatctctattcgtatgtgggtaacgatagcatgtttctattgttctcttattcttcattggtcacgataacctgctaattatgccacgattgagatgaaaagtaacgaactagtaaaccatagtgagaagaacatttcgctactattgttgaaacgtttacaccaggcacttgagtatgatgcactatatttcaattaatgtaatttttcgctttgatgagaaacattctgattctgtgagtttagaaactattgctgataatccttgatttaagatttcagtcttgttcatgttcatttgaagtgttggtaataaaatgcactgatgtgtcatgtgca

SEQ ID NO: 17 - DNA, 438 bp, Gossypium sp.
taaatatatacttttttagtgttgtaaattttaatatgggtcggcccgggccgagctcgggcttagcaattttttccgggtcggacttggataaatttttaggctcatatttcgggccgggtcgaatccgacctaaaaaataagcataaaattttgtcttggatccagcccaaatctagcccgacccataatcacctctagtttaagcttcttcttttctttctttctttctttctttctttcttttttttttttaacattaaaaatatgtagagaaaatcagcaattaaaacaaaagttagggctaatgtgttaaagtagcaccaataaagtatccctctcaagtgaagtctttcacacttgcaaacaaaaataattaaaagacagaggagtctataaagttaaaagccgtccaaaacccaaaccaggaaaggcaaa

SEQ ID NO: 18 - DNA, 1191 bp, Linum sp.
gagctctcaatgtagtaacacaaactcttttttttccataacgttgaatgttagaactttgtctttttataactgtttctttcatgaagctgatcagctgatgttggagaaggatggagccacggagattcctgaaaagcaaaggatggaacgagaggagacggtgactcgagagtacagggaagcattgcacagagctgtcacgcttgcagtgcctcattcagagttcttgtctcggtatggaacatttagtggcggtgacgttgaagaagaggaagaaagatgctatggttcatcatctagtgggaaggattgatccagccggcatgttctcctcccgaaatcgggccgtcccaattgatgacaatgtaacatcaatgtcaatctctgcagatttttgttagcagcaggtcatgattcttttttggttgattcttgtgaatgtaagctatttgttgttgtaatatatgcattgattgtgattttgttttagctttgatcaatgaaataaatctcgttcaacccaaccatcaggctctttcatattcattttgacgactatatatacataatcgtacaaactattcggttaactaatctacagaaagtcggagttagctagagattgtcaaggaggaggagatcatacacctaattttgaagctgattcttcatctatgatttcgagttttgacttgatttggctcttcgatattcgaaattaaatgcctcaatgcctccaaagtgctctctacttgcgggtggacctacaaaactaggcaaacaggtgcaaaaaacatgtgtttacacgtccatgttatcttgcattggcccatgttttctgcattgtaaatctttccccaaacacatagttagacgaagtcgataatctagcaccatcaaatcaataacacgagcaaataataaagtaaatagtgaaaccatgaagcctaattggtcgagtggagctgaaagctttcatcggtatcgaacccaaccccccctgctacgaaacttaaaaatgggttacgctattacactcgatagaactgatgaaacgcaacgattgttaagtaaccattttgcagaaacgataattgacaagtgaccatttggataaatgaccagggaaaatacaagtggcgagtgctgacataataaaccgaatgcgggcgttaccatccaatttta

SEQ ID NO: 19 - DNA, 1693 bp, Linum sp.
gagctctcaatgtagtaacacaaagccttctgtcttctttctgtaacgttcaatgctagaacttgtcttcttataactgtttgtttgcttcttcagctaatgttggagaaggatggagccacggagatcccggtaaagcaaaggatggatcgagaggagacggtggctcgagagaacatggaagcattgcacagagccgtcacgttggaagtgcctcattcgcaggccccgtctcggtatggaacatttggtggtggtgaggttgaagaagaggagaaagatgccgtagttcatcatctactgggatggattgatccggccagcatgttctcctcccgaaatcgacctgtccctattgatgacaatgtaacatcaatgtcaatctctgcagatatctgttaggatcaggtcatgattcttttttggttgattcttgtgaatgtgtaacattgatgtaagctatttgttgttgtaatatctgattttgttgttgctttgatcaatcaaataaatctcgttcaacgcgatcataagcctctttcatattcattttgacgactatgtatagtcgtacaaactattcggttaactaatctacatcaagtcggaattagctagacattgtcaaggaggaggaaaatatcaagaaaattggatgaggaaatcatacacccaattctgaagctgattcttcatctatgatttcgagtttcgactttttttgagtctcaactgtgatttcgagtttcgacttgatttggctctttgatattcgaaattaaatgcctccaaagtgctctctacttgcggttggcctggttcaatggcgaatcattgaatgacagaactagacagctaccaggtgcaaaaaacatttgttaatgtcttcttgcattaatgtccatgttttctgcattttaatctttccccaaacacctaatatatagcttcattgatcctcctctcacggttgcagatctcgttgctgataacacatacatggctacaagactctaaaacggttcaaagtgaaattgttttggtggtagagttgtgtgtttggtgactcgaaagttctggattcgaatccagcattccccacaaaatagacaccaacgtagtgtttatttaccgtcttctatcttgtattgaccgagagttacgatatactccgacaaaaaaagacatcttccacatcatcaaatggatccgtagttagtgcagtggctcgattaacataaatgaaaaaaggaaaaaatttgcctgaaatcgatgctcaaaacaagtagaaattcattcaaacatatttagacaaacacgatcatttagcatcatcaaattaataacaagagcaaacaataaagcacatagcaaaacatacaatagtcgtcttgcaatgtcatatgataataagccagtgaaaccatgaagcccaagtgaagtggtcaagtgggagctgaaagcttccgaacccaagcccccgctaccgggttaggacatacgacacgcgacatgctacgaaacttaaaaatcggtcacgcagttaatggaacaaatgaaacgcaacgactattaagtgaccattttgcagaaatgatatgaaaaagtgaccatttagacaaatgagcaaagaaaatacaagtggcgagtgctgacataataaaccgaatgcaggcgttaccatccaatttta

SEQ ID NO: 20 - DNA, 1024 bp, Medicago sp.
aaatgaaagagagttaaggattgaaatgaaactggtaaaaaacagcttattttaaaacatcttattcaaaacaacttattttatttaaaacaatttattttattcaaaacatgttttgaataagttgttttttgaaaataagctgttttgaataagctgtttttaaaataaggtgtttttcataaaataagttgtttttgttaaaataagttgttttttcaaataagctgttttgaataagctgtttttttttaaataagttgttttgaataagctgttttttttaaataagttgtttttttaaataagctgttttgaataagttgttttaaaataaggtgttttgcataaaataagctgttttgaataagttgttttgaataagttgttttgaataagctgttttttttaaaaataaattgttttcataaaataagctgtttttaaaataaggtgttttgtataaataagctttttaaaataagctattcaaataagttgtttttttggaaagatccaacaaagagttcaagtggtttctttaaaataaaataaaaagttcaagtggtttggttcggttcaaacggttcggttcggttcaagatggttcggttatggttcaagaactgttaataaattaacggttcggttcgtgaaccattataacgattcggttatttttggttcggttcggttcgcgcggttcggttcggttcatggttctttttgcccacccctaaagaaaataaatgaatggtggttgagtattcttaaaatgatttgttttctagaataaagagttaataagggggtcaaaagagcaaccatctaaggtaaactctcacatttagagttgatgcggttaaaatttggatataacacttttgttgaccaaaatgtctcttatgaataagactgaaagaagtaataatttaaaaaaaaaaaatccggctgttgcattttttaaaacattaatccgaagaaaagatgtttgaaaattgtttataatgagaagttattttga

SEQ ID NO: 21 - DNA, 448 bp, Medicago sp.
caccaacatgatttttgtatgcttgtaaatgaaaagcttctagttatccagctcaacccgtgactaaggtctattcaatttgcttagaaatgaggcatcaattatgatgcaaatttttgtactcattactcaattcaaaaactatatgaacttatggtgtcacgtaagtgaataacactatctaaatttgagtacttctcctgtcacggggagaaaaacactcaaaatcaattgcatgcaacggcaacacatttctgtttacaattatattcggtgagtactcagtcagtataacccaattaccacatatgcacgaattctcttagtgggtccacattgtggtggttgagtgggacccaattgtaatggatggcccacatacaccaaactcaaccaaacaatttctcataaagttctatataatagcaatccactttgcatcattgag

SEQ ID NO: 22 - DNA, 1021 bp, Medicago sp.
atagtggaccagttaggtaggtggagaaagaaattattaaaaaaatatatttatatgttgtcaaataactcaaaaatcataaaagtttaagttagcaagtgtgcacatttttatttggacaaaagtattcacctactactgttataaatcattattaaacattagagtaaagaaatatggatgataagaataagagtagtgatattttgacaacaattttgttacaacatttgagaaaattttgttgttctctcttttcattggtcaaaaacaatagagagagagagagaaaaaggaagagggagaataaaaacataatgtgagtatgagagagaaagttgtacaaaagttgtaccaaaatggttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgagagagttggattaaagttgtattaatgattagaatttggtgtcaaatttaatttgacatttgatcttttcctatatattgccccatagagtcatttaactcatttttatatttcatagatcaaataagagaaataacggtatattaatccctccaacaaaaaaaaaaaaaaaacggtatatttactaaaaaatctaagccacgtaggaggataacatccaatccaaccaatcacaacaatcctgatgagataacccactttaagcccacgcactctgtggcacatctacattatctaaatcacacattcttccacacatctgagccacacaaaaaccaatccacatctttatcatccattctataaaaaatcacactttgtgagtctacactttgattcccttcaaacacatacaaagagaagagactaattaattaattaatcatcttgagagaaagcc

SEQ ID NO: 23 - DNA, 858 bp, Medicago sp.
agagaggaggcagtgtacacaggggcagagagaggtgagtcgtctttctggtagggctggtgttggggatagtggttggtttgagagtcaggtggtgaggagggttggcgatggggttgatacgttgttttggttggataggtggttaggagatgctcctttttgtgtttgtttcaggaggttgtttgagttaacagagaacaaatttgtgtctgtggctaatttgttatctgttgactcggagcagtggggggaggtgttgaggtgaagcgtatggtggcagaggtggtggcagaggtgaagcgtatggtggcagctgagggaggcagtgtacacagaggtggagagagaggagagagaagagagaagagagagaaaatggagaagagagaagagaagagagagaagacaaatttttgtgtgtgtgaccaaaccaaaattcttggtcctggtccacacaagattttctcccaaccaaggtacaagaataccacgatccaagagtgccacgttgcaacatcataaccgttcaatagtaagagataatcgaacggccataattaattttcaacaaacccacttttttcctcctacttttgcaacttgtccctcatcacctaccaaacacacatagcacaccaacacacataataatattataataattgtaaatatatgtagcctccaaattagaaagaaacctctatataaagcctaactacttccttcacaaatcaggaaattcacaactctaatattcatttctttcctaatcattagaatttccattcttataaaattctaggtaccaccacacaacaaataaaggaacattaatcaatactattaagatggatc

SEQ ID NO: 24 - DNA, 1008 bp, Medicago sp.
cttctattaatgatttaatcaaccttttttaaaatacgaaggtgaccttattttgcaaataatccatgcatggaaatgcatcatccttttgaaaatgggattatctgaattcttaagttacgtgaaaatttaatacatttcattttagataaatttattattaaaattcacacttagatggcctaaaaattaacacttatttttaacaattcaaataaaatatacgacgaaatgagtgtaatttagttggttaagcatcgtcaagcttggagagaaagatcatagtttgatctttgaaaactacactattgaaaagggtgaagatatctaaacatccaaacaaaatttattttgatagtcgattcaaattatcaaaatttgtgaaaatattttgtaaattgttaagttggcaaaaatatgttaattttcaaattaccatttgcacatttttctaatctcaaatcacatttaagggatgttgactactttagttttgtacaaatctttacaattttaacatttataaaatgtgtttcggtagataaaaagtgtgagtattgtttataagagattgtgtttttcttttgtttaaacttataaaataaatatatattttattttattttaatgtgagattgtaagaattcattataagattatgtcattccctcaaaagaaaattagatgatgtcattttcataactcattttctataaatacagaaaatcctcaaaaatgaaaaacctcagtcaaaaaataaaagaaaaacatcaatagtggactggcccacactcattgctttgctttagtataagaaagtagacctcaccaaccacgaaccggacgccaaccggttcaaccaaacattacaccaattttccttaaccataccggtttttccctcccttatataaccatcttcctacctcttatctaaccaagctccattcaactcttcaacacatatcagaaacagaaaaagaagcaaaacattccaagaatttaaca

SEQ ID NO: 25 - DNA, 171 bp, Medicago sp.
catcaatagtggactggcccacactcattgctttgctttagtataagaaagtagacctcaccaaccacgaaccggacgccaaccggttcaaccaaacattacaccaattttccttaaccataccggtttttccctcccttatataaccatcttcctacctcttatctaacc

SEQ ID NO: 26 - DNA, 289 bp, Medicago truncatula
tgtacattagaagttcccatcatatactactgtctaaagaaatgcattaagttttgtcctatttatttgatttttttcctttctttcaatttcaactgttattttgattttttgtaaccggaacgagttcatgacatactgttacttatctcttcacttttatggtttttacattttttttttttttttttttttttttcggcaatgattttcacttttatagatatataattagaaacctctactcctatttttatctccctatcaatgatgatagcaaaattgtata

SEQ ID NO: 27 - DNA, 794 bp, Pea sativum
acatgcaccgccaccaagatatcctactttctagtgtgtcattcaagacttattatggtgtatcatacggaaagaagaaaaataggagagtgtatggtgttgaattattgaccatacaaaacaaaatgaggttagatttgcgaaggataaaacctttgacaattaccaatgcgataaatccctcacgaatatttattttgtgatgaatttttgcacttgtgagagatttaaccctcacaaaagagtcttatagtgttatttttatattaatttgttaattaatatgtaggaatgtagtataattaaaaaggtgtagtcatttatcctattacttacaatattgtgatttgagacactctttaagtaaatgatgattgataagtatagtagtataaaaatttataaataatataatgtatgcattgggttgaccgacatttagagttgaatctaaagtcatggtcatgcatggttgcttccaccatatttcttgccaactacctcgtgtttctcttagtctattgccatccacccatatgcatctatctaccaacccaaaaacaaagaaaaccaaaaccctagattgccacgttacaaaatcttaactgttcattagtaagtgatgatcaaacggccataattaatattcaacaaaccacttttctttttttctacttgtgcaacttgtctttcctcacctaccaaactcacatatcacaccaacacacatgcaatgcacaatactacatttcaaagtctctatataaagcttaaccactcttccttcacatctc

SEQ ID NO: 28 - DNA, 211 bp, Trifolium subterraneum
ctcataattaattttcaactaacccacttattttctctacgtactgcttgtgcaacttgtctctccctacctaccaaacccacacatgcataataataagagagagttaataatattacaataatgcatattaatgtagcctccaaaatatactttatattttattttattttgatgccaaacacacctctatataaagctcaacaactct

SEQ ID NO: 29 - DNA, 658 bp, Populus kitakamiensis
ataatatatatttttaatatagttataatatttgcaaattaaaacaataagaaaacattaaattgccacaaaaaataaaaaaatttaaaaacatcatttatgtcgaaaaacaaacatgtatttattctttaactaattagattttagatttgttttttaaaaattatcaatttgaatcatttcaaattactggagacttacataatcattaattaaagacccatataattaatcaagatatatataaattcatctcgatatctatataaaaatccagcaggccatttgcatgattattaggaggatccatgtggttttattaattacaggagcacatatatatatatatctatatataaaagaagggcaagacgaaatttctcatttctcatttctcaccaaccacaacctcatcaccatgcatcacactgcacgatagtcaaatttacccttctacgccaatcgccaatatggatccacaaagagaccacgctccataatattgacccttgagattattcaatatcaatggtaacaattgagtttcaacaaacccactttgtcccctcatgcttacctaccgacctccatgtctctatgcatagtattcaagactcccaacgatctatttaaacctccttccctccctctcttctcc

SEQ ID NO: 30 - DNA, 590 bp, Arabidopsis sp.
tggggtggagaagatgacaatgagaaagtcgtcgtacatataatttaagaaaatactattctgactctggaacgtgtaaataattatctaaacagattgcgaatgttctctactttttttttgtttacattaaaaatgcaaattttataacattttacatcgcgtaaatattcctgttttatctataattaatgaaagctactgaaaaaaaacatccaggtcaggtacatgtatttcacctcaacttagtaaataaccagtaaaatccaaagtaattaccttttctctggaaattttcctcagtagtttataccagtcaaattaaaacctcaaatctgaatgttgaaaatttgatatccaagaaattttctcattggaataaaagttcaatctgaaaatagatatttctctacctctgtttttttttttctccaccaactttcccctacttatcactatcaataatcgacattatccatcttttttattgtcttgaactttgcaatttaattgcatactagtttcttgttttacataaaagaagtttggtggtagcaaatatatatgtctgaaattgattatttaaaaac

SEQ ID NO: 31 - DNA, 848 bp, Medicago truncatula
catgtccctaaaagagaccccgcctaaccatgagtttgtccgaaaaaaatgtattgacccattgcttatctcccgtcaaacattaacgtcgaaccaacttctgatccctaaaccaattgtatccctcacctttgccatctcattccaccactcagacccattcttatctctattcatcaacctccctccctcctcatcgtacctcgccaccaacattctattccacaactcatccatatccatcaacactatttttctaacaatgcaatattaaaatcccacatcttgcagagatcattacatgaagttatacttgtacgggtcttgaagaagaaaagtgtgttaatagttagtttattagattaatatttattcatttgtgccggatttgaattcaaaacattcaactcttttatcttaattcagaccggttgaactatttaatctctagataaaattagatgttgttgaatgaatattcaaaattaatgggtgttaaatccttacaaagtgagttcggtcaaaaaaaaaaaaccatacaaagtgagttacacttttttttttttgagagataagttattataccaaaaaatacccaaacataacacaaaaatgaattaattactttttacaaagaccatccaaccatgaaccattaactcgatgagaaaagagaatgcaattcttagtttaatctacacacaaaaaaagacaacacacaccaaggccacaaaccccacctaaccctctacagtaaatccacctaaccaaaaccccatacacatcatcatcatcatcatcatcatcaaaacctctctataaaaacccaacaaccactccaaacattt

SEQ ID NO: 32 - DNA, 747 bp, Populus kitakamiensis
attaataaacgcaaagtagtttgtcacactataggagaaaatatctaataaaaagtaagaccttatagtttcaagaggttaggttgatatttaaagagagatttctttcattaactttttaggttgaaatcttgaaattaatattaaaaagatttgataatccttttactgtgaatactttggattgggattcacatttaaaattattcttaaatgaaactttatgttatatgtttgatactgtatttttacttgtttttaaaatgtatctgttttttaaaaatatcaaattattaattttttattgttttttaaaagattttaatgtattaattttaaaaataaaataaaattattttaagtgtatttttaaataaaaaatattttctaataaaagatttgaaaaaaaaaaggataggaaaaaaactttcttggtggagagccttgtccctcgaagcttaaatcatcatagattagtggcgcccacattacatcttgtatagaaatacaaaaaggccagggaaattaattaatatgatgaccatatgacattttcggccaccaacccgccttacctactactatccatgattatcaatgacactctcctaccacctcaaatgtaacgccgttaactctctctctctcccccacacacacaacccaacgcgtgaaattcaacttcatttcctctctaatttttgcagttataaaacccaagctctcctcatcctgttgctcccatcc

SEQ ID NO: 33 - DNA, 535 bp, Populus kitakamiensis
attattcttaaatgaaacatgacgtgtgtgagtttggtattgtattttcacatgtttttaaaatgaatttgtttttaaaaaatattaaattaataattttttattgcttttcaaagattttaatgtattagttttaaaaataaaataaaaattattttaatgtatattttttaaaaaaatattttcaaataaaagaattaaaaaaaaaggataggaaaaaaactttcctggttgagagcctatcccttgaagcttaaatcatcatagattagtggcgcccacattacatattgtatagaaatacaaaaaggccaggcaaattaattaatatggtgaccatatgacattttcggccaccaacccgccttacctactactatccatgattatcaatgacactctcctaccacctcaaatgtaacgccgttaactctctctctcccccccaaacacacaacccaacgtgtgaaattcaacttcatttcctctctaatttttgcagcttataaaacccaagctctcctcatcctgttgc

SEQ ID NO: 34 - DNA, 399 bp, Medicago truncatula
tcttgtttaatttaattattctccagaacaatctagtccttgttaattaaattaattcagagtgttttggtcctaaattaactgttaatattatattttgtttaatttaatcattctccagaatgttctggtcctacatatattaagtactatttattttgttgaactaacgtaaactaaaatcaagaggttctcgtagagtactacgaatatatagggtgctaataccttccctaaaaatataatcaacccccgaaccctaaatcttttcaaaatgggttgttttgaactttttccccttttaaaaaaaaattgttcagtcgtgaaataaaagtgagtcaaacgctaatcaaatggtcttgatctccaaaaaatggcgcgacaaaaattaagcaatgt

SEQ ID NO: 35 - DNA, 1024 bp, Lycopersicon sp.
aagcttcttaaaaaggcaaattgattaatttgaagtcaaaataattaattataacaatggtaaagcaccttaagaaaccatagtttgaaaggttaccaatgcgctatatattaatcaacttgataatataaaaaaaatttcaattcgaaaagggcctaaaatattctcaaagtattcgaaatggtacaaaactaccatccgtccacctattgactccaaaataaaattattatccacctttgagtttaaaattgactacttatataacaattctaaatttaaactattttaatacttttaaaaatacatggcgttcaaatatttaatataatttaatttatgaatatcatttataaaccaaccaactaccaactcattaatcattaaatcccacccaaattctactatcaaaattgtcctaaacactactaaaacaagacgaaattgttcgagtccgaatcgaagcaccaatctaatttaggttgagccgcatatttaggaggacactttcaatagtatttttttcaagcatgaatttgaaatttaagattaatggtaaagaagtagtacacccgaattaattcatgccttttttaaatataattatataaatatttatgatttgttttaaatattaaaacttgaatatattatttttaaaaaaattatctattaagtaccatcacataattgagacgaggaataattaagatgaacatagtgtttaattagtaatggatgggtagtaaatttatttataaattatatcaataagttaaattataacaaatatttgagcgccatgtattttaaaaaatattaaataagtttgaatttaaaaccgttagataaatggtcaattttgaacccaaaagtggatgagaagggtattttagagccaatagggggatgagaaggatattttgaagccaatatgtgatggatggaggataattttgtatcatttctaatactttaaagatattttaggtcattttcccttctttagtttatagactatagt

SEQ ID NO: 36 - DNA, 1927 bp, Lycopersicon sp.
tggcatgatctcagtaaatgtagtgtagtgtgtacatgaattatacatcagttttgaagaggtagtataatggaagtatcatatcaagggtatggccatatttgcaatgacaaatgtaaaatgtgatgagccacattaggagtgattccggcgtccgttgtcaaagttaaatttgtttctacttattatgcaacaatcaaaaacttctttaacttctgcagaatgatataaaatgagagaaagatgcaccaacctatgtacagtttttacttttgtcatatcgcatactttttttctttttgcttttccttatctgccatggaaaaaagatgtcccctaattatacacaaattaggggtgtcaagtgtcaaaaagggcggattatgtttgaaattgatcaagttaaaatgagttgaattcacaaataggttggttaaagtcaacccaatagttgcttcatgcttgggctaaaaatgggttggttatgatccactaatttgacccaattttttctaatggtggtccactcctaatacccgagaatcgagccttgtctcgacacttgggacataagacttgtataccaattgtaaaaaactcatttatgattttatgtataattttatataaaatcaatttatctctcctatcccaattacatagtttttctcctaaaaccactcctccaatctattttgaattttaaatttcataagatttcatgaacttccttttgtcttgctctcaattttcgcaggaaacccatgaatctatttttatttttttccccttcatcaacaattgtatacgtattatgcttcttagtttttcatataattttttttaaaaatctttctttctcatcatattacaagttgtttaaaatcagaatgaaagattcatcttaatatgtaagaattacctgtttgaatgtcatgtatatagttgtttgcacaatgaattattctatacaaaacttgatcaaggtagtttgtattgttatactcatattttaagtttttttgtatattcaactagttatatatgtatataagtaattacttttaaaaaagatacacttatttgtataataatttgttttaaatcacaatttttttatactttacgttattatatacaaactgcttaatggatttgtgtatatacaagtactatattcatatttttatttatacatatacaattacttatatatgtatataataattaatttaataaaaatcaaacaatttatattcattttatttacatttgtatataaatttgtttatacgtatacaattttttgtatatttattttattaacattcgtatataaacttaaacttttttttatacatatacaatttttttttatatattcaactagttatatatgtatataagtaattacttttaaaattttggtacaattatttgtataataattgttttaaatcatattttttttgtatttcatattattatatacaaaactgcttgagggattcgtgtgtatatgtatataataattaatttacaatttggtgcaaattaaataacttatattcaatttatttacattcatatataaactttatatatattaagagtttaatttccccataaacaagttttttatgaattttcagtcacaatagaatttttttaaaaaaaatatttttaaatgtttaacttaaattatgaaatgtgtaaatgtttgttaaccatatttagggctattgttattatttaatgaaaaataaaatataatataattcttaagaaagtattatatataaaataaaaaattacgtaacaaattatactatacccacaaaatataattatgtaaactataccatataatattatttcgtaaatttagtttgtcatataaaattttccctaaaatgaacagaaaccc

SEQ ID NO: 37 - DNA, 1045 bp, Medicago sp.
cgaggggactctattgatgatttgaagacacaacttaacacttattttgagcatcttggtgaaaatcaatatacacgtcacttgtctgctctaatgccaatgatagacctaggagaagatagagatgaattcacatggaaaacggcaagctatatgccttggcttattaaagacgatagcgacgtcggatttatgtttaggaatatggtggaaaataatgtattatatatatctgttcgttccatatgcaattgtaatgaatgtaagtagggatttaatttaatgatgtgtaatgatgtgtaatgacttgtaatgtgttgtttgattatggacactatgttccgttttgatgaatttcaaacttttgtgtggtttgaaccaaatgtcggtttgatttaattatggacatatgtaaaagatattgtatttttcttgtttatgactgagtttcattgttgtataatttgaattgcatatggaaatgctctggtaaaattacaggtaaaaactggccgaaaaatggcttggaaatgcttagcattaatgcagaacctgctgtctgcataaatgctttcctcggcagttaactaccgaggaattcctcggcagttaactgcagccggatttcaaattcctcggcagttaactgccgagggggcaaaagcgtattttacatgtgtgtcccagccttctttaatgtgtgaacaacaattttctaaaattaaaccctactctaggtttaacataccagtaaatttttgctttttgtatgtgttaacccttctccaatcccttgcacaaccatctcctcaaaccttcttcttctggagcaaagtcgccattccctacctccttcttcattcttattctctataacaaacggtccgaccggatccaagttgcaccggttcgaaccgctttagttactactaacggttcgaaccgttatttttcaacccgtgacgaacgtggaaggcttcgttgtttcttcttcttcttcttcttcttcttattaattaccatgcgtttttgtttttcttttgag

SEQ ID NO: 38 - DNA, 1218 bp, Medicago sp.
gatgggggtgacccacgatcggcttctggatcactttatgagtttgtcatgtttctcttttcaaactccttgacttgctcacttccagcttgctaggcaaaaccatgtatgtttcaacttagtgggtgtttggattaacatttggaggctcatttccatttctcagtgcactttaaacatgaaaattgtgaagcagaaatttctagcttttagaaaaacgcgcgtctaaaagccttccaccgcagtcctaaacagtcacctaatcttttaagtccaaacatctattgatagtagtgattcacatacttgaaaccttactatttaggagggggggttccattgaattacatgcaaaaataatttggagagcatgacatatacatacatacttttatatatataagtgtgtttcaaattatataatttaaggattaatagcagttttggcccccaaacttttcaaaaattacgattttggtcccctaagaaaaaaaactacaaaaccgccccctaagttttgcacctgtggcagttttggcccccaatgccaattttgactcggtctacgctgacatgacaccctaagtgaggtgccacgtgtttttttttctttttattttttaccttggggggccaaaactgctacagttgcaaaacttagagggcagttttgtagttttttttaaagggttaaaatcgcaacttcatgaaagttaaggggcgaaaactgctattaagcctataatttaaaatacgttttataattcaaaatggattgaattgaaagaaaaaaaagaagagggcgcttggagcgtaaaaaaaaatctcgttaattttttttttaaggaaaaatctcgttaatttatttactattggcccatgagaaaaagtccgataaaattaaaccctactctaggtttaacataccagtaaatttttgctttttatttgtgttaacccttctccaattccttgcacaaccatctcctcaaaccttcttcttctggagcaaagtcgccattccctacctccttcttcattcttattctctataacaaacggtccgaccggatccaagttgcaccggttcgaaccgctttagttactactaacggttcgaaccgttatttttcaacccgtgacaaacgtggaaggcttcgttgtttcttcttcttcttcttattattaattaccatgcgtttttgtttttcttttgag

SEQ ID NO: 39 - DNA, 634 bp, Solanum sp.
ggttggggtaccgattatgttcggatcagtttacacatattttgattaattttaagaaatacttgttatttttcatcaatacaaatattggataaattcattcacaaagtaatattctccccctctattaagtagtacaatttctatttcaatttatgtagcgatgtttgactgaacacaaagtttcagaaaaaaagaaagaaagagactttagaaatttacgatcaaaaacaaacacccacatttgtccgggtaaatataattggatccttacataaaaataaatagctgtcagattcattattattattattttgtcagtatacataagttaagcattggttatatatagatattatctccaatttaagctattaaattgaacaactattcaaattaattctttcagtatttaattgcagccacaatcactttaaatgcaactaatccactatgaaatgtttgaacggtagatacaaaaaagttcaacgtgacattcacttactaatttaatacctaccaaacccctatgtccattttttttaaaaataaaataaaattcaacttctcattcattttccttctacttcattctcactctctctatataaagaaattgtgatattgaaaaact

SEQ ID NO: 40 - DNA, 335 bp, Solanum sp.
aagagactttagaaatttacgatcaaaaacaaacacccacatttgtccgggtaaatataattggatccttacataaaaataaatagctgtcagattcattattattattattttgtcagtatacataagttaagcattggttatatatagatattatctccaatttaagctattaaattgaacaactattcaaattaattctttcagtatttaattgcagccacaatcactttaaatgcaactaatccactatgaaatgtttgaacggtagatacaaaaaagttcaacgtgacattcacttactaatttaatacctaccaaacccctatgtccat

SEQ ID NO: 41 - DNA, 634 bp, Unknown Organism; identical to SEQ ID NO: 39
  Description of Unknown Organism Unknown promoter sequence

SEQ ID NO: 42 - DNA, 578 bp, Solanum sp.
taagtatctttttaaaaaaaatctaatttcaatataatttaaatttttttttactattgtgacaataaatttgataaaaaaaattatttgccaactttcacaaaaatattttgacgcaatagtataactatttaatactatttttttattttttatttataaaaaagatgaagagttaatgatgttttaacaaagaatttttttttgatgttttagcaaaaaactttcttgcaaaggaagtgtacaaataaataaagtgtgaagggtatttttgtaaacatatattatttaatagtaattatgcaagatttattatttttaatacatcaaaccaaacaatgtataagaaataatacttgcataactaatgcacgcactactaatgcaagcattactaatgcaccatattttgtatttgttcttatacactctaccaaacgaccccttagagtgtgggtaagtaattaagttagggatttgtgggaaatggacaaatataagagagtgcaggggagtagtgcaggagattttcgtgcttttattgataaataaaaaaagggtgacatttaatttccaca

SEQ ID NO: 43 - DNA, 363 bp, Zea mays
  modified_base (102)..(102): a, c, g, t, unknown or other
gtggggttcctttcatttcgtgctctcctttctctgccagccagtccgtccgtccttgcgtccactgcacctgcacacaggtcaccccgacccgcactgttntagactccattagaaaaaaaaaggtntgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcag

SEQ ID NO: 44 - DNA, 999 bp, Brassica sp.
gtcgacctgcagccagaaggataaagaaattttggacgcctgaagaagaggcagttctgagggaaggagtaaaagagtatgtctccttaactctactatcaagtttcaagaagctgagcttggctctaccttgatatgtttattgctgttgtgcaggtatggtaaatcatggaaagagataaagaatgcaaaccctgaagtattcgcagagaggactgaggtgagagagcatgtcacttttgtgttactcatctgaattatcttatatgcgaattgtgagtggtactaaaaaaggttgtaacttttggtaggttgatttgaaggataaatggaggaacttggttcggtagccgtaacaagtttttgggaatctcttgggttttaaattgctatggagtttttttttgcctgcgtgacaacatatcatcagctgttgagaaggaagatggtattagaaagggtctttctttcacattttgtgttgtggacaaatattaaagtcaaatgtggcacatggattttaattcggccggtatggtttggttaagactggtttaacatgtataattagtctttgttttatttggctcagcggtttgttggtgttggttaggaacttaggcttgtctctttctgataagatctgattggtaagatatgggtactgtttggtttatatgttttgactattcagtcactatggcccccataaattttaattcggctggtatgtctcggttaagaccggtttgacatggttcatttcagttcaattatgtgaatctggcacgtgatatgtttaccttcacacgaacattagtaatgatgggctaatttaagacttaacagcctagaaaggcccatcttattacgtaacgacatcgtttagagtgcaccaagcttataaatgacgacgagctacctcggggcatcacgctctttgtacactccgccatctctctctccttcgagcacagatctctctcgtgaatatcgaca

SEQ ID NO: 45 - DNA, 834 bp, Nicotiana sp.
ggaagctttacaatgggttacatgtatggatccgagtatgaagaatgttgggaatcagtgatgcttcgcgcgttaggactttttcttcctggtatttctgcccacagcccagttgattatgtgaactccatcagacttggaaaggcgagaagtacacagatgtcatccttttagaaagctttttgtcgcaaatagtggttttatagctggacaatatcatgcattccttatgaggcttatgcagtatgtgtcctgtttgatttttgaaggtttgcttttagtgtttatgtattgacaataaacttatttcagttcttttattaagagatggatttgcataaaagatattgttcctctggtaatcgtattaaacttgttatgtcttcagtgaggcgaatagatataagattgttagatggtgttaataatttggtgacattgcaatttgcaaaactgtaaaaggatttttgctttactattttgtctatgttgactatatcccgtgaactatgaaaatgaaacaagcaagtaacactctatatattgtttccttgctagaacactcattcaacttttctttttcacccgagagaaaaaaatattcactatatttaaagtcggtattattcgtaagaacaaattataatctcgaaaagagtaaattgcacgtggtaaaaaaattgtaagattttaaatagtctctataaattaggtacaaacttaggcataaaaaaaaggttgatataaattaccttttatataaaaaatgtaatttacagaagaaacaattactactactactactaaaaaacatgggtcaggttggattacgtg

SEQ ID NO: 46 - DNA, 328 bp, Unknown Organism
  Description of Unknown Organism Unknown nucleotide fragment
ctagtaatactgagattagttacctgagactatttcctatcttctgttttgatttgatttattaaggaaaattatgtttcaacggccatgcttatccatgcattattaatgatcaatatattactaaatgctattactataggttgcttatatgttctgtaatactgaatatgatgtataactaatacatacattaaattctctaataaatctatcaacagaagcctaagagattaacaaatactactattatccagactaagttatttttctgtttactacagatccttccaagaacaaaaacttaataattgtatggctgctatac

SEQ ID NO: 47 - DNA, 1361 bp, Medicago sp.
agtgaaatatattgtattgggaatgataaaagtagtattatttagtgttatattgtattgggaatgatgaaaattgtattgaaaattgaaatgggtcagttattttggaacacttttttttagaaaatgggtcagttattccgggacggagggagtaataattatcttaaaagcattttaaaacaaaaagcaagaaacttcatattaaaaacaataatttttaaacatttaaaaagttaaatatgcactttctcaccgtttctcaaaataaaaaaaatctttattttaatttccttgagatatcctaacaaaaaagcaacaacttcagcgtgtgattcacacacaaacacaccaaccctgaacaatcaattgtccttctctccaactccaatagtccactaggaaggaagggtctttatggggtgtacaatgtgccagtggagtggaggggtctacatcctcaccaaactttgattcttcttcaacaatccaaaacccgtatgcatcatgagttgagtggttcaaaaaagtctctctttcactcaccaaatacgtaacagaacactttagctttgatgatgattcaatgcatcctaacgcaacgccacctatgtcccattaaacacatcagttcaccccttgcaaaatatatgaaagagattgaaagaaacagtgacttaacaatgttggatgttggaatagttattactcattcattcatataagttgttttcaaaataaacggtgtgatatacaaaaatacaacgttcaagattctacaaattgcaaataatttagcagaatttgttgcaatgcataatttatatttttagtatactatcatgtaggacatttcttaaaaaagaaacaattctttacaatgaccttcaaaaaatactatacgacctactttgcgtaagcagtatacattttcgcctacctttattttaaatgattcaatttcatttgccttaactttatttttcattttcgaattaagggattagcgtcaaattcaactttcatttttgttcaaaaaaactttcatttgtattttgttttatgaagtatttagtaaccgaaatttcattagttaaagtgaataagtaaagaatattgacttcgatttctacgtattataatgtttctacaaacttttgtttgtattaaaattaaattattatttttcataaataaaatatagaaaatttagtgatttttttaaggaaaaaaaattagtgatttgtttttttggtcaagaaaattaagtgatttaatcccttactatatatcatgcaatacctttttttcctttaggaaattacgcaatacctgtatggttggtaaatcaaataattctt

SEQ ID NO: 48 - DNA, 763 bp, Medicago sp.
aagggggactcattcctatctcccccatcaacctccctccctcatcaccgtacctcgccaccaacactttatacaacaacccgtccatatccaccaacattcgccaacatcatttttctaacaatgcaatattaaaatcccacatcttcctgacccccaaacctttgtactcctttttcaagtagaggaaattatacgtgtgagccatgaagaaggaatgaaagtagaccgcaagagaggacatgacaaacttcacgagaatcatacgaccacgcatttattattattattattaataatttttgaatgacaaatgttaattgttagtttgtttgagttttgaattcaaaacatttaactcttttctattcattcaaatcagttggactacttaatccttcccaaaaaaatgtgatagatcacactaacatgataaaaagagataaaattagatgttgaatgaatattcacaattacattttttttgctgataaagttatacttaaaaatagccaaacataacacaataattaattaattactttcttacaaagaccatccaaccatgaaatgaaccatattaactcgatgacaaaagagaatgcaatttttagtttaatctacacacaaaaaaagacaacacacaccaaggccacaaaccccacctaaccctctacagtaattccacctaactaaaaacccatacacatcatcatcatcatcaaaacctctctataaaaacccaacaaccactcctaacatt

SEQ ID NO: 49 - DNA, 431 bp, Lycopersicon sp.
ctgcttgagggattcgtgtgtatatgtatataataattaatttacaatttggtgcaaattaaataacttatattcaatttatttacattcatatataaactttatatatattaagagtttaatttccccataaacaagttttttatgaattttcagtcacaatagaatttttttaaaaaaaatatttttaaatgtttaacttaaattatgaaatgtgtaaatgtttgttaaccatatttagggctattgttattatttaatgaaaaataaaatataatataattcttaagaaagtattatatataaaataaaaaattacgtaacaaattatactatacccacaaaatataattatgtaaactataccatataatattatttcgtaaatttagtttgtcatataaaattttccctaaaatgaacagaaaccc

SEQ ID NO: 50 - DNA, 336 bp, Solanum sp.
aagagactttagaaatttacgatcaaaaacaaacacccacatttgtccgggtaaatataattggatccttacataaaaataaatagctgtcagattcattattattattattttgtcagtatacataagttaagcattggttatatatagatattatctccaatttaagctattaaattgaacaactattcaaattaattctttcagtatttaattgcagccacaatcactttaaatgcaactaatccactatgaaatgtttgaacggtagatacaaaaaagttcaacgtgacattcacttactaatttaatacctaccaaacccctatgtccatt

SEQ ID NO: 51 - DNA, 1859 bp, Solanum sp.
gatcttctttcatctaaactgacactaaactcttttttcttcccttctccaatatccaacatgcaattagacgatgaacgaaatgtgatgaaaaatttgataaatgagagttcaaattttaacaaaattaaataaaaaacataatcaattttttaaattttagaaatagagttattgtttaaatgatacattgaaattgcagtatatatcttatgaaataatggagataacttaaattgaccaaacattattattatttacacaaaagggggaaatagcaatttttggaccaaatattatactaaggaataggatgaaattataaaatgatttgctcgtttttttttcttctcaaaaacgaaagaacgcacaagttgcggatctcatgagatcattacccaatgcattaggtagagtaagatccacatcactaaccttttctccgtcaatttttatttggcccatatattaaaaaaatatttatttaaaaaattagaagctaatatattattatgaagtttaatttattgttattattaactatagtaattatttcaagtatattttttaaaatattaaatttattatattcgaaagaagatgtaataaatgtatcaatctttctgtttcaatttatataattcatgttattttagtttgcctaaaaagaatgatacatttgcagtggtgacacgatttgtaaaaatttatgcgtactcattgtctatatgtatgtatcgcagcggcaagcgagatgaaagagatgcaagaagatttgttatctatttcaaaatatatatgaatcttacttagacacaatgtatatagaacaaattatatgtaatagttgaccctatatatgtggtaaaatacttgactattaggggttgtttggtagagtgtattaagaaatataatgcatatattaggtgtgtgtattagtagtaccttgtttggcacactttttcatgccatgtataactaatgcatgtgtattactaataccaaggaattctaggtattagtaataaatagcattttaacacttgcattagatcaaataattacaaaactacccttaaagcattttcattttctttgttgtcataagtttttatttttatttttatttgcttttcggtatcttttaatttgttggtgtcttaatagactttatggccttttaagtatctttttaaaaaaaatctaatttcaatataatttaaatttttttttactattgtgacaataaatttgataaaaaaaattatttgccaactttcacaaaaatattttgacgcaatagtataactatttaatactatttttttattttttatttataaaaaagatgaagagttaatgatgttttaacaaagattttttttttgatgttttagcaaaaaactttcttgcaaaggaagtgtacaaataaataaagtgtgaagggtatttttgtaaacatatattatttaatagtaattatgcaagatttattatttttaatacatcaaaccaaacaatgtataagaaataatacttgcataactaatgcacgcactactaatgcaagcattactaatgcaccatattttgtatttgttcttatacactctaccaaacgaccccttagagtgtgggtaagtaattaagttagggatttgtgggaaatggacaaatataagagagtgcaggggagtagtgcaggagattttcgtgcttttattgataaataaaaaaagggtgacatttaatttccacaagaggaccgaacacaacacacttaattcctgtgtgtgaatcaataattgacttctccaatcttcatcaataaaataattcacaatcctcactctctt

SEQ ID NO: 52 - DNA, 1045 bp, Medicago sp.; identical to SEQ ID NO: 37

SEQ ID NO: 53 - DNA, 315 bp, Medicago sp.
ctaccgaggaattcctcggcagttaactgcagccggatttcaaattcctcggcagttaactgccgagggggcaaaagcgtattttacatgtgtgtcccagccttctttaatgtgtgaacaacaattttctaaaattaaaccctactctaggtttaacataccagtaaatttttgctttttgtatgtgttaacccttctccaatcccttgcacaaccatctcctcaaaccttcttcttctggagcaaagtcgccattccctacctccttcttcattcttattctctataacaaacggtccgaccggatccaagttg

SEQ ID NO: 54 - DNA, 797 bp, Medicago sp.
ctaccgaggaattcctcggcagttaactgcagccggatttcaaattcctcggcagttaactgccgagggggcaaaagcgtattttacatgtgtgtcccagccttctttaatgtgtgaacaacaattttctaaaattaaaccctactctaggtttaacataccagtaaatttttgctttttgtatgtgttaacccttctccaatcccttgcacaaccatctcctcaaaccttcttcttctggagcaaagtcgccattccctacctccttcttcattcttattctctataacaaacggtccgaccggatccaagttgcctcgtagtaatatttaagcgagttagaccgcgaggctttaaatacaaagattcaataaaacctcattaccatgtatgtgatttcgtcaaatttgttgttatttcaaacatgcgcgcataatgagttcaaatgaatatatgctaatagttgtgaactttgtcgcaggcaacttggatccggtcggaccgtttgttatagagaataagaatgaagaaggaggtagggaatggcgactttgctccagaagaagaaggtttgaggagatggttgtgcaagggattggagaagggttaacacatacaaaaagcaaaaatttactggtatgttaaacctagagtagggtttaattttagaaaattgttgttcacacattaaagaaggctgggacacacatgtaaaatacgcttttgccccctcggcagttaactgccgaggaatttgaaatccggctgcagttaactgccgaggaattcctcggtag

SEQ ID NO: 55 - DNA, 445 bp, Medicago sp.
ctaccgaggaattcctcggcagttaactgcagccggatttcaaattcctcggcagttaactgccgagggggcaaaagcgtattttacatgtgtgtcccagccttctttaatgtgtgaacaacaattttctaaaattaaaccctactctaggtttaacataccagtaaatttttgctttttgtatgtgttaacccttctccaatcccttgcacaaccatctcctcaaaccttcttcttctggagcaaagtcgccattccctacctccttcttcattcttattctctataacaaacggtccgaccggatccaagttgcaccggttcgaaccgctttagttactactaacggttcgaaccgttatttttcaacccgtgacgaacgtggaaggcttcgttgtttcttcttcttcttcttcttcttcttattaattaccatgcgtttttg

SEQ ID NO: 56 - DNA, 793 bp, Brassica sp.
ggatggggtcaccttatcctagtcaataaataatcaacaaaattttagggaacaaaatatatatgctagaggatcgttatgtttgtcttccatttcactgcatctacatatggaattgattctagagtaagaaacacaaataaatttatttggtacaatcctcccgtccaaggaaaatctaaaaatagaaaagaaatcttagtgaagttatagattatggtagcttatatttttttaaaaaaacgattatggtagcttctatttataccctactttaaatatatatgattgtcctataacgtattgaatagaaaatatcttcgaatatcatatatatgaaactagtgtaaattttaaacgtaaacaatttatacgaccacagttcgaagaaaaaaaacaatttatacgaccagaaatggcaaaatgttgttcttagaatttttttctactttacttttgcgtaaaacacatttctccaatttggtttcattgcgttgaacgacgtaacaaagtaatacacccaacccttttttttggaacattatgcacccaacccattgtacaaaagttacagctaattaccatttttattcttttgataaatacaaaaataaattattaatcattaaaaaaaaatttggaatattttctcaatgtccatatatacatcttctccctttatataagccaacctcacacacccaaaaaatccatcaaacctttctccaccacatttcactgaaaggccacacatctagagagagaaacttcgtccaaatctctctctccagca

SEQ ID NO: 57 - DNA, 1635 bp, Brassica sp.
aggggggactcttcatattatttttggtgagtagcgtaatcatagatagttttcttaattcttgaacttgggtaacatcgtgggtatctacgaaatgattcctttcgacgtacacgatttatagataaacacgtagagacgtgtataataagcgagaaacttatttagcagtgttagagaaatatttgagttaacagactatagaacatttataaattagtattcaataaattaatatttttaatattcaataattaatattttaatcttcagtaaaaaaatataatattcgataacttagtattcaataaattaatattttcaataaattaatattcaaaaaattaacatttataaaaaatcattaaattatattgtctcattacaattgtaaattaataactgatgtataaaaattatataaacataacaaaatattgttatgtatggtttttatttaaaatgaaactaattctaattttttcaacacttcaaagtattttataattatatatttaaaaatattaacattatgtgattcatattatatatatgtcaaataatttaataaacactatgaaagctaagtttacaaaacttaattaatatataattcacgaaaaaatctattccttttattttacatataaacatattttaaaatatataaatctaagtatgatattttgataaattactaattttataaattaaatattatagttcattaagtattttgaataattattggatctttaagtattttgaataattattcaaaattgactcattttgttttttaagatttttaaaaaattgagtttttttttcgatctccgttagaatttgatttgggtaaaaactaaaatctgaaataccatagaataataaccatttggatacttatgtcgaattcaaaacagtttaattctcaggttcaaattttcatattgttttttcataccatagaataatagccatttggatacttatgtctaaaagtaatataatctgagacaaaatataaaaatataaggatttatatatttcaaccatatggatatggttgtgtgatacgaaagtgttagacattatcgatttgaaatctatcattcagatttgtcttttacatggttaaagggtgtgtgaatataaaactttcacgtagaacaacggatttatctgttgcctgaaaaacaggctaaacactctattatgattagtcttagatttaggacacccctggtccataaaaaaggtcttacatatttactttcgcatacatatttttctaatttaatttcactgaatagaacgatgtaacaaagtaaccaaacccattgcatttaaaattacagcaaaattatcctttttttaaaatatataattatttctttaaatatatatatattttttttatttttttttcaacaaatatataattattaaaaaaaaacagttttgagtatctcaatcaattctacagacttacacatcctccttcccctttatataaagaaacttcagacctcaaaatacatcgaaccctttcttcaccacattccacttcccacactctctttttttttgaattatagagagagaatcctcctccaaatctctctctctcccagg

SEQ ID NO: 58 - DNA, 605 bp, Brassica sp.
gattatgctgagtgatatcccaaccgggcatgcagagtggaggcgatggaagaaagcggtgccggagaccgttcgactgcagcaaaattaccagagaagttaaaaggggaagatgtgaacaagggtaagacacgagttacttttcaacggtgaataattaaaatatttaattatttttttgtagcaggttgagccggttgtgttttaggaatattacagtattattttatatttgtaacagcgtgtataagatcgttaggttaaatggctagacggtgaattacgtttttttttgtggttatagccttcaatttcccatttaatttcaccgaatagaacgatgtaacaaaataacaaacccattgcatttaaaattacagcaaattaccctttttattctttaaatatataattatttaataaaaacagtttgagcatctcaatgtctacagactacacatcttccttcccctttatataaacaaacttcacagaccgcaaaatacatcgaaccctttcttcaccacattccagttcccacactttcttttttttgaattatagagagagaatcttcctccaaatctctctctctctctcccagg

SEQ ID NO: 59 - DNA, 647 bp, Brassica sp.
gacgaagatcttctcctggtaatctaaggaaacatgaatatttgttgagttttggcttgtgaagatgctctttgttcatctgctgttttcgatggatttgtgcagattaacttggagaacatgaagaagcagaaagaatagttccctatcttcttcatcatcatcaaatgagtgtggattaaaatgaaacccacccgagtgttctatcccagaagagcaatactagtttacatatacatatatatatatatatacgtataaatggatgttgcccaacatattcatatagaggttcatggatcataagtgagtataggtttgacattgatcagatttgtctctgtttctaagctgttatagttattccttgttgtacaaatcggttttgccataaaagtccctttaggatgtgaatgcaatataagatttgattgattcaagttttccagtaataacaagactaattccactacgttaaaacaaaagtacaatcgaccgtaccggatcgaaccgaaccgaaccaataccaacatatccaattcgcgtcataccagaacattcttaaaccggaattagattcggaccaaacacatcatcataagattcgttaagaagatggttgtgtctttttccctgtctgctactag

SEQ ID NO: 60 - DNA, 773 bp, Brassica sp.
  modified_base (12)..(12): a, c, g, t, unknown or other
acagagaaaatntcttgcaggatgcacgagagganatcgtcaaaatgtctagagaatgcccggaaatcgtttggtacagacgaagatcttctcctggtaatctaaggaaacatgaatattcgttgggttttggctttgtgcagttgctctttgttcatctgttgttttcgatggatttgtgcagatcaacttggagaacatgaagaagcagaaagaatagttctctatcttcatcatcatcatcattatcaaatcagtgtggattaaaatgaaaccacccgagtgttctatcccagaagagcaatactagtttacacatacatatatacgtataatggatgttgcccaaacatattcatatagagaggtgcatggatcatcagtgaactcaagagtataggctttgacaatgatcagattcatctgtttctaagcagttaatagttattccttgttgtacaaatcggttttgtcataaagtccctttaggatgtgaatgcatataagatttgattgattcaagttttggagtaataacaagagtaattccactgtgttcaaaaaaaaaaagaaaaaaaagagtaattccactcgacgaaccggtaaatatcggagtacaatcgagcgtaccggatcgaaccgaaccagactaataccaccgtacccaattcgcgtcataccagaacattcttaaaccggaattagattcggaccgaacacatcatcataagattcgtttggaagatggttgtgtctttttccctgtctgctaa

SEQ ID NO: 61 - DNA, 1435 bp, Brassica sp.
tgagcttgaagggacgtttgagcagataaacgaagcgagtgtgatggttagagagctgattgggaggcttaactctgcagctagtaggagaccacctggtggtggtggtgggattggtggtggggttggttcggaagggaaaccacatccagggagcaacttcaagacgaagatgtgtgagaggttcgcgaaagggaactgtacgtttggggataggtgtcactttgcgcacggggaagcagagctgcgcaggtcaggaattgcctaagttgctgtttgtggagtttgctgtcttttcttttgtgtgtggtggtgatctctaatatcatccatcttcttcatctattttgcttttgttttatgaaaatacaatgttagtttcattgtctttgtaagttttctttctctctgtgtggtgattcttagaatatagttttttttgctgttaaattgagtttgaattggtgagagacttggtggatggattgacagacggtggttaggatttgtatgctgccttaattttcttacagtcatgcttgctctgatttgtctgttgtgcgtgagtcagacacatcatctttgataccaaaaaaacatgttataaaacccgtcactggtagtaacaatcagctgaataaatataacattcctaatggtgggtgtgtgatcttaaacaaaaaattttgaaagaaaagtgtgttgttgttagaggtaatgcttagacaaatcaaactctaatcatcttctaagtctagtataatacaagagatctcaatctaatcaatcactagtttcttttcgtctgccaacaaatttgattattataagtatcaaagatgattacacatacataacaaattgtaataagaaaaagaaaagagagagaaatcctcacgtgagcatcaccacaatttgtctgttacatatttctgtaagttcttgtgtgttcacatgggcaaaagtgagaagaagccaaacacgatactccattttcaggcatcaactaccatcttcttcttcttcttctttatcaagttgtttctaatgtcatattaagaaatgatacatgattgacttacgtagagaaaaactgattcaaacaagtaccgcatgtgtcattgcgttccaaagtgattaagtcaataacatgatacgaccttttttattacattacatacataaccaagataacgtggacgagaaaaagagagaacgtcgtagtaatatcaccttttcatcactctaacttttacattttggtaaattctaaattaatggtcgttccttgagttaaatatcagatattttgaacagaggggcccagttgtaaaaataagagaaaagaggggccagttgtaagaataagagatgtcattcaaatgccttcctgtctctcatcaatttaaaaacggccctgcctattgccactcgc

SEQ ID NO: 62 - DNA, 482 bp, Brassica sp.
gagaagaagccaaacacgatactccatttccaggcatcaactaccatcttcttcttcttcttctttatcaagttgtttctaatgtcatattaagaaatgatacatgattgacttacgtagagaaaaactgattcaaacaagtaccgcatgtgtcattgcgttccaaagtgattaagtcaataacatgatacgaccttttttattacattacatacataaccaagataacgtggacgagaaaaagagagaacgtcgtagtaatatcaccttttcatcactctaacttttacattttggtaaattctaaattaatggtcgttccttgagttaaatatcagatattttgaacagaggggcccagttgtaaaaataagagaaaagaggggccagttgtaagaataagagatgtcattcaaatgccttcctgtctctcatcaatttaaaaacggccctgcctattgccactcgcatctgaccagaca

SEQ ID NO: 63 - DNA, 1494 bp, Brassica sp.
ttacacattcgcaaccctggaggatactccaagagactacgatcccaaaggacaacctatacaattgtggagagtgacaaagaagggagagcatatgaatggataatactagcactgcatagcttaacttgtatcgttttttctccttaggttagtaggtatgttttacaaaaattaatttctatgaattttaaatataatataaaataatatgttttaggtgaaacaaatttataagtccaacggtggacttcatgttctacaaaaaaaagtatagttaaacgaaccaaccaaataaactgttagaaatgcataatgttaggttttgtataaatgttatgtttcaatttgagctttgataaaatacacacgagtaaagaaagaggtaagatgcacatgtaccttgtttgttgtacactcagcccactcaactattattactaaaacgtcggtgccaaagttgacaattctctgctaaatacaatctgatatacgtctctttctccacaacaatatgttgattggttagtgtaattagcaatcctcacatatagggaggaaatcaaatattcaaatccaaatgaaatttccacggaagcaagtaatcaagtcttgcgtgcttacataacgagtgaccaataatataaaaaagaattgaattagattagcctagttaggttaacaatcttttaacaagaaaagggtataattggaaatacaagaaaatttaaaaatatggttttgaaactacgagaaggaaggagaaaggaagaagaagaagaaggggagtgcaatttatataagaaaaggcctctcgtccacatctctctctctcacaccccaccctacagagactctctctcccccttttatctctctctctctacgccaaatttttaaatatttttttttcctacaaaaaagaagtattgagaatcgcaaacaaaagtaaaaaaaatattaaacaaaaggaggagaggagaggagatcgtgagggaggcacaaccgaagaagtagggactttggagaaaattagcgttaccatttttgagattttcatcctccattctacacctgaaggtggtaccatctctctctcttcttcttcgtgtgttcttcgttaatatcttcatcgcttggttcggattccttattcaaattcaatgctttatcgaaaataataatattccaattatcttttttttgataaaaagttttgatttttatcggtttacctttgtagtttcaaaattccagatctgaatttttttctctctgcttgttacacaaaaaaaaagttttgattttgattttttgttattgttgttgtgtttttgattatagacttgtagcatttttgttgttgttgattaattgattagctaattgttacaaagatgtagactttgtaataatacgtcactcactttgttatgttttgttgtgttttttttttgttttatagtgtctttgaaacgctcatctcctcaagcc

SEQ ID NO: 64 - DNA, 1542 bp, Brassica sp.
cacagggtatcaaaattcaaaactttctaaatgaataaacagaaacaaaataatcttacattaacaaacaaaaacagaaacaacaaacgaaaccaaaatcatctaaatcgttctaaattagcatacgaaaccaaaatcatcatccatcaataaaaaaaacaaaaaaaaagaaacggagccaaaatcatcaaagctttttaaatcaataaacaatacccaaatcatcttacatcaacaaacaaaaaccaaatcaataaacgtaaccaaaatcattctcctgtaaaaaaaatttcaaaagttattaggatttgttgggatgatgttcacgggatgaagccatacctttttttatagttgtgatccaccgcttgtaagaaatataaaaatcattgaatgattgattgtggtgcagtgggatgaaagagttaataaatttttaatggcgtcgaatcaatgcaacttgtaacgccttcgaggaggggagaagaaccgcagacgaaacgacataaaaccgcaaaggacgcaaagactactcatgaatactcgtctcttacaaccttgagaacatctatttttggtttatcgtaatcagagcttgcaggagaagatgaaccctaaagttgagtggcggctccacgttgaaaaagtttgtgactacaggacaagctttaatttgtttatgcccggatgaaattatgcaaatcccacaaaataatggtgtaagcccaaaaccgaacataacaaattgaatgatttttaacgaagggagacacgtgtcgtcgcgacgtcgtccgatttattaacgtgaatgctgaagtagcgcaacatgagggaggcaaacatttttttatatatagatagatactttcactctaaaagtattattgagaattgccaaaaaagacctgaattaaaaaataaatataactgagaaagaaaagaaaatacagagagacaaatttaaacaaaaggaaagggagatcgagagaggcacacacacacaaaggagaattttagggtttggggagactccgaagagattggcgtaaccttcattgtacacttcgtaggatctctcttccttaaatctcgtttgaatttcgttatctgtttgctttcgattcaatcgctttatcgaaataatgtgtattcgaatggagcctccacgatctgattttatagattctccgttgttttgatttcagatctggattttttcccccaatatctctaattgaaaattgtcgatttcgagtgtcagctgagagtattgtgaacctgcagctgtggtttggattgtttatagctcaatggttgaaacttgatcattcttacacataaaaattgttcctttacttccgttgattacttggtgagcttatccatctttctagttgttaaaggtgttagcttttgaagtatgccactctcttttgtgtgctcgttttacagacatcattcattttgttgattaacttggtcctctttattgttttttttttgtgtggtgtttagtgtctttgaaagctcatcttcctcgtc

SEQ ID NO: 65 - DNA, 362 bp, Brassica sp.
gcaaaggacgcaaagactactcatgaatactcgtctcttacaaccttgagaacatctatttttggtttatcgtaatcagagcttgcaggagaagatgaaccctaaagttgagtggcggctccacgttgaaaaagtttgtgactacaggacaagctttaatttgtttatgcccggatgaaattatgcaaatcccacaaaataatggtgtaagcccaaaaccgaacataacaaattgaatgatttttaacgaagggagacacgtgtcgtcgcgacgtcgtccgatttattaacgtgaatgctgaagtagcgcaacatgagggaggcaaacatttttttatatatagatagatactttcactcta

SEQ ID NO: 66 - DNA, 604 bp, Brassica sp.
actacgatcccaaaggacaacctatacaattgtggagagtgacaaagaagggagagcatatgaatggataatactagcactgcatagcttaacttgtatcgttttttctccttaggttagtaggtatgttttacaaaaattaatttctatgaattttaaatataatataaaataatatgttttaggtgaaacaaatttataagtccaacggtggacttcatgttctacaaaaaaaagtatagttaaacgaaccaaccaaataaactgttagaaatgcataatgttaggttttgtataaatgttatgtttcaatttgagctttgataaaatacacacgagtaaagaaagaggtaagatgcacatgtaccttgtttgttgtacactcagcccactcaactattattactaaaacgtcggtgccaaagttgacaattctctgctaaatacaatctgatatacgtctctttctccacaacaatatgttgattggttagtgtaattagcaatcctcacatatagggaggaaatcaaatattcaaatccaaatgaaatttccacggaagcaagtaatcaagtcttgcgtgcttacataacgagtgaccaa

SEQ ID NO: 67 - DNA, 515 bp, Solanum sp.
gtggaacggagacatgttatgatgtatacgggaagctcgttaaaaaaaaaatacaataggaagaaatgtaacaaacattgaatgttgtttttaaccacccttccttttagcagtgtaccaattttgtaatagaaccatgcatctcaatcttaatactaaaaaatgcaacaaaattctagtggagggaccagtaccagtacattagatattattttttattactataataataatttaactaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgttgatgagcatttccctataatacagtgtccacagttgccttccgctaagggatagccacccgctattctcttgacacgtgtcactgaaacctgctacaaataaggcaggcacctcctcattctcac

SEQ ID NO: 68 - DNA, 775 bp, Solanum sp.
taacgagatagaaaattatattactccgttttgttcattacttaacaaatgcaacagtatcttgtaccaaatcctttctctcttttcaaacttttctatttggctgttgacagagtaatcaggatacaaaccacaagtatttaattgactcatccaccagatattatgatttatgaatcctcgaaaagcctatccattaagtcctcatctatggatatacttgacagtttcttcctatttgggtatttttttcctgccaagtggaacggagacatgttatgttgtatacgggaagctcgttaaaaaaaaaatacaataggaagaaatgtaacaaacattgaatgttgtttttaaccatccttccttttagcagtgtaccaattttgtaatagaaccatgcatctcaatcttaatactaaaaaatgcaacaaaattctagtggagggaccagtaccagtacattagatattattttttattactataataataatttaactaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgttgatgagcatttccctataatacagcgtccacagttgccttccgctaagggatagccacccgcaattctcttgacacgtgtcactgaaacctgctacaaataaggcaggcacctcctcattctcac

SEQ ID NO: 69 - DNA, 961 bp, Solanum sp.
taatcgcgtaattttccccattaattatatataaaattcttaagaaattctcgaggcagtaaaggttccacaaattgaaatcaggaagaaactattaactaatctattttcttttcttcaacgactactacttattatattggctctaaagataagaggataatgaaacaaaggaagaagctttaacgagatagaaaattatattactccgttttgttcattacttaacaaatgcaacagtatcttgtaccaaatcctttctctcttttcaaacttttctatttggctgttgacagagtaatcaggatacaaaccacaagtatttaattgactcatccaccagatattatgatttatgaatcctcgaaaagcctatccattaagttctcatctatggatatacttgacagtttcttcctatttgggtatttttttttcctgccaagtggaacggagacatgttatgttgtatacgggaagctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcac

SEQ ID NO: 70 - DNA, 781 bp, Solanum sp.
aagctttaacgagatagaaaattataatactccgttttgttcattacttaacaaatgcaacagtatcttgtaccaaatcctctctcttttcaaacttttctatttggctgttgacagagtaatcaggatacaaaccacaagtatttaattgactcatccaccagatattatgatttatgaatcctcgaaaagcctatccattaagtcctcatctatggatatacttgacagtttcttcctatttgggtttttttttttcctgccaagtggaacggagacatgttatgttgtatacgggaatctcgttaaaaaaaaaaatacaataggaagaaatgtaacaaacattgaatgttgtttttaaccatccttccttttagcagtgtatcaattttgtaatagaaccatgcatctcaatcttaatactaaaaaatgcaacaaaattctagtggagggaccagtaccagtacattagatattattttttattactataataatattttaattaacacgagacataggaatgtcaagtggtagcggtaggagggagttggtttagtttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcac

SEQ ID NO: 71 - DNA, 529 bp, Solanum sp.
gaaccatgcatctcaatcttaatactaaaatgcaacttaatataggctaaaccaagtaaagtaatgtattcaacctttagaattgtgcattcataattagatcttgtttgtcgtaaaaaattagaaaatatatttacagtaatttggcatacaaagctaagggggaagtaactactaatattctagtggagggaccagtaccagtaccagtacctagatattattttttattactataataataatttaattaacacgagactgataggaatgtcaagtggtagcggtaggagggagttggtttagttttttagatactaggagacagaaccggacgggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcac

SEQ ID NO: 72 - DNA, 520 bp, Solanum sp.
gaaccatgcatctcaatcttaatactaaaatgcaacttaatataggctaaaccaagtaaagtaatgtattcaacctttagaattgtgcattcataattagatcttgtttgtcgtaaaaaattagaaaatatatttacagtaatttggcatacaaagctaagggggaagtaactactaatattctagtggagggaccagtaccagtaccagtacctagatattattttttattactataataataatttaattaacacgagactgataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagttgccttccgctaagggatagccacccgctattctcttgacacgtgtcactgaaacctgctacaaataaggcaggcacctcctcattctcac

SEQ ID NO: 73 - DNA, 343 bp, Solanum sp.
attctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcac

SEQ ID NO: 74 - DNA, 785 bp, Solanum sp.
attctagtggagggaccagtaccagtacattagatattattttttattactataataatattttaattaacacgagacataggaatgtcaagtggtagcggtaggagggagttggtttagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacgtcctcatctatggatatacttgacagtttcttcctatttgggtatttttttcctgccaagtggaacggagacatgttatgttgtatacgggaagctcggtgagaatgaggaggtgcctgccttatttgtagcaggtttcagtgacacgtgtcaagagaatagcgggtggctatcccttagcggaaggcaactgtggacactgtattatagggaaatgctcatcgacagtattatgggccctctctttgttgattcacggctggacttcaacttgggccttgcaatgggcccctccggttctgtctcctagtatctaaaaaactaaaccaactccctcctaccgctaccacttgacattcctatgtctcgtgttaattaaaatattattatagtaataaaaaataatatctaatgtactggtactggtccctccactagaat

SEQ ID NO: 75 - DNA, 1481 bp, Solanum sp.
aaaaacctcctccactcagtcttgggatctctctctctcttcacgcttctcttggggccttgaactcagcaatttgacactcagttagttacactcctatcactcatcagatctctattttttctcttaattccaaccaaggaatgaattaaaagattagatttgaaggagagaagaagaaagatggtgtatacactctctggagttcgttttcctactgttccatcagtgtacaaatctaatggattcagcagtaatggtgatcggaggaatgctaatgtttctgtattcttgaaaaagcactctctttcacggaagatcttggctgaaaagtcttcttacgattccgaatcccgaccttctacagttgcagcatcggggaaagtccttgtacctggaatccagagtgatagctcctcatcctcaacagaccaatttgagttcactgagacagctccagaaaattccccagcatcaactgatgtggatagttcaacaatggaacacgctagccagattaaaactgagaacgatgacgttgagccgtcaagtgatcttacaggaagtgttgaagagttggattttgcttcatcactacaactacaagaaggtggtaaactggaggagtctaaaacattaaatacttctgaagagacaattattgatgaatctgataggatcagagagaggggcatccctccacctggacttggtcagaagatttatgaaatagacccccttttgacaaactatcgtcaacaccttgattacaggtattcacagtacaagaaactgagggaggcaattgacaagtatgagggtggtttggaagctttttctcgtggttatgaaaaaatgggtttcactcgtagtgctacaggtatcacttaccgtgagtgggctcctggtgcccagtcagctgctctcattggagatttcaacaattgggacgcaaatgctgacattatgactcggaatgaatttggtgtctgggagatttttctgccaaataatgtggatggttctcctgcaattcctcatgggtccagagtgaagatacgcatggacacttcatcaggtgttaaggattccattcctgcttggatcaactactctttacagcttcctgatgaaattccatataatggaatatattatgatccacccgaagaggagaggtatgtcttccaacacccacggccaaagaaaccaaagtcgctgagaatatatgaatctcatattggaatgagtagtccggagcctaaaattaactcatacgtgaattttagagatgaagttcttcctcgcataaaaaaccttgggtacaatgcggtgcaaattatggctattcaagagcattcttattatgctagttttggttatcatgtcacaaatttttttgcaccaagcagccgttttggaacgcccgacgaccttaagtctttgattgataaagctcatgagctaggaattgttgttct

SEQ ID NO: 76 - DNA, 237 bp, Lycopersicon sp.
ccatttaactttgattgtaattaatttttaaaaattaccaacatataaataaaattaatatttaacaaagaattgtaacataatatttttttaattattcaaaataaatatttttaaacatcatataaaagaaatacgacaaaaaaattgagacgggagaagacaagccagacaaaaatgtccaagaaactctttcgtctaaatatctctcatccaaactaatataatacccattac

SEQ ID NO: 77 - DNA, 458 bp, Medicago sp.
ctaccgaggaattcctcggcagttaactgcagccggatttcaaattcctcggcagttaactgccgagggggcaaaagcgtattttacatgtgtgtcccagccttctttaatgtgtgaacaacaattttctaaaattaaaccctactctaggtttaacataccagtaaatttttgctttttgtatgtgttaacccttctccaatcccttgcacaaccatctcctcaaaccttcttcttctggagcaaagtcgccattccctacctccttcttcattcttattctctataacaaacggtccgaccggatccaagttgcaccggttcgaaccgctttagttactactaacggttcgaaccgttatttttcaacccgtgacgaacgtggaaggcttcgttgtttcttcttcttcttcttcttcttcttattaattaccatgcgtttttgtttttcttttgag

SEQ ID NO: 78 - DNA, 4721 bp, Artificial Sequence
  Description of Artificial Sequence Synthetic construct
caagtgtctgagacaaccaaaactgaaagtgggaaaccaaactctaagtcaaagactttatatacaaaatggtataaatataattatttaatttactatcgggttatcgattaacccgttaagaaaaaacttcaaaccgttaagaaccgataacccgataacaaaaaaaatctaaatcgttatcaaaaccgctaaactaataacccaatattgataaaccaataactttttttattcgggttatcggtttcagttctgtttggaacaatcctagtgtcctaattattgttttgagaaccaagaaaacaaaaacttacgtcgcaaatatttcagtaaatacttgtatatctcagtgataattgatttccaacatgtataattatcatttacgtaataatagatggtttccgaaacttacgcttcccttttttcttttgcagtcgtatggaataaaagttggatatggaggcattcccgggccttcaggtggaagagacggagctgcttcacaaggagggggttgttgtacttgaaaatgggcatttattgttcgcaaacctatcatgttcctatggttgtttatttgtagtttggtgttcttaatatcgagtgttctttagtttgttccttttaatgaaaggataatatctgtgcaaaaataagtaaattcggtacataaagacatttttttttgcattttctgtttatggagttgtcaaatgtgaatttatttcatagcatgtgagtttcctctcctttttcatgtgcccttgggccttgcatgtttcttgcaccgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaacggatccctagtaatactgagattagttacctgagactatttcctatcttctgttttgatttgatttattaaggaaaattatgtttcaacggccatgcttatccatgcattattaatgatcaatatattactaaatgctattactataggttgcttatatgttctgtaatactgaatatgatgtataactaatacatacattaaattctctaataaatctatcaacagaagcctaagagattaacaaatactactattatccagactaagttatttttctgtttactacagatccttccaagaacaaaaacttaataattgtatggctgctataccatcaaaccaaacaatgtataagaaataatacttgcataactaatgcacgcactactaatgcaagcattactaatgcaccatattttgtatttgttcttatacactctaccaaacgaccccttagagtgtgggtaagtaattaagttagggatttgtgggaaatggacaaatataagagagtgcaggggagtagtgcaggagattttcgtgcttttattgataaataaaaaaagggtgacatttaatttccacaaaattcttatgttaaccaaataaattgagacaaattaattcagttaaccagagttaagagtaaagtactattgcaagaaaatatcaaaggcaaaagaaaagatcatgaaagaaaatatcaaagaaaaagaagaggttacaatcaaactcccataaaactccaaaaataaacattcaaattgcaaaaacatccaatcaaattgctctacttcacggggcccacgccggctgcatctcaaactttcccacgtgacatcccataacaaatcaccaccgtaacccttctcaaaactcgacacctcactctttttctctatattacaataaaaaatatacgtgtccgtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagggacacgtatattttttattgtaatatagagaaaaagagtgaggtgtcgagttttgagaagggttacggtggtgatttgttatgggatgtcacgtgggaaagtttgagatgcagccggcgtgggccccgtgaagtagagcaatttgattggatgtttttgcaatttgaatgtttatttttggagttttatgggagtttgattgtaacctcttctttttctttgatattttctttcatgatcttttcttttgcctttgatattttcttgcaatagtactttactcttaactctggttaactgaattaatttgtctcaatttatttggttaacataagaattttgtggaaattaaatgtcaccctttttttatttatcaataaaagcacgaaaatctcctgcactactcccctgcactctcttatatttgtccatttcccacaaatccctaacttaattacttacccacactctaaggggtcgtttggtagagtgtataagaacaaatacaaaatatggtgcattagtaatgcttgcattagtagtgcgtgcattagttatgcaagtattatttcttatacattgtttggtttgatggtatagcagccatacaattattaagtttttgttcttggaaggatctgtagtaaacagaaaaataacttagtctggataatagtagtatttgttaatctcttaggcttctgttgatagatttattagagaatttaatgtatgtattagttatacatcatattcagtattacagaacatataagcaacctatagtaatagcatttagtaatatattgatcattaataatgcatggataagcatggccgttgaaacataattttccttaataaatcaaatcaaaacagaagataggaaatagtctcaggtaactaatctcagtattactagttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggaattcgcggtac

SEQ ID NO: 79 - DNA, 520 bp, Figwort mosaic virus
atttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaaga

SEQ ID NO: 80 - DNA, 545 bp, Brassica sp.
caccggctgcagatatttttttaagttttcttctcacatgggagaagaagaagccaagcacgatcctccatcctcaactttatagcatttttttcttttctttccggctaccactaacttctacagttctacttgtgagtcggcaaggacgtttcctcatattaaagtaaagacatcaaataccataatcttaatgctaattaacgtaacggatgagttctataacataacccaaactagtctttgtgaacattaggattgggtaaaccaatatttacattttaaaaacaaaatacaaaaagaaacgtgataaactttataaaagcaattatatgatcacggcatctttttcacttttccgtaaatatatataagtggtgtaaatatcagatatttggagtagaaaaaaaaaaaaagaaaaaagaaatatgaagagaggaaataatggaggggcccacttgtaaaaaagaaagaaaagagatgtcactcaatcgtctcacacgggcccccgtcaatttaaacggcctgccttctgcccaatcgca

SEQ ID NO: 81 - DNA, 337 bp, Unknown Organism
  Description of Unknown Organism Unknown nucleotide fragment
tcgaagaaaaaaaacaatttatacgaccagaaatggcaaaatgttgttcttagaatttttttctactttacttttgcgtaaaacacatttctccaatttggtttcattgcgttgaacgacgtaacaaagtaatacacccaacccttttttttggaacattatgcacccaacccattgtacaaaagttacagctaattaccatttttattcttttgataaatacaaaaataaattattaatcattaaaaaaaaatttggaatattttctcaatgtccatatatacatcttctccctttatataagccaacctcacacacccaaaaaatccatcaaacc

SEQ ID NO: 82 - DNA, 314 bp, Unknown Organism
  Description of Unknown Organism Unknown nucleotide fragment
cccctggtccataaaaaaggtcttacatatttactttcgcatacatatttttctaatttaatttcactgaatagaacgatgtaacaaagtaaccaaacccattgcatttaaaattacagcaaaattatcctttttttaaaatatataattatttctttaaatatatatatattttttttatttttttttcaacaaatatataattattaaaaaaaaacagttttgagtatctcaatcaattctacagacttacacatcctccttcccctttatataaagaaacttcagacctcaaaatacatcgaaccctttct

SEQ ID NO: 83 - DNA, 417 bp, Unknown Organism
  Description of Unknown Organism Unknown nucleotide fragment
taaaaggggaagatgtgaacaagggtaagacacgagttacttttcaacggtgaataattaaaatatttaattatttttttgtagcaggttgagccggttgtgttttaggaatattacagtattattttatatttgtaacagcgtgtataagatcgttaggttaaatggctagacggtgaattacgtttttttttgtggttatagccttcaatttcccatttaatttcaccgaatagaacgatgtaacaaaataacaaacccattgcatttaaaattacagcaaattaccctttttattctttaaatatataattatttaataaaaacagtttgagcatctcaatgtctacagactacacatcttccttcccctttatataaacaaacttcacagaccgcaaaatacatcgaaccctt

SEQ ID NO: 84 - DNA, 3605 bp, Solanum sp.
gtaaattaagcgtctaataaatgaaataactatttgtcggtctgtatgcatgctaaacctgtctttcaattggagcatgactatacaaaatgtctaaaagccgatgaagttctctgtgtcttatgataatagatttcagcatcgaaaatcaagttttaaggagctgctctacatatgcgatggagatagcaacggggtcctttattttgctggcacatcatatgggaaacaccagtgggtgaatcctgttttgtccaaggtaaatccacagctgcaataagcaatttaccttccttcttttgacttgttaccgttctaaaaaatatacaattgtttaccatctcattttgtcatctgtttaacattggtaattcatgtttcagagagtaattatcacggctagtagccccatttcaagatgcactgatcccaaggtgttagtatcgaggaacttccaggtttgaatagatgacatccaattaatgtgaaggatcttctccttctagattaatttgagaaaaaaaaagaaatattcttttgctctctctctctttttcatcgatggcatgaagaagaggaagtcgatacacaaaagagagtgttagctccataatgtgaaggatgaaatatttttttggtctcagggtacatctgttgctggacctcaggtggagggcggaagaaacgcttcgtggtggatggttgatattggtccggatcaccaggttagatttattggtttgtgtataatttaattgtgtgtacataagggagatggaaagaagtttttgtaaaataagatgtatgttgtaacttagacaatcacttcgtccgtgctgattctcagattcatctgtatttttaattgacttgtgaaagtgaacatttaaaattgaacatcggtaacttgcatttctcattgtaagggcattgcatgatatcatggttgtctagagtagtgctgatcagtatacctcgtggacaagatactgaaagtgaacactcatctctgctcttttggtttcgttaaaagtactctctctctcagtttatagcacactcaaattgtgtgtcaatatccctgattgattttctcatttggtattcaactagaagatgaaacttctgacgcatttaatattagatgaatcgatgcagctcatgtgtaactactacacatcaagacaggacggatcaagagcatttatcagacgttggaactttcaggtaagcagtgcactcaacattcacaaaccagtatacacatcatctctaatggatctgtggatgcactcgtaactcgtctatagattatacatatatacatacatatatacgtaccaacatctccattttgtagaactggaaacgttgttaaaattggcgttacaataacaaatttttatgcattgcattctcagggctctttggatgggaaaaattggacaaacctgagagtacatgagaatgatcaaactatttgcaagccaggtcaatttgcatcatggccaattactggttcaaatgcattacttcctttcagattctttcgagttctcatgaccggtcctactacagacgctactaacccgtggaactgttgcatctgcttcttagaactctatggctattttcgttagcttggcgtcggtttgaacatagtttttgttttcaaactcttcatttacagtcaaaatgttgtatggtttttgtattcctcaatgatgtttacagtgttgtgttgtcatctgtactctttgcctgttacttgttttgagttacatgtttaaaaaagtgtctttctgccatattttgttctcttattattattattgttattatcatacatacatattaaaagggaaatgacaagtacacaaatcttagaccgtttatgttcaatcaacttttggaggcattgacaggtccaaaattttgagtttatgattaagttcaatcttagaatatgaatttaacatctattatagatacataaaaatagctaatgatagaacattgacatttggcagagcttagggtatggtatatccaacgttaattttagtaatttttgttacgtacgtatattaaatgttgaattaatcacatgaacggtggatattatattatgaattggcatcagcaaaattattagtgtagttgacttgtagttgcagttttaataataaaatggtaattaacggtcgatattaaaataactctcatttcaagtgggattagaactagttattaaaaaaatgtatactttaagtgatttgatggcttataatttaaagtttttcatttcatgctaaaattgttaatcattgtaatgtagactgcgactggaattattatagtgtaaatttatgcattcggtgtaaaattaatgtattgaacttgtcttttttagaaaatactttgtactttaatataggattctgtcatgggaatttaaattaatcgatatcgaacacggatggaataccaaaattaaaaaaaatacacatggccttcatatgaaccgtgaacctttgataacgtggaagttcaaagaagtaaagtttaagaataaactgacaaattaatttcttttatttggcccactactaaatttgccttactttctaacatgtcaagttgtctcctcgtagttgaatgatattcatttttcatcccttaagttcaatttgattgtcatactcacccatgatgttctgaaaaatgcttggccattcacaaattttatcttagttcctatgaactttataagaagctttaatttgacatgttatattattagataatataatccataacccaataaacaagtgtattaatattgtaactttgtaattgagtgcgtccacatcttattcaatcatttaaggtcattaaaaaaaattattttttgacattctaaaactttgagttgaataaatagttcatcaattattaatacataccaatgaaaagaacaaaaatgacttatttataaatcaacaaacaattttagattgctccaacatattttccaaaattaacatttaaattttaatgcaagaaaatgcataattttttacttgatctttatagcttattttttcagtctaatcaacgaatatttgaaactcgcaacttgattaaagggatttacaacaagatatatataagtagtgacaaatcttgattttaaatattttaatttggaggtcaaaattttaccataaccatttgatttataactaaattttaaatatattatttatacatatctagtaaatttttaaatatatgtatatacaaaatataaaattattgtgttcatatatgtcgataaatccttaaataatatctgcctttaccactagagaaagtaaaaaactctttaccaaaaatacatgtattatgtatacaaaaagttgatttgataactattgaaattgtatacgagtaagtaatagaaatataaaaaactacaaaactaaaaaaatatatgttttactttaatttcgaaactaatagggtctgagtgaaatattcagaaagtggactacagagggtcataatgtttttttattaaaagccactaaagtgaggaaatc

SEQ ID NO: 85 - DNA, 2399 bp, Solanum sp.
gtactaaatgataattatattaaattgatgaatatatgacatatataaatatatagacatttattatttaatcatgaataatattatttttttacttcactaaattatttcaccagaataaatttgatttaattcagataaacgagttggtaattaccctatcacaaatttggaattagtgaatgaaattttgatccaatagcaaagccaaagataaaacttttcaactcattcaggtggcacttaaaatcaagatattcttggtatcttttcaatatataagtatatgatgacgaattagtggaactaaaagaatatcccatcaaaatgctttacaacagaaacactttaacttttagtagacattttcaaaattgaaaaataatatttaaaaattaaaattgtatttagttataaatacaaaatagaatgtttttttaattgtgaataatttaaagtgaaaacactatttttgacattttaaatttttttgaattcaaagcttttgttcaagctttaactacaacttttgaattttgaatattatgcaactcaaatatgaatattagtttgtgattccaatagatatattgtatagaaatgaaaaaaatgaataatgccacaaattttactaatggtcaagatgagtggtaaatggtaagtaacctccatcctcaactgaaggtgactagtttgagctgttgaaaatagagcacttataatagcaatcactttactcttcgaagtaaaaaaaaatgaaatgatccaaatccgtattaatccaacttcaaaatggttaacccgacattgaatacctcaacgttcagattccagcaaacacacaacaatatttggtgatttcttttcaagtgttttagtcttgatgcagagtcactcaatacatgtgttagtaaaatataataactattacatcaaaattagcataggattgttgggttctgaaggtgaatagggcgtcatgcggaagcttgcaatttgcaaatcatattgttgataaatcagataacaaaaacttatactaaaaatcaaaatattattatatcaaattaatataaagaaaaacattgaaactttagagagaataaatctccccataaacaaaagtcttaaacgactacattgtggattcttattgttattgtgttagaagaaacaaacctaacaaggatctgactgaaacaatttctctacttctcgtaagtatacaaataaaatgtgcatacaccatattaattttctcaaactctacacatatcaaacactcacaagctgatttaaacacgactatttttataaaggaatatgatggaataatgccattaagattcacaaaaagatcataatgaaacttgaaaccccacaagatagaaaaagacagctaatcacttgcacatggacttacattagtagcctttcattcctcatctttttttaagatttcaataatattatcattttctacaaaaataaaataaaattgtgggcccatttggctctatagaactccacctttttaatggaaaaaaataaatatcaaattgacgatggagaaatttgtgtgtggacccattcactccaatctccatgcgacccatcacaataaatttggaagtttccacaaaatatggactctataaactcatttcccaaaaagaaaaagatcctcaattttatttatattcatatttatcactaataataattgtggttaattaatcactttaactaatactactatattgcttaatcatggtaaaattaaaaaaaggcccttaagaagatatctatgctcaatagtgaaattagaaaaaaattaaagtagattaaaaaaagtaacataaattcgtataataatttgtagcatgtttcgaactatctttatcactacaaaggaatttaaaaattaatatataagatttgaatagaaaaaacataataacaaatatatctcaaattatttagagatctcatgcgttattttttcccttactatttgtaaatgatctttataattgaagtaatactcgtaacagatttgcataatcgtatctctcaagagaataatcaaaaggccacaattcaaattcgaacaaacagtttcacaatcaatatattatttaagaaaataattttaaaattaaaacaacatttataatgaattacataatcaaatctctcgaaataatggtcaaaagatcataattcaaataataatatttaaggatcgaagatagaatatatttattattccaagcatcttactgtaggtgaatcattcttcttaaaacttaaatataaaattataaataaaaaaataatatgacataaaataaaatattagaaatgataaagaaatggagtgaaaaaaagtataaaat

SEQ ID NO: 86 - DNA, 265 bp, Brassica sp.
gacgaagatcttctcctggtaatctaaggaaacatgaatatttgttgagttttggcttgtgaagatgctctttgttcatctgctgttttcgatggatttgtgcagattaacttggagaacatgaagaagcagaaagaatagttccctatcttcttcatcatcatcaaatgagtgtggattaaaatgaaacccacccgagtgttctatcccagaagagcaatactagtttacatatacatatatatatatatatacgtataaatgg

SEQ ID NO: 87 - DNA, 61 bp, Figwort mosaic virus
agataaagcagattcctctagtacaagtggggaacaaaataacgtggaaaagagctgtcct

SEQ ID NO: 88 - DNA, 60 bp, Figwort mosaic virus
agagctgtcctgacagcccactcactaatgcgtatgacgaacgcagtgacgaccacaaaa

SEQ ID NO: 89 - DNA, 40 bp, Figwort mosaic virus
attccctctatataagaaggcattcattcccatttgaagg

SEQ ID NO: 90 - DNA, 250 bp, Lycopersicon sp.
ctgcttgagggattcgtgtgtatatgtatataataattaatttacaatttggtgcaaattaaataacttatattcaatttatttacattcatatataaactttatatatattaagagtttaatttccccataaacaagttttttatgaattttcagtcacaatagaatttttttaaaaaaaatatttttaaatgtttaacttaaattatgaaatgtgtaaatgtttgttaaccatatttagggctattgt

SEQ ID NO: 91 - DNA, 198 bp, Lycopersicon sp.
atatttagggctattgttattatttaatgaaaaataaaatataatataattcttaagaaagtattatatataaaataaaaaattacgtaacaaattatactatacccacaaaatataattatgtaaactataccatataatattatttcgtaaatttagtttgtcatataaaattttccctaaaatgaacagaaaccc

SEQ ID NO: 92 - DNA, 61 bp, Figwort mosaic virus; identical to SEQ ID NO: 87

SEQ ID NO: 93 - DNA, 60 bp, Figwort mosaic virus; identical to SEQ ID NO: 88

SEQ ID NO: 94 - DNA, 40 bp, Figwort mosaic virus; identical to SEQ ID NO: 89

SEQ ID NO: 95 - DNA, 1109 bp, Sesamum sp.
tattatttatgtctaaaaaaatttaataaactttgacaaagaaaaagtaaaaaataaaattttattttatttctacaatttatctacaatgtaaataattataatttaaaaattatttaataaaaagtttatctaatacttttattcaaaaataaattctactttttatagtttgtgctcacatattaatatatttttagaccaaataataatttaatttcaaaaatagtataatagatcctagaaattatctaaaaataaaataattataattttagaaccattttattatatatattaaaatataatttttttaatatttctatttttgtaaaaataaaaattcttatagtttgtggccaaagttggtcaaaatatttttttttcttttaatggtacttaaaaaacacgtttcttttattttttggtacctttaaataggtatttgaagttcaaagtcatgttagtcaatagaagtttactaccgttaacggccacgtgcgggacacatggcctctgttgttaacttgggacaaaaaagtatgttttttgtgttttatagtaccaaaagtgacacttgccacaattatggtacccaaaataaaatcaactttttttaacggaatcaaaaaaaaaaaattttgcccttacataatatatgtactaatcaacggattgaattttctattgtaatattcatttcattttctatttcgttcaacatatacaattatgtatatttgaacgaaatcatatattttattttgaaaaataaaaaaaaattaacacatgctatgtatatattgattgtaataaaaaataaaataattaaaatttgcaacaaatgcaatccaaccaaacataatcgccacatacccattaggtgtaagcagagcagcatttccatacatgcaacctcatgatgatcataacaaaacaaaagcccatgcacaatagataccgccaaatgtcgctcgtttctcaccatctcacactcgacgtgtcgacctcaacccaccaatttcaactataaatccccacccttctctattccccgcttcacatccatcatcagccccctcaaactactaatcccagcacctccaaac

SEQ ID NO: 96 - DNA, 484 bp, Brassica sp.
gtatataaataaacaaaaacctcaaaagcaatcaagggcaaatctccaaaatagcatatttctaaatttatatcacaaaaatagcaatcaaaaactaaaatgactaaaatgaccaaaatgatacttttctaagtttatcctttgaaaattttaatttttttatttttcaaaatttgaaatcttatccccaaaacctcatttctcaactctaaaccctaaaccataaaccctaaaatctaaaccttaaaccctaaaccccaaaccctaaaccctaaaccctaaatcctaaaccccagcctttaactctaaaccctaagtttgtgacttttgataaaacattaagtgctatttttgtgacttttgaccttgggtgctagtttgagaacataaacttgatttagtgctatttttgtctttttctcatcatataacttcttttataattacagaatatcaaaaatatggttttctgttttatctgtag

SEQ ID NO: 97 - DNA, 574 bp, Brassica sp.
aactggatcagacaaatttgtgtgtttatctttaaaatttagtgcatgggcatatttggtctgttggtttactgttcttggattggtgaaagaaattctcaagccttcttttgtgtcattaatctagaaatgtgtcaactgctcagacatcagagtcgtgttactatccaaattcatcgagtttcagtctcattgttctacaaattggtctttgataaacgctaaaactagaacaaataatatagctccaagattccgatcctagcaaacaataatgatataaatctagttaacaaaacatcgcttaaatttccaagatgcttgccgtttgtagattccacactatttttcgtctcaactaaagcagtctccaagtacacaaaatatgtgtatatacaacagaagtcgaacttgttatagaaactaagaactgaaaaccaaagaccaaaccactgctcttggaaggccaaatgtaacaatacacttgtttcttgtcttctctttttctttttttctttttcacattctactataaaaaaaaggcgaaaaacttagatataattttgctaccaac

SEQ ID NO: 98 - DNA, 782 bp, Medicago sp.
acttttatcattcccaatacaatatattccactttcccctttatttatacacttttcttaatctgtgtgaaaaaccaaagtaggtcaattaaaccgggacggagggagtacaaaaatacaacgttcaagattctacaaattgcaaataatttagcagaatttgcaatgcataatttatatttttagtatactatcatgtaggacatttcttaaaaaagaaacaattctttacaatgaccttcaaaaaatactatacgacctactttgcgtaagcagtatacattttccacattgagccaacacgaatagaatagaactactctgcctacctcattatcacgtcaaaaaaataaaagcctacctttattttaaatgattcaatttcatttgccttaactttatttttcattttcgaattaagggattagcgtcaaattcaactttcatttttgttcaaaaaaactttcatttgtattttgttttatgaagtatttagtaaccgaaatttcattagttaaagtgaataagtaaagaatattgacttcgatttctacgtattataatgtttctacaaacttttgtttgtattaaaattaaattattatttttcataaataaaatatagaaaatttagtgatttttttaaggaaaaaaaattagtgatttgtttttttggtcaagaaaattaagtgatttaatcccttactatatatcatgcaatacctttttttcctttaggaaattacgcaatacctgtatggttggtaaatcaaataattctt

SEQ ID NO: 99 - DNA, 350 bp, Medicago sp.
attcaatttcatttgccttaactttatttttcattttcgaattaagggattagcgtcaaattcaactttcatttttgttcaaaaaaactttcatttgtattttgttttatgaagtatttagtaaccgaaatttcattagttaaagtgaataagtaaagaatattgacttcgatttctacgtattataatgtttctacaaacttttgtttgtattaaaattaaattattatttttcataaataaaatatagaaaatttagtgatttttttaaggaaaaaaaattagtgatttgtttttttggtcaagaaaattaagtgatttaatcccttactatatatcatgcaatacct

SEQ ID NO: 100 - DNA, 1622 bp, Brassica sp.
tcagacactcaatacgtgggaacttattcactttcgtgtaggaaagtggaacctaaacgaaattgcagtgtgttaatatgcccatactacattgacgatattatagtctattttggtgtctattcacaagccagatatgggaaattatctattttggtggctaccaccccgttattcataactccactgcacttgttactgatgcttcgaatacttacaatttagagtttagtttcaaactgagcggaaaattacaatattttaaataattaaatttggcgttaggacataaaagtgagactattctacccatatgtttagtacaacgcaattaagcacatggatattacattccgtcggcttccacacgcgcacgcgcttgcagggtgatttttgtcaatttttgacaaaacttgtcacttggatgagtccgtactctagcatggctatattgtacattttttttgcctcttatgaatatcccataaattctctcatctataataagtagtaacatggacgtttcaggtttgggatctgttgaaacttcattttttcagtttcttctgtttaagtaaatgtggcaaattcaaaccaaaacttctttacagttttgatgacttgtatttcttgtatttcgagaaaaataaaccaagctcaaaagataaaatacagtttagttttactaaattaattcaacttggttgttgtactagacttggttacgttcaaatgccactattcacgttggtgtgaaataagtttttgttaaacaataaatatgaacgcagatagatggtgagaggagcagcatctataattcattgaaaacgcagaagggttaccaaaaaaggggagtttccaaaagatggtgctgatgagaaacagagcccatccctctccttttttcctttctcatgaaagaaattggatggccctccttcaatgtcctccacctacttaccactcatttttttttccttattatttcaataattgattaataattagtttctaatttcaacttccagttctgtaaacagcaaaaattatatatacaatctaacatctcacttgtatatacctatataaatattcgtatctatttatatgcatgtctagaggataaaaagtgtgagctttgttgtgtatatgtgctttttgacagttgctagataattggtatgcctgtttttctttttctgctatttataaatacatctcagctaagaaagaacttgtaaccttctgttttctgcaagtggggtcaaagtaccttcagagaaatattctttcaagtgaaactcgtaaaccaaaaaaaaatttacacaaagaaagagagatatttttcaagaacattattattacgaaagcagaaccaagacttaagttacactgagatcaataataattataatatatattatcgcttcaaaaccagtttctcattagtaacttctccttgtgtcctgatctccaggtaaggttgtgaatgatacagtatatatattaaccctaaaaacaaggtttatgataaaatatctgatccttgatttaacaattcgtgggtctgatatcgttcttggtttatttgtttataatgtataaattaaagagttcta

SEQ ID NO: 101 - DNA, 1062 bp, Brassica sp.
ctgtcccctgcatgatgcaatttcttgcttaaattaatatgtggatgatattacggcaaaacaataaacctctaatattcaagatgccgttggactaaccaattttccaaggataagactctcaaacataagatttcgaaaagacaaaaccaattaaactatttatcgagcaattgttcctaaatcttaacccaaaccattattatttttcttaagttctgcgtttgattttacattttagtctaagaacactaatattttatgttttttttttaatttaacttgaagtatcttttttttttgaatgaatgttaaatttattcatgcaaaaacatatttacatcatgtgcaactgtttatgaatcaaagaatcagctcatgaaactaagaacagaattccgaagttaaggatccactctaaattcctaacttgaaatatcacacttagtatccaaacgtaaacacaaattcaaaatgtataaaagggcaattaattaaacctgaattatctcattcattggctctcatgatacatgataagttgtaaaacttcatgtcagttgggttaagttttgtttaattggaatacaataattcaaaaatataatagcattaatactataccagcttcatattaatgtaggagtagggcaataaaaagaaaagaagaaataaaaaaaaggatttacccaaaaaggagaatttccagaagttgattctgatgagaaacagagcccatacctctctttttttccgtagacatgaaagaaaaattggatggtcctccttcaatgctctctcccacccaatccaaacccaactctcttcgtcttctttatttttctattttgttattttctactccttaattcccatcaattttcagattgcgatctaaatgtatatatatacatagagaattaaaagaattaggtatgagatttttgttttagagtaatggtccattttctttctttatttttcttttataacatttcagtttgaataaaactaccaaaccttctgttttctgcaagtgggtttttaaatactttcaaggaa

SEQ ID NO: 102 - DNA, 611 bp, Solanum sp.
aatacataccaatgaaaagaacaaaaatgacttatttataaatcaacaaacaattttagattgctccaacatattttccaaaattaacatttaaattttaatgcaagaaaatgcataattttttacttgatctttatagcttattttttcagtctaatcaacgaatatttgaaactcgcaacttgattaaagggatttacaacaagatatatataagtagtgacaaatcttgattttaaatattttaatttggaggtcaaaattttaccataaccatttgatttataactaaattttaaatatattatttatacatatctagtaaatttttaaatatatgtatatacaaaatataaaattattgtgttcatatatgtcgataaatccttaaataatatctgcctttaccactagagaaagtaaaaaactctttaccaaaaatacatgtattatgtatacaaaaagttgatttgataactattgaaattgtatacgagtaagtaatagaaatataaaaaactacaaaactaaaaaaatatatgttttactttaatttcgaaactaatagggtctgagtgaaatattcagaaagtggactacagagggtcata

SEQ ID NO: 103 - DNA, 585 bp, Solanum sp.
gatatctatgctcaatagtgaaattagaaaaaaattaaagtagattaaaaaaagtaacataaattcgtataataatttgtagcatgtttcgaactatctttatcactacaaaggaatttaaaaattaatatataagatttgaatagaaaaaacataataacaaatatatctcaaattatttagagatctcatgcgttattttttcccttactatttgtaaatgatctttataattgaagtaatactcgtaacagatttgcataatcgtatctctcaagagaataatcaaaaggccacaattcaaattcgaacaaacagtttcacaatcaatatattatttaagaaaataattttaaaattaaaacaacatttataatgaattacataatcaaatctctcgaaataatggtcaaaagatcataattcaaataataatatttaaggatcgaagatagaatatatttattattccaagcatcttactgtaggtgaatcattcttcttaaaacttaaatataaaattataaataaaaaaataatatgacataaaataaaatattagaaatgataaagaaatggagtgaa

SEQ ID NO: 104 - DNA, 1123 bp, Solanum sp.
agtggagwagcaaagggctatccggaacctctttaatgtaaggtttgcatacattctatactctctttactcaactcatggaatcacactgaatgtaytgttgatgtaccttactcagtggcggatctatgaagtgctgtggggrtgccacgccacccccgaacttcgacggaaactctatatatacataggtatatatgtataatatttatatacatataaagcgtgccacccacagaacaaaattggcttgtggtgccacggtaggagggcgactttagaaggttgaggttgcgggtttgaatcccatttgacacccacggactctaaatcctggatccgccactgaccttacttattatccttcccttaatatagtcaattttttttaacgacctcgtttgttcggaacacaattttttctttttcattttttattctccacagaaacttttctttttcatttgatagtataaaaaattcaaaaaaatatttttgtcgtatttccctcattattaattgttgataataatacttggaggctatcgctatcattgtgctctcaaaccaacgtgggcacacacctaaagaagataatatatgcacaaaaaagagtacattttatacacattcataaatttagttaatctacaccttccattttgtacttatcctttatcaaccattctgatctctccatgtcatcactatatatcctctaaattttccttttatatttttccaatttccatctccatccttttccgctcgccctttaattgagagtctttccataacaacttttctatttctcaatatataagaataagatctgcatatatttcactacatttattgtattatttcatagattaattgagatgctcgtaagctcaccctccaatcgaaagtctttccgaaataacttttttatttctcaacagataagaatgatctgcatatatttcattgcatttgttatattatttcgtagattaatcgaggtgctagtaagcaaaaagtagaaggaaaaagaaagtcaattgagggcattattgtaaataagtccaatagtgtgccttatcttttactatataaacacgagaacgtgactcttattact

SEQ ID NO: 105 - DNA, 1329 bp, Solanum sp.
stcgagtatggwgttgcagaatcggttgtccaaatttggaactctgttagaaatgctactaactcaaaacagtaatagaccataaatcttgttggttagcaatgctgcttgtagtcatggtttttctacttctgaagtagagttttgttgaacttctgatatgccaaaaaatagaaaattgttytcttaaggccctttcttttatgaacattgtgcaacctagtgtcatgtatctttagcatrtatcacaaattttggctgatatacagttgttgtcactcaagatctatggtctttatctagacccgatgaaaaaagtgggtcacctacgtttgttggttatacttgtacctactttcttaccratagtattagcaagggtctatcggaaacctctttatttctaccaattcactagtgattagaggagtagcaaaggtctattggaaacctctttatttctttatttctaccagatggatgtaaggtctgtatacactctatactctctctacgcaatttatggaatcacactgaatatattgttgatgtaccttgcttataattctttccttaatataattaaatttctctataacgacctcgtttgttcggaacacaagtttttctttttcatttttattctccacataaacttttctttttcatttgatattataaaatattcaaaaaaatatttttgtcgtatttccctcattattaattgttgataataacacttggaggctatcactatcattgtgctctcaaaccaacgtgggcactcacctaaagaagataatatatgcacaaaaaagagtacattttatacacattcataaatttagttaatctacaccttccattttgtacttatcctttatcaaccattctgatctctccatgtcatcactatttatcctccaaattttccttttatatttttccaatttccgtctctatcctttttctgctcgccctctaatcaagagtctttccgaaataacttttctatttctcaatatataagaataagatctgcatatatctcattgtatttattatattatttcatagattagttaagatgctcgtaaatttgacctcctattgagagttttcaaaataatttttttatttttcaataaataagaataagatctacgtatatttcactctatttgctgtattatttcgtagattagtcgaggtgctcttaagcaaagagtagcaggaaaaagaaagtcaattgagggcattattgtaaataagtccaatagtgtgccttatcttttactatataaacacgagaacgtgactctaattact

SEQ ID NO: 106 - DNA, 660 bp, Solanum sp.
acgacctcgtttgttcggaacacaattttttctttttcattttttattctccacagaaacttttctttttcatttgatagtataaaaaattcaaaaaaatatttttgtcgtatttccctcattattaattgttgataataatacttggaggctatcgctatcattgtgctctcaaaccaacgtgggcacacacctaaagaagataatatatgcacaaaaaagagtacattttatacacattcataaatttagttaatctacaccttccattttgtacttatcctttatcaaccattctgatctctccatgtcatcactatatatcctctaaattttccttttatatttttccaatttccatctccatccttttccgctcgccctttaattgagagtctttccataacaacttttctatttctcaatatataagaataagatctgcatatatttcactacatttattgtattatttcatagattaattgagatgctcgtaagctcaccctccaatcgaaagtctttccgaaataacttttttatttctcaacagataagaatgatctgcatatatttcattgcatttgttatattatttcgtagattaatcgaggtgctagtaagcaaaaagtagaaggaaaaagaaagtcaattgagggc

SEQ ID NO: 107 - DNA, 2451 bp, Lycopersicon sp.
ctcgagtccattgtggggctcccatttctctttgcatttcaagagggagccataaaggctctaaatgtcattcatcgagtcaattcgtcaaaatcggcgtatgaagtcaaatttcaaagtttaggagattgaagaaatttgaagaagactaactagaagacttctttagttttttttttatattttgtgtttcttttgtaatggcctaagcccttatggttttattttcttgtacctattcttgtatgtctagactaggacaggtacaaaagaaagaaatgggtcgaaaatccaaaaaacaggcggatccaaaacttggtcaaggcgaacagaacctgagtttggacccaaatctctctctctcactttactatttgtttacgtatttttgcttaaatgtcgttagcttaggattagaaactccaaaccccgtcgaacgccttttaaattttcgtcaaacttaaaattaactttttaacgataatttgtttcaaatttgcaaagcttgttagataaaaccttaggaaagtttaactttgaaatagattcgcaaaattgtgaaataaacaataaagattgcaaaacttgtcgacttgtttaaatgaaataaaagttcaacttcaaattgcaaaagttacaaaaaatagtcaaataagttaatcgccggaaaatcgtatttaacggagtgtcaccttcctaagacactaataggaatcccgaactctttaacattttccaaacaattttcctgttttaaagttgtttagaaaataagttttcttaattttctcaaaattaagtggcgactcctaaaaagtcgaaaatcctctgagataaaacaaactcttttcgaaaatcatttttttcgataaaacaaaataaattaaaatgaatagaaagaaaagttaaaacagtgggagtactaagaattgtatgcgtctatatcttttttttatatcatttaacttagtggtacaagctttctgcctattatatagaacgagtaagcgccatttgttgcaagatatctttttataacaaaatacaagttaattttcagattaaaaaatatttaagaagtttttgaaaagggagttacatgaattttattattttaggagttaataacttagttacactttagtttgtaatattaaatattttattaaattttggtgccccaaagacgtccaaatacatgttacttgaggtcaaatttaagtgtaatttgaaaaaaaaaagatcgttgtaaccaagtgtattagcatatatttaggatacatagtaaatctccttcacctctttcccatcttgcttgccactctctcgtatatctaatattctagatacatgtgaatcactcctgatatatgtacatagtttgattcacataatatatgtataggatacatacaaatttcacttgtttttttttctattttttgtgtatcacgtaacaaaaatatatatatctcagtgtagaatacataaaaaaaattttaattagtgataaaatatataatatgattaaaaatataaataataataatatatataataataaagtatgtctaattaggtagtttttctttttgaaaactgaaatgagaaaaagcaaaacataaaattgacttgaatgacagctacatgacattttcatcttgtagtagggacatatgatttgtttttttcctttgccacatgtgttctgttatccttaatctccaagtaatcccatattttggttgatgattcacaatataatctatctaattatgcacctccttctacttaaagaagaaaaatgtgatggcgattggcaattgggaagataattaaaatctgttgagtactctttcatccgcaatggcattcagtcgatggaacaatagtgaaagagatgtttaaaaaaattatttacatttaaaatgattttagatttgacgcaatccgaaaaaattagtctataaaaaaaattatttaaaatcatgcaagagctcaattaacttcatccgcctttgatgtgagtttttctacattcatcacgcttcccatccccgaaccccaacactctatactccgatccatgacgtgaacaaattattcaagcgttcaatttgactctaatatcatactaaataaacctaatttaatagtaaaaattagcttaacaatttactaatttcacacaattttttatattgttgtcttgtcattatctttaggtaataatagtgtaaaaattatcttacacgattatactacataatttatacgattcgttgataaattgtataccaaagtgccacctcatcacacaataatttaatttggactaagttcactattagtgaatgaatgaattttaattataaatagaggacttgacaagatcatatttgtatcaaacaccatacactttctaaattatcgatagatttattgtttcag

SEQ ID NO: 108 - DNA, 19 bp, Artificial Sequence
  Description of Artificial Sequence Synthetic oligonucleotide
yyyyynyyyctatawawas

SEQ ID NO: 109 - DNA, 12 bp, Artificial Sequence
  Description of Artificial Sequence Synthetic oligonucleotide
cccactcactaa

SEQ ID NO: 110 - DNA, 10 bp, Artificial Sequence
  Description of Artificial Sequence Synthetic oligonucleotide
agttagtggg

(19) United States
(12) Patent Application Publication
(10) Pub. No.: US 2008/0301837 A1
(43) Pub. Date: Dec. 4, 2008

(54) Promoter-based gene silencing

(75) Inventors: Caius Rommens, Boise, ID (US); Hua Yan, Boise, ID (US); Oleg Bougri, Boise, ID (US); Jingsong Ye, Boise, ID (US)

Correspondence Address: FOLEY AND LARDNER LLP;SUITE 500, 3000 K STREET NW, WASHINGTON, DC, 20007, US

(73) Assignee: J.R. SIMPLOT COMPANY

(21) Appl. No.: 11/727,128

(22) Filed: Mar. 23, 2007

Related U.S. Application Data
(60) Provisional application No. 60/784,754, filed on Mar. 23, 2006.
(60) Provisional application No. 60/801,094, filed on May 18, 2006.
(60) Provisional application No. 60/815,251, filed on Jun. 21, 2006.
(60) Provisional application No. 60/860,492, filed on Nov. 22, 2006.

(51) Int. Cl. C12N 15/29; C12N 15/11; C12Q 1/68

(52) U.S. Cl. 800/281; 536/24.1; 435/468; 800/278; 800/284; 435/6

(57) ABSTRACT

The present invention relates to unique strategies and constructs for altering expression of a desired gene by designing a construct designed to specifically target the non-transcribed 5′-regulatory sequences of that gene.

### CROSS-REFERENCE TO RELATED APPLICATIONS

This regular U.S. patent application claims priority to U.S. Provisional Application Ser. Nos. 60/860,492, filed on Nov. 22, 2006, 60/815,251, filed on Jun. 21, 2006, 60/801,094, filed on May 18, 2006, and 60/784,754, filed on Mar. 23, 2006, which are all incorporated herein by reference.

### FIELD OF THE INVENTION

The present invention relates to unique constructs for producing a nucleic acid product that downregulates or prevents expression of a desired target gene by targeting one or more the gene's promoter sequences.

### BACKGROUND OF THE INVENTION

Suppression of gene expression may be accomplished by constructs that trigger post-transcriptional or transcriptional gene silencing. These silencing mechanisms may downregulate desired polynucleotide or gene expression by chromatin modification, RNA cleavage, translational repression, or via hitherto unknown mechanisms. See Meister G. and Tuschl T., Nature, vol. 431, pp. 343-349, 2004.

A construct that is typically used in this regard is one that expresses a polynucleotide that shares some sequence identity with at least part of a target gene. Typical methods for downregulating gene expression transgenic plants, therefore, are based on transforming a plant with a construct that expresses at least one fragment of a target gene in the plant. Conventional silencing constructs produce double-stranded RNA, which is an effective molecule for downregulating gene expression.

One of these approaches expresses a polynucleotide that comprises both promoter and gene sequences. Mette et al., EMBO J 18: 241-248, 1999, expressed a polynucleotide comprising (i) the non-transcribed 5′ regulatory sequence of the nopaline synthase gene including TATA box and transcription start, and (ii) about 24-bp of the downstream leader sequence that is part of the target gene for silencing.

Mette et al., EMBO J 19: 5194-5201, 2000, expressed a polynucleotide comprising (i) the non-transcribed 5′ regulatory sequence of the nopaline synthase gene including TATA box and transcription start, and (ii) about 34-bp of the downstream leader sequence that is part of the target gene for silencing.

Berlinda et al., Mol Gen Genomics 275: 437-449, 2006, expressed a polynucleotide comprising (i) the non-transcribed 5′ regulatory sequence of the granule bound starch synthase gene including TATA box and transcription start, and (ii) about 207-bp of the downstream intron-containing leader that is part of the target gene for silencing. Berlinda could not trigger effective gene silencing when the construct comprised only non-transcribed 5′ regulatory sequences.

Sijen et al., Curr Biol 11: 436-440, 2001, expressed a polynucleotide comprising (i) the non-transcribed 5′ regulatory sequence of the dihydroflavonol reductase gene including TATA box and transcription start, and (ii) about 54-bp of the downstream intron-containing leader that is part of the target gene for silencing. Sijen could not trigger effective gene silencing when the construct comprised only non-transcribed 5′ regulatory sequences.

Jones et al., Plant Cell 11, 2291-2301, 1999, expressed a polynucleotide comprising (i) the non-transcribed 5′ regulatory sequence of the 35S promoter of cauliflower including TATA box and transcription start, and (ii) about 11-bp of the downstream leader that is part of the target gene for silencing (for sequences of this construct, see also Guerineau et al., Plant Mol Biol 18, 815-818, 1992, and Guerineau et al, Nucl Acids Res 16, 11380, 1988).

Kanno et al., Curr Biol 14, 801-805, 2004, expressed a polynucleotide comprising (i) the non-transcribed 5′ regulatory sequence of the seed-specific alpha prime promoter including TATA box and transcription start, and (ii) about 13-bp of the downstream leader that is part of the target gene for silencing (see also supplementary data, accessible at http://download.current-biology.com/supplementarydata/curbio/14/9/801/DC1/Kanno.pdf).

It appears that some transgenes and endogenous genes can be silenced by producing RNAs that target the transcription site region. This finding may reveal a mechanism similar to that described for the silencing of human genes. Janowski et al., Nature Chemical Biology 1: 216-222, 2005, for instance, demonstrated that small RNAs with complementarity to the transcription start can silence some human genes.

In contrast, sporadic efforts to employ only sequences from the non-transcribed 5′ regulatory sequences preceding a gene to silence that gene have proven unsuccessful. For instance, Belinda concluded that it is important to include sequences in the vicinity of the transcription initiation site to trigger effective silencing.

Indeed, all data indicate that the effective silencing of endogenous plant genes requires at least some endogenous gene sequences. There are disadvantages attributable to methods that are based on the expression of sequences that are, at least in part, derived from genes, such as

(i) the reductions in gene expression can be small,

(ii) homology among different genes can result in undesirable and inadvertent cross-silencing, and

(iii) such constructs have generally been applied to down-regulate the expression of transgenes rather than genes that are naturally expressed in plants, i.e., endogenous genes have generally not been targeted successfully (with the exception of the above-described construct that contains a potato Gbss promoter linked to an extensive amount of gene sequences (Berlinda et al., Mol Gen Genomics 275: 437-449, 2006).

The present invention relates to new strategies and constructs for endogenous gene silencing that are based on the expression of specific non-transcribed 5′ regulatory sequences (SNTs). The invention also teaches how to identify such functionally active sequences.

### SUMMARY OF THE INVENTION

Strategies and constructs of the present invention can be characterized by certain features. A construct may be characterized by the presence, absence, and arrangement of at least one promoter that is operably linked to a desired polynucleotide.

In a preferred embodiment of the present invention, the desired polynucleotide comprises non-transcribed 5′ regulatory sequences that precede a target gene but does not comprise sequences derived from that target gene itself. Hence, a desired polynucleotide of the present invention contains a specific fragment of non-transcribed 5′ regulatory sequences.

According to the present invention, a gene promoter polynucleotide comprises one or more specific non-transcribed 5′-regulatory fragments (“SNTs”). An SNT may have certain characteristics and permutations of elements as described in more detail below. A gene promoter polynucleotide of the present invention may comprise multiple copies of SNT sequences in direct orientation or in inverted repeat orientation. According to the present invention, a gene promoter polynucleotide may comprise (i) a sequence from the promoter, which comprises an SNT sequence, of a target gene, and (ii) an inverted repeat of that promoter/SNT sequence, wherein (a) the gene promoter polynucleotide does not comprise a sequence naturally found downstream of the target gene's transcription site and (b) transcription of the gene promoter polynucleotide produces a double stranded RNA molecule that comprises the promoter sequence and its inverted repeat.

Not only does a gene promoter polynucleotide of the present invention not comprise a sequence naturally found downstream of the target gene's transcription site, but it may also not comprise any sequences upstream from the promoter sequence's 5′-end that is a gene sequence of a preceding gene. That is, the gene promoter polynucleotide does not comprise any sequences at its 5′-end or its 3′-end that are from any untranslated region of any gene that flanks the promoter's endogenous position in the genome. Nor does the gene promoter polynucleotide comprise any sequences at its 5′-end or its 3′-end that are from any coding or noncoding region of any gene that flanks the promoter's endogenous position in the genome.

In another embodiment, however, a gene promoter polynucleotide may comprise, at its 5′-end, one or more gene sequences from a structural gene other than the target gene.

According to the present invention, an SNT sequence may be identified by essentially fragmenting, amplifying, or otherwise isolating promoter fragments from a genome and then testing a fragment that does not contain any sequence that is naturally found downstream of the relevant gene's transcription site for its ability to bring about downregulation of the gene from which it was isolated when the fragment is expressed in a cell containing a functional copy of that gene.

In other words, the present invention contemplates a method for identifying a gene promoter polynucleotide by (a) isolating a promoter fragment from a target gene, wherein the promoter fragment does not contain any sequence downstream of the target gene transcription start site, (b) introducing an expression cassette comprising a functional promoter and regulatory elements operably linked to either (i) the promoter fragment or (ii) inverted copies of the promoter fragment into a cell that contains the target gene, and (c) determining whether expression of the target gene in the cell is downregulated compared to a cell containing the target gene but not the expression cassette, wherein the transcription of a promoter fragment or inverted copies thereof which brings about downregulation of the target gene is a gene promoter polynucleotide.

Another method for identifying an SNT sequence useful for down-regulating expression of a target gene is to:

(1) Select the gene to be silenced (“the target gene”);

(2) Define the most upstream transcription start site of the target gene by employing standard methods such as rapid amplification of 5′ complementary DNA ends (Schaefer B C, Revolutions in rapid amplification of cDNA ends: new strategies for polymerase chain reaction cloning of full-length cDNA ends. Anal Biochem 1995, 227:255-273, 1995);

(3) Determine the non-transcribed 5′ regulatory sequences, which are immediately upstream from the transcription start site of the target gene, by using standard methods such as Thermal Asymmetric Interlaced (TAIL) PCR (Liu and Huang, Efficient amplification of insert end sequences from bacterial artificial chromosome clones by thermal asymmetric interlaced PCR, Plant Mol Biol Rep 16: 175-181, 1998);

(4) Identify an SNT region within the non-transcribed 5′ regulatory sequence. SNTs are characterized according to the presence of certain motifs as explained in more detail below.

Once obtained and isolated, a polynucleotide comprising the SNT region may be manipulated in a number of ways. For instance, one or more copies of an SNT-containing polynucleotide may be inserted as an inverted repeat or direct repeat between regulatory sequences that are known to promote expression of the gene promoter polynucleotide in an organism of interest to produce a silencing cassette. An inverted repeat may comprise two copies of the SNT region. A direct repeat may comprise at least four copies of the SNT region.

The resulting silencing cassettes can then be introduced into an organism of interest using any transformation method. The transformed organism can then be screened to determine whether the target gene of interest is silenced, such as by either employing molecular methods to analyze transcript levels for the selected gene or assaying for a biochemical or phenotypic trait that is associated with the selected gene.

According to the present invention, an SNT region may be characterized in terms of certain sequence motifs and their positional spacing within a desired prescribed size range delineated within the length of the isolated non-transcribed 5′ regulatory sequence. Thus, in one embodiment, an SNT region may be located no more than 150 base pairs from the target gene's transcription start site.

In another embodiment, an SNT may contain at least two CAC trinucleotides or at least two GTG trinucleotides or a combination of CAC and GTG trinucleotides. The trinucleotides may be separated from one another by at least 50 base pairs. Furthermore, any one of these trinucleotides may reside in an A/C-rich or G/T-rich region within the non-transcribed 5′ regulatory sequence. The length of the A/C-rich or G/T-rich region may be about 5-15 nucleotides, about 5-14 nucleotides, about 5-13 nucleotides, about 5-12 nucleotides, about 5-11 nucleotides, about 5-10 nucleotides, about 5-9 nucleotides, about 5-8 nucleotides, about 5-7 nucleotides, or about 5-6 nucleotides in length.

In another embodiment, an SNT region may be at least about 40 contiguous base pairs long, at least about 50 contiguous base pairs long, at least about 60 contiguous base pairs long, at least about 70 contiguous base pairs long, at least about 80 contiguous base pairs long, at least about 90 contiguous base pairs long, at least about 100 contiguous base pairs long, at least about 10 contiguous base pairs long, at least about 120 contiguous base pairs long, or more in length. In one preferred embodiment, an SNT region is at least about 80 contiguous base pairs long.

In another embodiment, an SNT may or may not comprise an 19-bp TATA box region that has the consensus sequence 5′-YYYYYNYYYCTATAWAWAS, whereby Y=C or T, N=A, C, G, or T, and W=A or T.

Generally, an SNT of the present invention also is characterized by having a local low helical stability (LHS) region that can be identified using programs such as Stress-Induced (DNA) Duplex Destabilization (Bi and Benham, Bioinformatics, 20, 1477-1479, 2004) and WEB-THERMODYN (Huang and Kowalski, Nucleic Acids Res 31, 3819-3821, 2003).

Accordingly, an SNT region of the present invention may comprise one or multiple or all of such characteristics. In essence, an SNT region is a portion of the target gene's promoter. Thus, the expression and silencing constructs of the present invention contemplate the synthesis of nucleic acid transcripts, such as single- and double-stranded RNA molecules that comprise sequences from the target gene's promoter region. Those molecules bring about down-regulation of target gene expression by targeting the endogenous promoter that normally drives expression of that target gene.

Various permutations of an SNT can be engineered together using standard molecular cloning techniques. Thus, an SNT of the present invention may be designed and created synthetically or it may be a polynucleotide that is isolated directly from a genome either by fragmentation or other isolation method, such as by PCR amplification.

Hence, in one embodiment of the present invention is an SNT fragment that comprises an STN region sequence (a) whose 3′-end is located not further than 150-250 bp upstream from the transcription start site of a target gene in the non-transcribed 5′ regulatory sequence that precedes that target gene, (b) which comprises at least two CAC or GTG trinucleotide codons that are separated by at least 20, 30, 40, 50, 60, 70, 80, 90, 100, or more base pairs, (c) consists of at least 30, 40, 50, 60, 70, 80, 90, 100, or more contiguous base pairs that may or may not contain an extended 19-bp TATA box region, and (d) that does not contain any sequences from target gene downstream of the transcription start site.

In another embodiment of the present invention is an SNT fragment that comprises an STN region sequence (a) whose 3′-end is located not further than 150 bp upstream from the transcription start site of a target gene in the non-transcribed 5′ regulatory sequence that precedes that target gene, (b) which comprises at least two CAC or GTG trinucleotide codons that are separated by at least 50 base pairs, (c) consists of at least 80 contiguous base pairs that may or may not contain an extended 19-bp TATA box region, and (d) that does not contain any sequences from target gene downstream of the transcription start site.

A desired polynucleotide of the present invention may comprise one or more copies of the SNT fragment. The orientation of SNT fragments within the desired polynucleotide may be the same as one another or different. That is, two SNT fragments may be oriented as direct repeats or inverted repeats of one another. Where there are more than two copies of an SNT fragment in a desired polynucleotide, there may be various permutations of fragment orientations so that both direct and inverted repeats of the fragments exist in the same desired polynucleotide.

Furthermore, in another embodiment, the desired polynucleotide may comprise SNT fragments of the same or different target promoters. Hence, a single desired polynucleotide may comprise portions of a first promoter, “A,” and second promoter, “B.” Thus, it is possible to target and thereby silence multiple genes with one construct.

The desired polynucleotide also may comprise sequences that share sequence identity with different regions of the same gene promoter. Hence, all of the fragments in the desired polynucleotide may target a different site of the same endogenous promoter.

The desired polynucleotide may be operably linked to one or more functional promoters. Various constructs contemplated by the present invention include, but are not limited to (1) a construct where the desired polynucleotide comprises one or more promoter fragment sequences and is operably linked at both ends to functional “driver” promoters. Those two functional promoters are arranged in a convergent orientation so that each strand of the desired polynucleotide is transcribed; (2) a construct where the desired polynucleotide is operably linked to one functional promoter at either its 5′-end or its 3′-end, and the desired polynucleotide is also operably linked at its non-promoter end by a functional terminator sequence; (3) a construct where the desired polynucleotide is operably linked to one functional promoter at either its 5′-end or its 3′-end, but where the desired polynucleotide is not operably linked to a terminator; (4) a cassette, where the desired polynucleotide comprises one or more promoter fragment sequences but is not operably linked to any functional promoters or terminators.

Hence, a construct of the present invention may comprise two or more “driver” promoters which flank one or more desired polynucleotides or which flank copies of a desired polynucleotide, such that both strands of the desired polynucleotide are transcribed. That is, one driver promoter may be oriented to initiate transcription of the 5′-end of a desired polynucleotide, while a second driver promoter may be operably oriented to initiate transcription from the 3′-end of the same desired polynucleotide. The oppositely-oriented promoters may flank multiple copies of the desired polynucleotide. Hence, the “copy number” may vary so that a construct may comprise 2, 3, 4, 5, 6, 7, 8, 9, 10, 15, 20, 30, 40, 50, 60, 70, 80, 90, or 100, or more than 100 copies, or any integer in-between, of a desired polynucleotide, which may be flanked by the driver promoters that are oriented to induce convergent transcription.

If neither cassette comprises a terminator sequence, then such a construct, by virtue of the convergent transcription arrangement, may produce RNA transcripts that are of different lengths.

In this situation, therefore, there may exist subpopulations of partially or fully transcribed RNA transcripts that comprise partial or full-length sequences of the transcribed desired polynucleotide from the respective cassette. Alternatively, in the absence of a functional terminator, the transcription machinery may proceed past the end of a desired polynucleotide to produce a transcript that is longer than the length of the desired polynucleotide.

In a construct that comprises two copies of a desired polynucleotide, therefore, where one of the polynucleotides may or may not be oriented in the inverse complementary direction to the other, and where the polynucleotides are operably linked to promoters to induce convergent transcription, and there is no functional terminator in the construct, the transcription machinery that initiates from one desired polynucleotide may proceed to transcribe the other copy of the desired polynucleotide and vice versa. The multiple copies of the desired polynucleotide may be oriented in various permutations: in the case where two copies of the desired polynucleotide are present in the construct, the copies may, for example, both be oriented in same direction, in the reverse orientation to each other, or in the inverse complement orientation to each other, for example.

In an arrangement where one of the desired polynucleotides is oriented in the inverse complementary orientation to the other polynucleotide, an RNA transcript may be produced that comprises not only the “sense” sequence of the first polynucleotide but also the “antisense” sequence from the second polynucleotide. If the first and second polynucleotides comprise the same or substantially the same DNA sequences, then the single RNA transcript may comprise two regions that are complementary to one another and which may, therefore, anneal. Hence, the single RNA transcript that is so transcribed, may form a partial or full hairpin duplex structure.

On the other hand, if two copies of such a long transcript were produced, one from each promoter, then there will exist two RNA molecules, each of which would share regions of sequence complementarity with the other. Hence, the “sense” region of the first RNA transcript may anneal to the “antisense” region of the second RNA transcript and vice versa. In this arrangement, therefore, another RNA duplex may be formed which will consist of two separate RNA transcripts, as opposed to a hairpin duplex that forms from a single self-complementary RNA transcript.

Alternatively, two copies of the desired polynucleotide may be oriented in the same direction so that, in the case of transcription read-through, the long RNA transcript that is produced from one promoter may comprise, for instance, the sense sequence of the first copy of the desired polynucleotide and also the sense sequence of the second copy of the desired polynucleotide. The RNA transcript that is produced from the other convergently-oriented promoter, therefore, may comprise the antisense sequence of the second copy of the desired polynucleotide and also the antisense sequence of the first polynucleotide. Accordingly, it is likely that neither RNA transcript would contain regions of exact complementarity and, therefore, neither RNA transcript is likely to fold on itself to produce a hairpin structure. On the other hand the two individual RNA transcripts could hybridize and anneal to one another to form an RNA duplex.

Hence, in one aspect, the present invention provides a construct that lacks a terminator or lacks a terminator that is preceded by self-splicing ribozyme encoding DNA region, but which comprises a first promoter that is operably linked to the desired polynucleotide.

As mentioned, the desired polynucleotide may comprise SNT fragments that are perfect or imperfect inverted repeats of one another, or perfect or imperfect direct repeats of one another.

The sequence of the target SNT fragment that is in the desired polynucleotide may either be naturally present in a cell genome, that is, the target promoter is endogenous to the cell genome, or it may be introduced into that genome through transformation. The SNT fragment sequence of the desired polynucleotide may or may not be functionally active and may or may not contain a TATA box or TATA box-like sequence. Thus, the promoter fragment sequence may be functionally inactive by the absence of a TATA box. In one embodiment of the present invention, no promoter fragment of a desired polynucleotide is functionally active. Hence, transcription of that expression cassette will produce RNA transcripts, which comprise the RNA sequence for a partial promoter sequence.

When a desired polynucleotide comprises a sequence that is homologous to a fragment of a target promoter sequence, then it may be desirable that the nucleotide sequence of the SNT fragment is specific to the promoter of the target gene, and/or the partial perfect or imperfect sequence of the target that is present in the desired polynucleotide is of sufficient length to confer target-specificity. Hence the portion of the desired polynucleotide that shares sequence identity with a part of a target sequence may comprise a characteristic domain, binding site, or nucleotide sequence typically conserved by isoforms or homologs of the target sequence. It is possible, therefore, to design a desired polynucleotide that is optimal for targeting a target promoter nucleic acid in a cell.

In another embodiment, the desired polynucleotide comprises an SNT sequence of preferably between 80 and 5,000 nucleotides, more preferably between 150 and 1,000 nucleotides, and most preferably between 250 and 800 nucleotides that share sequence identity with the DNA or RNA sequence of a target promoter nucleic acid sequence. The desired polynucleotide may share sequence identity with at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 300, 400, 500, or more than 500 contiguous nucleotides, or any integer in between, that are 100% identical in sequence with a sequence in a target sequence, or a desired polynucleotide comprises a sequence that shares about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 8%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% nucleotide sequence identity with a sequence of the target promoter sequence. In other words the desired polynucleotide may be homologous to, or share homology with, a fragment thereof of a target promoter sequence.

The length of the sequence of the desired polynucleotide, which shares sequence identity with a target promoter region may be 14, 15, 16, 17, 18, 19, 20, 21, 22, 23, 24, 25, 26, 27, 28, 29, 30, 31, 32, 33, 34, 35, 36, 37, 38, 39, 40; 41, 42, 43, 44, 45, 46, 47, 48, 49, 50, 60, 70, 80, 90, 100, 110, 120, 130, 140, 150, 160, 170, 180, 190, 200, 300, 400, 500, or more than 500 contiguous nucleotides in length.

Hence, the present invention provides an isolated nucleic acid molecule comprising a polynucleotide that shares homology with a target sequence and which, therefore, may hybridize under stringent or moderate hybridization conditions to a portion of a target sequence described herein. By a polynucleotide which hybridizes to a “portion” of a polynucleotide is intended a polynucleotide (either DNA or RNA) hybridizing to at least about 15 nucleotides, and more preferably at least about 20 nucleotides, and still more preferably at least about 30 nucleotides, and even more preferably more than 30 nucleotides of the reference polynucleotide. For the purpose of the invention, two sequences that share homology, i.e., a desired polynucleotide and a target sequence, may hybridize when they form a double-stranded complex in a hybridization solution of 6×SSC, 0.5% SDS, 5×Denhardt's solution and 100 μg of non-specific carrier DNA. See Ausubel et al., section 2.9, supplement 27 (1994). Such sequence may hybridize at “moderate stringency,” which is defined as a temperature of 60° C. in a hybridization solution of 6×SSC, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSC plus 0.05% SDS for five times at room temperature, with subsequent washes with 0.1×SSC plus 0.1% SDS at 60° C. for 1 h. For high stringency, the wash temperature is increased to typically a temperature that is about 68° C. Hybridized nucleotides may be those that are detected using 1 ng of a radiolabeled probe having a specific radioactivity of 10,000 cpm/ng, where the hybridized nucleotides are clearly visible following exposure to X-ray film at −70° C. for no more than 72 hours.

In one embodiment, a construct of the present invention may comprise an expression cassette that produces a nucleic acid that reduces the expression level of a target gene that is normally expressed by a cell containing the construct, by 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% in comparison to a cell that does not contain the construct.

Accordingly, depending on any of (i) the convergent arrangement of promoters and desired polynucleotides, (ii) the copy number of the desired polynucleotides, (iii) the absence of a terminator region from the construct, and (iv) the complementarity and length of the resultant transcripts, various populations of RNA molecules may be produced from the present constructs.

Hence, a single construct of the present invention may produce (i) a single stranded “sense” RNA transcript, (ii) a single-stranded “antisense” RNA transcript, (iii) a hairpin duplex formed by a single-stranded RNA transcript that anneals to itself, or (iv) an RNA duplex formed from two distinct RNA transcripts that anneal to each other. A single construct may be designed to produce only sense or only antisense RNA transcripts from each convergently-arranged promoter.

The present invention also provides a method of reducing expression of a gene normally capable of being expressed in a plant cell, by stably incorporating any of the constructs described herein into the genome of a cell.

In this regard, any type of cell from any species may be exposed to or stably- or transiently-transformed with a construct of the present invention. Hence, a bacterial cell, viral cell, fungal cell, algae cell, worm cell, plant cell, insect cell, reptile cell, bird cell, fish cell, or mammalian cell may be transformed with a construct of the present invention. The target sequence, therefore, may be located in the nucleus or a genome of any on of such cell types. The target sequence, therefore, may be located in the promoter of a gene in the cell genome.

The present invention also contemplates in vitro, ex vivo, ex planta and in vivo exposure and integration of the desired construct into a cell genome or isolated nucleic acid preparations.

The constructs of the present invention, for example, may be inserted into Agrobacterium-derived transformation plasmids that contain requisite T-DNA border elements for transforming plant cells. Accordingly, a culture of plant cells may be transformed with such a transformation construct and, successfully transformed cells, grown into a desired transgenic plant that expresses the convergently operating promoter/polynucleotide cassettes.

The functional promoters of the constructs that are used to transcribe the desired polynucleotide that contains the partial target gene promoter sequences, may be constitutive or inducible promoters or permutations thereof, and functional in plants. “Strong” promoters, for instance, can be those isolated from viruses, such as rice tungro bacilliform virus, maize streak virus, cassava vein virus, mirabilis virus, peanut chlorotic streak caulimovirus, figwort mosaic virus and chlorella virus. Other promoters can be cloned from bacterial species such as the promoters of the nopaline synthase and octopine synthase gene. Furthermore, numerous plant promoters can be used to drive expression. Such promoters include, for instance, the potato ubiquitin-7 promoter, the maize ubiquitin-1 promoter, the alfalfa PetE promoter, the canola Fad2 promoter. There are various inducible promoters, but typically an inducible promoter can be a temperature-sensitive promoter, a chemically-induced promoter, or a temporal promoter. Specifically, an inducible promoter can be a Ha hsp17.7 G4 promoter, a wheat wcs120 promoter, a Rab 16A gene promoter, an α-amylase gene promoter, a pin2 gene promoter, or a carboxylase promoter. Additional promoters can be used to trigger tissue-specific gene silencing. Such promoters include the potato Gbss promoter, the potato Agp promoter, the tomato 2A11 promoter, the tomato E8 promoter, the tomato P119 promoter, the soybean alpha prime promoter, the canola cruciferin promoter, and the canola napin promoter.

In one embodiment, the target promoter(s) from which a partial sequence is designed, is/are the 5′-regulatory sequences preceding a gene selected from the group consisting of, but not limited to a COMT gene involved in lignin biosynthesis, a CCOMT gene involved in lignin biosynthesis, any other gene involved in lignin biosynthesis, an R1 gene involved in starch phosphorylation, a phosphorylase gene involved in starch phosphorylation, a PPO gene involved in oxidation of polyphenols, a polygalacturonase gene involved in pectin degradation, a gene involved in the production of allergens, a gene involved in fatty acid biosynthesis such as FAD2.

In a further embodiment, therefore, a partial sequence, i.e., a promoter fragment, is designed from a target promoter selected from the group consisting of (1) a starch-associated R1 gene promoter, (2) a polyphenol oxidase gene promoter, (3) a fatty acid desaturase 12 gene promoter, (4) a microsomal omega-6 fatty acid desaturase gene promoter, (5) a cotton stearoyl-acyl-carrier protein delta 9-desaturase gene promoter, (6) an oleoyl-phosphatidylcholine omega 6-desaturase gene promoter, (7) a Medicago truncatula caffeic acid/5-hydroxyferulic acid 3/5-O-methyltransferase (COMT) gene promoter, (8) a Medicago sativa (alfalfa) caffeic acid/5-hydroxyferulic acid 3/5-O-methyltransferase (COMT) gene promoter, (9) a Medicago truncatula caffeoyl CoA 3-O-methyltransferase (CCOMT) gene promoter, (10) a Medicago sativa (alfalfa) caffeoyl CoA 3-O-methyltransferase (CCOMT) gene promoter, (11) a major apple allergen Mal d 1 gene promoter, (12) a major peanut allergen Ara h 2 gene promoter, (13) a major soybean allergen Gly m Bd 30 K gene promoter, and (14) a polygalacturonase gene promoter. Examples of specific partial sequences of promoters that may be used according to the present invention are provided below.

In a particular embodiment, the target promoter is located in the genome of a cell. Hence, the cell may be a cell from a bacteria, virus, fungus, yeast, plant, reptile, bird, fish, or mammal.

In a preferred embodiment, the expression cassette is located between transfer-DNA border sequences of a plasmid that is suitable for bacterium-mediated plant transformation. In yet another embodiment, the bacterium is Agrobacterium, Rhizobium, or Phyllobacterium. In one embodiment, the bacterium is Agrobacterium tumefaciens, Rhizobium trifolii, Rhizobium leguminosarum, Phyllobacterium myrsinacearum, SinoRhizobium meliloti, and MesoRhizobium loti.

Another aspect of the present invention is a method of reducing expression of a gene normally capable of being expressed in a plant cell, comprising exposing a plant cell to any construct described herein, wherein the construct is maintained in a bacterium strain, wherein the desired polynucleotide comprises a partial target promoter sequence or a sequence that shares sequence identity to a portion of a target promoter sequence in the plant cell genome.

Another aspect of the present invention is a construct, comprising an expression cassette which comprises in the 5′ to 3′ orientation (i) a first promoter, (ii) a first polynucleotide that comprises a sequence that shares sequence identity with at least a part of a promoter sequence of a target gene, (iii) a second polynucleotide comprising a sequence that shares sequence identity with the inverse complement of at least part of the promoter of the target gene, and (iv) a second promoter, wherein the first promoter is operably linked to the 5′-end of the first polynucleotide and the second promoter is operably linked to the 3′-end of the second polynucleotide.

Another aspect of the present invention is a construct, comprising an expression cassette which comprises in the 5′ to 3′ orientation (i) a first promoter, (ii) a first polynucleotide that comprises a sequence that shares sequence identity with at least a part of a promoter sequence of a target gene, (iii) a second polynucleotide comprising a sequence that shares sequence identity with the inverse complement of at least part of the promoter of the target gene, (iv) a terminator, wherein the first promoter is operably linked to the 5′-end of the first polynucleotide and the second polynucleotide is operably linked to the terminator.

Another aspect of the present invention is a method for reducing cold-induced sweetening in a tuber, comprising expressing any construct described herein in a cell of a tuber, wherein the desired polynucleotide comprises one or more direct or indirect copies of a portion of an R1 gene promoter sequence.

Another aspect of the present invention is a method for enhancing tolerance to black spot bruising in a tuber, comprising expressing any construct described herein in a cell of a tuber, wherein the desired polynucleotide comprises one or more direct or indirect copies of a portion of a polyphenol oxidase gene promoter.

Another aspect of the present invention is a method for increasing oleic acid levels in an oil-bearing plant, comprising expressing any construct described herein in a cell of a seed of an oil-bearing plant, wherein the desired polynucleotide comprises one or more direct or indirect copies of a portion of a Fad2 gene promoter. In one embodiment, the oil-bearing plant is a Brassica plant, canola plant, soybean plant, cotton plant, or a sunflower plant.

Another aspect of the present invention is a method for reducing lignin content in a plant, comprising expressing any construct described herein in a cell of the plant, wherein the desired polynucleotide comprises one or more direct or indirect copies of a portion of a caffeic acid/5-hydroxyferulic acid 3/5-O-methyltransferase (COMT) gene promoter.

Another aspect of the present invention is a method for reducing the degradation of pectin in a fruit of a plant, comprising expressing any construct described herein in a fruit cell of the plant, wherein the desired polynucleotide comprises one or more direct or indirect copies of a portion of a polygalacturonase gene promoter.

Another aspect of the present invention is a method for reducing the allergenicity of a food produced by a plant, comprising expressing any construct described herein in a cell of a plant, wherein the desired polynucleotide comprises one or more direct or indirect copies of a portion of any promoter of any gene that encodes an allergen. In one embodiment, (a) the plant is an apple plant, (b) the food is an apple, (c) the first polynucleotide comprises a sequence from the Mal d I gene promoter, and (d) expression of the construct in the apple plant reduces transcription and/or translation of Mal d I in the apple. In another embodiment, (a) the plant is a peanut plant, (b) the food is a peanut, (c) the first polynucleotide comprises a sequence from the Ara h 2 gene promoter, and (d) expression of the construct in the peanut plant reduces transcription and/or translation of Ara h 2 in the peanut. In another embodiment, (a) the plant is a soybean plant, (b) the food is a soybean, (c) the first polynucleotide comprises a sequence from the Gly m Bd gene promoter, and (d) expression of the construct in the soybean plant reduces transcription and/or translation of Gly m Bd in the soybean.

Another aspect of the present invention is a method for downregulating the expression of multiple genes in a plant, comprising expressing in a cell of a plant a construct comprising a desired polynucleotide, which comprises promoter sequence fragments of promoters that drive the endogenous expression of polyphenol oxidase, phosphorylase L gene, and the R1 gene in the plant cell.

Another aspect of the present invention is a construct, comprising two desired promoters that are operably linked to a promoter and a terminator, wherein the desired promoters share sequence identity with a target promoter in a genome of interest. In one embodiment, the two desired promoters share, over at least a part of their respective lengths, sequence identity with each other and where one of the desired promoters is oriented as the inverse complement of the other.

In another aspect is a construct, comprising two desired promoters that are operably linked to a promoter and a terminator, wherein the desired promoters share sequence identity with a target promoter in a genome of interest. In one embodiment, the two desired promoters share, over at least a part of their respective lengths, sequence identity with each other and where one of the desired promoters is oriented as the inverse complement of the other.

The present invention also provides a method for reducing the expression level of an endogenous gene in an alfalfa plant, comprising introducing a cassette into an alfalfa cell, wherein the cassette comprises two alfalfa-specific promoters arranged in a convergent orientation to each other, wherein the activity of the promoters in the cassette reduces the expression level of an endogenous alfalfa gene, which is operably linked in the alfalfa genome to a promoter that has a sequence that shares sequence identity with at least a part of one of the promoters in the cassette.

In one aspect of the present invention is a silencing construct, which contains two SNT fragments as inverted repeats of each other. In one embodiment, the polynucleotide which contains the two SNT fragments comprises the nucleotide sequence depicted in SEQ ID NO: 77. In one embodiment, the inverted repeat may be positioned between appropriate regulatory sequences. In one embodiment, by selecting the appropriate SNT fragments, it is possible to use the resulting silencing construct to effect various phenotypes, such as delaying natural leaf senescence, delaying bolting, increasing leaf and root biomass, and enhancing seed yield. Other phenotypic embodiments which may result include delayed premature leaf senescence induced by drought stress. Consequently, that transgenic plant may in turn exhibit enhanced survival in comparison with wild-type plants. In addition, detached leaves from DHS-suppressed plants will exhibit delayed post-harvest senescence.

In another embodiment, a silencing construct comprises a larger part of the promoter, e.g., such as that depicted in the nucleotide sequence of SEQ ID NO. 41. In one embodiment, transcription of such a sequence can prevent anthocyanin accumulation in varieties such as “All Blue” and “Purple Valley.” Thus, in one embodiment, the silencing construct for F35H can be used as an effective screenable marker for transformation.

In another embodiment, the present invention provides a construct which is used to target multiple promoters simultaneously. Hence, in one embodiment is an R1 promoter SNT fragment linked to the SNT fragment of the PPO and phosphorylase-L promoters. Two copies of the resulting DNA segment can be operably linked, as inverted repeats, to appropriate regulatory sequences. For instance, in one embodiment, the inverted repeat can be inserted between the AGP promoter and the terminator of the ubiquitin-7 gene. In one embodiment, such an arrangement is depicted in SEQ ID NO. 78. In one embodiment, this construct is introduced into potato to simultaneously silence the R1, phosphorylase and PPO genes. In an another embodiment, the present invention provides a tuber that displays reduced cold-sweetening, reduced starch phosphate levels, increased bruise tolerance, increased starch levels, and reduced processing-induced acrylamide accumulation.

Other embodiments of multigene promoter-based silencing include, but are not limited to (i) the simultaneous silencing of the tomato deoxyhypusine synthase and polygalacturonase genes by creating a polynucleotide that contains fragments of both the corresponding promoters. Two copies of this polynucleotide inserted as inverted repeat between either two fruit-specific promoters or a single fruit-specific promoter and a terminator represents a construct that can be introduced into tomato to silence the two genes and enhance shelf life to a greater extend than is possible through silencing of only one of the genes; and (ii) the simultaneous silencing of specific genes for Fad2, Fad3 and FatB by producing a polynucleotide that contains fragments of the three or more corresponding genes. Insertion of two copies of this polynucleotide as inverted repeat between a seed-specific promoter and terminator produces a construct that can be introduced into crops such as canola or soybean to increase oil quality to a generally higher degree than is accomplished through silencing of one of the genes. One aspect of this quality is that the oil will contain a higher content of oleic acid than the oil of untransformed plants.

In another embodiment, the sequence of the promoter that is used to silence a phosphorylase-L gene is shown in SEQ ID NO. 51. In another embodiment, a silencing construct comprises two fragments of the promoter inserted as inverted repeat between either two tuber-specific promoters or a promoter and terminator can be introduced into potato. Expression of the inverted repeat will reduce phosphorylase-L gene expression levels and consequently (1) limit starch to sugar conversion, (2) enhance bruise tolerance, and (3) increase total starch content.

Another aspect of the present invention provides an alternative approach to the use of silencing constructs. In one embodiment, that alternative approach uses promoter fragments that are oriented as direct repeats. In one embodiment, two or more fragments of the FMV promoter (SEQ ID NO. 3) can be inserted in the same orientation between two driver promoters. Introduction of this construct into plants containing the GUS gene driven by the FMV promoter will, in some plants, result in downregulated GUS gene expression. In these cases, the silencing is not triggered by hairpin RNA but rather by double-stranded RNA obtained through the annealing of RNAs produced by the two oppositely oriented driver promoters. In other words, convergent transcription produces two groups of variably-sized RNAs that will produce, in part, double-stranded RNA. An example of such a direct-repeat silencing construct is shown in FIG. 1 as pSIM150.

In another embodiment, two or more fragments of the F35H promoter (SEQ ID NO: 40) can be used to produce silencing constructs that comprise direct repeats. Introduction of such constructs into potato varieties that display purple coloration in tissue culture (such as Bintje) will result in at least partial loss of the purple color.

In another embodiment of the present invention is a construct, which comprises two copies of a non-functional FMV promoter positioned as an inverted repeat. In one embodiment, the non-functional FMV promoter has the sequence depicted in SEQ ID NO 79. In another embodiment, the construct is pSIM1113B. In another embodiment, a plant that is transformed with this construct does not display GUS activity. Construct pSIM1113B does not contain any regulatory elements that would transcribe the inverted repeat sequence. Interestingly, retransformation of tobacco plants expressing the GUS gene with pSIM1113B resulted in GUS gene silencing. Thus, promoter-based silencing constructs do not need to be transcribed in order to trigger gene silencing. Hence, one embodiment of the present invention is a construct wherein the desired targeting polynucleotide, e.g., a non-functional promoter inverted repeat, is not operably linked to any transcriptional regulatory elements.

In one embodiment is a construct for altering the expression of a target gene, comprising a desired polynucleotide that comprises at least one nucleotide sequence that shares sequence identity with a portion of a sequence of a target gene promoter. In one embodiment, the desired polynucleotide comprises two nucleotide sequences that share sequence identity with a portion of a sequence of a target gene promoter. In another embodiment, the two nucleotide sequences are identical to each other or share sequence identity with each other. In another embodiment, the two nucleotide sequences are arranged as direct repeats or inverted repeats to one another. In another embodiment, the nucleotide sequence shares 90% sequence identity with the portion of the sequence of a target gene promoter. In another embodiment, the portion of the sequence of a target gene promoter is 15-300 nucleotides in length.

In another embodiment, the desired polynucleotide is operably linked to at least one functional promoter. In another embodiment, the desired polynucleotide is operably linked to two promoters, wherein one functional promoter is operably linked to the 5′-end of the desired polynucleotide and the other functional promoter is operably linked to the 3′-end of the desired polynucleotide. In another embodiment, the desired polynucleotide comprises multiple partial nucleotide sequences of a target gene promoter. In another embodiment, the partial nucleotide sequences share at least 90% sequence identity with portions of the same or different target gene promoter.

In one embodiment, the target gene is endogenous to a plant cell. In another embodiment, the desired polynucleotide is operably linked to a terminator sequence.

In another embodiment, any one of the present constructs comprises a target gene promoter is a promoter selected from the group consisting of (1) a starch-associated R1 gene promoter, (2) a polyphenol oxidase gene promoter, (3) a fatty acid desaturase 12 gene promoter, (4) a microsomal omega-6 fatty acid desaturase gene promoter, (5) a cotton stearoyl-acyl-carrier protein delta 9-desaturase gene promoter, (6) an oleoyl-phosphatidylcholine omega 6-desaturase gene promoter, (7) a Medicago truncatula caffeic acid/5-hydroxyferulic acid 3/5-O-methyltransferase (COMT) gene promoter, (8) a Medicago sativa (alfalfa) caffeic acid/5-hydroxyferulic acid 3/5-O-methyltransferase (COMT) gene promoter, (9) a Medicago truncatula caffeoyl CoA 3-O-methyltransferase (CCOMT) gene promoter, (10) a Medicago sativa (alfalfa) caffeoyl CoA 3-O-methyltransferase (CCOMT) gene promoter, (11) a major apple allergen Mal d 1 gene promoter, (12) a major peanut allergen Ara h 2 gene promoter, (13) a major soybean allergen Gly m Bd 30 K gene promoter, and (14) a polygalacturonase gene promoter.

Another aspect of the present invention is a method for altering the expression of at least one target gene in a cell, comprising expressing the construct of claim 1 in the cell. In one embodiment, the expression of the target gene is reduced after the construct is expressed. In another embodiment, the expression of at least one of a (1) starch-associated R1 gene, (2) a polyphenol oxidase gene, (3) a fatty acid desaturase 12 gene, (4) a microsomal omega-6 fatty acid desaturase gene, (5) a cotton stearoyl-acyl-carrier protein delta 9-desaturase gene, (6) an oleoyl-phosphatidylcholine omega 6-desaturase gene, (7) a Medicago truncatula caffeic acid/5-hydroxyferulic acid 3/5-O-methyltransferase (COMT) gene, (8) a Medicago sativa (alfalfa) caffeic acid/5-hydroxyferulic acid 3/5-O-methyltransferase (COMT) gene, (9) a Medicago truncatula caffeoyl CoA 3-O-methyltransferase (CCOMT) gene, (10) a Medicago sativa (alfalfa) caffeoyl CoA 3-O-methyltransferase (CCOMT) gene, (11) a major apple allergen Mal d 1 gene, (12) a major peanut allergen Ara h 2 gene, (13) a major soybean allergen Gly m Bd 30 K gene, and (14) a polygalacturonase gene is reduced.

Another aspect of the present invention is a method for modifying a trait in a plant, comprising stably expressing the construct of claim 1 in a plant that is transformed with the construct, wherein the plant that is stably transformed with the construct expresses a trait phenotype that is different from the phenotype of that trait in a plant of the same species that does not comprise the construct. In one embodiment, the trait is modified starch and (b) the desired polynucleotide comprises at least one nucleotide sequence that shares sequence identity with a portion of a sequence of a target gene promoter selected from the group consisting of an R1 gene promoter and a phosphorylase-L gene promoter. In another embodiment, the desired polynucleotide comprises all or part of at least one of SEQ ID NO. 4, SEQ ID NO. 5, SEQ ID NO. 6, or SEQ ID NO. 42.

In another embodiment, (a) the trait is reduced lignin and (b) the desired polynucleotide comprises at least one nucleotide sequence that shares sequence identity with a portion of a sequence of a target gene promoter selected from the group consisting of an COMT gene promoter, a petE gene promoter, a Pal gene promoter, and a CCOMT gene promoter.

In another embodiment, (a) the trait is reduced lignin and (b) the desired polynucleotide comprises at least one nucleotide sequence that shares sequence identity with at least one sequence selected from the group consisting of SEQ ID NOs 20-34.

In another embodiment, (a) the trait is improved oil content and (b) the desired polynucleotide comprises at least one nucleotide sequence that shares sequence identity with a portion of a sequence of an Fad2 gene promoter,

In one embodiment, the desired polynucleotide comprises at least one nucleotide sequence that shares sequence identity with all or part of a sequence selected from the group consisting of SEQ ID NOs. 10, 11, 14, 15, and 16.

In another embodiment, the desired polynucleotide of the construct comprises at least one nucleotide sequence that shares sequence identity with a portion of a sequence of at least one of SEQ ID NOS. 1-46.

Thus, according to one aspect of the present invention, is an isolated or synthesized gene promoter polynucleotide, comprising two copies of a sequence from the promoter of at least one target gene that are positioned as inverted repeats, wherein (a) the gene promoter polynucleotide does not comprise a sequence naturally found downstream of the target gene's transcription site and (b) transcription of the gene promoter polynucleotide produces a double stranded RNA molecule.

In one embodiment, the sequence of either DNA strand of target gene promoter in the gene promoter polynucleotide comprises a specific non-transcribed sequence (“SNT”) which comprises copies of at least one of a CAC- or GTG trinucleotide, or a combination thereof.

In another embodiment, the SNT sequence comprises at least about 50-100 contiguous nucleotides of the target gene promoter sequence. In another embodiment, either strand of the SNT sequence comprises copies of at least one of a CAC trinucleotide a GTG trinucleotide. In another embodiment, at least one CAC trinucleotide is located in an A/C-rich or G/T-rich region. In another embodiment, the SNT sequence does not comprise a TATA box motif.

The present invention also provides a gene silencing construct, comprising any gene promoter polynucleotide described herein that is operably linked to a functional promoter and regulatory elements for expressing the gene promoter polynucleotide in a cell. In one embodiment, the gene promoter polynucleotide comprises multiple copies of the SNT sequence.

Another aspect of the present invention is a method for downregulating a target gene in a cell, comprising introducing the gene silencing construct of claim 7 into a cell, wherein the SNT sequence of the gene promoter polynucleotide comprises a sequence that is identical to or similar to a sequence located upstream of the transcription start site of a target gene, wherein expression of the gene promoter polynucleotide brings about downregulation of expression of the target gene in the cell. In one embodiment, the cell is a plant cell.

In another embodiment, the functional promoter is selected from the group consisting of a potato Agp promoter, a potato Gbss promoter, a potato Ubi7 promoter, an alfalfa petE promoter, a canola Fad2 promoter, and a tomato P119 promoter.

In a particular embodiment of this method, (a) the plant cell is in a plant, (b) the gene promoter polynucleotide is integrated into the plant genome, and (c) downregulation of expression of the target gene in the plant cell modifies a trait of the plant compared to a plant that does not have the gene promoter polynucleotide integrated into its genome.

In another embodiment, the modified trait of the plant containing the gene promoter polynucleotide is at least one of a modified oil content, reduced cold-sweetening, reduced starch phosphate levels, increased bruise tolerance, increased starch levels, delayed postharvest softening and senescence, prevention of anthocyanin production, and reduced processing-induced acrylamide accumulation.

In a further embodiment, the gene promoter polynucleotide comprises inverted copies of a deoxyhypusine synthase gene promoter, which is expressed in a cell from an alfalfa or canola plant.

In another embodiment, the gene promoter polynucleotide comprises inverted copies of at least one of (i) a shatterproof gene 1 promoter or (ii) a shatterproof gene 2 promoter, which is expressed in a cell of a canola plant.

In another embodiment, the gene promoter polynucleotide comprises inverted copies of at least one of (i) a Fad2-1 promoter, (ii) a Fad2-2 promoter, (iii) a Fad3 promoter, and (iv) a FatB promoter, which is expressed in a cell of a canola, soybean, cotton, safflower, or sunflower plant.

In one embodiment, the gene promoter polynucleotide comprises inverted copies of at least one of (i) a C3H promoter or (ii) a C4H promoter, which is expressed in a cell of an alfalfa plant.

Another aspect of the present invention is a method for downregulating a target gene in a cell, comprising introducing into a cell a gene silencing construct that comprises the gene promoter polynucleotide of claim 1, wherein the gene promoter polynucleotide (a) is not operably linked to a functional promoter or to any other regulatory elements, and wherein the presence of the construct in the cell brings about downregulation of expression of the target gene in the cell.

Another aspect of the present invention is a method for identifying a gene promoter polynucleotide, comprising (a) isolating a promoter fragment from a target gene, wherein the promoter fragment does not contain any sequence downstream of the target gene transcription start site, (b) introducing an expression cassette comprising a functional promoter and regulatory elements operably linked to either (i) the promoter fragment or (ii) inverted copies of the promoter fragment into a cell that contains the target gene, and (c) determining whether expression of the target gene in the cell is downregulated compared to a cell containing the target gene but not the expression cassette, wherein the transcription of a promoter fragment or inverted copies thereof which brings about downregulation of the target gene is a gene promoter polynucleotide.

Another aspect of the present invention is an isolated or synthesized gene promoter polynucleotide, comprising (i) at least one sequence from the promoter of a target gene, wherein (a) the gene promoter polynucleotide does not comprise a sequence naturally found downstream of the target gene's transcription site and (b) the gene promoter polynucleotide is positioned between functional promoters that are operably linked to the gene promoter polynucleotide in convergent orientation. In one embodiment, the promoter sequence of the isolated or synthesized gene promoter polynucleotide comprises an SNT sequence that comprises copies of a CAC- or GTG trinucleotide, or a combination thereof. In another embodiment, the gene promoter polynucleotide comprises promoter sequences from more than one target gene. In another embodiment, the promoter sequences are from different target genes.

Another aspect of the present invention is a method for downregulating at least one target gene in a plant cell, comprising (i) introducing the gene promoter polynucleotide of claim 1 or 18 into a plant cell or (ii) integrating the gene promoter polynucleotide of claim 1 or 18 into a plant cell genome, wherein (a) the gene promoter polynucleotide is operably linked to at least one functional promoter and (b) expression of the gene promoter polynucleotide brings about downregulation of at least one endogenous target gene in the plant cell.

Another aspect of the present invention is a method for downregulating more than one target gene in a cell, comprising introducing any one of the gene silencing constructs of the present invention into a cell, wherein SNT sequences of the gene promoter polynucleotide comprise sequences that are identical to or similar to sequences located upstream of the transcription start site of at least two target genes, wherein expression of the gene promoter polynucleotide brings about downregulation of expression of the target genes in the cell. In this respect, the present invention contemplates targeting and downregulating multiple target genes in a cell. Thus, 2, 3, 4, 5, 6, 7, 8, 9, 10 or more target genes can be targeted simultaneously by one or more gene promoter polynucleotides that contain appropriate SNT sequences from promoters that are operably linked to their respective target genes.

A target gene of the present invention may be located in the cell or cell type in which it normally exists in its natural genomic environment, or the target gene may be a transgene that has been previously introduced into a host cell. Thus, the cells which contain the target gene of interest may be cells that are in an in vitro environment or may be cells that are within a particular organism in vivo. Accordingly, the downregulation that is brought about by expression of one or more of the gene promoter polynucleotides of the present invention may be effected in vitro or in vivo.

In terms of downregulating multiple genes, the present invention contemplates using multiple gene promoter polynucleotides, each of which contains SNT sequences that are specific for one gene and then introducing each gene promoter polynucleotide separately into the desired cells simultaneously or sequentially. Alternatively, each target gene SNT sequence may be positioned in a gene promoter polynucleotide and then a construct containing that gene promoter polynucleotide with every SNT sequence introduced into a cell to effect downregulation of each of the specified target genes. Accordingly, various permutations of gene promoter polynucleotides and gene silencing constructs that contain those gene promoter polynucleotides may be employed simultaneously or in some sequential order to bring about downregulation of expression of multiple genes in a cell or in cells of an organism.

The present invention also contemplates an organism whose genome comprises a gene promoter polynucleotide integrated into it. Hence, the present invention contemplates a plant and progeny plants that comprise in their genomes a gene promoter polynucleotide that expresses one or more SNT sequences. Hence, a plant comprising a gene promoter polynucleotide in its genome may have lower or no expression of one or more target genes. Thus, such a transgenic plant may have different traits or phenotypes compared to a plant of the same species or variety that does not express the gene promoter polynucleotide or does not comprise the gene promoter polynucleotide in its genome. The present invention is not limited to transgenic organisms that are only transgenic plants. The genomes and genetic materials of mammals, fungi, bacteria, viruses, invertebrates, and vertebrate organisms also may be modified in such fashion to comprise or express a desired gene promoter polynucleotide.

The present invention thus explicitly encompasses transgenic plants and other organisms that comprise a gene promoter polynucleotide in their genomes or genetic material.

Any number of standard methods can be used to introduce one or more gene promoter polynucleotides into a cell or to integrate a gene promoter polynucleotide into a genome such as Agrobacterium-mediated transformation, particle bombardment, transposon-based integration, homologous recombination, nuclear transfer, naked DNA insertions, viral- or bacterial-based insertion.

### BRIEF DESCRIPTION OF THE DRAWINGS

FIG. 1: schematic representations of promoter-based silencing constructs.

FIG. 2: Glucose tuber assay. Glucose levels in minitubers, harvested from five-week old greenhouse-grown plants and stored for 4 weeks at 4° C. C=tubers from control plants (3 untransformed plants and 2 plants transformed with an empty vector combined); gR1=tubers from plants transformed with a conventional silencing construct carrying two copies of a fragment of the R1 gene inserted between Gbss promoter and terminator (see: Rommens et al., J. Agric. Food Chem 54: 9882-9887, 2006, which is incorporated herein by reference, for further details on this construct); pR1=plants transformed with constructs carrying two copies of a fragment of the R1 promoter inserted either between two convergently-oriented Gbss promoters (in pSIM1038) or between a Gbss and Agp promoter (in pSIM1043). Eleven of fifteen analyzed pSIM1038 plants did not display reduced cold sweetening. These plants are not shown. Similarly, eight of fifteen pSIM1043 plants are not shown because they contained the same glucose levels as controls.

FIG. 3: PPO tuber assay. The non-transcribed 5′ regulatory sequences preceding the PPO gene lack CAC/GTG trinucleotides. This deficiency is correlated with poor gene silencing triggered by silencing constructs that express fragments of these non-transcribed 5′ regulatory sequences (using binary vector pSIM1098). In contrast, PPO gene silencing is accomplished effectively by expressing inverted repeats carrying parts of the PPO gene (using binary vector pSIM217; see: Yan and Rommens, Plant Physiol 143: 570-578, which is incorporated herein by reference).

FIG. 4: Schematic representation of one particular embodiment of the present invention.

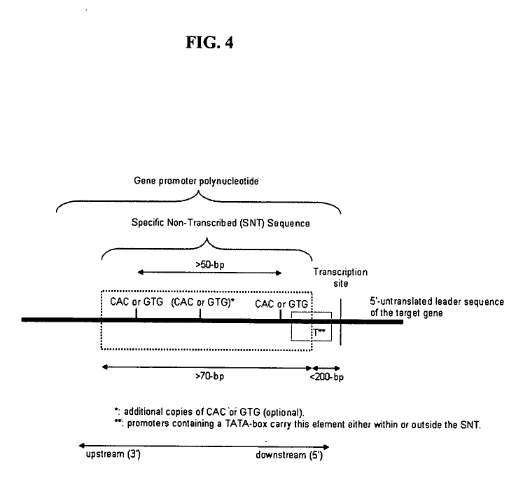

### DETAILED DESCRIPTION OF THE PREFERRED EMBODIMENTS

The present invention concerns altering the expression of a target gene in a plant, by expressing a desired polynucleotide in a plant cell, where the desired polynucleotide comprises at least one partial sequence of the target gene's promoter.

It is well accepted that a gene is a hereditary unit that occupies a specific position, i.e., a locus, within the genome or chromosome of an organism. See A DICTIONARY OF GENETICS, 4^{th} Ed., King & Stansfield. This unit may have one or more specific effects upon the phenotype of the organism and may mutate to create various allelic forms or isoforms. Three classes of genes are typically recognized by those skilled in the art of genetics, namely (1) structural genes that are transcribed into mRNAs, which are then translated to polypeptide chains, (2) structural genes that are transcribed into rRNA or tRNA molecules that are used in the cellular transcription/translation machinery, and (3) regulatory genes that are not transcribed but which serve as recognition sites for enzymes.

In each of these categories, there exist various sequence elements that facilitate and control expression of the gene in question. For that reason, a gene is typically delineated by a transcription start site at its 5′-end, and a polyadenylation signal and termination stop codon at its 3′-end. At its 5′-end, a gene may include a leader or 5′-untranslated region. At its 3′-end, a gene may include a trailer or 3′-untranslated region. A gene also comprises a coding region denoted by encoding exons and, typically, to-be-spliced-out introns.

Accordingly, a target gene of the present invention comprises (i) one or more transcription start sites, (ii) a 5′-untranslated region or leader sequence, (iii) exons, (iv) introns, (v) a 3′-untranslated region or trailer sequence, (vi) a termination sequence, and (vii) a polyadenylation sequence. Accordingly, a gene promoter polynucleotide of the present invention (A) does not comprise any of these sequences from a target gene or (B) does not comprise any sequence that is (i) downstream of the target gene's transcription site or (ii) downstream of the target gene's most upstream transcription site in instances where the gene contains more than one transcription site.

With regard to the latter, transcription start sites are sections of the DNA genome, directed by promoter regions, which initiate the production of RNA copies of the downstream target gene via the transcription process. In this regard, sometimes a gene may comprise multiple transcription start sites in the vicinity of the gene's 5-end. Typically, in that situation, one of the transcription start sites is the main or established transcription start site from which transcription begins, while other transcription start sites are cryptic start sites from which transcription does not begin.

The gene promoter polynucleotide of the present invention excludes any sequences of the target gene that lies downstream of the target gene's transcription site or downstream of the main or established transcription start site in situations where the gene has multiple transcription start sites. Where a gene has multiple transcription start sites, the present invention also contemplates that a gene promoter polynucleotide comprises no sequences that lie downstream of the 5′-most transcription start site, even if that “first” transcription start site from the 3′-end of the promoter is a cryptic transcription site from which cellular transcription is negligible or non-existent.

According to the present invention, the promoter of the target gene lies upstream of the target gene's transcription start site or upstream of the 5′-most transcription site associated with the target gene in instances where the target gene comprises multiple transcription sites.

A promoter may comprise a core promoter sequence, which is the minimal portion of the promoter that is usually required to initiate transcription of the target gene to which it is operably linked. The core promoter may be situated about 30-40 nucleotides from the transcription start site and may serve as binding sites for various RNA polymerases and general transcription factors.

A proximal promoter is understood to be a sequence in the promoter that also is situated upstream of the target gene (about 250 bp from the transcription start site) and which usually contains primary regulatory elements. It also may serve as the binding site for specific transcription factors.

A distal promoter is a sequence upstream of the target gene that may contain additional regulatory elements that are typically have a lesser effect on transcription than the regulatory elements positioned in the proximal promoter

There exist promoters in both prokaryotic and eukaryotic organisms. In prokaryotes, the promoter consists of two short sequences at −10 (The Pribnow box, TATAAT) and −35 (denoted by TTGACA) positions upstream from the transcription start site. Sigma factors not only help in enhancing RNAP binding to the promoter but helps RNAP target which genes to transcribe.

Eukaryotic promoters are diverse. They typically lie upstream of the gene and can have regulatory elements several kilobases away from the transcriptional start site. In eukaryotes, the transcriptional complex can cause the DNA to bend back on itself, which allows for placement of regulatory sequences far from the actual site of transcription. Many eukaryotic promoters, but necessarily all, contain a TATA box (TATAAA), which binds a TATA binding protein which assists in the formation of the RNA polymerase transcriptional complex. The TATA box typically is positioned close to the transcriptional start site, such as within 50 bases of the start site. Eukaryotic promoters also contain regulatory sequences that bind transcription factors that form the transcriptional complex.

In the context of the present invention, sequences from any one or type of these promoters described herein are used to design a gene promoter polynucleotide of the present invention, which, when transcribed, brings about downregulation of the target gene to which the full-length promoter is typically operably linked to in its natural genomic environment. According to the present invention, the gene promoter polynucleotide does not comprise any sequences downstream from the transcription start site, also referenced in the art as “TSS.”

Computational analysis methods are useful for identifying transcription start sites based on the availability of promoter sequence data. See Halees, et al., Nucleic Acids Res. 2003 Jul. 1; 31 (13): 3554-3559. Halees describes a freely and publicly available computer algorithm for identifying transcription start sites, The service is publicly available at http://biowulf.bu.edu/zlab/PromoSer/ and is useful for assessing and comparing promoter and upstream gene sequences from publicly available databases for identifying transcription start sites. See also Downs and Hubbard, METHODS, Vol. 12, Issue 3, 458-461, March 2002, for computational algorithms. See also Fujimori, BMC Genomics. 2005; 6: 26., (published online 2005 Feb. 28), which describes identification of transcription start sites in plants.

Transcription start sites and other upstream gene sequences and promoter sequences also can be identified and isolated from a genome using experimental techniques, such as the Rapid Amplification of cDNA ends (5′-RACE). RACE is a polymerase chain reaction-based technique developed to facilitate the cloning of the 5′-ends of messages. Today, many commercially available kits and reagents are available to conduct 5′-RACE analysis. See, for instance, Ambion's TechNotes 7 (3), http://www.ambion.com/techlib/tn/73/731.html. Generally, 5′-RACE entails performing a randomly-primed reverse transcription reaction, adding an adapter to the 3′-end of the synthesized cDNA, which is the 5′-end of the gene sequence, by ligation or polymerase extension, and amplifying by PCR with a gene specific primer and a primer that recognizes the adapter sequence. See also “Classic Protocols,” Nature Methods 2, 629-630 (2005) entitled “Rapid amplification of 5′ complementary DNA ends (5′ RACE)” and Schramm, et al., Nucleic Acids Research, 2000, Vol. 28, No. 22. Commercial suppliers of RACE kits include Invitrogen, Roche Applied Science, and Ambion.

Accordingly, therefore, it is possible to identify and get the sequence of various promoter sequences from any of the categories described herein that are operably linked to any type of target genes, as well as to identify the position and sequence of transcription start sites associated with the target gene and its promoter. Hence, it is possible to ensure that a gene promoter polynucleotide of the present invention does not include any sequences that are downstream of the target gene's transcription start site. Thus, it is possible to cleave or digest by enzymatic restriction fragmentation an isolated promoter DNA fragment that does contain sequences downstream from the transcription start site and thereby exclude those sequences for purposes of designing a gene promoter polynucleotide of the present invention. Similarly, other methods, such as PCR can be used to specifically amplify subportions of a genomic DNA fragment, or directly from the organism's genome, to produce a PCR product that contains promoter sequences but no sequences downstream from the amplified template's transcription start site.

The preceding information helps to identify the structural end-points, particularly the 3′-end of a promoter-based target gene fragment useful for designing a gene promoter polynucleotide of the present invention. The following details explain, according to the present invention, those sequence elements within the promoter region of the gene promoter polynucleotide that are useful for downregulating the expression of that target gene when the polynucleotide is expressed in a cell containing that target gene.

According to the present invention, therefore, a promoter fragment contains a specific non-transcribed 5′ regulatory sequence—the SNT sequence—which is located within and in the promoter sequence. The SNT sequence may typically be located 150-250 bp upstream of the transcription start site. According to the present invention, a gene promoter polynucleotide is a polynucleotide that contains that part of a gene's promoter that includes at least one SNT sequence but does not include any of the sequences that are naturally located downstream of the transcription start site.

A promoter, in this regard, therefore, is a nucleic acid sequence that enables a gene with which it is associated to be transcribed. Although eukaryotic promoters are diverse and difficult to characterize, there are certain fundamental characteristics. For instance, eukaryotic promoters lie upstream of the gene to which they are most immediately associated. Promoters can have regulatory elements located several kilobases away from their transcriptional start site, although certain tertiary structural formations by the transcriptional complex can cause DNA to fold, which brings those regulatory elements closer to the actual site of transcription. Many eukaryotic promoters contain a “TATA box” sequence, typically denoted by the nucleotide sequence, TATAAA. This element binds a TATA binding protein, which aids formation of the RNA polymerase transcriptional complex. The TATA box typically lies within 50 bases of the transcriptional start site.

Eukaryotic promoters also are characterized by the presence of certain regulatory sequences that bind transcription factors involved in the formation of the transcriptional complex. An example is the E-box denoted by the sequence CACGTG, which binds transcription factors in the basic-helix-loop-helix family. There also are regions that are high in GC nucleotide content.

Hence, according to the present invention, a partial sequence, or a specific promoter (SNT) fragment of a promoter that may be used in the design of a desired polynucleotide of the present invention may or may not comprise one or more of these elements or none of these elements. In one embodiment, a promoter fragment sequence of the present invention is not functional and does not contain a TATA box.

Another characteristic of the construct of the present invention is that it promotes convergent transcription of one or more copies of polynucleotide that is or are not directly operably linked to a terminator, via two opposing promoters. Due to the absence of a termination signal, the length of the pool of RNA molecules that is transcribed from the first and second promoters may be of various lengths.

Occasionally, for instance, the transcriptional machinery may continue to transcribe past the last nucleotide that signifies the “end” of the desired polynucleotide sequence. Accordingly, in this particular arrangement, transcription termination may occur either through the weak and unintended action of downstream sequences that, for instance, promote hairpin formation or through the action of unintended transcriptional terminators located in plant DNA flanking the transfer DNA integration site.

The desired polynucleotide may be linked in two different orientations to the promoter. In one orientation, e.g., “sense”, at least the 5′-part of the resultant RNA transcript will share sequence identity with at least part of at least one target transcript. In the other orientation designated as “antisense”, at least the 5′-part of the predicted transcript will be identical or homologous to at least part of the inverse complement of at least one target transcript.

As used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

Any or all of the elements and DNA sequences that are described herein may be endogenous to one or more plant genomes. Accordingly, in one particular embodiment of the present invention, all of the elements and DNA sequences, which are selected for the ultimate transfer cassette are endogenous to, or native to, the genome of the plant that is to be transformed. For instance, all of the sequences may come from a potato genome. Alternatively, one or more of the elements or DNA sequences may be endogenous to a plant genome that is not the same as the species of the plant to be transformed, but which function in any event in the host plant cell. Such plants include potato, tomato, and alfalfa plants. The present invention also encompasses use of one or more genetic elements from a plant that is interfertile with the plant that is to be transformed.

Public concerns were addressed through development of an all-native approach to making genetically engineered plants, as disclosed by Rommens et al. in WO2003/069980, US-2003-0221213, US-2004-0107455, and WO2005/004585, which are all incorporated herein by reference. Rommens et al. teach the identification and isolation of genetic elements from plants that can be used for bacterium-mediated plant transformation. Thus, Rommens teaches that a plant-derived transfer-DNA (“P-DNA”), for instance, can be isolated from a plant genome and used in place of an Agrobacterium T-DNA to genetically engineer plants.

In this regard, a “plant” of the present invention includes, but is not limited to angiosperms and gymnosperms such as potato, tomato, tobacco, avocado, alfalfa, lettuce, carrot, strawberry, sugarbeet, cassava, sweet potato, soybean, pea, bean, cucumber, grape, brassica, maize, turf grass, wheat, rice, barley, sorghum, oat, oak, eucalyptus, walnut, and palm. Thus, a plant may be a monocot or a dicot. “Plant” and “plant material,” also encompasses plant cells, seed, plant progeny, propagule whether generated sexually or asexually, and descendents of any of these, such as cuttings or seed. “Plant material” may refer to plant cells, cell suspension cultures, callus, embryos, meristematic regions, callus tissue, leaves, roots, shoots, gametophytes, sporophytes, pollen, seeds, germinating seedlings, and microspores. Plants may be at various stages of maturity and may be grown in liquid or solid culture, or in soil or suitable media in pots, greenhouses or fields. Expression of an introduced leader, trailer or gene sequences in plants may be transient or permanent.

Thus, any one of such plants and plant materials may be transformed according to the present invention. In this regard, transformation of a plant is a process by which DNA is stably integrated into the genome of a plant cell. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. See, for instance, METHODS IN PLANT MOLECULAR BIOLOGY AND BIOTECHNOLOGY, Bernard R. Glick and John E. Thompson (eds), CRC Press, Inc., London (1993); Chilton, Scientific American, 248) (6), pp. 36-45, 1983; Bevan, Nucl. Acids. Res., 12, pp. 8711-8721, 1984; and Van Montague et al., Proc R Soc Lond B Biol Sci., 210 (1180), pp. 351-65, 1980. Plants also may be transformed using “Refined Transformation” and “Precise Breeding” techniques. See, for instance, Rommens et al. in WO2003/069980, US-2003-0221213, US-2004-0107455, WO2005/004585, US-2004-0003434, US-2005-0034188, WO2005/002994, and WO2003/079765, which are all incorporated herein by reference.

One or more traits of a tuber-bearing plant of the present invention may be modified using the transformation sequences and elements described herein. A “tuber” is a thickened, usually underground, food-storing organ that lacks both a basal plate and tunic-like covering, which corms and bulbs have. Roots and shoots grow from growth buds, called “eyes,” on the surface of the tuber. Some tubers, such as caladiums, diminish in size as the plants grow, and form new tubers at the eyes. Others, such as tuberous begonias, increase in size as they store nutrients during the growing season and develop new growth buds at the same time. Tubers may be shriveled and hard or slightly fleshy. They may be round, flat, odd-shaped, or rough. Examples of tubers include, but are not limited to ahipa, apio, arracacha, arrowhead, arrowroot, baddo, bitter casava, Brazilian arrowroot, cassava, Chinese artichoke, Chinese water chestnut, coco, cocoyam, dasheen, eddo, elephant's ear, girasole, goo, Japanese artichoke, Japanese potato, Jerusalem artichoke, jicama, lilly root, ling gaw, mandioca, manioc, Mexican potato, Mexican yam bean, old cocoyam, potato, saa got, sato-imo, seegoo, sunchoke, sunroot, sweet casava, sweet potatoes, tanier, tannia, tannier, tapioca root, topinambour, water lily root, yam bean, yam, and yautia. Examples of potatoes include, but are not limited to Russet Potatoes, Round White Potatoes, Long White Potatoes, Round Red Potatoes, Yellow Flesh Potatoes, and Blue and Purple Potatoes.

Tubers may be classified as “microtubers,” “minitubers,” “near-mature” tubers, and “mature” tubers. Microtubers are tubers that are grown on tissue culture medium and are small in size. By “small” is meant about 0.1 cm-1 cm. A “minituber” is a tuber that is larger than a microtuber and is grown in soil. A “near-mature” tuber is derived from a plant that starts to senesce, and is about 9 weeks old if grown in a greenhouse. A “mature” tuber is one that is derived from a plant that has undergone senescence. A mature tuber is, for example, a tuber that is about 12 or more weeks old.

In this respect, a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterium in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. See Rommens et al. in WO2003/069980, US-2003-0221213, US-2004-0107455, and WO2005/004585, which are all incorporated herein by reference.

Thus, a P-DNA border sequence is different by 1, 2, 3, 4, 5, 6, 7, 8, 9, 10, 11, 12, 13, 14, 15, 16, 17, 18, 19, 20, or more nucleotides from a known T-DNA border sequence from an Agrobacterium species, such as Agrobacterium tumefaciens or Agrobacterium rhizogenes.

A P-DNA border sequence is not greater than 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51% or 50% similar in nucleotide sequence to an Agrobacterium T-DNA border sequence.

Methods were developed to identify and isolate transfer DNAs from plants, particularly potato and wheat, and made use of the border motif consensus described in US-2004-0107455, which is incorporated herein by reference.

In this respect, a plant-derived DNA of the present invention, such as any of the sequences, cleavage sites, regions, or elements disclosed herein is functional if it promotes the transfer and integration of a polynucleotide to which it is linked into another nucleic acid molecule, such as into a plant chromosome, at a transformation frequency of about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 15%, or about 5% or at least about 1%.

Any of such transformation-related sequences and elements can be modified or mutated to change transformation efficiency. Other polynucleotide sequences may be added to a transformation sequence of the present invention. For instance, it may be modified to possess 5′- and 3′-multiple cloning sites, or additional restriction sites. The sequence of a cleavage site as disclosed herein, for example, may be modified to increase the likelihood that backbone DNA from the accompanying vector is not integrated into a plant genome.

Any desired polynucleotide may be inserted between any cleavage or border sequences described herein. For example, a desired polynucleotide may be a wild-type or modified gene that is native to a plant species, or it may be a gene from a non-plant genome. For instance, when transforming a potato plant, an expression cassette can be made that comprises a potato-specific promoter that is operably linked to a desired potato gene or fragment thereof and a potato-specific terminator. The expression cassette may contain additional potato genetic elements such as a signal peptide sequence fused in frame to the 5′-end of the gene, and a potato transcriptional enhancer. The present invention is not limited to such an arrangement and a transformation cassette may be constructed such that the desired polynucleotide, while operably linked to a promoter, is not operably linked to a terminator sequence.

In addition to plant-derived elements, such elements can also be identified in, for instance, fungi and mammals. Several of these species have already been shown to be accessible to Agrobacterium-mediated transformation. See Kunik et al., Proc Natl Acad Sci USA 98: 1871-1876, 2001, and Casas-Flores et al., Methods Mol Biol 267: 315-325, 2004, which are incorporated herein by reference.

When a transformation-related sequence or element, such as those described herein, are identified and isolated from a plant, and if that sequence or element is subsequently used to transform a plant of the same species, that sequence or element can be described as “native” to the plant genome.

Thus, a “native” genetic element refers to a nucleic acid that naturally exists in, originates from, or belongs to the genome of a plant that is to be transformed. In the same vein, the term “endogenous” also can be used to identify a particular nucleic acid, e.g., DNA or RNA, or a protein as “native” to a plant. Endogenous means an element that originates within the organism. Thus, any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. In this respect, a “native” nucleic acid may also be isolated from a plant or sexually compatible species thereof and modified or mutated so that the resultant variant is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in nucleotide sequence to the unmodified, native nucleic acid isolated from a plant. A native nucleic acid variant may also be less than about 60%, less than about 55%, or less than about 50% similar in nucleotide sequence.

A “native” nucleic acid isolated from a plant may also encode a variant of the naturally occurring protein product transcribed and translated from that nucleic acid. Thus, a native nucleic acid may encode a protein that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60% similar in amino acid sequence to the unmodified, native protein expressed in the plant from which the nucleic acid was isolated.

In a terminator-free construct that so comprises two copies of the desired polynucleotide, one desired polynucleotide may be oriented so that its sequence is the inverse complement of the other. The schematic diagram of pSIM717 illustrates such an arrangement (see: Yan and Rommens, Plant Physiol 143: 570-578). That is, the “top,” “upper,” or “sense” strand of the construct would comprise, in the 5′- to 3′-direction, (1) a target gene fragment, and (2) the inverse complement of a target gene fragment. In this arrangement, a second promoter that is operably linked to that inverse complement of the desired polynucleotide will likely produce an RNA transcript that is at least partially identical in sequence to the transcript produced from the other desired polynucleotide.

The desired polynucleotide and its inverse complement may be separated by a spacer DNA sequence, such as an intron, that is of any length. It may be desirable, for instance, to reduce the chance of transcribing the inverse complement copy of the desired polynucleotide from the opposing promoter by inserting a long intron or other DNA sequence between the 3′-terminus of the desired polynucleotide and the 5′-terminus of its inverse complement. For example, in the case of pSIM717 the size of the intron (“I”) may be lengthened so that the transcriptional complex of P1 is unlikely to reach the sequence of the inverse complement of gus-S before becoming interrupted or dislodged. Accordingly, there may be about 50, 100, 250, 500, 2000 or more than 2000 nucleotides positioned between the sense and antisense copies of the desired polynucleotide.

A desired polynucleotide of the present invention, e.g., a “first” or “second” polynucleotide as described herein may share sequence identity with all or at least part of a sequence of a structural gene or regulatory element. For instance, a first polynucleotide may share sequence identity with a coding or non-coding sequence of a target gene or with a portion of a promoter of the target gene. In one embodiment, the polynucleotide in question shares about 100%, 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 15%, or about 5% or at least about 1% sequence identity with a target gene or target regulatory element, such as a target promoter.

A plant of the present invention may be a monocotyledonous plant, for instance, alfalfa, canola, wheat, turf grass, maize, rice, oat, barley, sorghum, orchid, iris, lily, onion, banana, sugarcane, and palm. Alternatively, the plant may be a dicotyledonous plant, for instance, potato, tobacco, tomato, avocado, pepper, sugarbeet, broccoli, cassava, sweet potato, cotton, poinsettia, legumes, alfalfa, soybean, pea, bean, cucumber, grape, brassica, carrot, strawberry, lettuce, oak, maple, walnut, rose, mint, squash, daisy, and cactus.

The location of the target promoter sequence, therefore, may be in, but is not limited to, (i) the genome of a cell; (ii) at least one RNA transcript normally produced in a cell; or (iii) in a plasmid, construct, vector, or other DNA or RNA vehicle. The cell that contains the genome or which produces the RNA transcript may be the cell of a bacteria, virus, fungus, yeast, fly, worm, plant, reptile, bird, fish, or mammal.

Hence, the target nucleic acid may be one that is normally transcribed into RNA from a cell nucleus, which is then in turn translated into an encoding polypeptide. Alternatively, the target nucleic acid may not actually be expressed in a particular cell or cell type. For instance, a target nucleic acid may be a genomic DNA sequence residing in a nucleus, chromosome, or other genetic material, such as a DNA sequence of mitochondrial DNA. Such a target nucleic acid may be of, but not limited to, a regulatory region, an untranslated region of a gene, or a non-coding sequence.

Alternatively, the target promoter sequence may be foreign to a host cell but is present or expressed by a non-host organism. For instance, a target nucleic acid may be the DNA or RNA molecule endogenous to, or expressed by, an invading parasite, virus, or bacteria.

Furthermore, the target promoter sequence may be a DNA or RNA molecule present or expressed by a disease cell. For instance, the disease cell may be a cancerous cell that expresses an RNA molecule that is not normally expressed in the non-cancerous cell type.

In plants, the desired polynucleotide may share sequence identity with a target promoter sequence that is responsible for a particular trait of a plant. For instance, a desired polynucleotide may produce a transcript that targets and reduces the expression of a polyphenol oxidase gene promoter in a plant and, thereby, modifies one or more traits or phenotypes associated with black spot bruising. Similarly, a desired polynucleotide may produce a transcript that targets and reduces the expression of a starch-associated R1 gene or phosphorylase gene in a plant, thereby modifying one or more traits or phenotypes associated with cold-induced sweetening.

All of the published documents, literature, papers and website hyperlinks are explicitly incorporated herein by reference. The following examples serve to provide exemplary details of certain embodiments described herein.

### EXAMPLES

### Example 1

### Characteristics of Promoter Fragments for Silencing a Heterologous Gene

A tobacco plant expressing the beta glucuronidase (gus) gene represents our heterologous test gene system. This plant contains the gus gene driven by the strong 35S promoter of figwort mosaic virus (FMV). It was retransformed with three different silencing constructs. Each of these silencing constructs contained two “target” FMV promoter fragments positioned as inverted repeat between two “driver promoters. The fragments of the inverted repeats were derived from the upstream (SEQ ID NO. 1), middle (SEQ ID NO. 2), and downstream (SEQ ID NO. 3) part of the FMV promoter. Interestingly, the first two constructs did not trigger any gus gene silencing whereas the third construct was extremely effective. This third fragment is characterized in that it (a) comprises a 301-bp sequence from the non-transcribed 5′ regulatory sequences that precede the target gus gene, wherein the 3′-end of the sequence is 41-bp upstream from the transcription start, and wherein the sequence comprises 12 CAC/GTG trinucleotides, whereby two of these trinucleotides are positioned within extended A/C-rich (CCCACTCACTAA) or G/T-rich (AGTTAGTGGG) regions, and (b) neither comprises the extended 19-bp TATA box region nor sequences derived from the target gene itself.

To understand the minimum size of an SNT fragment, we produced new silencing constructs that contained two copies of parts of SEQ ID NO. 3 as inverted repeat between the 35S promoter of cauliflower mosaic virus and a terminator. The first promoter fragment used for attempted gene silencing is 61-base pairs and shown in SEQ ID NO: 92; the second fragment consists of 60-base pairs (SEQ ID NO: 93). None of the resulting constructs triggered any gus gene silencing in tobacco. Equally ineffective was a 40-bp fragment comprising the TATA box region. This finding indicates that promoter-based gene silencing is not simply the result of the direct or indirect recognition of a DNA sequence by a single antigene RNA (agRNA) as described for the silencing of certain human genes by, for instance, Janowski and coworkers (Nature Chemical Biology 1: 216-222, 2005). Instead, promoter-based gene silencing in plants is associated with the direct or indirect targeting of a broader region of the 5′-untranscribed regulatory sequences that precede the target gene.

Specific fragments that are useful for silencing gene expression can be larger than 60-bp and may also contain 5-15-nucleotide sequence that is A/C rich or G/T rich.

### Example 2

### General Concept of the Promoter-Based Silencing of Endogenous Genes

Gene silencing is accomplished by defining the promoter of the target gene, and identifying an SNT fragment (a) comprising a sequence from the non-transcribed 5′ regulatory sequences that precede a target gene, wherein the 3′-end of the sequence may not be further than 150-250 bp upstream from the transcription start, preferably not more than 150-bp upstream, and wherein the sequence comprises at least two CAC/GTG trinucleotides that are separated by at least 50 base pairs; consists of at least 80 contiguous base pairs that may or may not contain an extended 19-bp TATA box region, and (b) not comprising sequences derived from that target gene itself. The SNT fragment is used to produce a silencing construct, which would typically contain two copies as inverted repeat or at least four copies as direct repeat. These structures are operably linked to regulatory sequences that would promote expression of this sequence in tissues where silencing is to be accomplished.

### Example 3

### First Example of an Effective Transgenic Approach Towards the Silencing on an Endogenous Gene

### The Potato Tuber-Expressed R1 Gene

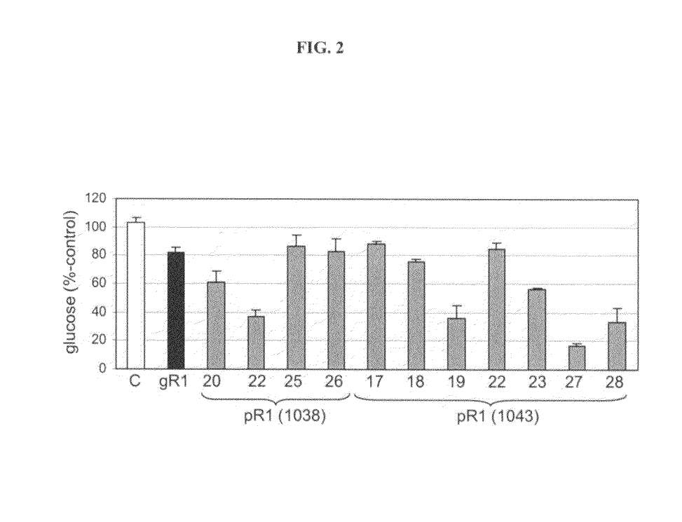

The sequence of the promoter of the potato starch-associated R1 gene together with leader and start codon, is shown in SEQ ID NO: 4. Two copies of an (342-bp) R1 SNT fragment (SEQ ID NO: 5) were inserted as inverted repeat between either two convergently oriented promoters of the GBSS promoter (in plasmid pSIM1038) or a GBSS and AGP promoter in convergent orientation (in plasmid pSIM1043). The resulting binary vectors were used to produce transformed potato plants. Transgenic pSIM1043 plants were allowed to develop min-tubers tubers, which were stored for a month at 4° C. Glucose analysis of the cold-stored tubers (Megazyme, Ireland) demonstrated that the transformed plants accumulated less glucose than untransformed control plants (FIG. 2). Multiple genes are involved in the degradation of starch into reducing sugars and therefore the present invention contemplates targeting one or more of those genes, in addition to silencing the R1 gene, to lowers cold-induced sweetening levels Further.

This assay was performed as follows:

Step 1: Preparation of Standard Curve

(1) Dissolve 1 g glucose in 1 ml dH2O to make stock solution. Prepare 1 ml dilutions of 5, 10, 20, 30, 40, 50 μg/ml from stock solution; (2) Add each dilution to a 15 ml tube containing 3 ml of the GOPOD reagent (from Amylose assay kit); vortex briefly, a pink color may develop. Prepare a blank reaction with water substituted for glucose; (3) Incubate at 50° C. for 20 min with shaking; (4) Measure the absorbance at OD510 nm; (5) Graph standard curve absorbance vs. concentration, making sure to include many different concentrations to encompass the whole range of absorbencies from the test samples.

Step 2: Tuber Preparation

(1) Wash tuber and dry thoroughly. Cut in half lengthwise, then cut a slice from the middle (cross-section of the tuber covering both ends). Cut these slices into small cubes and weigh 4-6 g into a 50 ml Falcon tube; (2) Add 2 times the weight in volumes of dH2O (ex. Tuber pieces weigh 4 g, add 8 ml H2O); (3) Grind the fresh tuber pieces with homogenizer for 20 sec on setting 4; (4) Vortex tubes vigorously to resuspend the homogenate. Transfer 1.5 ml of the homogenate to a 1.7 ml eppendorf tube; (5) Centrifuge the tube 2 min at maximum speed to pellet. Transfer supernatant to fresh eppendorf tube; (6) Dilute the samples 10× (100 μl supernatant in 900 μl H2O) in a new eppendorf tube. Maintain undiluted supernatant tubes at 4° C.

Step 3: Glucose Assay

(1) Transfer 0.1 ml of the diluted supernatant to a 15 ml tube containing 3 ml of GOPOD reagent (from Amylose Assay kit); vortex briefly, a pink color may develop; (2) Incubate at 50° C. for 20 min with shaking; (3) Measure the absorbance at OD510 nm against the blank (0.1 ml of 0.1 M sodium acetate buffer, pH 4.5); (4) Calculate glucose concentration in mg/g tuber or % of WT glucose level.

The reduced accumulation of glucose will lower color formation during French fry processing and, thus, make it possible to reduce blanch time and preserve more of the original potato flavor. Furthermore, promoter-mediated R1 gene silencing will limit starch phosphorylation and, therefore, reduce the environmental issues related to the release of waste water containing potato starch. Other benefits of the transformed tubers include: (1) resulting French fries will contain lower amounts of the toxic compound acrylamide, which is formed through a reaction between glucose and asparagine, and (2) resulting fries will display a crisper phenotype, as evaluated by professional sensory panels, due to the slightly altered structure of the starch.

A shorter (151-bp) part of the R1 promoter, such as that shown in SEQ ID NO. 6, may be used to determine what size of SNT fragment is desirable for optimal silencing, such as a size preferably greater than about 80-bp and most preferably greater than about 250-bp. Binary vector pSIM1056 comprises two copies of this SNT fragment inserted as inverted repeat between two convergently oriented GBSS promoters; pSIM1062 comprises the fragments inserted between convergently oriented GBSS and AGP promoters. This vector was used to produce 25 transformed plants, which displays reduced cold-induced glucose accumulation and all benefits associated with that trait.

### Example 4

### Second Example of an Effective Transgenic Approach Towards the Silencing on an Endogenous Gene

### The Potato Tuber-Expressed Polyphenol Oxidase Gene

The sequence of the promoter, leader, and start codon of the potato tuber-expressed polyphenol oxidase (PPO) gene is shown in SEQ ID NO: 7. The non-transcribed 5′ regulatory sequences lack CAC/GTG trinucleotides.

Two copies of a 200-bp PPO promoter fragment that includes a few base pairs of the leader (SEQ ID NO: 8) were inserted as inverted repeat between convergent GBSS and AGP promoters. A binary vector comprising this silencing construct, designated pSIM1046, was used to produce twenty-five transformed potato plants. The plants were allowed to develop mini-tubers, which were assayed for PPO activity. This assay was performed as follows:

(1) Supplies Preparation

(a) Organized, cleaned (washed in water and dried) tubers according to line and replicate; (b) 1 set labeled 50 ml Falcon tubes, 1 for each tuber; (c) 1 set labeled 1.7 ml Eppendorf tubes; (d) 1 set labeled 1.7 ml Eppendorf tubes filled with 500 μl 2× reaction buffer and appropriate amount of H2O (during transfer and 2 min spin); (e) Spectrophometric cuvettes, 1 for each sample.

(2) Solution Preparation

(a) MOPS 0.5 M pH 6.5 (10×); (b) For 500 m: Dissolve 52.33 g MOPS (fw=209.3 g) and 6 pellets of NaOH in 350 ml NANOpure H2O. Add ˜20 ml 1 M NaOH and adjust to pH 6.5, then adjust volume to 500 ml with NANOpure H2O. Filter sterilize using a 0.22 μm syringe filter. Store in a foil-covered bottle at 4° C.; (c) Catechol 0.4 M (20×); For 50 ml: Dissolve 2.2 g in 40 ml NANOpure H2O, adjust volume to 50 ml with NANOpure H2O, Store in a foil-covered tube at 4° C.; 1× buffer: 50 mM MOPS pH 6.5+20 mM Catechol (final reaction volume) to make 60 ml 2× buffer: 12 ml 0.5 M MOPS pH 6.5+6 ml 0.4 M Catechol+42 ml; (d) NANOpure H2O, Note: Prepare 2× buffer and store at 4° C. Make a fresh 1× dilution for each set of samples.

(3) Tuber Preparation

(a) Cut tuber in half lengthwise, and then cut a cross-sectional slice of the tuber covering both ends. Excise any rotted, insect-damaged or hollow-hearted areas. Cut these slices into small cubes and weigh 5 g into a 50 ml Falcon tube. Add 10 ml ice cold NANOpure H2O, store on ice until all line replicates have been cut; (b) Keeping tube on ice, homogenize tuber pieces for 30-40 s on setting 4. Return tube to ice; (c) Vortex each 50 ml tube vigorously, transfer 1.5 ml of the homogenate to a labeled 1.7 ml Eppendorf tube. Centrifuge at max speed 2 min; (d) Add supernatant to a labeled 1.7 ml tube containing reaction buffer; (e) Incubate at RT with rotation for at least 30 min; (f) Transfer reaction to cuvette, measure absorbance at OD520 against a blank; (g) Calculate PPO as % of WT.

General guidelines for volumes for reaction buffer:

(a) For each set of reactions: 500 μl 2× reaction buffer+450 μl H2O+˜50 μL supernatant (transgenic); (b) 500 μl 2× reaction buffer+490 μl H2O+˜10 μl supernatant (WT); (c) 500 μl 2× reaction buffer+400 μl H2O (blank)

(4) General Absorbance Guidelines

(a) 10 μl WT shows A520˜0.200 after 30 min; (b) 50 μl transgenic shows A520˜0.100 after 30 min (good); (c) 50 μl transgenic shows A520˜0.550 after 30 min (bad); This assay is accurate between absorbance 0.350 and 0.050 OD520.

The analysis demonstrated that the activity of the targeted PPO gene was strongly reduced if compared to levels in untransformed controls (Table 2).

In a similar way, plasmid pSIM1045, which contains two copies of a 460-bp PPO promoter fragment including a few base pairs of the leader (SEQ ID NO: 9) inserted between two convergent GBSS promoters, was used to lower PPO activity (Table 3).

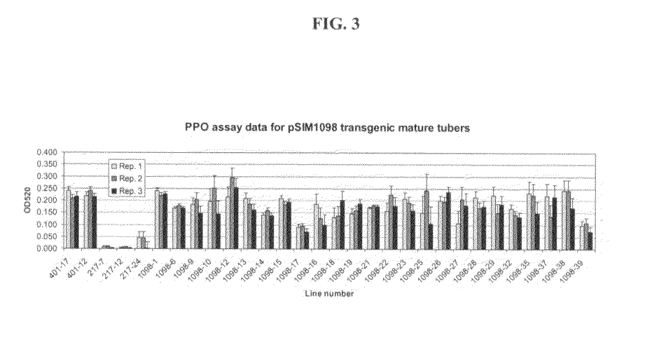

A fragment lacking any gene-derived sequences that was used to silence the PPO gene is shown in SEQ ID NO: 46. This fragment does not contain CAC/GTG trinucleotides. Consequently, we predicted a low efficacy of gene silencing. Indeed, FIG. 3 indicates much lower reductions in PPO activity than obtained with the conventional construct pSIM217, which contains parts of the PPO gene.

The “promoter” control construct that was tested contained not only sequences from the actual promoter but also from the leader (SEQ ID NO: 8). Two copies of this sequence positioned as inverted repeat between the Gbss promoter and Ubi terminator proved highly efficacious in reducing PPO gene expression levels. This type of construct is similar to the prior art “promoter” constructs that contain gene-derived sequences.

Greater reductions in reducing PPO activity can therefore be obtained in other crops using CAC/GTG-containing SNT fragments. For instance, the promoter of the leaf-expressed PPO gene of lettuce is used to reduce bruise in lettuce leaves, the promoter of the fruit-expressed PPO gene of apple is used to reduce bruise in apple fruit, and the promoter of the seed-expressed PPO gene of wheat is used to reduce bruise in wheat grains. In all these and other cases, the promoter is isolated straightforwardly by designing primers that anneal to the known PPO gene sequences, and performing well-known DNA isolation methods such as inverse PCR.

### Example 5

### Expression of Promoter Fragments of Genes Involved in Fatty Acid Biosynthesis is Used to Silence these Endogenous Genes and Improve Oil Composition

The sequence of the promoter of the Brassica Fad2-1 gene together with leader, intron, and start codon, is shown in SEQ ID NO: 10. The promoter itself is shown in SEQ ID NO: 80. Two copies of an SNT fragment of this promoter lacking any transcribed sequences such as the 515-bp fragment shown in SEQ ID NO. 11 is placed as inverted repeat between two convergently oriented promoters that are expressed in Brassica seeds. Examples of “driver” promoters are: the promoter of a napin (1.7S seed storage protein gene) gene shown in SEQ ID NO: 12. As an alternative to the napin promoter, it is possible to use, for instance, the cruciferin promoter shown in SEQ ID NO: 13.

A vector for down-regulation of Fad2-1 gene expression is pSC14. This vector contains a silencing construct comprising, from 5′ to 3′, the sesame promoter (SEQ ID NO. 95), SEQ ID NO. 11 in sense orientation, a spacer shown in SEQ ID NO.: 96, SEQ ID NO. 11 in antisense orientation, and the canola terminator shown in SEQ ID NO: 97.

Additional Brassica Fad2 gene promoters include the Fad2-2 (SEQ ID NO. 61). Parts of these promoters are used, either alone or in combinations to modify fatty acid profiles. An example of such a fragment is shown in SEQ ID NO: 62.

In one construct, SNT fragments from both the Fad2-1 and Fad2-2 promoters are fused together. Two copies of the resulting DNA segment are inserted as inverted repeat between regulatory elements for expression in canola seed. The resulting seeds will display reduced expression levels of Fad2-1 and Fad2-2 and, consequently contain high levels of oleic acid.

Similarly, the sequence of the Brassica FatB-1 promoter are used to downregulate the expression of the FatB-1 gene. A DNA fragment comprising the promoter of FatB-1 and its downstream leader is shown in SEQ ID NO. 64. An SNT fragment for this promoter is shown in SEQ ID NO. 65.

Furthermore, the FatB-2 promoter shown in SEQ ID NO 63 are used to modify fatty acid profiles. An SNT sequence of this promoter is shown in SEQ ID NO. 66.

Other preferred promoters for the modification of fatty acid content in Brassica oilseed, shown with their downstream leaders, are the Fad3-1 promoter (SEQ ID NO 56), Fad3-2 promoter (SEQ ID NO 57), Fad3-3 promoter (SEQ ID NO. 58). Putative SNT fragments that is tested for efficacy are shown in SEQ ID NO. 81, 82, and 83, respectively.

The silencing cassette is placed within the transfer DNA sequence of a binary vector, and this binary vector is used to transform Brassica. Some of the resulting plants will produce seed that contains increased amounts of oleic acid.

Similarly, a fragment of the promoter of the cotton Fad2 gene is used to improve oil composition in cottonseed (SEQ ID NO. 14). Fragment of the Sesamum and soybean Fad2 promoter (SEQ ID NO. 15 and 16) is used to improve oil composition in these plant species, respectively.

Furthermore, promoters of the stearoyl-acyl-carrier protein delta 9-desaturase gene are used to increase stearic acid levels. Examples of three such promoters are show in SEQ ID NOs. 17 (for cotton), and 18 and 19 (for flax). Other promoters are identified by performing methods such as inverse PCR using the known sequence of the target genes (Liu et al., Plant Physiol 129:1732-43, 2002). Two copies of the newly isolated promoter can then be used in strategies similar to that shown for pSIM773 whereby the ‘driver’ seed-specific promoters can either represent foreign DNA or native DNA.

It is also possible to use the promoter of an oleoyl-phosphatidylcholine omega 6-desaturase gene to increase oleic acid levels.

### Example 6

### Expression of Promoter Fragments of Genes Involved in Lignin Biosynthesis are Used to Silence these Endogenous Genes and Reduce Lignin Content

The promoter of the Medicago sativa (alfalfa) caffeic acid/5-hydroxyferulic acid 3/5-O-methyltransferase (COMT) gene, including leader, is shown in SEQ ID NO.: 20. Two copies of a 448-bp SNT fragment that lacks transcribed sequences (SEQ ID NO: 21) were inserted as inverted repeat between two convergently oriented driver promoters. The first driver promoter is the promoter of the petE gene shown in SEQ ID NO: 22; the second promoter is the promoter of the Pal gene shown in SEQ ID NO: 23. A binary vector comprising this silencing construct, designated pSIM1117, was used to produce transformed alfalfa plants. Stem tissues of the plants are assayed and shown to contain reduced levels of lignin.

Reduced lignin content is determined according to the following protocol: (i) cut stem sections and place them on watch glass, (ii) immerse the cut stems in 1% potassium permanganate for 5 min at room temperature, (iii) discard the potassium permanganate solution using a disposable pipette and wash the samples twice with water to remove excess potassium permanganate, (iv) add 6% HCl (V/V) and let the color of the sections turn from black or dark brown to light brown, (v) if necessary, add additional HCl to facilitate the removal of dark color, (vi) discard the HCl and wash the samples twice with water, (vii) add few drops of 15% sodium bicarbonate solution (some times it may not go into solution completely), a dark red or red-purple color develops for hardwoods (higher in S units) and brown color for softwood (higher in G units). Nineteen transformed alfalfa lines were tested for reduced lignin content, and six plants were found to accumulate reduced amounts of the S-unit of lignin.

Instead of the promoter of the COMT gene, it is also possible to use the promoter of the caffeoyl CoA 3-O-methyltransferase (CCOMT) gene. The sequence of this promoter, together with downstream leader, is shown in SEQ ID NO: 24. A fragment of SEQ ID NO: 29 that lacks transcribed sequences as depicted in SEQ ID NO.: 25 are used as SNT fragment to lower lignin content.

Lignin levels are reduced by targeting the promoter of various genes involved in lignin biosynthesis. In addition to the above-described COMT and CCOMT genes, these genes include genes that encode proteins such as 4-coumarate 3-hydroxylase (C3H), phenylalanine ammonia-lyase (PAL), cinnamate 4 hydroxylase (C4H), hydroxycinnamoyl transferase (HCT), and ferulate 5-hydroxylase (F5H). Examples of promoter sequences that are used to create silencing constructs to reduce lignin content in plants include the following:

(1) The promoter of the Medicago truncatula F5H gene shown in SEQ ID NO. 26;

(2) The promoter of the Pea sativum PAL gene shown in SEQ ID NO. 27;

(3) The promoter of the Trifolium subterraneum PAL gene shown in SEQ ID NO. 28;

(4) The promoter of the Populus kitakamiensis PAL gene shown in SEQ ID NO. 29;

(5) The promoter of the Arabidopsis C3H gene shown in SEQ ID NO. 30;

(6) The promoter of the Medicago truncatula C4H gene shown in SEQ ID NO. 31;

(7) The promoter of the Populus kitakamiensis C4H genes shown in SEQ ID NO. 32 and 33;

(8) The promoter of the Medicago truncatula HCH gene shown in SEQ ID NO. 34.

Preferred promoters for gene silencing in alfalfa are the promoters of the C3H gene. In fact, there are two alfalfa C3H promoters. These promoters are shown as SEQ ID NO. 47 and 98. Given the high degree of sequence homology among these two promoters, it is possible to silence the C3H gene by using a single promoter fragment, shown in SEQ ID NO: 99. Similarly, the C4H gene is silenced using a fragment of the 5′ untranscribed regulatory sequences shown in SEQ ID NO. 48.

Any other promoter of a known lignin biosynthetic gene is isolated by employing simple methods such as inverse PCR.

### Example 7

### Expression of Promoter Fragments to Increase Shelf Life

A promoter of a target polygalacturonase gene such as the tomato promoter shown in SEQ ID NO: 35 is used to reduce breakdown of pectin, thus slowing cell wall degradation, delaying softening, enhancing viscosity characteristics, and increasing shelf life in tomato by inserting two copies of the promoter fragment as inverted repeat between convergent fruit-specific driver promoters. An SNT fragment for the PG promoter that is used to produce a silencing construct for enhanced shelf life is shown in SEQ ID NO: 76.

Similarly, a promoter of a deoxyhypusine synthase (DHS) gene is used to delay postharvest softening and senescence and, thus, extend shelf life of tomato fruits. This promoter is shown in SEQ ID NO. 36. One SNT fragment is shown in SEQ ID NO. 49; two smaller alternative fragments are shown in SEQ ID NO: 90 and 91. The corresponding silencing construct comprises two copies of this fragment, inserted as inverted repeat between regulatory elements that are appropriate for either global or fruit-specific gene silencing. For instance, such regulatory elements may consist of the 2A11, E8, and P119 promoter. The latter promoter is shown as SEQ ID NO.: 107. DHS gene silencing triggered in tomato plants expressing a promoter inverted repeat sequence also has a positive effect on plants grown in soil with low nutrient levels and in the absence of commercial fertilizer.

Alfalfa promoters of the DHS gene are shown in SEQ ID NO. 37 and 38. A silencing construct containing two SNT fragments (SEQ ID NO: 77) as inverted repeat between appropriate regulatory sequences is used to delay natural leaf senescence, delay bolting, increase leaf and root biomass, and enhance seed yield. It will also result in delayed premature leaf senescence induced by drought stress, resulting in enhanced survival in comparison with wild-type plants. In addition, detached leaves from DHS-suppressed plants will exhibit delayed post-harvest senescence.

### Example 8

### Additional Example of an Effective Transgenic Approach Towards the Silencing on an Endogenous Gene

### The Potato F3,5H Gene

Some potato plants produce purple anthocyanins during at least one phase of their development. For instance, shoots of the potato variety Bintje produce anthocyanins in tissue culture. The promoter of the flavonoid 3′5′-hydroxylase (F3′5′H) gene shown in SEQ ID NO. 39 is used to prevent anthocyanin production. A silencing construct that contains two SNT fragments (SEQ ID NO. 40) inserted between two driver promoters are used to prevent this purple formation. Examples of such driver promoters are the potato ubiquitin-7 promoter and the 35S promoter of cauliflower mosaic virus. As an alternative to SEQ ID NO. 39, it is also possible to use a shorter promoter fragment shown in SEQ ID 50. Silencing constructs comprising either SEQ ID NO. 39 or 50 are introduced to potato varieties that produce anthocyanin. This anthocyanin production is then inhibited. Consequently, the plants will accumulate flavonoid precursors such as flavonols.

Transformation of Bintje stem explants with T-DNA carrying this silencing construct resulted in a high frequency of green shoots. As shown in Table 4, these shoots were confirmed by PCR to contain the construct in almost all cases. A similar silencing construct containing a larger part of the promoter (SEQ ID NO. 41) can also function effectively in limiting or preventing anthocyanin accumulation in varieties including “All Blue” and “Purple Valley”. Thus, the silencing construct for F35H is used as an effective screenable marker for transformation. If applied to potato plants that produce purple tubers, the block in the flavonoid pathway towards anthocyanins will also result in an accumulation of flavonols, which are colorless antioxidants, in tubers. In some cases, inhibition of anthocyanin biosynthesis is enhanced by employing promoters of the dihydroflavonol 4-reductase (DFR) gene.

### Example 9

### Expression of Promoter Fragments to Modify Starch

Apart from the above-described R1 promoter, there are a number of other promoters that are used to modify starch composition. The promoter of the potato starch-associated phosphorylase-L gene is used to silence this gene and, thereby, reduce the starch-to-sugar mobilization during cold storage. Thus, potato plants expressing the promoter fragments produce tubers that, after cold storage, contain lower levels of reducing sugars than the tubers of untransformed plants. These tubers allow reduced blanch times, will display a lighter fry color, and will accumulate reduced levels of acrylamide. The phosphorylase-L promoter sequence is shown in SEQ ID NO. 42. An inverted repeat containing two promoter fragments is operably linked to the appropriate regulatory sequences for expression in tubers. For instance, the inverted repeat is inserted between two tuber-specific promoters or between one tuber-specific promoter and a terminator.

Another promoter that is used to modify starch composition is the promoter of the maize shrunken gene shown in SEQ ID NO. 43. A silencing construct is used to alter the amylose/amylopectin-ratio in maize.

It is also possible to silence the two starch branching enzyme genes of potato to increase amylose levels. In contrast, amylose levels are reduced by silencing the waxy genes of plants such as maize, barley, and rice.

Preferred promoters for silencing in potato to modify starch include the promoters of the granule-bound starch synthase gene and debranching enzyme genes. Examples of GBSS promoters are shown in SEQ ID 67-72. An example of a promoter fragment that is used for silencing is shown in SEQ ID NO: 73. A sandwich construct containing two copies of this sequence, separated by a short spacer and positioned as inverted repeat is shown in SEQ ID 74. This sequence is inserted between two promoters that are functionally active in tubers. The resulting silencing construct is used to reduce expression of GBSS genes and consequently limit synthesis of amylose. Thus, the starch of GBSS-silenced potato tubers will contain more amylopectin than starch of untransformed tubers. The modified tubers are used to extract specialty starch for industrial applications. Alternatively, the tubers are used for new food applications.

The promoter of the starch branching enzyme I and II genes (shown with their downstream leaders in SEQ ID Nos: 84 and 85, respectively) were cloned by employing inverse PCR reactions with primers designed to anneal to the sequence shown in SEQ ID NO. 75. Expression of a silencing construct comprising SNT fragments for both the SBEI and SBEII promoter will increase the amylose:amylopectin ratio. Fragments of the SBEI and SBEII promoters are shown in SEQ ID NO: 102 and 103, respectively. These fragments are fused, and two copies of the resulting DNA segment is inserted as inverted repeat between the Agp promoter and a terminator. The binary vector pSIM1437 contains such a resulting silencing cassette. The increased levels of amylose in transgenic potato tubers will reduce the glycemic index of that tuber.

### Example 10

### Multi-Promoter Silencing Constructs

It is possible to target multiple promoters simultaneously. For instance, a SNT fragment of the R1 promoter is linked to the SNT fragment of the PPO and phosphorylase-L promoters. Two copies of the resulting DNA segment are linked, as inverted repeat, to the appropriate regulatory sequences. For instance, the inverted repeat is inserted between the AGP promoter and the terminator of the ubiquitin-7 gene. The resulting sequence is shown as SEQ ID NO: 78. This construct will be introduced into potato to simultaneously silence the R1, phosphorylase and PPO genes. Consequently, tubers will display reduced cold-sweetening, reduced starch phosphate levels, increased bruise tolerance, increased starch levels, and reduced processing-induced acrylamide accumulation.

Other examples of multigene promoter-based silencing include: (1) the simultaneous silencing of the tomato deoxyhypusine synthase and polygalacturonase genes by creating a polynucleotide that contains fragments of both the corresponding promoters. Two copies of this polynucleotide inserted as inverted repeat between either two fruit-specific promoters or a single fruit-specific promoter and a terminator represents a construct that is introduced into tomato to silence the two genes and enhance shelf life to a greater extend than is possible through silencing of only one of the genes; and (2) the simultaneous silencing of specific genes for Fad2, Fad3 and FatB by producing a polynucleotide that contains fragments of the three or more corresponding genes. Insertion of two copies of this polynucleotide as inverted repeat between a seed-specific promoter and terminator produces a construct that is introduced into crops such as canola or soybean to increase oil quality to a generally higher degree than is accomplished through silencing of one of the genes. One aspect of this quality is that the oil will contain a higher content of oleic acid than the oil of untransformed plants.

### Example 11

### Additional Promoters that is Used for Endogenous Gene Silencing

The brassica promoter shown in SEQ ID NO. 44 is used to improve lipid composition. The promoter of the tobacco phytoene desaturase (PDS) gene shown in SEQ ID 45 is used to enhance growth.

### Example 12

### Regulatory Sequences Driving Expression of a Target Sequence

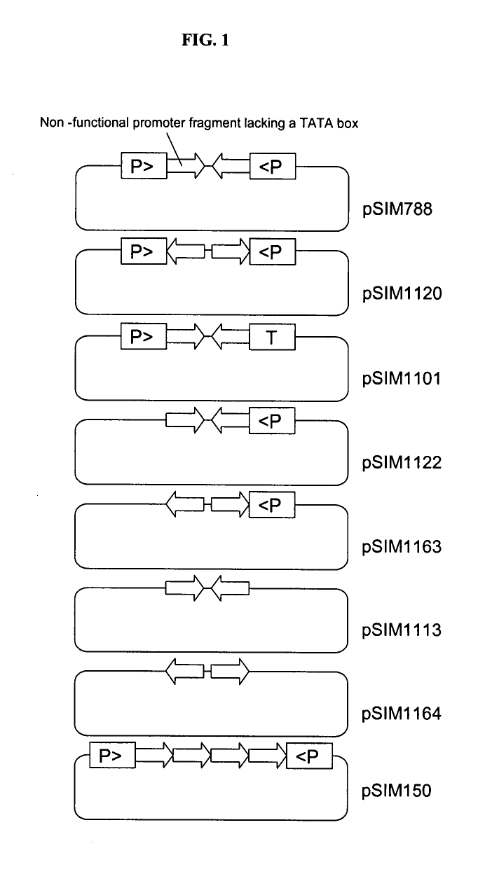

There are several different ways to arrange the regulatory sequences. A first approach inserts the target sequences between two convergent promoters. A second approach operably links the target sequences between a promoter and terminator. A third approach links the target sequences to one promoter. A fourth approach employs no regulatory sequences. The efficacy of these approaches was demonstrated by retransforming a transgenic tobacco (Nicotiana tabacum) plant that constitutively expressed the beta glucuronidase (gus) gene. The constructs used for this purpose are shown in FIG. 1, and contain two copies of a non-functional fragment of the promoter of the gus gene (i) inserted between two promoters as convergent (pSIM788) or divergent (pSIM1120) repeat, (ii) inserted between a promoter and terminator (pSIM1101), (iii) linked to one promoter as convergent (pSIM1122) or divergent (pSIM1163) repeat, and (iv) not linked to any regulatory element as convergent (pSIM1113) or divergent (pSIM1164) repeat. The frequency of gus gene silencing for the various constructs is shown in Table 5.

### Example 13

### Promoter Approach to Silence the Potato Phosphorylase-L Gene

The promoter used to silence the phosphorylase-L gene is shown in SEQ ID NO. 51. A silencing construct comprising two fragments of the promoter inserted as inverted repeat between either two tuber-specific promoters or a promoter and terminator is introduced into potato. Expression of the inverted repeat will reduce phosphorylase-L gene expression levels and consequently (1) limit starch to sugar conversion, (2) enhance bruise tolerance, and (3) increase total starch content.

### Example 14

### Promoter Silencing Approach to Increase Yield in Alfalfa and Canola

Yield is enhanced by silencing the deoxyhypusine synthase gene (DHS) of crops such as alfalfa and canola. This silencing is accomplished by expressing an inverted repeat comprising two copies of a fragment of the DHS promoter. The alfalfa DHS promoter is shown in SEQ ID NO. 52. The fragment shown in SEQ ID NO. 53 is used for silencing, and a sandwich construct comprising two copies of this fragment positioned as an inverted repeat that is separated by a spacer is shown in SEQ ID NO. 54. An alternative and more preferred fragment of the DHS promoter is shown in SEQ ID 55 and is used for silencing.

Two canola DHS promoters are shown in SEQ ID NO. 59 (BnDHS1) and SEQ ID NO. 60 (BnDHS2), respectively. An SNT fragment for the BnDHS1 promoter is shown in SEQ ID NO: 86.

### Example 15

### Promoter Silencing Constructs that do not Produce Hairpin RNA

As an alternative to silencing constructs that contain promoter fragments oriented as inverted repeat, it is also possible to position such fragments as direct repeats. For instance, two or more fragments of the FMV promoter (SEQ ID NO. 3) is inserted in the same orientation between two driver promoters. Introduction of this construct into plants containing the GUS gene driven by the FMV promoter will, in some plants, result in downregulated GUS gene expression. In these cases, the silencing is not triggered by hairpin RNA but rather by double-stranded RNA obtained through the annealing of RNAs produced by the two oppositely oriented driver promoters. In other words, convergent transcription produces two groups of variably-sized RNAs that will produce, in part, double-stranded RNA. An example of such a direct-repeat silencing construct is shown in FIG. 1 as pSIM150.

Similarly, two or more fragments of the F35H promoter (SEQ ID NO: 40) are useful for producing silencing constructs that comprise direct repeats. Introduction of such constructs into potato varieties that display purple coloration in tissue culture (such as Bintje) will result in at least partial loss of the purple color.

### Example 16

### Silencing Constructs that do not Produce RNA

Construct pSIM1113B comprises two copies of a non-functional FMV promoter (SEQ ID NO 79) positioned as inverted repeat. The employed promoter fragment was confirmed to lack functionality by linking it to the GUS gene. Plants transformed with this construct did not display GUS activity. Construct pSIM1113B did not contain any regulatory elements that would transcribe the inverted repeat sequence. Interestingly, retransformation of tobacco plants expressing the GUS gene with pSIM1113B resulted in GUS gene silencing. Thus, promoter-based silencing constructs do not need to be transcribed in order to trigger gene silencing.

### Example 17

### High-Copy Promoter-Based Gene Silencing

It may in some cases be beneficial to use small promoter fragments for gene silencing. By targeting small (about 30 to 200 base pairs) promoter regions, it is less likely that other genes with similar promoter sequences are inadvertently co-silenced. Silencing constructs comprise multiple copies of the small SNT fragment to ensure adequate expression. The number of copies that is inserted between two convergent promoters is preferably at least four, and most preferably at least eight.

The concept of high-copy promoter-based silencing is demonstrated by producing a silencing construct comprising eight copies of a 61-base pair fragment of the FMV promoter (as direct repeats) shown in SEQ ID NO: 87. This DNA segment is inserted between two convergent promoters, and introduced into a tobacco plant containing the gus gene operably linked to the FMV promoter. Introduction of the silencing construct will in some plants result in a reduction of gus gene expression levels.

Alternatively, a silencing construct is used that contains eight copies of a 60-base pair or 41-base pair promoter fragment shown in SEQ ID NO: 88 and 89, respectively.

### Example 18

### Shatterproof

It is possible to reduce shatter in canola by reducing expression of shatterproof (Shp) genes (see Liljegren et al., Nature 404: 766-770). The promoters of the canola Shp1 and Shp2 gene are shown as SEQ ID NO: 100 and 101, respectively.

### Example 19

### Modified Potato Tuber Size and Set

It is possible to increase tuber number while reducing tuber size by silencing the Gal83 gene (Lovas et al., Plant J 33: 139-147). Instead of using gene-derived sequences, Gal83 gene expression levels can be lowered by inserting two copies of a promoter fragment positioned as inverted repeat between regulatory sequences for expression in tubers. The promoters of the Gal83-1 and Gal83-2 genes are shown in SEQ ID NO: 104 and 105, respectively. A fragment that can be used to produce a silencing construct is shown in SEQ ID NO: 106.

Tables

TABLE 1
Glucose content in mini-tubers after one-month storage at 4° C.

| | OD510 raw data | | | Glucose, ug/ul | | | | Glucose, % of WT | | |
|---|---|---|---|---|---|---|---|---|---|---|
| Line | I | II | III | I | II | III | Line | I | II | III |
| RR-2 | 0.236 | 0.232 | 0.258 | 25.8 | 25.4 | 28.0 | RR-2 | 102.8 | 101.2 | 111.6 |
| RR-5 | 0.19 | 0.214 | 0.209 | 21.2 | 23.6 | 23.1 | RR-5 | 84.5 | 94.1 | 92.1 |
| RR-6 | 0.241 | 0.253 | 0.227 | 26.3 | 27.5 | 24.9 | RR-6 | 104.8 | 109.6 | 99.2 |
| 401-1 | 0.242 | 0.234 | 0.235 | 26.4 | 25.6 | 25.7 | 401-1 | 105.2 | 102.0 | 102.4 |
| 401-2 | 0.238 | 0.239 | 0.22 | 26.0 | 26.1 | 24.2 | 401-2 | 103.6 | 104.0 | 96.5 |
| 401-3 | 0.175 | 0.263 | 0.243 | 19.6 | 28.5 | 26.5 | 401-3 | 78.5 | 113.6 | 105.6 |
| 332-10 | 0.155 | 0.11 | | 17.6 | 13.1 | | 332-10 | 70.5 | 52.5 | |
| 332-22 | 0.14 | 0.142 | 0.154 | 16.1 | 16.3 | 17.5 | 332-22 | 64.5 | 65.3 | 70.1 |
| 332-41 | 0.22 | 0.184 | 0.185 | 24.2 | 20.5 | 20.7 | 332-41 | 96.5 | 82.1 | 82.5 |
| 1038-2 | 0.18 | 0.204 | | 20.1 | 22.6 | | 1038-2 | 80.5 | 90.1 | |
| 1038-3 | 0.262 | | | 28.4 | | | 1038-3 | 113.2 | | |
| 1038-5 | 0.276 | | | 29.8 | | | 1038-5 | 118.8 | | |
| 1037-6 | 0.272 | 0.227 | 0.26 | 29.4 | 24.9 | 28.2 | 1037-6 | 117.2 | 99.2 | 112.4 |
| 1038-9 | 0.144 | 0.158 | 0.195 | 16.5 | 17.9 | 21.7 | 1038-9 | 66.1 | 71.7 | 86.5 |
| 1043-2 | 0.192 | 0.211 | 0.235 | 21.4 | 23.3 | 25.7 | 1043-2 | 85.3 | 92.9 | 102.4 |
| 1043-3 | 0.183 | 0.247 | 0.219 | 20.4 | 26.9 | 24.1 | 1043-3 | 81.7 | 107.2 | 96.1 |
| 1043-4 | 0.189 | 0.164 | 0.185 | 21.1 | 18.5 | 20.7 | 1043-4 | 84.1 | 74.1 | 82.5 |
| 1043-7 | 0.274 | 0.227 | 0.264 | 29.6 | 24.9 | 28.6 | 1043-7 | 118.0 | 99.2 | 114.0 |
| 1043-8 | 0.202 | 0.199 | 0.11 | 22.4 | 22.1 | 13.1 | 1043-8 | 89.3 | 88.1 | 52.5 |
| 1043-9 | 0.178 | 0.173 | 0.186 | 19.9 | 19.4 | 20.8 | 1043-9 | 79.7 | 77.7 | 82.9 |
| 1043-11 | 0.221 | | | 24.3 | | | 1043-11 | 96.9 | | |
| 1043-12 | 0.25 | 0.207 | | 27.2 | 22.9 | | 1043-12 | 108.4 | 91.3 | |

TABLE 2
PPO activity of three 1-month old tubers.

| Line | Rep. 1 | Rep. 2 | Rep. 3 | Av | SD |
|---|---|---|---|---|---|
| WT-2 | 0.135 | 0.141 | 0.138 | 0.138 | 0.003 |
| WT-3 | 0.143 | 0.121 | 0.165 | 0.143 | 0.022 |
| 401-1 | 0.155 | 0.173 | 0.094 | 0.141 | 0.041 |
| 401-2 | 0.197 | 0.197 | 0.212 | 0.202 | 0.009 |
| 217-7 | 0.039 | 0.046 | 0.054 | 0.046 | 0.007 |
| 217-12 | 0.037 | 0.043 | 0.034 | 0.038 | 0.004 |
| 217-24 | 0.038 | 0.040 | 0.034 | 0.037 | 0.003 |
| 1047-4 | 0.111 | 0.106 | 0.092 | 0.103 | 0.009 |
| 1047-5 | 0.032 | 0.033 | 0.033 | 0.033 | 0.000 |
| 1047-6 | 0.035 | 0.039 | 0.043 | 0.039 | 0.004 |
| 1047-7 | 0.050 | 0.042 | 0.052 | 0.048 | 0.005 |
| 1047-9 | 0.030 | 0.030 | 0.038 | 0.033 | 0.004 |
| 1047-10 | 0.055 | 0.048 | 0.062 | 0.055 | 0.007 |
| 1047-11 | 0.034 | 0.023 | 0.027 | 0.028 | 0.005 |
| 1047-12 | 0.031 | 0.039 | 0.033 | 0.034 | 0.004 |
| 1047-13 | 0.059 | 0.056 | 0.069 | 0.061 | 0.007 |
| 1047-15 | 0.056 | 0.056 | 0.056 | 0.056 | 0.000 |
| 1047-17 | 0.032 | 0.028 | 0.032 | 0.031 | 0.002 |
| 1047-18 | 0.047 | 0.042 | 0.041 | 0.043 | 0.003 |
| 1047-19 | 0.050 | 0.052 | 0.052 | 0.051 | 0.001 |
| 1047-20 | 0.044 | 0.039 | 0.041 | 0.041 | 0.003 |
| 1047-21 | 0.056 | 0.061 | 0.062 | 0.060 | 0.003 |
| 1047-26 | 0.058 | 0.068 | 0.062 | 0.063 | 0.005 |
| 1047-28 | 0.030 | 0.051 | 0.038 | 0.039 | 0.010 |
| 1047-29 | 0.039 | 0.043 | 0.045 | 0.042 | 0.003 |
| 1047-30 | 0.042 | 0.048 | 0.051 | 0.047 | 0.005 |
| 1047-31 | 0.044 | 0.046 | 0.048 | 0.046 | 0.002 |
| 1047-33 | 0.034 | 0.038 | 0.041 | 0.038 | 0.003 |
| 1047-34 | 0.062 | 0.061 | 0.000 | 0.041 | 0.036 |
| 1047-36 | 0.050 | 0.052 | 0.055 | 0.052 | 0.003 |
| 1047-37 | 0.041 | 0.033 | 0.039 | 0.038 | 0.004 |
| 1047-38 | 0.033 | 0.030 | 0.032 | 0.032 | 0.002 |

TABLE 3
PPO activity of three 1-month old tubers.

| Line | Rep. 1 | Rep. 2 | Rep. 3 | Av | SD |
|---|---|---|---|---|---|
| C-2 | 0.135 | 0.141 | 0.138 | 0.138 | 0.003 |
| C-3 | 0.143 | 0.121 | 0.165 | 0.143 | 0.022 |
| 401-1 | 0.155 | 0.173 | 0.094 | 0.141 | 0.041 |
| 401-2 | 0.197 | 0.197 | 0.212 | 0.202 | 0.009 |
| 217-7 | 0.020 | 0.023 | 0.027 | 0.023 | 0.004 |
| 217-12 | 0.018 | 0.021 | 0.017 | 0.019 | 0.002 |
| 217-24 | 0.019 | 0.020 | 0.017 | 0.019 | 0.002 |
| 1045-2 | 0.036 | 0.034 | 0.048 | 0.039 | 0.008 |
| 1045-3 | 0.044 | 0.042 | 0.028 | 0.038 | 0.009 |
| 1045-4 | 0.042 | 0.036 | 0.044 | 0.040 | 0.004 |
| 1045-5 | 0.036 | 0.028 | 0.031 | 0.032 | 0.004 |
| 1045-7 | 0.052 | 0.051 | 0.061 | 0.055 | 0.005 |
| 1045-8 | 0.050 | 0.049 | 0.046 | 0.048 | 0.002 |
| 1045-9 | 0.041 | 0.043 | 0.037 | 0.040 | 0.003 |
| 1045-10 | 0.104 | 0.097 | 0.096 | 0.099 | 0.005 |
| 1045-12 | 0.032 | 0.035 | 0.037 | 0.035 | 0.003 |
| 1045-13 | 0.050 | 0.046 | 0.040 | 0.045 | 0.005 |
| 1045-18 | 0.037 | 0.039 | 0.045 | 0.040 | 0.004 |
| 1045-19 | 0.027 | 0.034 | 0.030 | 0.030 | 0.003 |
| 1045-20 | 0.037 | 0.050 | 0.048 | 0.045 | 0.007 |
| 1045-21 | 0.100 | 0.103 | 0.104 | 0.103 | 0.002 |
| 1045-22 | 0.051 | 0.042 | 0.037 | 0.044 | 0.007 |
| 1045-23 | 0.033 | 0.040 | 0.033 | 0.035 | 0.004 |
| 1045-24 | 0.029 | 0.032 | 0.028 | 0.029 | 0.002 |
| 1045-25 | 0.047 | 0.048 | 0.044 | 0.046 | 0.002 |
| 1045-26 | 0.022 | 0.021 | 0.027 | 0.023 | 0.003 |
| 1045-28 | 0.044 | 0.040 | 0.052 | 0.045 | 0.006 |
| 1045-31 | 0.047 | 0.046 | 0.000 | 0.031 | 0.027 |
| 1045-33 | 0.024 | 0.023 | 0.032 | 0.026 | 0.005 |
| 1045-34 | 0.035 | 0.036 | 0.032 | 0.034 | 0.002 |
| 1045-36 | 0.029 | 0.034 | 0.028 | 0.030 | 0.003 |
| 1045-37 | 0.039 | 0.033 | 0.048 | 0.040 | 0.008 |

C = untransformed control; 401-lines represent transgenic lines only containing the neomycin phosphotransferase (nptII) gene; 217-lines represent transgenic lines also containing a silencing construct comprising two copies of the 3′-untranslated trailer sequence of the PPO gene inserted between the GBSS promoter and ubiquitin terminator; transgenic plants containing both the nptII gene and a promoter silencing construct are indicated as 1045 lines.

TABLE 4
Use of a silencing construct containing F3′5′H promoter
sequences to prevent anthocyanin production in Bintje shoots

| | | | F3′5′H-positive |
|---|---|---|---|
| construct | Total shoots | Green shoots | (PCR) |
| pSIM1165 | 43 | 31 | 32 |
| pSIM1166 | 48 | 37 | 37 |

TABLE 5
Efficacy of various silencing constructs
targeting the promoter of the gus gene

| construct | Total plants analyzed | Silencing-% |
|---|---|---|
| pSIM788 | 35 | 60 |
| pSIM1101 | 34 | 59 |
| pSIM1122 | 35 | 73 |
| pSIM1163 | 35 | 60 |
| pSIM1113 | 35 | 30 |
| pSIM1164 | 35 | 39 |

SEQ ID NO. numbers

SEQ ID 1
ATTTAGCAGCATTCCAGATTGGGTTCAATCAACAAGGTACGAGCCATATCACTTTATTCAAATTGGTAT
CGCCAAAACCAAGAAGGAACTCCCATCCTCAAAGGTTTGTAAGGAAGAATTCTCAGTCCAAAGCCTCAA
CAAGGTCAGGGTACAGAGTCTCCAAACCATTAGCCAAAAGCTACAGGAGATCAATGAAGAATCTTCAAT
CAAAGTAAACTACTGTTCCAGCACATGCATCATGGTCAGTAAGTTTCAGAAAAAGACATCCACCGAAGA
CTTAAAGTTAGTGGGCATCTTTGA
SEQ ID 2
GCCTCAACAAGGTCAGGGTACAGAGTCTCCAAACCATTAGCCAAAAGCTACAGGAGATCAATGAAGAAT
CTTCAATCAAAGTAAACTACTGTTCCAGCACATGCATCATGGTCAGTAAGTTTCAGAAAAAGACATCCA
CCGAAGACTTAAAGTTAGTGGGCATCTTTGAAAGTAATCTTGTCAACATCGAGCAGCTGGCTTGTGGGG
ACCAGACAAAAAAGGAATGGTGCAGAATTGTTAGGCGCACCTACCAAAAGCATCTTTGCCTTTATTGCA
AAGATAAAGCAGATTCCTCTAGTA
SEQ ID 3
CTGTTCCAGCACATGCATCATGGTCAGTAAGTTTCAGAAAAAGACATCCACCGAAGACTTAAAGTTAGT
GGGCATCTTTGAAAGTAATCTTGTCAACATCGAGCAGCTGGCTTGTGGGGACCAGACAAAAAAGGAATG
GTGCAGAATTGTTAGGCGCACCTACCAAAAGCATCTTTGCCTTTATTGCAAAGATAAAGCAGATTCCTC
TAGTACAAGTGGGGAACAAAATAACGTGGAAAAGAGCTGTCCTGACAGCCCACTCACTAATGCGTATGA
CGAACGCAGTGACGACCACAAAAGA
SEQ ID 4
TTCAAATTTCATTTGTGTCATATAAATTGAGACATATAATTGTCGGCACATGCTCATGTATCCAAACAA
GGATAATTTGATCATCTATTCTTATATATTTGAAAATTACGATAATAATACTTTAAATCACAATAATTA
ACAAGTTAAAATATTTAAAAGTCATATAAAAAATTAATTGACTCTCAAAATTCTGTAAGTACTATAAAT
TAAAATAAATAACAACTTAAGAATTTCAAAGTCATAAAAAATTTGGTGGCTCTCTAAAATATATCAATG
TCACATAAAAAGTAACATATATTATTCAGAAATTACGTAAAAGATACCACAAATTACAATAATTAACAA
CTTGAAATATTTAAAATACATAAAAATAATTAATTTTAGAAATTCCAGGCGTGCCACATAAATTGGGAC
AACGAAATAATATATACTATTATTTTAAAATTATGTAAAAAAATAATTCTAAATCATGATAATTAATAA
CTTAAAATATTATTAAAAATCATATAAAAATTTAAATAATTGCTCAGGTTTCAGCCGTATTACATAAAT
TAGGATAAAAAATAATATATATTGGGCCCCGTGCTGGCACGGGGGCCCGTATCTAGTTTATATAATAAA
TATCGTTTCTAGTCTATCTCTTCTGATGCTAAATAAAGTCTGTGATTATCTTTTAATTTTTTCTACTCA
GCATGGGGTGCCGTATCTAGTTTATATAATAAATATCGTTTCTAGTCTATCTCTTCTGATGCTAAATAA
AGTCAGTGATTATTTTTTAATTTTTTCTACTAGGTAATGTAAAATTCTTATGTTAACCAAATAAATTGA
GACAAATTAATTCAGTTAACCAGAGTTAAGAGTAAAGTACTATTGCAAGAAAATATCAAAGGCAAAAGA
AAAGATCATGAAAGAAAATATCAAAGAAAAAGAAGAGGTTACAATCAAACTCCCATAAAACTCCAAAAA
TAAACATTCAAATTGCAAAAACATCCAATCAAATTGCTCTACTTCACGGGGCCCACGCCGGCTGCATCT
CAAACTTTCCCACGTGACATCCCATAACAAATCACCACCGTAACCCTTCTCAAAACTCGACACCTCACT
CTTTTTCTCTATATTACAATAAAAAATATACGTGTCCTTTACGTTATTTCACTACCACTTTCCACTCTC
CAATCCCCATACTCTCTGCTCCAATCTTCATTTTGCTTCGTGAATTCATCTTCATCGAATTTCTCGACG
CTTCTTCGCTAATTTCCTCGTTACTTCACTAGAAATCGACGTTTCTAGCTGAACTTGAGGTAAATTTCT
AGTGATTATACTGTACATTTCGCATAATTTAGGATCGTATTTGATGATATGTTTTACGCTTGATTGATC
GAGAACTTAAAGCTTTTTTGATCTGAAATTTGTTTTTTGGCATACTCGAGTTGAGATCCTGGTTAAATC
AGTGTTATTTCGATTGAATTTTAGAAAAATTTGGTGTTAATTTTCAGTATTTTCATGGTTTAATGTGTA
TAAACAAGCTTAATTTTTCAAATTCAGGCTCGTTTAACCTTTTAATTACAGCATATTTCTGGAAAAAAG
TTTGGTGATTTCTCTAGATGTTTTATTCGAGAAAAAAACAAAAACGAAAAAAGGGGAAATGTCGTTCTG
TATGTACAAAAAGTGATTGATCAGCTTTTGGTCACCGACATACATTTGATTAGTACATACACGAGTCAT
ACGAGTATATTTCCGTGTGCACTTTATTGTTTTGAAGGAATTCTGGATTTGGTTGATTCCTTTTTAAAA
CTTCTAAGTTTTTTTTGTTGCATTTTACTCTAATTAAGTCTTCTCTGTGAACTGACAAATACTCACCAG
GAACACATTACAACCTTCATTTGATTATCCGCGAACGATCCATTGCTTTTGTGTATATTGCTTTTGTAT
TGACTGATTTTGTATTGTATTAGCAGTGAATTAAGCCAGTGGGAGGATATG
SEQ ID 5
AAAATTCTTATGTTAACCAAATAAATTGAGACAAATTAATTCAGTTAACCAGAGTTAAGAGTAAAGTAC
TATTGCAAGAAAATATCAAAGGCAAAAGAAAAGATCATGAAAGAAAATATCAAAGAAAAAGAAGAGGTT
ACAATCAAACTCCCATAAAACTCCAAAAATAAACATTCAAATTGCAAAAACATCCAATCAAATTGCTCT
ACTTCACGGGGCCCACGCCGGCTGCATCTCAAACTTTCCCACGTGACATCCCATAACAAATCACCACCG
TAACCCTTCTCAAAACTCGACACCTCACTCTTTTTCTCTATATTACAATAAAAAATATACGTGTCC
SEQ ID 6
CATTCAAATTGCAAAAACATCCAATCAAATTGCTCTACTTCACGGGGCCCACGCCGGCTGCATCTCAAA
CTTTCCCACGTGACATCCCATAACAAATCACCACCGTAACCCTTCTCAAAACTCGACACCTCACTCTTT
TTCTCTATATTAC
SEQ ID 7
TAATATAACATACCATGGGTGGAGCTAGAAGTCTGATTACAAATTTCGTCAAATTCAACAATATTTGCT
TAAATAATATATTTGTATAGTAATTTTTTTTACAAAATATATACAAATTTAGGTCAAGGATTCAGTTAT
TAACCCTTTAAAATCGTGTCATAAAATTCAATGTTAAAATTCTGACTTTCCCCGTGCTTAACATTACTT
ATCAAATTTATGTTTCTGTGTAGAAAAGTACTAGTACTACTCTTTGACTCGTCTAGACGTCTACTATAG
ATCTCCTTAGATTAAAAACTCCAGTTTTAATATTTTCCTCACAATTATTATTCTTAATCTACCACCTAC
CGGAGTCACAAATATATTAAATGAAAATATTCTATCTATTAATTTATGATCTACCTATTGATAATTTGT
AATCTAGTCAAAATGATGGCAAAAAAAATATAATATCTAGACTGAAGTTCTTAGTCAATAGCGTAAATG
AAAGAAAAAAAAAAAAGCTCAAGAAGAAACATGATATCTTTGTTGCTCTGATTCGTAAAAAAAAAAACA
TAGTAACTTCATAAAATATCTTATCCTTTGGACAGAGCGATGAAAAAAATATATTACTAGTAATACTGA
GATTAGTTACCTGAGACTATTTCCTATCTTCTGTTTTGATTTGATTTATTAAGGAAAATTATGTTTCAA
CGGCCATGCTTATCCATGCATTATTAATGATCAATATATTACTAAATGCTATTACTATAGGTTGCTTAT
ATGTTCTGTAATACTGAATATGATGTATAACTAATACATACATTAAATTCTCTAATAAATCTATCAACA
GAAGCCTAAGAGATTAACAAATACTACTATTATCCAGACTAAGTTATTTTTCTGTTTACTACAGATCCT
TCCAAGAACAAAAACTTAATAATTGTATGGCTGCTATACATAATTCCCCACCTACCGCTTCCTGGAATA
ATTGATATGGAAGCCGCCTCTAAAATTGAATAATTATACTGTTTTACATATTATATAAAGCAAGGTATA
GCCCAATGAATTTTCATTCAAAAGCTAGCAATAATG
SEQ ID 8
AAGTTATTTTTCTGTTTACTACAGATCCTTCCAAGAACAAAAACTTAATAATTGTATGGCTGCTATACA
TAATTCCCCACCTACCGCTTCCTGGAATAATTGATATGGAAGCCGCCTCTAAAATTGAATAATTATACT
GTTTTACATATTATATAAAGCAAGGTATAGCCCAATGAATTTTCATTCAAAAGCTAGCAATA
SEQ ID 9
CTAGTAATACTGAGATTAGTTACCTGAGACTATTTCCTATCTTCTGTTTTGATTTGATTTATTAAGGAA
AATTATGTTTCAACGGCCATGCTTATCCATGCATTATTAATGATCAATATATTACTAAATGCTATTACT
ATAGGTTGCTTATATGTTCTGTAATACTGAATATGATGTATAACTAATACATACATTAAATTCTCTAAT
AAATCTATCAACAGAAGCCTAAGAGATTAACAAATACTACTATTATCCAGACTAAGTTATTTTTCTGTT
TACTACAGATCCTTCCAAGAACAAAAACTTAATAATTGTATGGCTGCTATACATAATTCCCCACCTACC
GCTTCCTGGAATAATTGATATGGAAGCCGCCTCTAAAATTGAATAATTATACTGTTTTACATATTATAT
AAAGCAAGGTATAGCCCAATGAATTTTCATTCAAAAGCTAGCAATA
SEQ ID 10
CACCGGCTGCAGATATTTTTTTAAGTTTTCTTCTCACATGGGAGAAGAAGAAGCCAAGCACGATCCTCC
ATCCTCAACTTTATAGCATTTTTTTCTTTTCTTTCCGGCTACCACTAACTTCTACAGTTCTACTTGTGA
GTCGGCAAGGACGTTTCCTCATATTAAAGTAAAGACATCAAATACCATAATCTTAATGCTAATTAACGT
AACGGATGAGTTCTATAACATAACCCAAACTAGTCTTTGTGAACATTAGGATTGGGTAAACCAATATTT
ACATTTTAAAAACAAAATACAAAAAGAAACGTGATAAACTTTATAAAAGCAATTATATGATCACGGCAT
CTTTTTCACTTTTCCGTAAATATATATAAGTGGTGTAAATATCAGATATTTGGAGTAGAAAAAAAAAAA
AAGAAAAAAGAAATATGAAGAGAGGAAATAATGGAGGGGCCCACTTGTAAAAAAGAAAGAAAAGAGATG
TCACTCAATCGTCTCACACGGGCCCCCGTCAATTTAAACGGCCTGCCTTCTGCCCAATCGCATCTTACC
AGAACCAGAGAGATTCATTACCAAAGAGATAGAGAGAGAGAGAAAGAGAGGAGACAGAGAGAGAGTTTG
AGGAGGAGCTTCTTCGTAGGGTTCATCGTTATTAACGTTAAATCTTCATCCCCCCCTACGTCAGCCAGC
TCAAGGTCCCTTTCTTCTTCCATTTCTTCTCATTTTTACGTTGTTTTCAATCTTGGTCTGTTCTTTTCT
TATCGCTTTTCTATTCTATCTATCATTTTTGCATTTCAGTCGATTTAATTCTAGATCTGTTAATATTTA
TTGCATTAAACTATAGATCTGGTCTTGATTCTCTGTTTTCATGTGTGAAATCTTGATGCTGTCTTTACC
ATTAATCTGATTATATTGTCTATACCGTGGAGAATATGAAATGTTGCATTTTCATTTGTCCGAATACAA
ACTGTTTGACTTTCAATCTTTTTTAATGATTTATTTTGATGGGTTGGTGGAGTTGAAAAATCACCATAG
CAGTCTCACGTCCTGGTCTTAGAAATATCCTTCCTATTCAAAGTTATATATATTTGTTTACTTGTCTTA
GATCTGGATCTGAGACATGTAAGTACCTATTTGTTGAATCTTTGGGTAAAAAACTTATGTCTCTGGGTA
AAATTTGCTTGGAGATTTGACCGATTCCTATTGGCTCTTGATTCTGTAGTTACCTAATACATGAAAAAG
TTTCATTTGGCCTATGCTCACTTCATGCTTACAAACTTTTCTTTGCAAATTAATTGGATTAGATGCTCC
TTCATAGATTCAGATGCAATAGATTTGCATGAAGAAAATAATAGGATTCATGACAGTAAAAAAGATTGT
ATTTTTGTTTGTTTGTTTATGTTTAAAAGTCTATATGTTGACAATAGAGTTGCTCTCAACTGTTTCATT
TAGCTTTTTGTTTTTGTCAAGTTGCTTATTCTTAGAGACATTGTGATTATGACTTGTCTTCTCTAACGT
AGTTTAGTAATAAAAGACGAAAGAAATTGATATCCACAAGAAAGAGATGTAAGCTGTAACGTATCAAAT
CTCATTAATAACTAGTAGTATTCTCAACGCTATCGTTTATTTCTTTCTTTGGTTTGCCACTATATGCCG
CTTCTCTCCTCTTTTGTCCCACGTACTATCCATTTTTTTGAAACTTTAATAACGTAACACTGAATATTA
ATTTGTTGGTTTTTTTAACTTTGAGTCTTTGCTTTTGGTTTATGCAGAAAC
SEQ ID 11
TGGGAGAAGAAGAAGCCAAGCACGATCCTCCATCCTCAACTTTATAGCATTTTTTTCTTTTCTTTCCGG
CTACCACTAACTTCTACAGTTCTACTTGTGAGTCGGCAAGGACGTTTCCTCATATTAAAGTAAAGACAT
CAAATACCATAATCTTAATGCTAATTAACGTAACGGATGAGTTCTATAACATAACCCAAACTAGTCTTT
GTGAACATTAGGATTGGGTAAACCAATATTTACATTTTAAAAACAAAATACAAAAAGAAACGTGATAAA
CTTTATAAAAGCAATTATATGATCACGGCATCTTTTTCACTTTTCCGTAAATATATATAAGTGGTGTAA
ATATCAGATATTTGGAGTAGAAAAAAAAAAAAAGAAAAAAGAAATATGAAGAGAGGAAATAATGGAGGG
GCCCACTTGTAAAAAAGAAAGAAAAGAGATGTCACTCAATCGTCTCACACGGGCCCCCGTCAATTTAAA
CGGCCTGCCTTCTGCCCAATCGCATCTTACCA
SEQ ID 12
AAGCTTTCTTCATCGGTGATTGATTCCTTTAAAGACTTATGTTTCTTATCTTGCTTCTGAGGCAAGTAT
TCAGTTACCACTTATATTCTGGACTTTCTGACTGCATCCTCATTTTTCCAACATTTTAAATTTCACTAT
TGGCTGAATGCTTCTTCTTTGAGGAAGAAACAATTCAGATGGCAGAAATGTATCAACCAATGCATATAT
ACAAATGTACCTCTTGTTCTCAAAACATCTATCGGATGGTTCCATTTGCTTTGTCATCCAATTAGTGAC
TACTTTATATTATTCACTCCTCTTTATTACTATTTTCATGCGAGGTTGCCATGTACATTATATTTGTAA
GGATTGACGCTATTGAGCGTTTTTCTTCAATTTTCTTTATTTTAGACATGGGTATGAAATGGTTGTTAG
AGTTGGGTTGAATGAGATATACGTTCAAGTGAATGGCATACCGTTCTCGAGTAAGGATGACCTACCCAT
TCTTGAGACAAATGTTACATTTTAGTATCAGAGTAAAATGTGTACCTATAACTCAAATTCGATTGACAT
GTATCCATTCAACATAAAATTAAACCAGCCTGCACCTGCATCCACATTTCAAGTATTTTCAAACCGTTC
GGCTCCTATCCACCGGGTGTAACAAGACGGATTCCGAATTTGGAAGATTTTGACTCAAATTCCCAATTT
ATATTGACCGTGACTAAATCAACTTTAACTTCTATAATTCTGATTAAGCTCCCAATTTATATTCCCAAC
GGCACTACCTCCAAAATTTATAGACTCTCATCCCCTTTTAAACCAACTTAGTAAACGTTTTTTTTTTTA
ATTTTATGAAGTTAAGTTTTTACCTTGTTTTTAAAAAGAATCGTTCATAAGATGCCATGCCAGAACATT
AGCTACACGTTACACATAGCATGCAGCCGCGGAGAATTGTTTTTCTTCGCCACTTGTCACTCCCTTCAA
ACACCTAAGAGCTTCTCTCTCACAGCACACACATACAATCACATGCGTGCATGCATTA
SEQ ID 13
TGATTCTATTGACTGCAGAATATTTGATAATACAGTTTTTTGTGTAACTTACTTAAATGTTTTGAACTA
CACGTTTTGAAAAGTTAACCTGTTGGTTAAATGGTTAGCTATGACTCTCGCAACAAACCCAACCCTTAA
GATGATGATGGTTTAACATTTGACAACATAGTTAAGACTGTGTCTATATAATAGTCAACAAATTCAGAT
TGTAGTATTATGGAGTCAACATATTTCGAGATCAAAAACATTCAAAACGTAAATCTATCGACGTCTCAC
ATAGTTTTGTTATGAAGCTGATGAAAAAAGTTGGAAGACATAGTTTTGCAAACATCATTTGTTGCTAAC
GTATAAACGTTGGTTTGATTAAATGTAATAGGATAAGGATATCCGTTTGTTCATATAATTGAGTTAAAT
TATATTTTGGTTATTATAATATGTTAAGTTGAAAATAAATAGGTCCAACAACCTTGTTTAAATAGATTT
TTTAGGAGTGATTCCCTTTTAATAGTATAGATTATACTCTCTTCCTAATCGACCTTCCGTGGGGTAAAG
TGGTCAATTATATTCTTTATGGATGAGCTTGATTGAGAATGGGTTTATGGGTTATGACAAGGGCATGTA
CAAATGTCACTGCCTCTTGACATGCAACCGAACAGTTGGCGACTCAAGTCGCAGAAGATACAACGGACC
AAACCCTCCGAGTGTCGCCGCGTCTGTTATGTGTCACCTTTTTGTCTCCTTTCCTTAAAAATTGGTAAC
TCATTTTTCAAAAAAAGAAGAGGATAGTTTTGGCTGTATCTCCTAAACTATTCGATCACAACGCCAGAT
ATTTTAATACTGGATACTAGTGATGTAATTTGATTTGTTAATTGTCAAAAAGTAGATTCTCCTATCTCG
TTTTTAGTTCAATTATTATATGGTTAAATGAATTTAAGTCGATTAGAAATGATTAGTTAATCAACCAGA
GTTGCTCTATAAGTCTATACTGATAACATGAACCATTTTCTAAAAATGAGATAGATACATTTGAATTTT
GTCGTGGTTTGGAGTATGCGGAGATAGTCGTACGCGCATGAACATCATGAGACACTTGCTTCAGCTCAC
AGAGTGACGTGTAAAGACCATAGACCCACGACTTCATGCAAACCCATTCCTACGTGGCACAAACCTTCA
TGCTCACTCCACATATATAAACTCCTACCAAGTCTCCATGTTTCTTCATCCATCTATCACAAAAACACA
CAAACAAT
SEQ ID 14
GTCGACTCGATCACGGCACGTGGATGAGAGAGAAAATGAGAAACAAGTGGTGGAGTAAAATGACGAAAA
TAGGTCCCTATTCCAAGGAGGGAAAGCTTAAAACAAAAAAGCTTAAATACAGGCGCCCCCCTTGAACAC
AGAAA
SEQ ID 15
CATATGTGAAATGTAATGGAAAATGCGACAAGAATTGCAATAGAGAAAATCCAATTTGCAGAGATTACA
TGAAAAGAATTTGTACAAATAGCATATATATGTTAAAATGAAATGGGACATGCCACATTATGTGGAATA
AAAAAGACAATTTGCTTGGAATTAATTATAGAATAAATGTGTTACATTTAATATGTGATTAATCACTTT
TTTTGAATTGTACATCTATCACATGACAAGTTCATTATATTTGACATATAATTTGTTTATGTCTAGTCA
AGCCTAATTAAATTTCTCGGAAAGCACAAAATTTTTTTGTCCTAACCAGGTTTGAACAACCAAACAAAT
CACAAAGCAGGTGTATCGCACTTGCGATGTGATCGGTCACTTTTTCTAAATTGTACATCATTCACACGA
CAACTGTATTGTGCTCCAAGTTCAATTGAGTGCGGTTGGAGCTATAATTTCCTTGAACACACAATGTGG
AATGTGCACACTCCATGTGGGCCAATGAGCGGATGACACGTGGCGGGCAACTTACCTCGTTACGTTGAG
GCATGCATGAAAGGGGGATCTCTTGAGGTGGAGGGGTGGGGGCGGGGGTTGGGGGGGGGCCCCTCCTCA
GACAGGTCTATATTTATGAGACCTCGTAAGGCAGAACGC
SEQ ID 16
TGTTTTGTTTTTGGTTATGGGATTAATTTTTTAATTACGAAGAAGCTTTTAGAGCATCACCCGAATCTA
ATTCGTTTTGGCTTTTGTGATCTTGATGTAAATCTATACTAACTTGGTTTGGGCAAGAGAAATTGGTCC
TTGCTCAAGTCCATTCTAGGACGAAAATAAAAATATAACAGGGTATAGCAGATCTCTATTCGTATGTGG
GTAACGATAGCATGTTTCTATTGTTCTCTTATTCTTCATTGGTCACGATAACCTGCTAATTATGCCACG
ATTGAGATGAAAAGTAACGAACTAGTAAACCATAGTGAGAAGAACATTTCGCTACTATTGTTGAAACGT
TTACACCAGGCACTTGAGTATGATGCACTATATTTCAATTAATGTAATTTTTCGCTTTGATGAGAAACA
TTCTGATTCTGTGAGTTTAGAAACTATTGCTGATAATCCTTGATTTAAGATTTCAGTCTTGTTCATGTT
CATTTGAAGTGTTGGTAATAAAATGCACTGATGTGTCATGTGCA
SEQ ID 17
TAAATATATACTTTTTTAGTGTTGTAAATTTTAATATGGGTCGGCCCGGGCCGAGCTCGGGCTTAGCAA
TTTTTTCCGGGTCGGACTTGGATAAATTTTTAGGCTCATATTTCGGGCCGGGTCGAATCCGACCTAAAA
AATAAGCATAAAATTTTGTCTTGGATCCAGCCCAAATCTAGCCCGACCCATAATCACCTCTAGTTTAAG
CTTCTTCTTTTCTTTCTTTCTTTCTTTCTTTCTTTCTTTTTTTTTTTTAACATTAAAAATATGTAGAGA
AAATCAGCAATTAAAACAAAAGTTAGGGCTAATGTGTTAAAGTAGCACCAATAAAGTATCCCTCTCAAG
TGAAGTCTTTCACACTTGCAAACAAAAATAATTAAAAGACAGAGGAGTCTATAAAGTTAAAAGCCGTCC
AAAACCCAAACCAGGAAAGGCAAA
SEQ ID 18
GAGCTCTCAATGTAGTAACACAAACTCTTTTTTTTCCATAACGTTGAATGTTAGAACTTTGTCTTTTTA
TAACTGTTTCTTTCATGAAGCTGATCAGCTGATGTTGGAGAAGGATGGAGCCACGGAGATTCCTGAAAA
GCAAAGGATGGAACGAGAGGAGACGGTGACTCGAGAGTACAGGGAAGCATTGCACAGAGCTGTCACGCT
TGCAGTGCCTCATTCAGAGTTCTTGTCTCGGTATGGAACATTTAGTGGCGGTGACGTTGAAGAAGAGGA
AGAAAGATGCTATGGTTCATCATCTAGTGGGAAGGATTGATCCAGCCGGCATGTTCTCCTCCCGAAATC
GGGCCGTCCCAATTGATGACAATGTAACATCAATGTCAATCTCTGCAGATTTTTGTTAGCAGCAGGTCA
TGATTCTTTTTTGGTTGATTCTTGTGAATGTAAGCTATTTGTTGTTGTAATATATGCATTGATTGTGAT
TTTGTTTTAGCTTTGATCAATGAAATAAATCTCGTTCAACCCAACCATCAGGCTCTTTCATATTCATTT
TGACGACTATATATACATAATCGTACAAACTATTCGGTTAACTAATCTACAGAAAGTCGGAGTTAGCTA
GAGATTGTCAAGGAGGAGGAGATCATACACCTAATTTTGAAGCTGATTCTTCATCTATGATTTCGAGTT
TTGACTTGATTTGGCTCTTCGATATTCGAAATTAAATGCCTCAATGCCTCCAAAGTGCTCTCTACTTGC
GGGTGGACCTACAAAACTAGGCAAACAGGTGCAAAAAACATGTGTTTACACGTCCATGTTATCTTGCAT
TGGCCCATGTTTTCTGCATTGTAAATCTTTCCCCAAACACATAGTTAGACGAAGTCGATAATCTAGCAC
CATCAAATCAATAACACGAGCAAATAATAAAGTAAATAGTGAAACCATGAAGCCTAATTGGTCGAGTGG
AGCTGAAAGCTTTCATCGGTATCGAACCCAACCCCCCCTGCTACGAAACTTAAAAATGGGTTACGCTAT
TACACTCGATAGAACTGATGAAACGCAACGATTGTTAAGTAACCATTTTGCAGAAACGATAATTGACAA
GTGACCATTTGGATAAATGACCAGGGAAAATACAAGTGGCGAGTGCTGACATAATAAACCGAATGCGGG
CGTTACCATCCAATTTTA
SEQ ID 19
GAGCTCTCAATGTAGTAACACAAAGCCTTCTGTCTTCTTTCTGTAACGTTCAATGCTAGAACTTGTCTT
CTTATAACTGTTTGTTTGCTTCTTCAGCTAATGTTGGAGAAGGATGGAGCCACGGAGATCCCGGTAAAG
CAAAGGATGGATCGAGAGGAGACGGTGGCTCGAGAGAACATGGAAGCATTGCACAGAGCCGTCACGTTG
GAAGTGCCTCATTCGCAGGCCCCGTCTCGGTATGGAACATTTGGTGGTGGTGAGGTTGAAGAAGAGGAG
AAAGATGCCGTAGTTCATCATCTACTGGGATGGATTGATCCGGCCAGCATGTTCTCCTCCCGAAATCGA
CCTGTCCCTATTGATGACAATGTAACATCAATGTCAATCTCTGCAGATATCTGTTAGGATCAGGTCATG
ATTCTTTTTTGGTTGATTCTTGTGAATGTGTAACATTGATGTAAGCTATTTGTTGTTGTAATATCTGAT
TTTGTTGTTGCTTTGATCAATCAAATAAATCTCGTTCAACGCGATCATAAGCCTCTTTCATATTCATTT
TGACGACTATGTATAGTCGTACAAACTATTCGGTTAACTAATCTACATCAAGTCGGAATTAGCTAGACA
TTGTCAAGGAGGAGGAAAATATCAAGAAAATTGGATGAGGAAATCATACACCCAATTCTGAAGCTGATT
CTTCATCTATGATTTCGAGTTTCGACTTTTTTTGAGTCTCAACTGTGATTTCGAGTTTCGACTTGATTT
GGCTCTTTGATATTCGAAATTAAATGCCTCCAAAGTGCTCTCTACTTGCGGTTGGCCTGGTTCAATGGC
GAATCATTGAATGACAGAACTAGACAGCTACCAGGTGCAAAAAACATTTGTTAATGTCTTCTTGCATTA
ATGTCCATGTTTTCTGCATTTTAATCTTTCCCCAAACACCTAATATATAGCTTCATTGATCCTCCTCTC
ACGGTTGCAGATCTCGTTGCTGATAACACATACATGGCTACAAGACTCTAAAACGGTTCAAAGTGAAAT
TGTTTTGGTGGTAGAGTTGTGTGTTTGGTGACTCGAAAGTTCTGGATTCGAATCCAGCATTCCCCACAA
AATAGACACCAACGTAGTGTTTATTTACCGTCTTCTATCTTGTATTGACCGAGAGTTACGATATACTCC
GACAAAAAAAGACATCTTCCACATCATCAAATGGATCCGTAGTTAGTGCAGTGGCTCGATTAACATAAA
TGAAAAAAGGAAAAAATTTGCCTGAAATCGATGCTCAAAACAAGTAGAAATTCATTCAAACATATTTAG
ACAAACACGATCATTTAGCATCATCAAATTAATAACAAGAGCAAACAATAAAGCACATAGCAAAACATA
CAATAGTCGTCTTGCAATGTCATATGATAATAAGCCAGTGAAACCATGAAGCCCAAGTGAAGTGGTCAA
GTGGGAGCTGAAAGCTTCCGAACCCAAGCCCCCGCTACCGGGTTAGGACATACGACACGCGACATGCTA
CGAAACTTAAAAATCGGTCACGCAGTTAATGGAACAAATGAAACGCAACGACTATTAAGTGACCATTTT
GCAGAAATGATATGAAAAAGTGACCATTTAGACAAATGAGCAAAGAAAATACAAGTGGCGAGTGCTGAC
ATAATAAACCGAATGCAGGCGTTACCATCCAATTTTA
SEQ ID 20
AAATGAAAGAGAGTTAAGGATTGAAATGAAACTGGTAAAAAACAGCTTATTTTAAAACATCTTATTCAA
AACAACTTATTTTATTTAAAACAATTTATTTTATTCAAAACATGTTTTGAATAAGTTGTTTTTTGAAAA
TAAGCTGTTTTGAATAAGCTGTTTTTAAAATAAGGTGTTTTTCATAAAATAAGTTGTTTTTGTTAAAAT
AAGTTGTTTTTTCAAATAAGCTGTTTTGAATAAGCTGTTTTTTTTTAAATAAGTTGTTTTGAATAAGCT
GTTTTTTTTAAATAAGTTGTTTTTTTAAATAAGCTGTTTTGAATAAGTTGTTTTAAAATAAGGTGTTTT
GCATAAAATAAGCTGTTTTGAATAAGTTGTTTTGAATAAGTTGTTTTGAATAAGCTGTTTTTTTTAAAA
ATAAATTGTTTTCATAAAATAAGCTGTTTTTAAAATAAGGTGTTTTGTATAAATAAGCTTTTTAAAATA
AGCTATTCAAATAAGTTGTTTTTTTGGAAAGATCCAACAAAGAGTTCAAGTGGTTTCTTTAAAATAAAA
TAAAAAGTTCAAGTGGTTTGGTTCGGTTCAAACGGTTCGGTTCGGTTCAAGATGGTTCGGTTATGGTTC
AAGAACTGTTAATAAATTAACGGTTCGGTTCGTGAACCATTATAACGATTCGGTTATTTTTGGTTCGGT
TCGGTTCGCGCGGTTCGGTTCGGTTCATGGTTCTTTTTGCCCACCCCTAAAGAAAATAAATGAATGGTG
GTTGAGTATTCTTAAAATGATTTGTTTTCTAGAATAAAGAGTTAATAAGGGGGTCAAAAGAGCAACCAT
CTAAGGTAAACTCTCACATTTAGAGTTGATGCGGTTAAAATTTGGATATAACACTTTTGTTGACCAAAA
TGTCTCTTATGAATAAGACTGAAAGAAGTAATAATTTAAAAAAAAAAAATCCGGCTGTTGCATTTTTTA
AAACATTAATCCGAAGAAAAGATGTTTGAAAATTGTTTATAATGAGAAGTTATTTTGA
SEQ ID 21
CACCAACATGATTTTTGTATGCTTGTAAATGAAAAGCTTCTAGTTATCCAGCTCAACCCGTGACTAAGG
TCTATTCAATTTGCTTAGAAATGAGGCATCAATTATGATGCAAATTTTTGTACTCATTACTCAATTCAA
AAACTATATGAACTTATGGTGTCACGTAAGTGAATAACACTATCTAAATTTGAGTACTTCTCCTGTCAC
GGGGAGAAAAACACTCAAAATCAATTGCATGCAACGGCAACACATTTCTGTTTACAATTATATTCGGTG
AGTACTCAGTCAGTATAACCCAATTACCACATATGCACGAATTCTCTTAGTGGGTCCACATTGTGGTGG
TTGAGTGGGACCCAATTGTAATGGATGGCCCACATACACCAAACTCAACCAAACAATTTCTCATAAAGT
TCTATATAATAGCAATCCACTTTGCATCATTGAG
SEQ ID 22
ATAGTGGACCAGTTAGGTAGGTGGAGAAAGAAATTATTAAAAAAATATATTTATATGTTGTCAAATAAC
TCAAAAATCATAAAAGTTTAAGTTAGCAAGTGTGCACATTTTTATTTGGACAAAAGTATTCACCTACTA
CTGTTATAAATCATTATTAAACATTAGAGTAAAGAAATATGGATGATAAGAATAAGAGTAGTGATATTT
TGACAACAATTTTGTTACAACATTTGAGAAAATTTTGTTGTTCTCTCTTTTCATTGGTCAAAAACAATA
GAGAGAGAGAGAGAAAAAGGAAGAGGGAGAATAAAAACATAATGTGAGTATGAGAGAGAAAGTTGTACA
AAAGTTGTACCAAAATGGTTGTACAAATATCATTGAGGAATTTGACAAAAGCTACACAAATAAGGGTTA
ATTGCTGTAAATAAATAAGGATGACGCATTAGAGAGATGTACCATTAGAGAATTTTTGGCAAGTCATTA
AAAAGAAAGAATAAATTATTTTTAAAATTAAAAGTTGAGTCATTTGATTAAACATGTGATTATTTAATG
AATTGATGAGAGAGTTGGATTAAAGTTGTATTAATGATTAGAATTTGGTGTCAAATTTAATTTGACATT
TGATCTTTTCCTATATATTGCCCCATAGAGTCATTTAACTCATTTTTATATTTCATAGATCAAATAAGA
GAAATAACGGTATATTAATCCCTCCAACAAAAAAAAAAAAAAAACGGTATATTTACTAAAAAATCTAAG
CCACGTAGGAGGATAACATCCAATCCAACCAATCACAACAATCCTGATGAGATAACCCACTTTAAGCCC
ACGCACTCTGTGGCACATCTACATTATCTAAATCACACATTCTTCCACACATCTGAGCCACACAAAAAC
CAATCCACATCTTTATCATCCATTCTATAAAAAATCACACTTTGTGAGTCTACACTTTGATTCCCTTCA
AACACATACAAAGAGAAGAGACTAATTAATTAATTAATCATCTTGAGAGAAAGCC
SEQ ID 23
AGAGAGGAGGCAGTGTACACAGGGGCAGAGAGAGGTGAGTCGTCTTTCTGGTAGGGCTGGTGTTGGGGA
TAGTGGTTGGTTTGAGAGTCAGGTGGTGAGGAGGGTTGGCGATGGGGTTGATACGTTGTTTTGGTTGGA
TAGGTGGTTAGGAGATGCTCCTTTTTGTGTTTGTTTCAGGAGGTTGTTTGAGTTAACAGAGAACAAATT
TGTGTCTGTGGCTAATTTGTTATCTGTTGACTCGGAGCAGTGGGGGGAGGTGTTGAGGTGAAGCGTATG
GTGGCAGAGGTGGTGGCAGAGGTGAAGCGTATGGTGGCAGCTGAGGGAGGCAGTGTACACAGAGGTGGA
GAGAGAGGAGAGAGAAGAGAGAAGAGAGAGAAAATGGAGAAGAGAGAAGAGAAGAGAGAGAAGACAAAT
TTTTGTGTGTGTGACCAAACCAAAATTCTTGGTCCTGGTCCACACAAGATTTTCTCCCAACCAAGGTAC
AAGAATACCACGATCCAAGAGTGCCACGTTGCAACATCATAACCGTTCAATAGTAAGAGATAATCGAAC
GGCCATAATTAATTTTCAACAAACCCACTTTTTTCCTCCTACTTTTGCAACTTGTCCCTCATCACCTAC
CAAACACACATAGCACACCAACACACATAATAATATTATAATAATTGTAAATATATGTAGCCTCCAAAT
TAGAAAGAAACCTCTATATAAAGCCTAACTACTTCCTTCACAAATCAGGAAATTCACAACTCTAATATT
CATTTCTTTCCTAATCATTAGAATTTCCATTCTTATAAAATTCTAGGTACCACCACACAACAAATAAAG
GAACATTAATCAATACTATTAAGATGGATC
SEQ ID 24
CTTCTATTAATGATTTAATCAACCTTTTTTAAAATACGAAGGTGACCTTATTTTGCAAATAATCCATGC
ATGGAAATGCATCATCCTTTTGAAAATGGGATTATCTGAATTCTTAAGTTACGTGAAAATTTAATACAT
TTCATTTTAGATAAATTTATTATTAAAATTCACACTTAGATGGCCTAAAAATTAACACTTATTTTTAAC
AATTCAAATAAAATATACGACGAAATGAGTGTAATTTAGTTGGTTAAGCATCGTCAAGCTTGGAGAGAA
AGATCATAGTTTGATCTTTGAAAACTACACTATTGAAAAGGGTGAAGATATCTAAACATCCAAACAAAA
TTTATTTTGATAGTCGATTCAAATTATCAAAATTTGTGAAAATATTTTGTAAATTGTTAAGTTGGCAAA
AATATGTTAATTTTCAAATTACCATTTGCACATTTTTCTAATCTCAAATCACATTTAAGGGATGTTGAC
TACTTTAGTTTTGTACAAATCTTTACAATTTTAACATTTATAAAATGTGTTTCGGTAGATAAAAAGTGT
GAGTATTGTTTATAAGAGATTGTGTTTTTCTTTTGTTTAAACTTATAAAATAAATATATATTTTATTTT
ATTTTAATGTGAGATTGTAAGAATTCATTATAAGATTATGTCATTCCCTCAAAAGAAAATTAGATGATG
TCATTTTCATAACTCATTTTCTATAAATACAGAAAATCCTCAAAAATGAAAAACCTCAGTCAAAAAATA
AAAGAAAAACATCAATAGTGGACTGGCCCACACTCATTGCTTTGCTTTAGTATAAGAAAGTAGACCTCA
CCAACCACGAACCGGACGCCAACCGGTTCAACCAAACATTACACCAATTTTCCTTAACCATACCGGTTT
TTCCCTCCCTTATATAACCATCTTCCTACCTCTTATCTAACCAAGCTCCATTCAACTCTTCAACACATA
TCAGAAACAGAAAAAGAAGCAAAACATTCCAAGAATTTAACA
SEQ ID 25
CATCAATAGTGGACTGGCCCACACTCATTGCTTTGCTTTAGTATAAGAAAGTAGACCTCACCAACCACG
AACCGGACGCCAACCGGTTCAACCAAACATTACACCAATTTTCCTTAACCATACCGGTTTTTCCCTCCC
TTATATAACCATCTTCCTACCTCTTATCTAACC
SEQ ID 26
TGTACATTAGAAGTTCCCATCATATACTACTGTCTAAAGAAATGCATTAAGTTTTGTCCTATTTATTTG
ATTTTTTTCCTTTCTTTCAATTTCAACTGTTATTTTGATTTTTTGTAACCGGAACGAGTTCATGACATA
CTGTTACTTATCTCTTCACTTTTATGGTTTTTACATTTTTTTTTTTTTTTTTTTTTTTTTTCGGCAATG
ATTTTCACTTTTATAGATATATAATTAGAAACCTCTACTCCTATTTTTATCTCCCTATCAATGATGATA
GCAAAATTGTATA
SEQ ID 27
ACATGCACCGCCACCAAGATATCCTACTTTCTAGTGTGTCATTCAAGACTTATTATGGTGTATCATACG
GAAAGAAGAAAAATAGGAGAGTGTATGGTGTTGAATTATTGACCATACAAAACAAAATGAGGTTAGATT
TGCGAAGGATAAAACCTTTGACAATTACCAATGCGATAAATCCCTCACGAATATTTATTTTGTGATGAA
TTTTTGCACTTGTGAGAGATTTAACCCTCACAAAAGAGTCTTATAGTGTTATTTTTATATTAATTTGTT
AATTAATATGTAGGAATGTAGTATAATTAAAAAGGTGTAGTCATTTATCCTATTACTTACAATATTGTG
ATTTGAGACACTCTTTAAGTAAATGATGATTGATAAGTATAGTAGTATAAAAATTTATAAATAATATAA
TGTATGCATTGGGTTGACCGACATTTAGAGTTGAATCTAAAGTCATGGTCATGCATGGTTGCTTCCACC
ATATTTCTTGCCAACTACCTCGTGTTTCTCTTAGTCTATTGCCATCCACCCATATGCATCTATCTACCA
ACCCAAAAACAAAGAAAACCAAAACCCTAGATTGCCACGTTACAAAATCTTAACTGTTCATTAGTAAGT
GATGATCAAACGGCCATAATTAATATTCAACAAACCACTTTTCTTTTTTTCTACTTGTGCAACTTGTCT
TTCCTCACCTACCAAACTCACATATCACACCAACACACATGCAATGCACAATACTACATTTCAAAGTCT
CTATATAAAGCTTAACCACTCTTCCTTCACATCTC
SEQ ID 28
CTCATAATTAATTTTCAACTAACCCACTTATTTTCTCTACGTACTGCTTGTGCAACTTGTCTCTCCCTA
CCTACCAAACCCACACATGCATAATAATAAGAGAGAGTTAATAATATTACAATAATGCATATTAATGTA
GCCTCCAAAATATACTTTATATTTTATTTTATTTTGATGCCAAACACACCTCTATATAAAGCTCAACAA
CTCT
SEQ ID 29
ATAATATATATTTTTAATATAGTTATAATATTTGCAAATTAAAACAATAAGAAAACATTAAATTGCCAC
AAAAAATAAAAAAATTTAAAAACATCATTTATGTCGAAAAACAAACATGTATTTATTCTTTAACTAATT
AGATTTTAGATTTGTTTTTTAAAAATTATCAATTTGAATCATTTCAAATTACTGGAGACTTACATAATC
ATTAATTAAAGACCCATATAATTAATCAAGATATATATAAATTCATCTCGATATCTATATAAAAATCCA
GCAGGCCATTTGCATGATTATTAGGAGGATCCATGTGGTTTTATTAATTACAGGAGCACATATATATAT
ATATCTATATATAAAAGAAGGGCAAGACGAAATTTCTCATTTCTCATTTCTCACCAACCACAACCTCAT
CACCATGCATCACACTGCACGATAGTCAAATTTACCCTTCTACGCCAATCGCCAATATGGATCCACAAA
GAGACCACGCTCCATAATATTGACCCTTGAGATTATTCAATATCAATGGTAACAATTGAGTTTCAACAA
ACCCACTTTGTCCCCTCATGCTTACCTACCGACCTCCATGTCTCTATGCATAGTATTCAAGACTCCCAA
CGATCTATTTAAACCTCCTTCCCTCCCTCTCTTCTCC
SEQ ID 30
TGGGGTGGAGAAGATGACAATGAGAAAGTCGTCGTACATATAATTTAAGAAAATACTATTCTGACTCTG
GAACGTGTAAATAATTATCTAAACAGATTGCGAATGTTCTCTACTTTTTTTTTGTTTACATTAAAAATG
CAAATTTTATAACATTTTACATCGCGTAAATATTCCTGTTTTATCTATAATTAATGAAAGCTACTGAAA
AAAAACATCCAGGTCAGGTACATGTATTTCACCTCAACTTAGTAAATAACCAGTAAAATCCAAAGTAAT
TACCTTTTCTCTGGAAATTTTCCTCAGTAGTTTATACCAGTCAAATTAAAACCTCAAATCTGAATGTTG
AAAATTTGATATCCAAGAAATTTTCTCATTGGAATAAAAGTTCAATCTGAAAATAGATATTTCTCTACC
TCTGTTTTTTTTTTTCTCCACCAACTTTCCCCTACTTATCACTATCAATAATCGACATTATCCATCTTT
TTTATTGTCTTGAACTTTGCAATTTAATTGCATACTAGTTTCTTGTTTTACATAAAAGAAGTTTGGTGG
TAGCAAATATATATGTCTGAAATTGATTATTTAAAAAC
SEQ ID 31
CATGTCCCTAAAAGAGACCCCGCCTAACCATGAGTTTGTCCGAAAAAAATGTATTGACCCATTGCTTAT
CTCCCGTCAAACATTAACGTCGAACCAACTTCTGATCCCTAAACCAATTGTATCCCTCACCTTTGCCAT
CTCATTCCACCACTCAGACCCATTCTTATCTCTATTCATCAACCTCCCTCCCTCCTCATCGTACCTCGC
CACCAACATTCTATTCCACAACTCATCCATATCCATCAACACTATTTTTCTAACAATGCAATATTAAAA
TCCCACATCTTGCAGAGATCATTACATGAAGTTATACTTGTACGGGTCTTGAAGAAGAAAAGTGTGTTA
ATAGTTAGTTTATTAGATTAATATTTATTCATTTGTGCCGGATTTGAATTCAAAACATTCAACTCTTTT
ATCTTAATTCAGACCGGTTGAACTATTTAATCTCTAGATAAAATTAGATGTTGTTGAATGAATATTCAA
AATTAATGGGTGTTAAATCCTTACAAAGTGAGTTCGGTCAAAAAAAAAAAACCATACAAAGTGAGTTAC
ACTTTTTTTTTTTTGAGAGATAAGTTATTATACCAAAAAATACCCAAACATAACACAAAAATGAATTAA
TTACTTTTTACAAAGACCATCCAACCATGAACCATTAACTCGATGAGAAAAGAGAATGCAATTCTTAGT
TTAATCTACACACAAAAAAAGACAACACACACCAAGGCCACAAACCCCACCTAACCCTCTACAGTAAAT
CCACCTAACCAAAACCCCATACACATCATCATCATCATCATCATCATCAAAACCTCTCTATAAAAACCC
AACAACCACTCCAAACATTT
SEQ ID 32
ATTAATAAACGCAAAGTAGTTTGTCACACTATAGGAGAAAATATCTAATAAAAAGTAAGACCTTATAGT
TTCAAGAGGTTAGGTTGATATTTAAAGAGAGATTTCTTTCATTAACTTTTTAGGTTGAAATCTTGAAAT
TAATATTAAAAAGATTTGATAATCCTTTTACTGTGAATACTTTGGATTGGGATTCACATTTAAAATTAT
TCTTAAATGAAACTTTATGTTATATGTTTGATACTGTATTTTTACTTGTTTTTAAAATGTATCTGTTTT
TTAAAAATATCAAATTATTAATTTTTTATTGTTTTTTAAAAGATTTTAATGTATTAATTTTAAAAATAA
AATAAAATTATTTTAAGTGTATTTTTAAATAAAAAATATTTTCTAATAAAAGATTTGAAAAAAAAAAGG
ATAGGAAAAAAACTTTCTTGGTGGAGAGCCTTGTCCCTCGAAGCTTAAATCATCATAGATTAGTGGCGC
CCACATTACATCTTGTATAGAAATACAAAAAGGCCAGGGAAATTAATTAATATGATGACCATATGACAT
TTTCGGCCACCAACCCGCCTTAOCTACTACTATCCATGATTATCAATGACACTCTCCTACCACCTCAAA
TGTAACGCCGTTAACTCTCTCTCTCTCCCCCACACACACAACCCAACGCGTGAAATTCAACTTCATTTC
CTCTCTAATTTTTGCAGTTATAAAACCCAAGCTCTCCTCATCCTGTTGCTCCCATCC
SEQ ID 33
ATTATTCTTAAATGAAACATGACGTGTGTGAGTTTGGTATTGTATTTTCACATGTTTTTAAAATGAATT
TGTTTTTAAAAAATATTAAATTAATAATTTTTTATTGCTTTTCAAAGATTTTAATGTATTAGTTTTAAA
AATAAAATAAAAATTATTTTAATGTATATTTTTTAAAAAAATATTTTCAAATAAAAGAATTAAAAAAAA
AGGATAGGAAAAAAACTTTCCTGGTTGAGAGCCTATCCCTTGAAGCTTAAATCATCATAGATTAGTGGC
GCCCACATTACATATTGTATAGAAATACAAAAAGGCCAGGCAAATTAATTAATATGGTGACCATATGAC
ATTTTCGGCCACCAACCCGCCTTACCTACTACTATCCATGATTATCAATGACACTCTCCTACCACCTCA
AATGTAACGCCGTTAACTCTCTCTCTCCCCCCCAAACACACAACCCAACGTGTGAAATTCAACTTCATT
TCCTCTCTAATTTTTGCAGCTTATAAAACCCAAGCTCTCCTCATCCTGTTGC
SEQ ID 34
TCTTGTTTAATTTAATTATTCTCCAGAACAATCTAGTCCTTGTTAATTAAATTAATTCAGAGTGTTTTG
GTCCTAAATTAACTGTTAATATTATATTTTGTTTAATTTAATCATTCTCCAGAATGTTCTGGTCCTACA
TATATTAAGTACTATTTATTTTGTTGAACTAACGTAAACTAAAATCAAGAGGTTCTCGTAGAGTACTAC
GAATATATAGGGTGCTAATACCTTCCCTAAAAATATAATCAACCCCCGAACCCTAAATCTTTTCAAAAT
GGGTTGTTTTGAACTTTTTCCCCTTTTAAAAAAAAATTGTTCAGTCGTGAAATAAAAGTGAGTCAAACG
CTAATCAAATGGTCTTGATCTCCAAAAAATGGCGCGACAAAAATTAAGCAATGT
SEQ ID 35
AAGCTTCTTAAAAAGGCAAATTGATTAATTTGAAGTCAAAATAATTAATTATAACAATGGTAAAGCACC
TTAAGAAACCATAGTTTGAAAGGTTACCAATGCGCTATATATTAATCAACTTGATAATATAAAAAAAAT
TTCAATTCGAAAAGGGCCTAAAATATTCTCAAAGTATTCGAAATGGTACAAAACTACCATCCGTCCACC
TATTGACTCCAAAATAAAATTATTATCCACCTTTGAGTTTAAAATTGACTACTTATATAACAATTCTAA
ATTTAAACTATTTTAATACTTTTAAAAATACATGGCGTTCAAATATTTAATATAATTTAATTTATGAAT
ATCATTTATAAACCAACCAACTACCAACTCATTAATCATTAAATCCCACCCAAATTCTACTATCAAAAT
TGTCCTAAACACTACTAAAACAAGACGAAATTGTTCGAGTCCGAATCGAAGCACCAATCTAATTTAGGT
TGAGCCGCATATTTAGGAGGACACTTTCAATAGTATTTTTTTCAAGCATGAATTTGAAATTTAAGATTA
ATGGTAAAGAAGTAGTACACCCGAATTAATTCATGCCTTTTTTAAATATAATTATATAAATATTTATGA
TTTGTTTTAAATATTAAAACTTGAATATATTATTTTTAAAAAAATTATCTATTAAGTACCATCACATAA
TTGAGACGAGGAATAATTAAGATGAACATAGTGTTTAATTAGTAATGGATGGGTAGTAAATTTATTTAT
AAATTATATCAATAAGTTAAATTATAACAAATATTTGAGCGCCATGTATTTTAAAAAATATTAAATAAG
TTTGAATTTAAAACCGTTAGATAAATGGTCAATTTTGAACCCAAAAGTGGATGAGAAGGGTATTTTAGA
GCCAATAGGGGGATGAGAAGGATATTTTGAAGCCAATATGTGATGGATGGAGGATAATTTTGTATCATT
TCTAATACTTTAAAGATATTTTAGGTCATTTTCCCTTCTTTAGTTTATAGACTATAGT
SEQ ID 36
TGGCATGATCTCAGTAAATGTAGTGTAGTGTGTACATGAATTATACATCAGTTTTGAAGAGGTAGTATA
ATGGAAGTATCATATCAAGGGTATGGCCATATTTGCAATGACAAATGTAAAATGTGATGAGCCACATTA
GGAGTGATTCCGGCGTCCGTTGTCAAAGTTAAATTTGTTTCTACTTATTATGCAACAATCAAAAACTTC
TTTAACTTCTGCAGAATGATATAAAATGAGAGAAAGATGCACCAACCTATGTACAGTTTTTACTTTTGT
CATATCGCATACTTTTTTTCTTTTTGCTTTTCCTTATCTGCCATGGAAAAAAGATGTCCCCTAATTATA
CACAAATTAGGGGTGTCAAGTGTCAAAAAGGGCGGATTATGTTTGAAATTGATCAAGTTAAAATGAGTT
GAATTCACAAATAGGTTGGTTAAAGTCAACCCAATAGTTGCTTCATGCTTGGGCTAAAAATGGGTTGGT
TATGATCCACTAATTTGACCCAATTTTTTCTAATGGTGGTCCACTCCTAATACCCGAGAATCGAGCCTT
GTCTCGACACTTGGGACATAAGACTTGTATACCAATTGTAAAAAACTCATTTATGATTTTATGTATAAT
TTTATATAAAATCAATTTATCTCTCCTATCCCAATTACATAGTTTTTCTCCTAAAACCACTCCTCCAAT
CTATTTTGAATTTTAAATTTCATAAGATTTCATGAACTTCCTTTTGTCTTGCTCTCAATTTTCGCAGGA
AACCCATGAATCTATTTTTATTTTTTTCCCCTTCATCAACAATTGTATACGTATTATGCTTCTTAGTTT
TTCATATAATTTTTTTTAAAAATCTTTCTTTCTCATCATATTACAAGTTGTTTAAAATCAGAATGAAAG
ATTCATCTTAATATGTAAGAATTACCTGTTTGAATGTCATGTATATAGTTGTTTGCACAATGAATTATT
CTATACAAAACTTGATCAAGGTAGTTTGTATTGTTATACTCATATTTTAAGTTTTTTTGTATATTCAAC
TAGTTATATATGTATATAAGTAATTACTTTTAAAAAAGATACACTTATTTGTATAATAATTTGTTTTAA
ATCACAATTTTTTTATACTTTACGTTATTATATACAAACTGCTTAATGGATTTGTGTATATACAAGTAC
TATATTCATATTTTTATTTATACATATACAATTACTTATATATGTATATAATAATTAATTTAATAAAAA
TCAAACAATTTATATTCATTTTATTTACATTTGTATATAAATTTGTTTATACGTATACAATTTTTTGTA
TATTTATTTTATTAACATTCGTATATAAACTTAAACTTTTTTTTATACATATACAATTTTTTTTTATAT
ATTCAACTAGTTATATATGTATATAAGTAATTACTTTTAAAATTTTGGTACAATTATTTGTATAATAAT
TGTTTTAAATCATATTTTTTTTGTATTTCATATTATTATATACAAAACTGCTTGAGGGATTCGTGTGTA
TATGTATATAATAATTAATTTACAATTTGGTGCAAATTAAATAACTTATATTCAATTTATTTACATTCA
TATATAAACTTTATATATATTAAGAGTTTAATTTCCCCATAAACAAGTTTTTTATGAATTTTCAGTCAC
AATAGAATTTTTTTAAAAAAAATATTTTTAAATGTTTAACTTAAATTATGAAATGTGTAAATGTTTGTT
AACCATATTTAGGGCTATTGTTATTATTTAATGAAAAATAAAATATAATATAATTCTTAAGAAAGTATT
ATATATAAAATAAAAAATTACGTAACAAATTATACTATACCCACAAAATATAATTATGTAAACTATACC
ATATAATATTATTTCGTAAATTTAGTTTGTCATATAAAATTTTCCCTAAAATGAACAGAAACCC
SEQ ID 37
CGAGGGGACTCTATTGATGATTTGAAGACACAACTTAACACTTATTTTGAGCATCTTGGTGAAAATCAA
TATACACGTCACTTGTCTGCTCTAATGCCAATGATAGACCTAGGAGAAGATAGAGATGAATTCACATGG
AAAACGGCAAGCTATATGCCTTGGCTTATTAAAGACGATAGCGACGTCGGATTTATGTTTAGGAATATG
GTGGAAAATAATGTATTATATATATCTGTTCGTTCCATATGCAATTGTAATGAATGTAAGTAGGGATTT
AATTTAATGATGTGTAATGATGTGTAATGACTTGTAATGTGTTGTTTGATTATGGACACTATGTTCCGT
TTTGATGAATTTCAAACTTTTGTGTGGTTTGAACCAAATGTCGGTTTGATTTAATTATGGACATATGTA
AAAGATATTGTATTTTTCTTGTTTATGACTGAGTTTCATTGTTGTATAATTTGAATTGCATATGGAAAT
GCTCTGGTAAAATTACAGGTAAAAACTGGCCGAAAAATGGCTTGGAAATGCTTAGCATTAATGCAGAAC
CTGCTGTCTGCATAAATGCTTTCCTCGGCAGTTAACTACCGAGGAATTCCTCGGCAGTTAACTGCAGCC
GGATTTCAAATTCCTCGGCAGTTAACTGCCGAGGGGGCAAAAGCGTATTTTACATGTGTGTCCCAGCCT
TCTTTAATGTGTGAACAACAATTTTCTAAAATTAAACCCTACTCTAGGTTTAACATACCAGTAAATTTT
TGCTTTTTGTATGTGTTAACCCTTCTCCAATCCCTTGCACAACCATCTCCTCAAACCTTCTTCTTCTGG
AGCAAAGTCGCCATTCCCTACCTCCTTCTTCATTCTTATTCTCTATAACAAACGGTCCGACCGGATCCA
AGTTGCACCGGTTCGAACCGCTTTAGTTACTACTAACGGTTCGAACCGTTATTTTTCAACCCGTGACGA
ACGTGGAAGGCTTCGTTGTTTCTTCTTCTTCTTCTTCTTCTTCTTATTAATTACCATGCGTTTTTGTTT
TTCTTTTGAG
SEQ ID 38
GATGGGGGTGACCCACGATCGGCTTCTGGATCACTTTATGAGTTTGTCATGTTTCTCTTTTCAAACTCC
TTGACTTGCTCACTTCCAGCTTGCTAGGCAAAACCATGTATGTTTCAACTTAGTGGGTGTTTGGATTAA
CATTTGGAGGCTCATTTCCATTTCTCAGTGCACTTTAAACATGAAAATTGTGAAGCAGAAATTTCTAGC
TTTTAGAAAAACGCGCGTCTAAAAGCCTTCCACCGCAGTCCTAAACAGTCACCTAATCTTTTAAGTCCA
AACATCTATTGATAGTAGTGATTCACATACTTGAAACCTTACTATTTAGGAGGGGGGGTTCCATTGAAT
TACATGCAAAAATAATTTGGAGAGCATGACATATACATACATACTTTTATATATATAAGTGTGTTTCAA
ATTATATAATTTAAGGATTAATAGCAGTTTTGGCCCCCAAACTTTTCAAAAATTACGATTTTGGTCCCC
TAAGAAAAAAAACTACAAAACCGCCCCCTAAGTTTTGCACCTGTGGCAGTTTTGGCCCCCAATGCCAAT
TTTGACTCGGTCTACGCTGACATGACACCCTAAGTGAGGTGCCACGTGTTTTTTTTTCTTTTTATTTTT
TACCTTGGGGGGCCAAAACTGCTACAGTTGCAAAACTTAGAGGGCAGTTTTGTAGTTTTTTTTAAAGGG
TTAAAATCGCAACTTCATGAAAGTTAAGGGGCGAAAACTGCTATTAAGCCTATAATTTAAAATACGTTT
TATAATTCAAAATGGATTGAATTGAAAGAAAAAAAAGAAGAGGGCGCTTGGAGCGTAAAAAAAAATCTC
GTTAATTTTTTTTTTAAGGAAAAATCTCGTTAATTTATTTACTATTGGCCCATGAGAAAAAGTCCGATA
AAATTAAACCCTACTCTAGGTTTAACATACCAGTAAATTTTTGCTTTTTATTTGTGTTAACCCTTCTCC
AATTCCTTGCACAACCATCTCCTCAAACCTTCTTCTTCTGGAGCAAAGTCGCCATTCCCTACCTCCTTC
TTCATTCTTATTCTCTATAACAAACGGTCCGACCGGATCCAAGTTGCACCGGTTCGAACCGCTTTAGTT
ACTACTAACGGTTCGAACCGTTATTTTTCAACCCGTGACAAACGTGGAAGGCTTCGTTGTTTCTTCTTC
TTCTTCTTATTATTAATTACCATGCGTTTTTGTTTTTCTTTTGAG
SEQ ID 39
GGTTGGGGTACCGATTATGTTCGGATCAGTTTACACATATTTTGATTAATTTTAAGAAATACTTGTTAT
TTTTCATCAATACAAATATTGGATAAATTCATTCACAAAGTAATATTCTCCCCCTCTATTAAGTAGTAC
AATTTCTATTTCAATTTATGTAGCGATGTTTGACTGAACACAAAGTTTCAGAAAAAAAGAAAGAAAGAG
ACTTTAGAAATTTACGATCAAAAACAAACACCCACATTTGTCCGGGTAAATATAATTGGATCCTTACAT
AAAAATAAATAGCTGTCAGATTCATTATTATTATTATTTTGTCAGTATACATAAGTTAAGCATTGGTTA
TATATAGATATTATCTCCAATTTAAGCTATTAAATTGAACAACTATTCAAATTAATTCTTTCAGTATTT
AATTGCAGCCACAATCACTTTAAATGCAACTAATCCACTATGAAATGTTTGAACGGTAGATACAAAAAA
GTTCAACGTGACATTCACTTACTAATTTAATACCTACCAAACCCCTATGTCCATTTTTTTTAAAAATAA
AATAAAATTCAACTTCTCATTCATTTTCCTTCTACTTCATTCTCACTCTCTCTATATAAAGAAATTGTG
ATATTGAAAAACT
SEQ ID 40
AAGAGACTTTAGAAATTTACGATCAAAAACAAACACCCACATTTGTCCGGGTAAATATAATTGGATCCT
TACATAAAAATAAATAGCTGTCAGATTCATTATTATTATTATTTTGTCAGTATACATAAGTTAAGCATT
GGTTATATATAGATATTATCTCCAATTTAAGCTATTAAATTGAACAACTATTCAAATTAATTCTTTCAG
TATTTAATTGCAGCCACAATCACTTTAAATGCAACTAATCCACTATGAAATGTTTGAACGGTAGATACA
AAAAAGTTCAACGTGACATTCACTTACTAATTTAATACCTACCAAACCCCTATGTCCAT
SEQ ID 41
GGTTGGGGTACCGATTATGTTCGGATCAGTTTACACATATTTTGATTAATTTTAAGAAATACTTGTTAT
TTTTCATCAATACAAATATTGGATAAATTCATTCACAAAGTAATATTCTCCCCCTCTATTAAGTAGTAC
AATTTCTATTTCAATTTATGTAGCGATGTTTGACTGAACACAAAGTTTCAGAAAAAAAGAAAGAAAGAG
ACTTTAGAAATTTACGATCAAAAACAAACACCCACATTTGTCCGGGTAAATATAATTGGATCCTTACAT
AAAAATAAATAGCTGTCAGATTCATTATTATTATTATTTTGTCAGTATACATAAGTTAAGCATTGGTTA
TATATAGATATTATCTCCAATTTAAGCTATTAAATTGAACAACTATTCAAATTAATTCTTTCAGTATTT
AATTGCAGCCACAATCACTTTAAATGCAACTAATCCACTATGAAATGTTTGAACGGTAGATACAAAAAA
GTTCAACGTGACATTCACTTACTAATTTAATACCTACCAAACCCCTATGTCCATTTTTTTTAAAAATAA
AATAAAATTCAACTTCTCATTCATTTTCCTTCTACTTCATTCTCACTCTCTCTATATAAAGAAATTGTG
ATATTGAAAAACT
SEQ ID 42
TAAGTATCTTTTTAAAAAAAATCTAATTTCAATATAATTTAAATTTTTTTTTACTATTGTGACAATAAA
TTTGATAAAAAAAATTATTTGCCAACTTTCACAAAAATATTTTGACGCAATAGTATAACTATTTAATAC
TATTTTTTTATTTTTTATTTATAAAAAAGATGAAGAGTTAATGATGTTTTAACAAAGAATTTTTTTTTG
ATGTTTTAGCAAAAAACTTTCTTGCAAAGGAAGTGTACAAATAAATAAAGTGTGAAGGGTATTTTTGTA
AACATATATTATTTAATAGTAATTATGCAAGATTTATTATTTTTAATACATCAAACCAAACAATGTATA
AGAAATAATACTTGCATAACTAATGCACGCACTACTAATGCAAGCATTACTAATGCACCATATTTTGTA
TTTGTTCTTATACACTCTACCAAACGACCCCTTAGAGTGTGGGTAAGTAATTAAGTTAGGGATTTGTGG
GAAATGGACAAATATAAGAGAGTGCAGGGGAGTAGTGCAGGAGATTTTCGTGCTTTTATTGATAAATAA
AAAAAGGGTGACATTTAATTTCCACA
SEQ ID 43
GTGGGGTTCCTTTCATTTCGTGCTCTCCTTTCTCTGCCAGCCAGTCCGTCCGTCCTTGCGTCCACTGCA
CCTGCACACAGGTCACCCCGACCCGCACTGTTNTAGACTCCATTAGAAAAAAAAAGGTNTGAACCTTTC
CGAAACCAGCCAGCCATTGGTCTGGCAGGCCAGCATATGCTAATTGGATTTTTTTGCCGCATCATTGAG
TGCGCCATCAGGATTTGGAAATCCTGGTTTTGAGTAATACAGTAATTTGGCATTATCCATTGCCGAATT
CCCAAGCTCCGTCAGCTTGAACGTGGACCCCTACCATCTGCACCAGCTCGGCACCTCACGCTCGCAGCG
CTAGGAGCCTAGGAGCAG
SEQ ID 44
GTCGACCTGCAGCCAGAAGGATAAAGAAATTTTGGACGCCTGAAGAAGAGGCAGTTCTGAGGGAAGGAG
TAAAAGAGTATGTCTCCTTAACTCTACTATCAAGTTTCAAGAAGCTGAGCTTGGCTCTACCTTGATATG
TTTATTGCTGTTGTGCAGGTATGGTAAATCATGGAAAGAGATAAAGAATGCAAACCCTGAAGTATTCGC
AGAGAGGACTGAGGTGAGAGAGCATGTCACTTTTGTGTTACTCATCTGAATTATCTTATATGCGAATTG
TGAGTGGTACTAAAAAAGGTTGTAACTTTTGGTAGGTTGATTTGAAGGATAAATGGAGGAACTTGGTTC
GGTAGCCGTAACAAGTTTTTGGGAATCTCTTGGGTTTTAAATTGCTATGGAGTTTTTTTTTGCCTGCGT
GACAACATATCATCAGCTGTTGAGAAGGAAGATGGTATTAGAAAGGGTCTTTCTTTCACATTTTGTGTT
GTGGACAAATATTAAAGTCAAATGTGGCACATGGATTTTAATTCGGCCGGTATGGTTTGGTTAAGACTG
GTTTAACATGTATAATTAGTCTTTGTTTTATTTGGCTCAGCGGTTTGTTGGTGTTGGTTAGGAACTTAG
GCTTGTCTCTTTCTGATAAGATCTGATTGGTAAGATATGGGTACTGTTTGGTTTATATGTTTTGACTAT
TCAGTCACTATGGCCCCCATAAATTTTAATTCGGCTGGTATGTCTCGGTTAAGACCGGTTTGACATGGT
TCATTTCAGTTCAATTATGTGAATCTGGCACGTGATATGTTTACCTTCACACGAACATTAGTAATGATG
GGCTAATTTAAGACTTAACAGCCTAGAAAGGCCCATCTTATTACGTAACGACATCGTTTAGAGTGCACC
AAGCTTATAAATGACGACGAGCTACCTCGGGGCATCACGCTCTTTGTACACTCCGCCATCTCTCTCTCC
TTCGAGCACAGATCTCTCTCGTGAATATCGACA
SEQ ID 45
GGAAGCTTTACAATGGGTTACATGTATGGATCCGAGTATGAAGAATGTTGGGAATCAGTGATGCTTCGC
GCGTTAGGACTTTTTCTTCCTGGTATTTCTGCCCACAGCCCAGTTGATTATGTGAACTCCATCAGACTT
GGAAAGGCGAGAAGTACACAGATGTCATCCTTTTAGAAAGCTTTTTGTCGCAAATAGTGGTTTTATAGC
TGGACAATATCATGCATTCCTTATGAGGCTTATGCAGTATGTGTCCTGTTTGATTTTTGAAGGTTTGCT
TTTAGTGTTTATGTATTGACAATAAACTTATTTCAGTTCTTTTATTAAGAGATGGATTTGCATAAAAGA
TATTGTTCCTCTGGTAATCGTATTAAACTTGTTATGTCTTCAGTGAGGCGAATAGATATAAGATTGTTA
GATGGTGTTAATAATTTGGTGACATTGCAATTTGCAAAACTGTAAAAGGATTTTTGCTTTACTATTTTG
TCTATGTTGACTATATCCCGTGAACTATGAAAATGAAACAAGCAAGTAACACTCTATATATTGTTTCCT
TGCTAGAACACTCATTCAACTTTTCTTTTTCACCCGAGAGAAAAAAATATTCACTATATTTAAAGTCGG
TATTATTCGTAAGAACAAATTATAATCTCGAAAAGAGTAAATTGCACGTGGTAAAAAAATTGTAAGATT
TTAAATAGTCTCTATAAATTAGGTACAAACTTAGGCATAAAAAAAAGGTTGATATAAATTACCTTTTAT
ATAAAAAATGTAATTTACAGAAGAAACAATTACTACTACTACTACTAAAAAACATGGGTCAGGTTGGAT
TACGTG
SEQ ID 46
CTAGTAATACTGAGATTAGTTACCTGAGACTATTTCCTATCTTCTGTTTTGATTTGATTTATTAAGGAA
AATTATGTTTCAACGGCCATGCTTATCCATGCATTATTAATGATCAATATATTACTAAATGCTATTACT
ATAGGTTGCTTATATGTTCTGTAATACTGAATATGATGTATAACTAATACATACATTAAATTCTCTAAT
AAATCTATCAACAGAAGCCTAAGAGATTAACAAATACTACTATTATCCAGACTAAGTTATTTTTCTGTT
TACTACAGATCCTTCCAAGAACAAAAACTTAATAATTGTATGGCTGCTATAC
SEQ ID 47
AGTGAAATATATTGTATTGGGAATGATAAAAGTAGTATTATTTAGTGTTATATTGTATTGGGAATGATG
AAAATTGTATTGAAAATTGAAATGGGTCAGTTATTTTGGAACACTTTTTTTTAGAAAATGGGTCAGTTA
TTCCGGGACGGAGGGAGTAATAATTATCTTAAAAGCATTTTAAAACAAAAAGCAAGAAACTTCATATTA
AAAACAATAATTTTTAAACATTTAAAAAGTTAAATATGCACTTTCTCACCGTTTCTCAAAATAAAAAAA
ATCTTTATTTTAATTTCCTTGAGATATCCTAACAAAAAAGCAACAACTTCAGCGTGTGATTCACACACA
AACACACCAACCCTGAACAATCAATTGTCCTTCTCTCCAACTCCAATAGTCCACTAGGAAGGAAGGGTC
TTTATGGGGTGTACAATGTGCCAGTGGAGTGGAGGGGTCTACATCCTCACCAAACTTTGATTCTTCTTC
AACAATCCAAAACCCGTATGCATCATGAGTTGAGTGGTTCAAAAAAGTCTCTCTTTCACTCACCAAATA
CGTAACAGAACACTTTAGCTTTGATGATGATTCAATGCATCCTAACGCAACGCCACCTATGTCCCATTA
AACACATCAGTTCACCCCTTGCAAAATATATGAAAGAGATTGAAAGAAACAGTGACTTAACAATGTTGG
ATGTTGGAATAGTTATTACTCATTCATTCATATAAGTTGTTTTCAAAATAAACGGTGTGATATACAAAA
ATACAACGTTCAAGATTCTACAAATTGCAAATAATTTAGCAGAATTTGTTGCAATGCATAATTTATATT
TTTAGTATACTATCATGTAGGACATTTCTTAAAAAAGAAACAATTCTTTACAATGACCTTCAAAAAATA
CTATACGACCTACTTTGCGTAAGCAGTATACATTTTCGCCTACCTTTATTTTAAATGATTCAATTTCAT
TTGCCTTAACTTTATTTTTCATTTTCGAATTAAGGGATTAGCGTCAAATTCAACTTTCATTTTTGTTCA
AAAAAACTTTCATTTGTATTTTGTTTTATGAAGTATTTAGTAACCGAAATTTCATTAGTTAAAGTGAAT
AAGTAAAGAATATTGACTTCGATTTCTACGTATTATAATGTTTCTACAAACTTTTGTTTGTATTAAAAT
TAAATTATTATTTTTCATAAATAAAATATAGAAAATTTAGTGATTTTTTTAAGGAAAAAAAATTAGTGA
TTTGTTTTTTTGGTCAAGAAAATTAAGTGATTTAATCCCTTACTATATATCATGCAATACCTTTTTTTC
CTTTAGGAAATTACGCAATACCTGTATGGTTGGTAAATCAAATAATTCTT
SEQ ID 48
AAGGGGGACTCATTCCTATCTCCCCCATCAACCTCCCTCCCTCATCACCGTACCTCGCCACCAACACTT
TATACAACAACCCGTCCATATCCACCAACATTCGCCAACATCATTTTTCTAACAATGCAATATTAAAAT
CCCACATCTTCCTGACCCCCAAACCTTTGTACTCCTTTTTCAAGTAGAGGAAATTATACGTGTGAGCCA
TGAAGAAGGAATGAAAGTAGACCGCAAGAGAGGACATGACAAACTTCACGAGAATCATACGACCACGCA
TTTATTATTATTATTATTAATAATTTTTGAATGACAAATGTTAATTGTTAGTTTGTTTGAGTTTTGAAT
TCAAAACATTTAACTCTTTTCTATTCATTCAAATCAGTTGGACTACTTAATCCTTCCCAAAAAAATGTG
ATAGATCACACTAACATGATAAAAAGAGATAAAATTAGATGTTGAATGAATATTCACAATTACATTTTT
TTTGCTGATAAAGTTATACTTAAAAATAGCCAAACATAACACAATAATTAATTAATTACTTTCTTACAA
AGACCATCCAACCATGAAATGAACCATATTAACTCGATGACAAAAGAGAATGCAATTTTTAGTTTAATC
TACACACAAAAAAAGACAACACACACCAAGGCCACAAACCCCACCTAACCCTCTACAGTAATTCCACCT
AACTAAAAACCCATACACATCATCATCATCATCAAAACCTCTCTATAAAAACCCAACAACCACTCCTAA
CATT
SEQ ID 49
CTGCTTGAGGGATTCGTGTGTATATGTATATAATAATTAATTTACAATTTGGTGCAAATTAAATAACTT
ATATTCAATTTATTTACATTCATATATAAACTTTATATATATTAAGAGTTTAATTTCCCCATAAACAAG
TTTTTTATGAATTTTCAGTCACAATAGAATTTTTTTAAAAAAAATATTTTTAAATGTTTAACTTAAATT
ATGAAATGTGTAAATGTTTGTTAACCATATTTAGGGCTATTGTTATTATTTAATGAAAAATAAAATATA
ATATAATTCTTAAGAAAGTATTATATATAAAATAAAAAATTACGTAACAAATTATACTATACCCACAAA
ATATAATTATGTAAACTATACCATATAATATTATTTCGTAAATTTAGTTTGTCATATAAAATTTTCCCT
AAAATGAACAGAAACCC
SEQ ID 50
AAGAGACTTTAGAAATTTACGATCAAAAACAAACACCCACATTTGTCCGGGTAAATATAATTGGATCCT
TACATAAAAATAAATAGCTGTCAGATTCATTATTATTATTATTTTGTCAGTATACATAAGTTAAGCATT
GGTTATATATAGATATTATCTCCAATTTAAGCTATTAAATTGAACAACTATTCAAATTAATTCTTTCAG
TATTTAATTGCAGCCACAATCACTTTAAATGCAACTAATCCACTATGAAATGTTTGAACGGTAGATACA
AAAAAGTTCAACGTGACATTCACTTACTAATTTAATACCTACCAAACCCCTATGTCCATT
SEQ ID 51
GATCTTCTTTCATCTAAACTGACACTAAACTCTTTTTTCTTCCCTTCTCCAATATCCAACATGCAATTA
GACGATGAACGAAATGTGATGAAAAATTTGATAAATGAGAGTTCAAATTTTAACAAAATTAAATAAAAA
ACATAATCAATTTTTTAAATTTTAGAAATAGAGTTATTGTTTAAATGATACATTGAAATTGCAGTATAT
ATCTTATGAAATAATGGAGATAACTTAAATTGACCAAACATTATTATTATTTACACAAAAGGGGGAAAT
AGCAATTTTTGGACCAAATATTATACTAAGGAATAGGATGAAATTATAAAATGATTTGCTCGTTTTTTT
TTCTTCTCAAAAACGAAAGAACGCACAAGTTGCGGATCTCATGAGATCATTACCCAATGCATTAGGTAG
AGTAAGATCCACATCACTAACCTTTTCTCCGTCAATTTTTATTTGGCCCATATATTAAAAAAATATTTA
TTTAAAAAATTAGAAGCTAATATATTATTATGAAGTTTAATTTATTGTTATTATTAACTATAGTAATTA
TTTCAAGTATATTTTTTAAAATATTAAATTTATTATATTCGAAAGAAGATGTAATAAATGTATCAATCT
TTCTGTTTCAATTTATATAATTCATGTTATTTTAGTTTGCCTAAAAAGAATGATACATTTGCAGTGGTG
ACACGATTTGTAAAAATTTATGCGTACTCATTGTCTATATGTATGTATCGCAGCGGCAAGCGAGATGAA
AGAGATGCAAGAAGATTTGTTATCTATTTCAAAATATATATGAATCTTACTTAGACACAATGTATATAG
AACAAATTATATGTAATAGTTGACCCTATATATGTGGTAAAATACTTGACTATTAGGGGTTGTTTGGTA
GAGTGTATTAAGAAATATAATGCATATATTAGGTGTGTGTATTAGTAGTACCTTGTTTGGCACACTTTT
TCATGCCATGTATAACTAATGCATGTGTATTACTAATACCAAGGAATTCTAGGTATTAGTAATAAATAG
CATTTTAACACTTGCATTAGATCAAATAATTACAAAACTACCCTTAAAGCATTTTCATTTTCTTTGTTG
TCATAAGTTTTTATTTTTATTTTTATTTGCTTTTCGGTATCTTTTAATTTGTTGGTGTCTTAATAGACT
TTATGGCCTTTTAAGTATCTTTTTAAAAAAAATCTAATTTCAATATAATTTAAATTTTTTTTTACTATT
GTGACAATAAATTTGATAAAAAAAATTATTTGCCAACTTTCACAAAAATATTTTGACGCAATAGTATAA
CTATTTAATACTATTTTTTTATTTTTTATTTATAAAAAAGATGAAGAGTTAATGATGTTTTAACAAAGA
TTTTTTTTTTGATGTTTTAGCAAAAAACTTTCTTGCAAAGGAAGTGTACAAATAAATAAAGTGTGAAGG
GTATTTTTGTAAACATATATTATTTAATAGTAATTATGCAAGATTTATTATTTTTAATACATCAAACCA
AACAATGTATAAGAAATAATACTTGCATAACTAATGCACGCACTACTAATGCAAGCATTACTAATGCAC
CATATTTTGTATTTGTTCTTATACACTCTACCAAACGACCCCTTAGAGTGTGGGTAAGTAATTAAGTTA
GGGATTTGTGGGAAATGGACAAATATAAGAGAGTGCAGGGGAGTAGTGCAGGAGATTTTCGTGCTTTTA
TTGATAAATAAAAAAAGGGTGACATTTAATTTCCACAAGAGGACCGAACACAACACACTTAATTCCTGT
GTGTGAATCAATAATTGACTTCTCCAATCTTCATCAATAAAATAATTCACAATCCTCACTCTCTT
SEQ ID 52
CGAGGGGACTCTATTGATGATTTGAAGACACAACTTAACACTTATTTTGAGCATCTTGGTGAAAATCAA
TATACACGTCACTTGTCTGCTCTAATGCCAATGATAGACCTAGGAGAAGATAGAGATGAATTCACATGG
AAAACGGCAAGCTATATGCCTTGGCTTATTAAAGACGATAGCGACGTCGGATTTATGTTTAGGAATATG
GTGGAAAATAATGTATTATATATATCTGTTCGTTCCATATGCAATTGTAATGAATGTAAGTAGGGATTT
AATTTAATGATGTGTAATGATGTGTAATGACTTGTAATGTGTTGTTTGATTATGGACACTATGTTCCGT
TTTGATGAATTTCAAACTTTTGTGTGGTTTGAACCAAATGTCGGTTTGATTTAATTATGGACATATGTA
AAAGATATTGTATTTTTCTTGTTTATGACTGAGTTTCATTGTTGTATAATTTGAATTGCATATGGAAAT
GCTCTGGTAAAATTACAGGTAAAAACTGGCCGAAAAATGGCTTGGAAATGCTTAGCATTAATGCAGAAC
CTGCTGTCTGCATAAATGCTTTCCTCGGCAGTTAACTACCGAGGAATTCCTCGGCAGTTAACTGCAGCC
GGATTTCAAATTCCTCGGCAGTTAACTGCCGAGGGGGCAAAAGCGTATTTTACATGTGTGTCCCAGCCT
TCTTTAATGTGTGAACAACAATTTTCTAAAATTAAACCCTACTCTAGGTTTAACATACCAGTAAATTTT
TGCTTTTTGTATGTGTTAACCCTTCTCCAATCCCTTGCACAACCATCTCCTCAAACCTTCTTCTTCTGG
AGCAAAGTCGCCATTCCCTACCTCCTTCTTCATTCTTATTCTCTATAACAAACGGTCCGACCGGATCCA
AGTTGCACCGGTTCGAACCGCTTTAGTTACTACTAACGGTTCGAACCGTTATTTTTCAACCCGTGACGA
ACGTGGAAGGCTTCGTTGTTTCTTCTTCTTCTTCTTCTTCTTCTTATTAATTACCATGCGTTTTTGTTT
TTCTTTTGAG
SEQ ID 53
CTACCGAGGAATTCCTCGGCAGTTAACTGCAGCCGGATTTCAAATTCCTCGGCAGTTAACTGCCGAGGG
GGCAAAAGCGTATTTTACATGTGTGTCCCAGCCTTCTTTAATGTGTGAACAACAATTTTCTAAAATTAA
ACCCTACTCTAGGTTTAACATACCAGTAAATTTTTGCTTTTTGTATGTGTTAACCCTTCTCCAATCCCT
TGCACAACCATCTCCTCAAACCTTCTTCTTCTGGAGCAAAGTCGCCATTCCCTACCTCCTTCTTCATTC
TTATTCTCTATAACAAACGGTCCGACCGGATCCAAGTTG
SEQ ID 54
CTACCGAGGAATTCCTCGGCAGTTAACTGCAGCCGGATTTCAAATTCCTCGGCAGTTAACTGCCGAGGG
GGCAAAAGCGTATTTTACATGTGTGTCCCAGCCTTCTTTAATGTGTGAACAACAATTTTCTAAAATTAA
ACCCTACTCTAGGTTTAACATACCAGTAAATTTTTGCTTTTTGTATGTGTTAACCCTTCTCCAATCCCT
TGCACAACCATCTCCTCAAACCTTCTTCTTCTGGAGCAAAGTCGCCATTCCCTACCTCCTTCTTCATTC
TTATTCTCTATAACAAACGGTCCGACCGGATCCAAGTTGCCTCGTAGTAATATTTAAGCGAGTTAGACC
GCGAGGCTTTAAATACAAAGATTCAATAAAACCTCATTACCATGTATGTGATTTCGTCAAATTTGTTGT
TATTTCAAACATGCGCGCATAATGAGTTCAAATGAATATATGCTAATAGTTGTGAACTTTGTCGCAGGC
AACTTGGATCCGGTCGGACCGTTTGTTATAGAGAATAAGAATGAAGAAGGAGGTAGGGAATGGCGACTT
TGCTCCAGAAGAAGAAGGTTTGAGGAGATGGTTGTGCAAGGGATTGGAGAAGGGTTAACACATACAAAA
AGCAAAAATTTACTGGTATGTTAAACCTAGAGTAGGGTTTAATTTTAGAAAATTGTTGTTCACACATTA
AAGAAGGCTGGGACACACATGTAAAATACGCTTTTGCCCCCTCGGCAGTTAACTGCCGAGGAATTTGAA
ATCCGGCTGCAGTTAACTGCCGAGGAATTCCTCGGTAG
SEQ ID 55
CTACCGAGGAATTCCTCGGCAGTTAACTGCAGCCGGATTTCAAATTCCTCGGCAGTTAACTGCCGAGGG
GGCAAAAGCGTATTTTACATGTGTGTCCCAGCCTTCTTTAATGTGTGAACAACAATTTTCTAAAATTAA
ACCCTACTCTAGGTTTAACATACCAGTAAATTTTTGCTTTTTGTATGTGTTAACCCTTCTCCAATCCCT
TGCACAACCATCTCCTCAAACCTTCTTCTTCTGGAGCAAAGTCGCCATTCCCTACCTCCTTCTTCATTC
TTATTCTCTATAACAAACGGTCCGACCGGATCCAAGTTGCACCGGTTCGAACCGCTTTAGTTACTACTA
ACGGTTCGAACCGTTATTTTTCAACCCGTGACGAACGTGGAAGGCTTCGTTGTTTCTTCTTCTTCTTCT
TCTTCTTCTTATTAATTACCATGCGTTTTTG
SEQ ID 56
GGATGGGGTCACCTTATCCTAGTCAATAAATAATCAACAAAATTTTAGGGAACAAAATATATATGCTAG
AGGATCGTTATGTTTGTCTTCCATTTCACTGCATCTACATATGGAATTGATTCTAGAGTAAGAAACACA
AATAAATTTATTTGGTACAATCCTCCCGTCCAAGGAAAATCTAAAAATAGAAAAGAAATCTTAGTGAAG
TTATAGATTATGGTAGCTTATATTTTTTTAAAAAAACGATTATGGTAGCTTCTATTTATACCCTACTTT
AAATATATATGATTGTCCTATAACGTATTGAATAGAAAATATCTTCGAATATCATATATATGAAACTAG
TGTAAATTTTAAACGTAAACAATTTATACGACCACAGTTCGAAGAAAAAAAACAATTTATACGACCAGA
AATGGCAAAATGTTGTTCTTAGAATTTTTTTCTACTTTACTTTTGCGTAAAACACATTTCTCCAATTTG
GTTTCATTGCGTTGAACGACGTAACAAAGTAATACACCCAACCCTTTTTTTTGGAACATTATGCACCCA
ACCCATTGTACAAAAGTTACAGCTAATTACCATTTTTATTCTTTTGATAAATACAAAAATAAATTATTA
ATCATTAAAAAAAAATTTGGAATATTTTCTCAATGTCCATATATACATCTTCTCCCTTTATATAAGCCA
ACCTCACACACCCAAAAAATCCATCAAACCTTTCTCCACCACATTTCACTGAAAGGCCACACATCTAGA
GAGAGAAACTTCGTCCAAATCTCTCTCTCCAGCA
SEQ ID 57
AGGGGGGACTCTTCATATTATTTTTGGTGAGTAGCGTAATCATAGATAGTTTTCTTAATTCTTGAACTT
GGGTAACATCGTGGGTATCTACGAAATGATTCCTTTCGACGTACACGATTTATAGATAAACACGTAGAG
ACGTGTATAATAAGCGAGAAACTTATTTAGCAGTGTTAGAGAAATATTTGAGTTAACAGACTATAGAAC
ATTTATAAATTAGTATTCAATAAATTAATATTTTTAATATTCAATAATTAATATTTTAATCTTCAGTAA
AAAAATATAATATTCGATAACTTAGTATTCAATAAATTAATATTTTCAATAAATTAATATTCAAAAAAT
TAACATTTATAAAAAATCATTAAATTATATTGTCTCATTACAATTGTAAATTAATAACTGATGTATAAA
AATTATATAAACATAACAAAATATTGTTATGTATGGTTTTTATTTAAAATGAAACTAATTCTAATTTTT
TCAACACTTCAAAGTATTTTATAATTATATATTTAAAAATATTAACATTATGTGATTCATATTATATAT
ATGTCAAATAATTTAATAAACACTATGAAAGCTAAGTTTACAAAACTTAATTAATATATAATTCACGAA
AAAATCTATTCCTTTTATTTTACATATAAACATATTTTAAAATATATAAATCTAAGTATGATATTTTGA
TAAATTACTAATTTTATAAATTAAATATTATAGTTCATTAAGTATTTTGAATAATTATTGGATCTTTAA
GTATTTTGAATAATTATTCAAAATTGACTCATTTTGTTTTTTAAGATTTTTAAAAAATTGAGTTTTTTT
TTCGATCTCCGTTAGAATTTGATTTGGGTAAAAACTAAAATCTGAAATACCATAGAATAATAACCATTT
GGATACTTATGTCGAATTCAAAACAGTTTAATTCTCAGGTTCAAATTTTCATATTGTTTTTTCATACCA
TAGAATAATAGCCATTTGGATACTTATGTCTAAAAGTAATATAATCTGAGACAAAATATAAAAATATAA
GGATTTATATATTTCAACCATATGGATATGGTTGTGTGATACGAAAGTGTTAGACATTATCGATTTGAA
ATCTATCATTCAGATTTGTCTTTTACATGGTTAAAGGGTGTGTGAATATAAAACTTTCACGTAGAACAA
CGGATTTATCTGTTGCCTGAAAAACAGGCTAAACACTCTATTATGATTAGTCTTAGATTTAGGACACCC
CTGGTCCATAAAAAAGGTCTTACATATTTACTTTCGCATACATATTTTTCTAATTTAATTTCACTGAAT
AGAACGATGTAACAAAGTAACCAAACCCATTGCATTTAAAATTACAGCAAAATTATCCTTTTTTTAAAA
TATATAATTATTTCTTTAAATATATATATATTTTTTTTATTTTTTTTTCAACAAATATATAATTATTAA
AAAAAAACAGTTTTGAGTATCTCAATCAATTCTACAGACTTACACATCCTCCTTCCCCTTTATATAAAG
AAACTTCAGACCTCAAAATACATCGAACCCTTTCTTCACCACATTCCACTTCCCACACTCTCTTTTTTT
TTGAATTATAGAGAGAGAATCCTCCTCCAAATCTCTCTCTCTCCCAGG
SEQ ID 58
GATTATGCTGAGTGATATCCCAACCGGGCATGCAGAGTGGAGGCGATGGAAGAAAGCGGTGCCGGAGAC
CGTTCGACTGCAGCAAAATTACCAGAGAAGTTAAAAGGGGAAGATGTGAACAAGGGTAAGACACGAGTT
ACTTTTCAACGGTGAATAATTAAAATATTTAATTATTTTTTTGTAGCAGGTTGAGCCGGTTGTGTTTTA
GGAATATTACAGTATTATTTTATATTTGTAACAGCGTGTATAAGATCGTTAGGTTAAATGGCTAGACGG
TGAATTACGTTTTTTTTTGTGGTTATAGCCTTCAATTTCCCATTTAATTTCACCGAATAGAACGATGTA
ACAAAATAACAAACCCATTGCATTTAALATTACAGCAAATTACCCTTTTTATTCTTTAAATATATAATT
ATTTAATAAAAACAGTTTGAGCATCTCAATGTCTACAGACTACACATCTTCCTTCCCCTTTATATAAAC
AAACTTCACAGACCGCAAAATACATCGAACCCTTTCTTCACCACATTCCAGTTCCCACACTTTCTTTTT
TTTGAATTATAGAGAGAGAATCTTCCTCCAAATCTCTCTCTCTCTCTCCCAGG
SEQ ID 59
GACGAAGATCTTCTCCTGGTAATCTAAGGAAACATGAATATTTGTTGAGTTTTGGCTTGTGAAGATGCT
CTTTGTTCATCTGCTGTTTTCGATGGATTTGTGCAGATTAACTTGGAGAACATGAAGAAGCAGAAAGAA
TAGTTCCCTATCTTCTTCATCATCATCAAATGAGTGTGGATTAAAATGAAACCCACCCGAGTGTTCTAT
CCCAGAAGAGCAATACTAGTTTACATATACATATATATATATATATACGTATAAATGGATGTTGCCCAA
CATATTCATATAGAGGTTCATGGATCATAAGTGAGTATAGGTTTGACATTGATCAGATTTGTCTCTGTT
TCTAAGCTGTTATAGTTATTCCTTGTTGTACAAATCGGTTTTGCCATAAAAGTCCCTTTAGGATGTGAA
TGCAATATAAGATTTGATTGATTCAAGTTTTCCAGTAATAACAAGACTAATTCCACTACGTTAAAACAA
AAGTACAATCGACCGTACCGGATCGAACCGAACCGAACCAATACCAACATATCCAATTCGCGTCATACC
AGAACATTCTTAAACCGGAATTAGATTCGGACCAAACACATCATCATAAGATTCGTTAAGAAGATGGTT
GTGTCTTTTTCCCTGTCTGCTACTAG
SEQ ID 60
ACAGAGAAAATNTCTTGCAGGATGCACGAGAGGANATCGTCAAAATGTCTAGAGAATGCCCGGAAATCG
TTTGGTACAGACGAAGATCTTCTCCTGGTAATCTAAGGAAACATGAATATTCGTTGGGTTTTGGCTTTG
TGCAGTTGCTCTTTGTTCATCTGTTGTTTTCGATGGATTTGTGCAGATCAACTTGGAGAACATGAAGAA
GCAGAAAGAATAGTTCTCTATCTTCATCATCATCATCATTATCAAATCAGTGTGGATTAAAATGAAACC
ACCCGAGTGTTCTATCCCAGAAGAGCAATACTAGTTTACACATACATATATACGTATAATGGATGTTGC
CCAAACATATTCATATAGAGAGGTGCATGGATCATCAGTGAACTCAAGAGTATAGGCTTTGACAATGAT
CAGATTCATCTGTTTCTAAGCAGTTAATAGTTATTCCTTGTTGTACAAATCGGTTTTGTCATAAAGTCC
CTTTAGGATGTGAATGCATATAAGATTTGATTGATTCAAGTTTTGGAGTAATAACAAGAGTAATTCCAC
TGTGTTCAAAAAAAAAAAGAAAAAAAAGAGTAATTCCACTCGACGAACCGGTAAATATCGGAGTACAAT
CGAGCGTACCGGATCGAACCGAACCAGACTAATACCACCGTACCCAATTCGCGTCATACCAGAACATTC
TTAAACCGGAATTAGATTCGGACCGAACACATCATCATAAGATTCGTTTGGAAGATGGTTGTGTCTTTT
TCCCTGTCTGCTAA
SEQ ID 61
TGAGCTTGAAGGGACGTTTGAGCAGATAAACGAAGCGAGTGTGATGGTTAGAGAGCTGATTGGGAGGCT
TAACTCTGCAGCTAGTAGGAGACCACCTGGTGGTGGTGGTGGGATTGGTGGTGGGGTTGGTTCGGAAGG
GAAACCACATCCAGGGAGCAACTTCAAGACGAAGATGTGTGAGAGGTTCGCGAAAGGGAACTGTACGTT
TGGGGATAGGTGTCACTTTGCGCACGGGGAAGCAGAGCTGCGCAGGTCAGGAATTGCCTAAGTTGCTGT
TTGTGGAGTTTGCTGTCTTTTCTTTTGTGTGTGGTGGTGATCTCTAATATCATCCATCTTCTTCATCTA
TTTTGCTTTTGTTTTATGAAAATACAATGTTAGTTTCATTGTCTTTGTAAGTTTTCTTTCTCTCTGTGT
GGTGATTCTTAGAATATAGTTTTTTTTGCTGTTAAATTGAGTTTGAATTGGTGAGAGACTTGGTGGATG
GATTGACAGACGGTGGTTAGGATTTGTATGCTGCCTTAATTTTCTTACAGTCATGCTTGCTCTGATTTG
TCTGTTGTGCGTGAGTCAGACACATCATCTTTGATACCAAAAAAACATGTTATAAAACCCGTCACTGGT
AGTAACAATCAGCTGAATAAATATAACATTCCTAATGGTGGGTGTGTGATCTTAAACAAAAAATTTTGA
AAGAAAAGTGTGTTGTTGTTAGAGGTAATGCTTAGACAAATCAAACTCTAATCATCTTCTAAGTCTAGT
ATAATACAAGAGATCTCAATCTAATCAATCACTAGTTTCTTTTCGTCTGCCAACAAATTTGATTATTAT
AAGTATCAAAGATGATTACACATACATAACAAATTGTAATAAGAAAAAGAAAAGAGAGAGAAATCCTCA
CGTGAGCATCACCACAATTTGTCTGTTACATATTTCTGTAAGTTCTTGTGTGTTCACATGGGCAAAAGT
GAGAAGAAGCCAAACACGATACTCCATTTTCAGGCATCAACTACCATCTTCTTCTTCTTCTTCTTTATC
AAGTTGTTTCTAATGTCATATTAAGAAATGATACATGATTGACTTACGTAGAGAAAAACTGATTCAAAC
AAGTACCGCATGTGTCATTGCGTTCCAAAGTGATTAAGTCAATAACATGATACGACCTTTTTTATTACA
TTACATACATAACCAAGATAACGTGGACGAGAAAAAGAGAGAACGTCGTAGTAATATCACCTTTTCATC
ACTCTAACTTTTACATTTTGGTAAATTCTAAATTAATGGTCGTTCCTTGAGTTAAATATCAGATATTTT
GAACAGAGGGGCCCAGTTGTAAAAATAAGAGAAAAGAGGGGCCAGTTGTAAGAATAAGAGATGTCATTC
AAATGCCTTCCTGTCTCTCATCAATTTAAAAACGGCCCTGCCTATTGCCACTCGC
SEQ ID 62
GAGAAGAAGCCAAACACGATACTCCATTTCCAGGCATCAACTACCATCTTCTTCTTCTTCTTCTTTATC
AAGTTGTTTCTAATGTCATATTAAGAAATGATACATGATTGACTTACGTAGAGAAAAACTGATTCAAAC
AAGTACCGCATGTGTCATTGCGTTCCAAAGTGATTAAGTCAATAACATGATACGACCTTTTTTATTACA
TTACATACATAACCAAGATAACGTGGACGAGAAAAAGAGAGAACGTCGTAGTAATATCACCTTTTCATC
ACTCTAACTTTTACATTTTGGTAAATTCTAAATTAATGGTCGTTCCTTGAGTTAAATATCAGATATTTT
GAACAGAGGGGCCCAGTTGTAAAAATAAGAGAAAAGAGGGGCCAGTTGTAAGAATAAGAGATGTCATTC
AAATGCCTTCCTGTCTCTCATCAATTTAAAAACGGCCCTGCCTATTGCCACTCGCATCTGACCAGACA
SEQ ID NO 63
TTACACATTCGCAACCCTGGAGGATACTCCAAGAGACTACGATCCCAAAGGACAACCTATACAATTGTG
GAGAGTGACAAAGAAGGGAGAGCATATGAATGGATAATACTAGCACTGCATAGCTTAACTTGTATCGTT
TTTTCTCCTTAGGTTAGTAGGTATGTTTTACAAAAATTAATTTCTATGAATTTTAAATATAATATAAAA
TAATATGTTTTAGGTGAAACAAATTTATAAGTCCAACGGTGGACTTCATGTTCTACAAAAAAAAGTATA
GTTAAACGAACCAACCAAATAAACTGTTAGAAATGCATAATGTTAGGTTTTGTATAAATGTTATGTTTC
AATTTGAGCTTTGATAAAATACACACGAGTAAAGAAAGAGGTAAGATGCACATGTACCTTGTTTGTTGT
ACACTCAGCCCACTCAACTATTATTACTAAAACGTCGGTGCCAAAGTTGACAATTCTCTGCTAAATACA
ATCTGATATACGTCTCTTTCTCCACAACAATATGTTGATTGGTTAGTGTAATTAGCAATCCTCACATAT
AGGGAGGAAATCAAATATTCAAATCCAAATGAAATTTCCACGGAAGCAAGTAATCAAGTCTTGCGTGCT
TACATAACGAGTGACCAATAATATAAAAAAGAATTGAATTAGATTAGCCTAGTTAGGTTAACAATCTTT
TAACAAGAAAAGGGTATAATTGGAAATACAAGAAAATTTAAAAATATGGTTTTGAAACTACGAGAAGGA
AGGAGAAAGGAAGAAGAAGJAGAAGGGGAGTGCAATTTATATAAGAAAAGGCCTCTCGTCCACATCTCT
CTCTCTCACACCCCACCCTACAGAGACTCTCTCTCCCCCTTTTATCTCTCTCTCTCTACGCCAAATTTT
TAAATATTTTTTTTTCCTACAAAAAAGAAGTATTGAGAATCGCAAACAAAAGTAAAAAAAATATTAAAC
AAAAGGAGGAGAGGAGAGGAGATCGTGAGGGAGGCACAACCGAAGAAGTAGGGACTTTGGAGAAAATTA
GCGTTACCATTTTTGAGATTTTCATCCTCCATTCTACACCTGAAGGTGGTACCATCTCTCTCTCTTCTT
CTTCGTGTGTTCTTCGTTAATATCTTCATCGCTTGGTTCGGATTCCTTATTCAAATTCAATGCTTTATC
GAAAATAATAATATTCCAATTATCTTTTTTTTGATAAAAAGTTTTGATTTTTATCGGTTTACCTTTGTA
GTTTCAAAATTCCAGATCTGAATTTTTTTCTCTCTGCTTGTTACACAAAAAAAAAGTTTTGATTTTGAT
TTTTTGTTATTGTTGTTGTGTTTTTGATTATAGACTTGTAGCATTTTTGTTGTTGTTGATTAATTGATT
AGCTAATTGTTACAAAGATGTAGACTTTGTAATAATACGTCACTCACTTTGTTATGTTTTGTTGTGTTT
TTTTTTTGTTTTATAGTGTCTTTGAAACGCTCATCTCCTCAAGCC
SEQ ID 64
CACAGGGTATCAAAATTCAAAACTTTCTAAATGAATAAACAGAAACAAAATAATCTTACATTAACAAAC
AAAAACAGAAACAACAAACGAAACCAAAATCATCTAAATCGTTCTAAATTAGCATACGAAACCAAAATC
ATCATCCATCAATAAAAAAAACAAAAAAAAAGAAACGGAGCCAAAATCATCAAAGCTTTTTAAATCAAT
AAACAATACCCAAATCATCTTACATCAACAAACAAAAACCAAATCAATAAACGTAACCAAAATCATTCT
CCTGTAAAAAAAATTTCAAAAGTTATTAGGATTTGTTGGGATGATGTTCACGGGATGAAGCCATACCTT
TTTTTATAGTTGTGATCCACCGCTTGTAAGAAATATAAAAATCATTGAATGATTGATTGTGGTGCAGTG
GGATGAAAGAGTTAATAAATTTTTAATGGCGTCGAATCAATGCAACTTGTAACGCCTTCGAGGAGGGGA
GAAGAACCGCAGACGAAACGACATAAAACCGCAAAGGACGCAAAGACTACTCATGAATACTCGTCTCTT
ACAACCTTGAGAACATCTATTTTTGGTTTATCGTAATCAGAGCTTGCAGGAGAAGATGAACCCTAAAGT
TGAGTGGCGGCTCCACGTTGAAAAAGTTTGTGACTACAGGACAAGCTTTAATTTGTTTATGCCCGGATG
AAATTATGCAAATCCCACAAAATAATGGTGTAAGCCCAAAACCGAACATAACAAATTGAATGATTTTTA
ACGAAGGGAGACACGTGTCGTCGCGACGTCGTCCGATTTATTAACGTGAATGCTGAAGTAGCGCAACAT
GAGGGAGGCAAACATTTTTTTATATATAGATAGATACTTTCACTCTAAAAGTATTATTGAGAATTGCCA
AAAAAGACCTGAATTAAAAAATAAATATAACTGAGAAAGAAAAGAAAATACAGAGAGACAAATTTAAAC
AAAAGGAAAGGGAGATCGAGAGAGGCACACACACACAAAGGAGAATTTTAGGGTTTGGGGAGACTCCGA
AGAGATTGGCGTAACCTTCATTGTACACTTCGTAGGATCTCTCTTCCTTAAATCTCGTTTGAATTTCGT
TATCTGTTTGCTTTCGATTCAATCGCTTTATCGAAATAATGTGTATTCGAATGGAGCCTCCACGATCTG
ATTTTATAGATTCTCCGTTGTTTTGATTTCAGATCTGGATTTTTTCCCCCAATATCTCTAATTGAAAAT
TGTCGATTTCGAGTGTCAGCTGAGAGTATTGTGAACCTGCAGCTGTGGTTTGGATTGTTTATAGCTCAA
TGGTTGAAACTTGATCATTCTTACACATAAAAATTGTTCCTTTACTTCCGTTGATTACTTGGTGAGCTT
ATCCATCTTTCTAGTTGTTAAAGGTGTTAGCTTTTGAAGTATGCCACTCTCTTTTGTGTGCTCGTTTTA
CAGACATCATTCATTTTGTTGATTAACTTGGTCCTCTTTATTGTTTTTTTTTTGTGTGGTGTTTAGTGT
CTTTGAAAGCTCATCTTCCTCGTC
SEQ ID 65
GCAAAGGACGCAAAGACTACTCATGAATACTCGTCTCTTACAACCTTGAGAACATCTATTTTTGGTTTA
TCGTAATCAGAGCTTGCAGGAGAAGATGAACCCTAAAGTTGAGTGGCGGCTCCACGTTGAAAAAGTTTG
TGACTACAGGACAAGCTTTAATTTGTTTATGCCCGGATGAAATTATGCAAATCCCACAAAATAATGGTG
TAAGCCCAAAACCGAACATAACAAATTGAATGATTTTTAACGAAGGGAGACACGTGTCGTCGCGACGTC
GTCCGATTTATTAACGTGAATGCTGAAGTAGCGCAACATGAGGGAGGCAAACATTTTTTTATATATAGA
TAGATACTTTCACTCTA
SEQ ID 66
ACTACGATCCCAAAGGACAACCTATACAATTGTGGAGAGTGACAAAGAAGGGAGAGCATATGAATGGAT
AATACTAGCACTGCATAGCTTAACTTGTATCGTTTTTTCTCCTTAGGTTAGTAGGTATGTTTTACAAAA
ATTAATTTCTATGAATTTTAAATATAATATAAAATAATATGTTTTAGGTGAAACAAATTTATAAGTCCA
ACGGTGGACTTCATGTTCTACAAAAAAAAGTATAGTTAAACGAACCAACCAAATAAACTGTTAGAAATG
CATAATGTTAGGTTTTGTATAAATGTTATGTTTCAATTTGAGCTTTGATAAAATACACACGAGTAAAGA
AAGAGGTAAGATGCACATGTACCTTGTTTGTTGTACACTCAGCCCACTCAACTATTATTACTAAAACGT
CGGTGCCAAAGTTGACAATTCTCTGCTAAATACAATCTGATATACGTCTCTTTCTCCACAACAATATGT
TGATTGGTTAGTGTAATTAGCAATCCTCACATATAGGGAGGAAATCAAATATTCAAATCCAAATGAAAT
TTCCACGGAAGCAAGTAATCAAGTCTTGCGTGCTTACATAACGAGTGACCAA
SEQ ID 67
GTGGAACGGAGACATGTTATGATGTATACGGGAAGCTCGTTAAAAAAAAAATACAATAGGAAGAAATGT
AACAAACATTGAATGTTGTTTTTAACCACCCTTCCTTTTAGCAGTGTACCAATTTTGTAATAGAACCAT
GCATCTCAATCTTAATACTAAAAAATGCAACAAAATTCTAGTGGAGGGACCAGTACCAGTACATTAGAT
ATTATTTTTTATTACTATAATAATAATTTAACTAACACGAGACATAGGAATGTCAAGTGGTAGCGGTAG
GAGGGAGTTGGTTTAGTTTTTTAGATACTAGGAGACAGAACCGGAGGGGCCCATTGCAAGGCCCAAGTT
GAAGTCCAGCCGTGAATCAACAAAGAGAGGGCCCATAATACTGTTGATGAGCATTTCCCTATAATACAG
TGTCCACAGTTGCCTTCCGCTAAGGGATAGCCACCCGCTATTCTCTTGACACGTGTCACTGAAACCTGC
TACAAATAAGGCAGGCACCTCCTCATTCTCAC
SEQ ID 68
TAACGAGATAGAAAATTATATTACTCCGTTTTGTTCATTACTTAACAAATGCAACAGTATCTTGTACCA
AATCCTTTCTCTCTTTTCAAACTTTTCTATTTGGCTGTTGACAGAGTAATCAGGATACAAACCACAAGT
ATTTAATTGACTCATCCACCAGATATTATGATTTATGAATCCTCGAAAAGCCTATCCATTAAGTCCTCA
TCTATGGATATACTTGACAGTTTCTTCCTATTTGGGTATTTTTTTCCTGCCAAGTGGAACGGAGACATG
TTATGTTGTATACGGGAAGCTCGTTAAAAAAAAAATACAATAGGAAGAAATGTAACAAACATTGAATGT
TGTTTTTAACCATCCTTCCTTTTAGCAGTGTACCAATTTTGTAATAGAACCATGCATCTCAATCTTAAT
ACTAAAAAATGCAACAAAATTCTAGTGGAGGGACCAGTACCAGTACATTAGATATTATTTTTTATTACT
ATAATAATAATTTAACTAACACGAGACATAGGAATGTCAAGTGGTAGCGGTAGGAGGGAGTTGGTTTAG
TTTTTTAGATACTAGGAGACAGAACCGGAGGGGCCCATTGCAAGGCCCAAGTTGAAGTCCAGCCGTGAA
TCAACAAAGAGAGGGCCCATAATACTGTTGATGAGCATTTCCCTATAATACAGCGTCCACAGTTGCCTT
CCGCTAAGGGATAGCCACCCGCAATTCTCTTGACACGTGTCACTGAAACCTGCTACAAATAAGGCAGGC
ACCTCCTCATTCTCAC
SEQ ID 69
TAATCGCGTAATTTTCCCCATTAATTATATATAAAATTCTTAAGAAATTCTCGAGGCAGTAAAGGTTCC
ACAAATTGAAATCAGGAAGAAACTATTAACTAATCTATTTTCTTTTCTTCAACGACTACTACTTATTAT
ATTGGCTCTAAAGATAAGAGGATAATGAAACAAAGGAAGAAGCTTTAACGAGATAGAAAATTATATTAC
TCCGTTTTGTTCATTACTTAACAAATGCAACAGTATCTTGTACCAAATCCTTTCTCTCTTTTCAAACTT
TTCTATTTGGCTGTTGACAGAGTAATCAGGATACAAACCACAAGTATTTAATTGACTCATCCACCAGAT
ATTATGATTTATGAATCCTCGAAAAGCCTATCCATTAAGTTCTCATCTATGGATATACTTGACAGTTTC
TTCCTATTTGGGTATTTTTTTTTCCTGCCAAGTGGAACGGAGACATGTTATGTTGTATACGGGAAGCTC
GTTAAAAAAAAAAATACAATAGGAAGAAATGTAACAAACATTGAATGTTGTTTTTAACCATCCTTCCTT
TTAGCAGTGTATCAATTTTGTAATAGAACCATGCATCTCAATCTTAATACTAAAAAATGCAACAAAATT
CTAGTGGAGGGACCAGTACCAGTACATTAGATATTATTTTTTATTACTATAATAATATTTTAATTAACA
CGAGACATAGGAATGTCAAGTGGTAGCGGTAGGAGGGAGTTGGTTTAGTTTTTTAGATACTAGGAGACA
GAACCGGAGGGGCCCATTGCAAGGCCCAAGTTGAAGTCCAGCCGTGAATCAACAAAGAGAGGGCCCATA
ATACTGTCGATGAGCATTTCCCTATAATACAGTGTCCACAGTTGCCTTCCGCTAAGGGATAGCCACCCG
CTATTCTCTTGACACGTGTCACTGAAACCTGCTACAAATAAGGCAGGCACCTCCTCATTCTCAC
SEQ ID 70
AAGCTTTAACGAGATAGAAAATTATAATACTCCGTTTTGTTCATTACTTAACAAATGCAACAGTATCTT
GTACCAAATCCTCTCTCTTTTCAAACTTTTCTATTTGGCTGTTGACAGAGTAATCAGGATACAAACCAC
AAGTATTTAATTGACTCATCCACCAGATATTATGATTTATGAATCCTCGAAAAGCCTATCCATTAAGTC
CTCATCTATGGATATACTTGACAGTTTCTTCCTATTTGGGTTTTTTTTTTTCCTGCCAAGTGGAACGGA
GACATGTTATGTTGTATACGGGAATCTCGTTAAAAAAAAAAATACAATAGGAAGAAATGTAACAAACAT
TGAATGTTGTTTTTAACCATCCTTCCTTTTAGCAGTGTATCAATTTTGTAATAGAACCATGCATCTCAA
TCTTAATACTAAAAAATGCAACAAAATTCTAGTGGAGGGACCAGTACCAGTACATTAGATATTATTTTT
TATTACTATAATAATATTTTAATTAACACGAGACATAGGAATGTCAAGTGGTAGCGGTAGGAGGGAGTT
GGTTTAGTTTTTAGATACTAGGAGACAGAACCGGAGGGGCCCATTGCAAGGCCCAAGTTGAAGTCCAGC
CGTGAATCAACAAAGAGAGGGCCCATAATACTGTCGATGAGCATTTCCCTATAATACAGTGTCCACAGT
TGCCTTCCGCTAAGGGATAGCCACCCGCTATTCTCTTGACACGTGTCACTGAAACCTGCTACAAATAAG
GCAGGCACCTCCTCATTCTCAC
SEQ ID 71
GAACCATGCATCTCAATCTTAATACTAAAATGCAACTTAATATAGGCTAAACCAAGTAAAGTAATGTAT
TCAACCTTTAGAATTGTGCATTCATAATTAGATCTTGTTTGTCGTAAAAAATTAGAAAATATATTTACA
GTAATTTGGCATACAAAGCTAAGGGGGAAGTAACTACTAATATTCTAGTGGAGGGACCAGTACCAGTAC
CAGTACCTAGATATTATTTTTTATTACTATAATAATAATTTAATTAACACGAGACTGATAGGAATGTCA
AGTGGTAGCGGTAGGAGGGAGTTGGTTTAGTTTTTTAGATACTAGGAGACAGAACCGGACGGGCCCATT
GCAAGGCCCAAGTTGAAGTCCAGCCGTGAATCAACAAAGAGAGGGCCCATAATACTGTCGATGAGCATT
TCCCTATAATACAGTGTCCACAGTTGCCTTCCGCTAAGGGATAGCCACCCGCTATTCTCTTGACACGTG
TCACTGAAACCTGCTACAAATAAGGCAGGCACCTCCTCATTCTCAC
SEQ ID 72
GAACCATGCATCTCAATCTTAATACTAAAATGCAACTTAATATAGGCTAAACCAAGTAAAGTAATGTAT
TCAACCTTTAGAATTGTGCATTCATAATTAGATCTTGTTTGTCGTAAAAAATTAGAAAATATATTTACA
GTAATTTGGCATACAAAGCTAAGGGGGAAGTAACTACTAATATTCTAGTGGAGGGACCAGTACCAGTAC
CAGTACCTAGATATTATTTTTTATTACTATAATAATAATTTAATTAACACGAGACTGATAGGAATGTCA
AGTGGTAGCGGTAGGAGGGAGTTGGTTTAGTTTTTTAGATACTAGGAGACAGAACCGGAGGGGCCCATT
GCAAGGCCCAAGTTGAAGTCCAGCCGTGAATCAACAAAGAGAGGGCCCATAATACTGTCGATGAGCATT
TCCCTATAATACAGTTGCCTTCCGCTAAGGGATAGCCACCCGCTATTCTCTTGACACGTGTCACTGAAA
CCTGCTACAAATAAGGCAGGCACCTCCTCATTCTCAC
SEQ ID 73
ATTCTAGTGGAGGGACCAGTACCAGTACATTAGATATTATTTTTTATTACTATAATAATATTTTAATTA
ACACGAGACATAGGAATGTCAAGTGGTAGCGGTAGGAGGGAGTTGGTTTAGTTTTTTAGATACTAGGAG
ACAGAACCGGAGGGGCCCATTGCAAGGCCCAAGTTGAAGTCCAGCCGTGAATCAACAAAGAGAGGGCCC
ATAATACTGTCGATGAGCATTTCCCTATAATACAGTGTCCACAGTTGCCTTCCGCTAAGGGATAGCCAC
CCGCTATTCTCTTGACACGTGTCACTGAAACCTGCTACAAATAAGGCAGGCACCTCCTCATTCTCAC
SEQ ID 74
ATTCTAGTGGAGGGACCAGTACCAGTACATTAGATATTATTTTTTATTACTATAATAATATTTTAATTA
ACACGAGACATAGGAATGTCAAGTGGTAGCGGTAGGAGGGAGTTGGTTTAGTTTTTTAGATACTAGGAG
ACAGAACCGGAGGGGCCCATTGCAAGGCCCAAGTTGAAGTCCAGCCGTGAATCAACAAAGAGAGGGCCC
ATAATACTGTCGATGAGCATTTCCCTATAATACAGTGTCCACAGTTGCCTTCCGCTAAGGGATAGCCAC
CCGCTATTCTCTTGACACGTGTCACTGAAACCTGCTACAAATAAGGCAGGCACCTCCTCATTCTCACGT
CCTCATCTATGGATATACTTGACAGTTTCTTCCTATTTGGGTATTTTTTTCCTGCCAAGTGGAACGGAG
ACATGTTATGTTGTATACGGGAAGCTCGGTGAGAATGAGGAGGTGCCTGCCTTATTTGTAGCAGGTTTC
AGTGACACGTGTCAAGAGAATAGCGGGTGGCTATCCCTTAGCGGAAGGCAACTGTGGACACTGTATTAT
AGGGAAATGCTCATCGACAGTATTATGGGCCCTCTCTTTGTTGATTCACGGCTGGACTTCAACTTGGGC
CTTGCAATGGGCCCCTCCGGTTCTGTCTCCTAGTATCTAAAAAACTAAACCAACTCCCTCCTACCGCTA
CCACTTGACATTCCTATGTCTCGTGTTAATTAAAATATTATTATAGTAATAAAAAATAATATCTAATGT
ACTGGTACTGGTCCCTCCACTAGAAT
SEQ ID 75
AAAAACCTCCTCCACTCAGTCTTGGGATCTCTCTCTCTCTTCACGCTTCTCTTGGGGCCTTGAACTCAG
CAATTTGACACTCAGTTAGTTACACTCCTATCACTCATCAGATCTCTATTTTTTCTCTTAATTCCAACC
AAGGAATGAATTAAAAGATTAGATTTGAAGGAGAGAAGAAGAAAGATGGTGTATACACTCTCTGGAGTT
CGTTTTCCTACTGTTCCATCAGTGTACAAATCTAATGGATTCAGCAGTAATGGTGATCGGAGGAATGCT
AATGTTTCTGTATTCTTGAAAAAGCACTCTCTTTCACGGAAGATCTTGGCTGAAAAGTCTTCTTACGAT
TCCGAATCCCGACCTTCTACAGTTGCAGCATCGGGGAAAGTCCTTGTACCTGGAATCCAGAGTGATAGC
TCCTCATCCTCAACAGACCAATTTGAGTTCACTGAGACAGCTCCAGAAAATTCCCCAGCATCAACTGAT
GTGGATAGTTCAACAATGGAACACGCTAGCCAGATTAAAACTGAGAACGATGACGTTGAGCCGTCAAGT
GATCTTACAGGAAGTGTTGAAGAGTTGGATTTTGCTTCATCACTACAACTACAAGAAGGTGGTAAACTG
GAGGAGTCTAAAACATTAAATACTTCTGAAGAGACAATTATTGATGAATCTGATAGGATCAGAGAGAGG
GGCATCCCTCCACCTGGACTTGGTCAGAAGATTTATGAAATAGACCCCCTTTTGACAAACTATCGTCAA
CACCTTGATTACAGGTATTCACAGTACAAGAAACTGAGGGAGGCAATTGACAAGTATGAGGGTGGTTTG
GAAGCTTTTTCTCGTGGTTATGAAAAAATGGGTTTCACTCGTAGTGCTACAGGTATCACTTACCGTGAG
TGGGCTCCTGGTGCCCAGTCAGCTGCTCTCATTGGAGATTTCAACAATTGGGACGCAAATGCTGACATT
ATGACTCGGAATGAATTTGGTGTCTGGGAGATTTTTCTGCCAAATAATGTGGATGGTTCTCCTGCAATT
CCTCATGGGTCCAGAGTGAAGATACGCATGGACACTTCATCAGGTGTTAAGGATTCCATTCCTGCTTGG
ATCAACTACTCTTTACAGCTTCCTGATGAAATTCCATATAATGGAATATATTATGATCCACCCGAAGAG
GAGAGGTATGTCTTCCAACACCCACGGCCAAAGAAACCAAAGTCGCTGAGAATATATGAATCTCATATT
GGAATGAGTAGTCCGGAGCCTAAAATTAACTCATACGTGAATTTTAGAGATGAAGTTCTTCCTCGCATA
AAAAACCTTGGGTACAATGCGGTGCAAATTATGGCTATTCAAGAGCATTCTTATTATGCTAGTTTTGGT
TATCATGTCACAAATTTTTTTGCACCAAGCAGCCGTTTTGGAACGCCCGACGACCTTPAGTCTTTGATT
GATAAAGCTCATGAGCTAGGAATTGTTGTTCT
SEQ ID 76
CCATTTAACTTTGATTGTAATTAATTTTTAAAAATTACCAACATATAAATAAAATTAATATTTAACAAA
GAATTGTAACATAATATTTTTTTAATTATTCAAAATAAATATTTTTAAACATCATATAAAAGAAATACG
ACAAAAAAATTGAGACGGGAGAAGACAAGCCAGACAAAAATGTCCAAGAAACTCTTTCGTCTAAATATC
TCTCATCCAAACTAATATAATACCCATTAC
>SEQ ID 77
CTACCGAGGAATTCCTCGGCAGTTAACTGCAGCCGGATTTCAAATTCCTCGGCAGTTAACTGCCGAGGG
GGCAAAAGCGTATTTTACATGTGTGTCCCAGCCTTCTTTAATGTGTGAACAACAATTTTCTAAAATTAA
ACCCTACTCTAGGTTTAACATACCAGTAAATTTTTGCTTTTTGTATGTGTTAACCCTTCTCCAATCCCT
TGCACAACCATCTCCTCAAACCTTCTTCTTCTGGAGCAAAGTCGCCATTCCCTACCTCCTTCTTCATTC
TTATTCTCTATAACAAACGGTCCGACCGGATCCAAGTTGCACCGGTTCGAACCGCTTTAGTTACTACTA
ACGGTTCGAACCGTTATTTTTCAACCCGTGACGAACGTGGAAGGCTTCGTTGTTTCTTCTTCTTCTTCT
TCTTCTTCTTATTAATTACCATGCGTTTTTGTTTTTCTTTTGAG
SEQ ID 78
CAAGTGTCTGAGACAACCAAAACTGAAAGTGGGAAACCAAACTCTAAGTCAAAGACTTTATATACAAAA
TGGTATAAATATAATTATTTAATTTACTATCGGGTTATCGATTAACCCGTTAAGAAAAAACTTCAAACC
GTTAAGAACCGATAACCCGATAACAAAAAAAATCTAAATCGTTATCAAAACCGCTAAACTAATAACCCA
ATATTGATAAACCAATAACTTTTTTTATTCGGGTTATCGGTTTCAGTTCTGTTTGGAACAATCCTAGTG
TCCTAATTATTGTTTTGAGAACCAAGAAAACAAAAACTTACGTCGCAAATATTTCAGTAAATACTTGTA
TATCTCAGTGATAATTGATTTCCAACATGTATAATTATCATTTACGTAATAATAGATGGTTTCCGAAAC
TTACGCTTCCCTTTTTTCTTTTGCAGTCGTATGGAATAAAAGTTGGATATGGAGGCATTCCCGGGCCTT
CAGGTGGAAGAGACGGAGCTGCTTCACAAGGAGGGGGTTGTTGTACTTGAAAATGGGCATTTATTGTTC
GCAAACCTATCATGTTCCTATGGTTGTTTATTTGTAGTTTGGTGTTCTTAATATCGAGTGTTCTTTAGT
TTGTTCCTTTTAATGAAAGGATAATATCTGTGCAAAAATAAGTAAATTCGGTACATAAAGACATTTTTT
TTTGCATTTTCTGTTTATGGAGTTGTCAAATGTGAATTTATTTCATAGCATGTGAGTTTCCTCTCCTTT
TTCATGTGCCCTTGGGCCTTGCATGTTTCTTGCACCGCAGTGTGCCAGGGCTGTCGGCAGATGGACATA
AATGGCACACCGCTCGGCTCGTGGAAAGAGTATGGTCAGTTTCATTGATAAGTATTTACTCGTATTCGG
TGTTTACATCAAGTTAATATGTTCAAACACATGTGATATCATACATCCATTAGTTAAGTATAAATGCCA
ACTTTTTACTTGAATCGCCGAATAAATTTACTTACGTCCAATATTTAGTTTTGTGTGTCAAACATATCA
TGCACTATTTGATTAAGAATAAATAAACGATGTGTAATTTGAAAACCAATTAGAAAAGAAGTATGACGG
GATTGATGTTCTGTGAAATCACTGGTAAATTGGACGGACGATGAAATTTGATCGTCCATTTAAGCATAG
CAACATGGGTCTTTAGTCATCATCATTATGTTATAATTATTTTCTTGAAACTTGATACACCAACTTTCA
TTGGGAAAGTGACAGCATAGTATAAACTATAATATCAATTCTGGCAATTTCGAATTATTCCAAATCTCT
TTTGTCATTTCATTTCCTCCCCTATGTCTGCAAGTACCAATTATTTAAGTACAAAAAATCTTGATTAAA
CAATTTATTTTCTCACTAATAATCACATTTAATCATCAACGGTTCATACACGTCTGTCACTCTTTTTTT
ATTCTCTCAAGCGCATGTGATCATACCAATTATTTAAATACAAAAAATCTTGATTAAACAATTCAGTTT
CTCACTAATAATCACATTTAATCATCAACGGTTCATACACATCCGTCACTCTTTTTTTATTCTCTCAAG
CGCATGTGATCATACCAATTATTTAAATACAAAAAATCTTGATTALACAATTCATTTTCTCACTAATAA
TCACATTTAATCATCAACGGTTTATACACGTCCGCCACTCTTTTTTTATTCTCTCAAGCGTATGTGATC
ATATCTAACTCTCGTGCAAACAAGTGAAATGACGTTCACTAATAAATAATCTTTTGAATACTTTGTTCA
GTTTAATTTATTTAATTTGATAAGAATTTTTTTATTATTGAATTTTTATTGTTTTAAATTAAAAATAAG
TTAAATATATCAAAATATCTTTTAATTTTATTTTTGAAAAATAACGTAGTTCAAACAAATTAAAATTGA
GTAACTGTTTTTCGAAAAATAATGATTCTAATAGTATATTCTTTTTCATCATTAGATATTTTTTTTAAG
CTAAGTACAAAAGTCATATTTCAATCCCCAAAATAGCCTCAATCACAAGAAATGCTTAAATCCCCAAAA
TACCCTCAATCACAAGACGTGTGTACCAATCATACCTATGGTCCTCTCGTAAATTCCGACAAAATCAGG
TCTATAAAGTTACCCTTGATATCAGTATTATAAAACTAAAAATCTCAGCTGTAATTCAAGTGCAATCAC
ACTCTACCACACACTCTCTAGTAGAGAGATCAGTTGATAACAAGCTTGTTAACGGATCCCTAGTAATAC
TGAGATTAGTTACCTGAGACTATTTCCTATCTTCTGTTTTGATTTGATTTATTAAGGAAAATTATGTTT
CAACGGCCATGCTTATCCATGCATTATTAATGATCAATATATTACTAAATGCTATTACTATAGGTTGCT
TATATGTTCTGTAATACTGAATATGATGTATAACTAATACATACATTAAATTCTCTAATAAATCTATCA
ACAGAAGCCTAAGAGATTAACAAATACTACTATTATCCAGACTAAGTTATTTTTCTGTTTACTACAGAT
CCTTCCAAGAACAAAAACTTAATAATTGTATGGCTGCTATACCATCAAACCAAACAATGTATAAGAAAT
AATACTTGCATAACTAATGCACGCACTACTAATGCAAGCATTACTAATGCACCATATTTTGTATTTGTT
CTTATACACTCTACCAAACGACCCCTTAGAGTGTGGGTAAGTAATTAAGTTAGGGATTTGTGGGAAATG
GACAAATATAAGAGAGTGCAGGGGAGTAGTGCAGGAGATTTTCGTGCTTTTATTGATAAATAAAAAAAG
GGTGACATTTAATTTCCACAAAATTCTTATGTTAACCAAATAAATTGAGACAAATTAATTCAGTTAACC
AGAGTTAAGAGTAAAGTACTATTGCAAGAAAATATCAAAGGCAAAAGAAAAGATCATGAAAGAAAATAT
CAAAGAAAAAGAAGAGGTTACAATCAAACTCCCATAAAACTCCAAAAATAAACATTCAAATTGCAAAAA
CATCCAATCAAATTGCTCTACTTCACGGGGCCCACGCCGGCTGCATCTCAAACTTTCCCACGTGACATC
CCATAACAAATCACCACCGTAACCCTTCTCAAAACTCGACACCTCACTCTTTTTCTCTATATTACAATA
AAAAATATACGTGTCCGTGGTAACTTTTACTCATCTCCTCCAATTATTTCTGATTTCATGCATGTTTCC
CTACATTCTATTATGAATCGTGTTATGGTGTATAAACGTTGTTTCATATCTCATCTCATCTATTCTGAT
TTTGATTCTCTTGCCTACTGAATTTGACCCTACTGTAATCGGTGATAAATGTGAATGCTTCCTCTTCTT
CTTCTTCTTCTCAGAAATCAATTTCTGTTTTGTTTTTGTTCATCTGTAGGGACACGTATATTTTTTATT
GTAATATAGAGAAAAAGAGTGAGGTGTCGAGTTTTGAGAAGGGTTACGGTGGTGATTTGTTATGGGATG
TCACGTGGGAAAGTTTGAGATGCAGCCGGCGTGGGCCCCGTGAAGTAGAGCAATTTGATTGGATGTTTT
TGCAATTTGAATGTTTATTTTTGGAGTTTTATGGGAGTTTGATTGTAACCTCTTCTTTTTCTTTGATAT
TTTCTTTCATGATCTTTTCTTTTGCCTTTGATATTTTCTTGCAATAGTACTTTACTCTTAACTCTGGTT
AACTGAATTAATTTGTCTCAATTTATTTGGTTAACATAAGAATTTTGTGGAAATTAAATGTCACCCTTT
TTTTATTTATCAATAAAAGCACGAAAATCTCCTGCACTACTCCCCTGCACTCTCTTATATTTGTCCATT
TCCCACAAATCCCTAACTTAATTACTTACCCACACTCTAAGGGGTCGTTTGGTAGAGTGTATAAGAACA
AATACAAAATATGGTGCATTAGTAATGCTTGCATTAGTAGTGCGTGCATTAGTTATGCAAGTATTATTT
CTTATACATTGTTTGGTTTGATGGTATAGCAGCCATACAATTATTAAGTTTTTGTTCTTGGAAGGATCT
GTAGTAAACAGAAAAATAACTTAGTCTGGATAATAGTAGTATTTGTTAATCTCTTAGGCTTCTGTTGAT
AGATTTATTAGAGAATTTAATGTATGTATTAGTTATACATCATATTCAGTATTACAGAACATATAAGCA
ACCTATAGTAATAGCATTTAGTAATATATTGATCATTAATAATGCATGGATAAGCATGGCCGTTGAAAC
ATAATTTTCCTTAATAAATCAAATCAAAACAGAAGATAGGAAATAGTCTCAGGTAACTAATCTCAGTAT
TACTAGTTTTAATGTTTAGCAAATGTCCTATCAGTTTTCTCTTTTTGTCGAACGGTAATTTAGAGTTTT
TTTTGCTATATGGATTTTCGTTTTTGATGTATGTGACAACCCTCGGGATTGTTGATTTATTTCAAAACT
AAGAGTTTTTGCTTATTGTTCTCGTCTATTTTGGATATCAATCTTAGTTTTATATCTTTTCTAGTTCTC
TACGTGTTAAATGTTCAACACACTAGCAATTTGGCTGCAGCGTATGGATTATGGAACTATCAAGTCTGT
GGGATCGATAAATATGCTTCTCAGGAATTTGAGATTTTACAGTCTTTATGCTCATTGGGTTGAGTATAA
TATAGTAAAAAAATAGGAATTCGCGGTAC
SEQ ID 79
ATTTAGCAGCATTCCAGATTGGGTTCAATCAACAAGGTACGAGCCATATCACTTTATTCAAATTGGTAT
CGCCAAAACCAAGAAGGAACTCCCATCCTCAAAGGTTTGTAAGGAAGAATTCTCAGTCCAAAGCCTCAA
CAAGGTCAGGGTACAGAGTCTCCAAACCATTAGCCAAAAGCTACAGGAGATCAATGAAGAATCTTCAAT
CAAAGTAAACTACTGTTCCAGCACATGCATCATGGTCAGTAAGTTTCAGAAAAAGACATCCACCGAAGA
CTTAAAGTTAGTGGGCATCTTTGAAAGTAATCTTGTCAACATCGAGCAGCTGGCTTGTGGGGACCAGAC
AAAAAAGGAATGGTGCAGAATTGTTAGGCGCACCTACCAAAAGCATCTTTGCCTTTATTGCAAAGATAA
AGCAGATTCCTCTAGTACAAGTGGGGAACAAAATAACGTGGAAAAGAGCTGTCCTGACAGCCCACTCAC
TAATGCGTATGACGAACGCAGTGACGACCACAAAAGA
SEQ ID 80
CACCGGCTGCAGATATTTTTTTAAGTTTTCTTCTCACATGGGAGAAGAAGAAGCCAAGCACGATCCTCC
ATCCTCAACTTTATAGCATTTTTTTCTTTTCTTTCCGGCTACCACTAACTTCTACAGTTCTACTTGTGA
GTCGGCAAGGACGTTTCCTCATATTAAAGTAAAGACATCAAATACCATAATCTTAATGCTAATTAACGT
AACGGATGAGTTCTATAACATAACCCAAACTAGTCTTTGTGAACATTAGGATTGGGTAAACCAATATTT
ACATTTTAAAAACAAAATACAAAAAGAAACGTGATAAACTTTATAAAAGCAATTATATGATCACGGCAT
CTTTTTCACTTTTCCGTAAATATATATAAGTGGTGTAAATATCAGATATTTGGAGTAGAAAAAAAAAAA
AAGAAAAAAGAAATATGAAGAGAGGAAATAATGGAGGGGCCCACTTGTAAAAAAGAAAGAAAAGAGATG
TCACTCAATCGTCTCACACGGGCCCCCGTCAATTTAAACGGCCTGCCTTCTGCCCAATCGCA
SEQ ID 81
TCGAAGAAAAAAAACAATTTATACGACCAGAAATGGCAAAATGTTGTTCTTAGAATTTTTTTCTACTTT
ACTTTTGCGTAAAACACATTTCTCCAATTTGGTTTCATTGCGTTGAACGACGTAACAAAGTAATACACC
CAACCCTTTTTTTTGGAACATTATGCACCCAACCCATTGTACAAAAGTTACAGCTAATTACCATTTTTA
TTCTTTTGATAAATACAAAAATAAATTATTAATCATTAAAAAAAAATTTGGAATATTTTCTCAATGTCC
ATATATACATCTTCTCCCTTTATATAAGCCAACCTCACACACCCAAAAAATCCATCAAACC
SEQ ID 82
CCCCTGGTCCATAAAAAAGGTCTTACATATTTACTTTCGCATACATATTTTTCTAATTTAATTTCACTG
AATAGAACGATGTAACAAAGTAACCAAACCCATTGCATTTAAAATTACAGCAAAATTATCCTTTTTTTA
AAATATATAATTATTTCTTTAAATATATATATATTTTTTTTATTTTTTTTTCAACAAATATATAATTAT
TAAAAAAAAACAGTTTTGAGTATCTCAATCAATTCTACAGACTTACACATCCTCCTTCCCCTTTATATA
AAGAAACTTCAGACCTCAAAATACATCGAACCCTTTCT
SEQ ID 83
TAAAAGGGGAAGATGTGAACAAGGGTAAGACACGAGTTACTTTTCAACGGTGAATAATTAAAATATTTA
ATTATTTTTTTGTAGCAGGTTGAGCCGGTTGTGTTTTAGGAATATTACAGTATTATTTTATATTTGTAA
CAGCGTGTATAAGATCGTTAGGTTAAATGGCTAGACGGTGAATTACGTTTTTTTTTGTGGTTATAGCCT
TCAATTTCCCATTTAATTTCACCGAATAGAACGATGTAACAAAATAACAAACCCATTGCATTTAAAATT
ACAGCAAATTACCCTTTTTATTCTTTAAATATATAATTATTTAATAAAAACAGTTTGAGCATCTCAATG
TCTACAGACTACACATCTTCCTTCCCCTTTATATAAACAAACTTCACAGACCGCAAAATACATCGAACC
CTT
SEQ ID 84
GTAAATTAAGCGTCTAATAAATGAAATAACTATTTGTCGGTCTGTATGCATGCTAAACCTGTCTTTCAA
TTGGAGCATGACTATACAAAATGTCTAAAAGCCGATGAAGTTCTCTGTGTCTTATGATAATAGATTTCA
GCATCGAAAATCAAGTTTTAAGGAGCTGCTCTACATATGCGATGGAGATAGCAACGGGGTCCTTTATTT
TGCTGGCACATCATATGGGAAACACCAGTGGGTGAATCCTGTTTTGTCCAAGGTAAATCCACAGCTGCA
ATAAGCAATTTACCTTCCTTCTTTTGACTTGTTACCGTTCTAAAAAATATACAATTGTTTACCATCTCA
TTTTGTCATCTGTTTAACATTGGTAATTCATGTTTCAGAGAGTAATTATCACGGCTAGTAGCCCCATTT
CAAGATGCACTGATCCCAAGGTGTTAGTATCGAGGAACTTCCAGGTTTGAATAGATGACATCCAATTAA
TGTGAAGGATCTTCTCCTTCTAGATTAATTTGAGAAAAAAAAAGAAATATTCTTTTGCTCTCTCTCTCT
TTTTCATCGATGGCATGAAGAAGAGGAAGTCGATACACAAAAGAGAGTGTTAGCTCCATAATGTGAAGG
ATGAAATATTTTTTTGGTCTCAGGGTACATCTGTTGCTGGACCTCAGGTGGAGGGCGGAAGAAACGCTT
CGTGGTGGATGGTTGATATTGGTCCGGATCACCAGGTTAGATTTATTGGTTTGTGTATAATTTAATTGT
GTGTACATAAGGGAGATGGAAAGAAGTTTTTGTAAAATAAGATGTATGTTGTAACTTAGACAATCACTT
CGTCCGTGCTGATTCTCAGATTCATCTGTATTTTTAATTGACTTGTGAAAGTGAACATTTAAAATTGAA
CATCGGTAACTTGCATTTCTCATTGTAAGGGCATTGCATGATATCATGGTTGTCTAGAGTAGTGCTGAT
CAGTATACCTCGTGGACAAGATACTGAAAGTGAACACTCATCTCTGCTCTTTTGGTTTCGTTAAAAGTA
CTCTCTCTCTCAGTTTATAGCACACTCAAATTGTGTGTCAATATCCCTGATTGATTTTCTCATTTGGTA
TTCAACTAGAAGATGAAACTTCTGACGCATTTAATATTAGATGAATCGATGCAGCTCATGTGTAACTAC
TACACATCAAGACAGGACGGATCAAGAGCATTTATCAGACGTTGGAACTTTCAGGTAAGCAGTGCACTC
AACATTCACAAACCAGTATACACATCATCTCTAATGGATCTGTGGATGCACTCGTAACTCGTCTATAGA
TTATACATATATACATACATATATACGTACCAACATCTCCATTTTGTAGAACTGGAAACGTTGTTAAAA
TTGGCGTTACAATAACAAATTTTTATGCATTGCATTCTCAGGGCTCTTTGGATGGGAAAAATTGGACAA
ACCTGAGAGTACATGAGAATGATCAAACTATTTGCAAGCCAGGTCAATTTGCATCATGGCCAATTACTG
GTTCAAATGCATTACTTCCTTTCAGATTCTTTCGAGTTCTCATGACCGGTCCTACTACAGACGCTACTA
ACCCGTGGAACTGTTGCATCTGCTTCTTAGAACTCTATGGCTATTTTCGTTAGCTTGGCGTCGGTTTGA
ACATAGTTTTTGTTTTCAAACTCTTCATTTACAGTCAAAATGTTGTATGGTTTTTGTATTCCTCAATGA
TGTTTACAGTGTTGTGTTGTCATCTGTACTCTTTGCCTGTTACTTGTTTTGAGTTACATGTTTAAAAAA
GTGTCTTTCTGCCATATTTTGTTCTCTTATTATTATTATTGTTATTATCATACATACATATTAAAAGGG
AAATGACAAGTACACAAATCTTAGACCGTTTATGTTCAATCAACTTTTGGAGGCATTGACAGGTCCAAA
ATTTTGAGTTTATGATTAAGTTCAATCTTAGAATATGAATTTAACATCTATTATAGATACATAAAAATA
GCTAATGATAGAACATTGACATTTGGCAGAGCTTAGGGTATGGTATATCCAACGTTAATTTTAGTAATT
TTTGTTACGTACGTATATTAAATGTTGAATTAATCACATGAACGGTGGATATTATATTATGAATTGGCA
TCAGCAAAATTATTAGTGTAGTTGACTTGTAGTTGCAGTTTTAATAATAAAATGGTAATTAACGGTCGA
TATTAAAATAACTCTCATTTCAAGTGGGATTAGAACTAGTTATTAAAAAAATGTATACTTTAAGTGATT
TGATGGCTTATAATTTAAAGTTTTTCATTTCATGCTAAAATTGTTAATCATTGTAATGTAGACTGCGAC
TGGAATTATTATAGTGTAAATTTATGCATTCGGTGTAAAATTAATGTATTGAACTTGTCTTTTTTAGAA
AATACTTTGTACTTTAATATAGGATTCTGTCATGGGAATTTAAATTAATCGATATCGAACACGGATGGA
ATACCAAAATTAAAAAAAATACACAPGGCCTTCATATGAACCGTGAACCTTTGATAACGTGGAAGTTCA
AAGAAGTAAAGTTTAAGAATAAACTGACAAATTAATTTCTTTTATTTGGCCCACTACTAAATTTGCCTT
ACTTTCTAACATGTCAAGTTGTCTCCTCGTAGTTGAATGATATTCATTTTTCATCCCTTAAGTTCAATT
TGATTGTCATACTCACCCATGATGTTCTGAAAAATGCTTGGCCATTCACAAATTTTATCTTAGTTCCTA
TGAACTTTATAAGAAGCTTTAATTTGACATGTTATATTATTAGATAATATAATCCATAACCCAATAAAC
AAGTGTATTAATATTGTAACTTTGTAATTGAGTGCGTCCACATCTTATTCAATCATTTAAGGTCATTAA
AAAAAATTATTTTTTGACATTCTAAAACTTTGAGTTGAATAAATAGTTCATCAATTATTAATACATACC
AATGAAAAGAACAAAAATGACTTATTTATAAATCAACAAACAATTTTAGATTGCTCCAACATATTTTCC
AAAATTAACATTTAAATTTTAATGCAAGAAAATGCATAATTTTTTACTTGATCTTTATAGCTTATTTTT
TCAGTCTAATCAACGAATATTTGAAACTCGCAACTTGATTAAAGGGATTTACAACAAGATATATATAAG
TAGTGACAAATCTTGATTTTAAATATTTTAATTTGGAGGTCAAAATTTTACCATAACCATTTGATTTAT
AACTAAATTTTAAATATATTATTTATACATATCTAGTAAATTTTTAAATATATGTATATACAAAATATA
AAATTATTGTGTTCATATATGTCGATAAATCCTTAAATAATATCTGCCTTTACCACTAGAGAAAGTAAA
AAACTCTTTACCAAAAATACATGTATTATGTATACAAAAAGTTGATTTGATAACTATTGAAATTGTATA
CGAGTAAGTAATAGAAATATAAAAAACTACAAAACTAAAAAAATATATGTTTTACTTTAATTTCGAAAC
TAATAGGGTCTGAGTGAAATATTCAGAAAGTGGACTACAGAGGGTCATAATGTTTTTTTATTAAAAGCC
ACTAAAGTGAGGAAATC
SEQ ID 85
GTACTAAATGATAATTATATTAAATTGATGAATATATGACATATATAAATATATAGACATTTATTATTT
AATCATGAATAATATTATTTTTTTACTTCACTAAATTATTTCACCAGAATAAATTTGATTTAATTCAGA
TAAACGAGTTGGTAATTACCCTATCACAAATTTGGAATTAGTGAATGAAATTTTGATCCAATAGCAAAG
CCAAAGATAAAACTTTTCAACTCATTCAGGTGGCACTTAAAATCAAGATATTCTTGGTATCTTTTCAAT
ATATAAGTATATGATGACGAATTAGTGGAACTAAAAGAATATCCCATCAAAATGCTTTACAACAGAAAC
ACTTTAACTTTTAGTAGACATTTTCAAAATTGAAAAATAATATTTAAAAATTAAAATTGTATTTAGTTA
TAAATACAAAATAGAATGTTTTTTTAATTGTGAATAATTTAAAGTGAAAACACTATTTTTGACATTTTA
AATTTTTTTGAATTCAAAGCTTTTGTTCAAGCTTTAACTACAACTTTTGAATTTTGAATATTATGCAAC
TCAAATATGAATATTAGTTTGTGATTCCAATAGATATATTGTATAGAAATGAAAAAAATGAATAATGCC
ACAAATTTTACTAATGGTCAAGATGAGTGGTAAATGGTAAGTAACCTCCATCCTCAACTGAAGGTGACT
AGTTTGAGCTGTTGAAAATAGAGCACTTATAATAGCAATCACTTTACTCTTCGAAGTAAAAAAAAATGA
AATGATCCAAATCCGTATTAATCCAACTTCAAAATGGTTAACCCGACATTGAATACCTCAACGTTCAGA
TTCCAGCAAACACACAACAATATTTGGTGATTTCTTTTCAAGTGTTTTAGTCTTGATGCAGAGTCACTC
AATACATGTGTTAGTAAAATATAATAACTATTACATCAAAATTAGCATAGGATTGTTGGGTTCTGAAGG
TGAATAGGGCGTCATGCGGAAGCTTGCAATTTGCAAATCATATTGTTGATAAATCAGATAACAAAAACT
TATACTAAAAATCAAAATATTATTATATCAAATTAATATAAAGAAAAACATTGAAACTTTAGAGAGAAT
AAATCTCCCCATAAACAAAAGTCTTAAACGACTACATTGTGGATTCTTATTGTTATTGTGTTAGAAGAA
ACAAACCTAACAAGGATCTGACTGAAACAATTTCTCTACTTCTCGTAAGTATACAAATAAAATGTGCAT
ACACCATATTAATTTTCTCAAACTCTACACATATCAAACACTCACAAGCTGATTTAAACACGACTATTT
TTATAAAGGAATATGATGGAATAATGCCATTAAGATTCACAAAAAGATCATAATGAAACTTGAAACCCC
ACAAGATAGAAAAAGACAGCTAATCACTTGCACATGGACTTACATTAGTAGCCTTTCATTCCTCATCTT
TTTTTAAGATTTCAATAATATTATCATTTTCTACAAAAATAAAATAAAATTGTGGGCCCATTTGGCTCT
ATAGAACTCCACCTTTTTAATGGAAAAAAATAAATATCAAATTGACGATGGAGAAATTTGTGTGTGGAC
CCATTCACTCCAATCTCCATGCGACCCATCACAATAAATTTGGAAGTTTCCACAAAATATGGACTCTAT
AAACTCATTTCCCAAAAAGAAAAAGATCCTCAATTTTATTTATATTCATATTTATCACTAATAATAATT
GTGGTTAATTAATCACTTTAACTAATACTACTATATTGCTTAATCATGGTAAAATTAAAAAAAGGCCCT
TAAGAAGATATCTATGCTCAATAGTGAAATTAGAAAAAAATTAAAGTAGATTAAAAAAAGTAACATAAA
TTCGTATAATAATTTGTAGCATGTTTCGAACTATCTTTATCACTACAAAGGAATTTAAAAATTAATATA
TAAGATTTGAATAGAAAAAACATAATAACAAATATATCTCAAATTATTTAGAGATCTCATGCGTTATTT
TTTCCCTTACTATTTGTAAATGATCTTTATAATTGAAGTAATACTCGTAACAGATTTGCATAATCGTAT
CTCTCAAGAGAATAATCAAAAGGCCACAATTCAAATTCGAACAAACAGTTTCACAATCAATATATTATT
TAAGAAAATAATTTTAAAATTAAAACAACATTTATAATGAATTACATAATCAAATCTCTCGAAATAATG
GTCAAAAGATCATAATTCAAATAATAATATTTAAGGATCGAAGATAGAATATATTTATTATTCCAAGCA
TCTTACTGTAGGTGAATCATTCTTCTTAAAACTTAAATATAAAATTATAAATAAAAAAATAATATGACA
TAAAATAAAATATTAGAAATGATAAAGAAATGGAGTGAAAAAAAGTATAAAAT
SEQ ID 86
GACGAAGATCTTCTCCTGGTAATCTAAGGAAACATGAATATTTGTTGAGTTTTGGCTTGTGAAGATGCT
CTTTGTTCATCTGCTGTTTTCGATGGATTTGTGCAGATTAACTTGGAGAACATGAAGAAGCAGAAAGAA
TAGTTCCCTATCTTCTTCATCATCATCAAATGAGTGTGGATTAAAATGAAACCCACCCGAGTGTTCTAT
CCCAGAAGAGCAATACTAGTTTACATATACATATATATATATATATACGTATAAATGG
SEQ ID 87
AGATAAAGCAGATTCCTCTAGTACAAGTGGGGAACAAAATAACGTGGAAAAGAGCTGTCCT
SEQ ID 88
AGAGCTGTCCTGACAGCCCACTCACTAATGCGTATGACGAACGCAGTGACGACCACAAAA
SEQ ID 89
ATTCCCTCTATATAAGAAGGCATTCATTCCCATTTGAAGG
SEQ ID 90
CTGCTTGAGGGATTCGTGTGTATATGTATATAATAATTAATTTACAATTTGGTGCAAATTAAATAACTT
ATATTCAATTTATTTACATTCATATATAAACTTTATATATATTAAGAGTTTAATTTCCCCATAAACAAG
TTTTTTATGAATTTTCAGTCACAATAGAATTTTTTTAAAAAAAATATTTTTAAATGTTTAACTTAAATT
ATGAAATGTGTAAATGTTTGTTAACCATATTTAGGGCTATTGT
SEQ ID 91
ATATTTAGGGCTATTGTTATTATTTAATGAAAAATAAAATATAATATAATTCTTAAGAAAGTATTATAT
ATAAAATAAAAAATTACGTAACAAATTATACTATACCCACAAAATATAATTATGTAAACTATACCATAT
AATATTATTTCGTAAATTTAGTTTGTCATATAAAATTTTCCCTAAAATGAACAGAAACCC
SEQ ID 92
AGATAAAGCAGATTCCTCTAGTACAAGTGGGGAACAAAATAACGTGGAAAAGAGCTGTCCT
SEQ ID 93
AGAGCTGTCCTGACAGCCCACTCACTAATGCGTATGACGAACGCAGTGACGACCACAAAA
SEQ ID 94
ATTCCCTCTATATAAGAAGGCATTCATTCCCATTTGAAGG
SEQ ID 95
TATTATTTATGTCTAAAAAAATTTAATAAACTTTGACAAAGAAAAAGTAAAAAATAAAATTTTATTTTA
TTTCTACAATTTATCTACAATGTAAATAATTATAATTTAAAAATTATTTAATAAAAAGTTTATCTAATA
CTTTTATTCAAAAATAAATTCTACTTTTTATAGTTTGTGCTCACATATTAATATATTTTTAGACCAAAT
AATAATTTAATTTCAAAAATAGTATAATAGATCCTAGAAATTATCTAAAAATAAAATAATTATAATTTT
AGAACCATTTTATTATATATATTAAAATATAATTTTTTTAATATTTCTATTTTTGTAAAAATAAAAATT
CTTATAGTTTGTGGCCAAAGTTGGTCAAAATATTTTTTTTTCTTTTAATGGTACTTAAAAAACACGTTT
CTTTTATTTTTTGGTACCTTTAAATAGGTATTTGAAGTTCAAAGTCATGTTAGTCAATAGAAGTTTACT
ACCGTTAACGGCCACGTGCGGGACACATGGCCTCTGTTGTTAACTTGGGACAAAAAAGTATGTTTTTTG
TGTTTTATAGTACCAAAAGTGACACTTGCCACAATTATGGTACCCAAAATAAAATCAACTTTTTTTAAC
GGAATCAAAAAAAAAAAATTTTGCCCTTACATAATATATGTACTAATCAACGGATTGAATTTTCTATTG
TAATATTCATTTCATTTTCTATTTCGTTCAACATATACAATTATGTATATTTGAACGAAATCATATATT
TTATTTTGAAAAATAAAAAAAAATTAACACATGCTATGTATATATTGATTGTAATAAAAAATAAAATAA
TTAAAATTTGCAACAAATGCAATCCAACCAAACATAATCGCCACATACCCATTAGGTGTAAGCAGAGCA
GCATTTCCATACATGCAACCTCATGATGATCATAACAAAACAAAAGCCCATGCACAATAGATACCGCCA
AATGTCGCTCGTTTCTCACCATCTCACACTCGACGTGTCGACCTCAACCCACCAATTTCAACTATAAAT
CCCCACCCTTCTCTATTCCCCGCTTCACATCCATCATCAGCCCCCTCAAACTACTAATCCCAGCACCTC
CAAAC
SEQ ID 96
GTATATAAATAAACAAAAACCTCAAAAGCAATCAAGGGCAAATCTCCAAAATAGCATATTTCTAAATTT
ATATCACAAAAATAGCAATCAAAAACTAAAATGACTAAAATGACCAAAATGATACTTTTCTAAGTTTAT
CCTTTGAAAATTTTAATTTTTTTATTTTTCAAAATTTGAAATCTTATCCCCAAAACCTCATTTCTCAAC
TCTAAACCCTAAACCATAAACCCTAAAATCTAAACCTTAAACCCTAAACCCCAAACCCTAAACCCTAAA
CCCTAAATCCTAAACCCCAGCCTTTAACTCTAAACCCTAAGTTTGTGACTTTTGATAAAACATTAAGTG
CTATTTTTGTGACTTTTGACCTTGGGTGCTAGTTTGAGAACATAAACTTGATTTAGTGCTATTTTTGTC
TTTTTCTCATCATATAACTTCTTTTATAATTACAGAATATCAAAAATATGGTTTTCTGTTTTATCTGTA
G
SEQ ID 97
AACTGGATCAGACAAATTTGTGTGTTTATCTTTAAAATTTAGTGCATGGGCATATTTGGTCTGTTGGTT
TACTGTTCTTGGATTGGTGAAAGAAATTCTCAAGCCTTCTTTTGTGTCATTAATCTAGAAATGTGTCAA
CTGCTCAGACATCAGAGTCGTGTTACTATCCAAATTCATCGAGTTTCAGTCTCATTGTTCTACAAATTG
GTCTTTGATAAACGCTAAAACTAGAACAAATAATATAGCTCCAAGATTCCGATCCTAGCAAACAATAAT
GATATAAATCTAGTTAACAAAACATCGCTTAAATTTCCAAGATGCTTGCCGTTTGTAGATTCCACACTA
TTTTTCGTCTCAACTAAAGCAGTCTCCAAGTACACAAAATATGTGTATATACAACAGAAGTCGAACTTG
TTATAGAAACTAAGAACTGAAAACCAAAGACCAAACCACTGCTCTTGGAAGGCCAAATGTAACAATACA
CTTGTTTCTTGTCTTCTCTTTTTCTTTTTTTCTTTTTCACATTCTACTATAAAAAAAAGGCGAAAAACT
TAGATATAATTTTGCTACCAAC
SEQ ID 98
ACTTTTATCATTCCCAATACAATATATTCCACTTTCCCCTTTATTTATACACTTTTCTTAATCTGTGTG
AAAAACCAAAGTAGGTCAATTAAACCGGGACGGAGGGAGTACAAAAATACAACGTTCAAGATTCTACAA
ATTGCAAATAATTTAGCAGAATTTGCAATGCATAATTTATATTTTTAGTATACTATCATGTAGGACATT
TCTTAAAAAAGAAACAATTCTTTACAATGACCTTCAAAAAATACTATACGACCTACTTTGCGTAAGCAG
TATACATTTTCCACATTGAGCCAACACGAATAGAATAGAACTACTCTGCCTACCTCATTATCACGTCAA
AAAAATAAAAGCCTACCTTTATTTTAAATGATTCAATTTCATTTGCCTTAACTTTATTTTTCATTTTCG
AATTAAGGGATTAGCGTCAAATTCAACTTTCATTTTTGTTCAAAAAAACTTTCATTTGTATTTTGTTTT
ATGAAGTATTTAGTAACCGAAATTTCATTAGTTAAAGTGAATAAGTAAAGAATATTGACTTCGATTTCT
ACGTATTATAATGTTTCTACAAACTTTTGTTTGTATTAAAATTAAATTATTATTTTTCATAAATAAAAT
ATAGAAAATTTAGTGATTTTTTTAAGGAAAAAAAATTAGTGATTTGTTTTTTTGGTCAAGAAAATTAAG
TGATTTAATCCCTTACTATATATCATGCAATACCTTTTTTTCCTTTAGGAAATTACGCAATACCTGTAT
GGTTGGTAAATCAAATAATTCTT
SEQ ID 99
ATTCAATTTCATTTGCCTTAACTTTATTTTTCATTTTCGAATTAAGGGATTAGCGTCAAATTCAACTTT
CATTTTTGTTCAAAAAAACTTTCATTTGTATTTTGTTTTATGAAGTATTTAGTAACCGAAATTTCATTA
GTTAAAGTGAATAAGTAAAGAATATTGACTTCGATTTCTACGTATTATAATGTTTCTACAAACTTTTGT
TTGTATTAAAATTAAATTATTATTTTTCATAAATAAAATATAGAAAATTTAGTGATTTTTTTAAGGAAA
AAAAATTAGTGATTTGTTTTTTTGGTCAAGAAAATTAAGTGATTTAATCCCTTACTATATATCATGCAA
TACCT
SEQ ID 100
TCAGACACTCAATACGTGGGAACTTATTCACTTTCGTGTAGGAAAGTGGAACCTAAACGAAATTGCAGT
GTGTTAATATGCCCATACTACATTGACGATATTATAGTCTATTTTGGTGTCTATTCACAAGCCAGATAT
GGGAAATTATCTATTTTGGTGGCTACCACCCCGTTATTCATAACTCCACTGCACTTGTTACTGATGCTT
CGAATACTTACAATTTAGAGTTTAGTTTCAAACTGAGCGGAAAATTACAATATTTTAAATAATTAAATT
TGGCGTTAGGACATAAAAGTGAGACTATTCTACCCATATGTTTAGTACAACGCAATTAAGCACATGGAT
ATTACATTCCGTCGGCTTCCACACGCGCACGCGCTTGCAGGGTGATTTTTGTCAATTTTTGACAAAACT
TGTCACTTGGATGAGTCCGTACTCTAGCATGGCTATATTGTACATTTTTTTTGCCTCTTATGAATATCC
CATAAATTCTCTCATCTATAATAAGTAGTAACATGGACGTTTCAGGTTTGGGATCTGTTGAAACTTCAT
TTTTTCAGTTTCTTCTGTTTAAGTAIATGTGGCAAATTCAAACCAAAACTTCTTTACAGTTTTGATGAC
TTGTATTTCTTGTATTTCGAGAAAAATAAACCAAGCTCAAAAGATAAAATACAGTTTAGTTTTACTAAA
TTAATTCAACTTGGTTGTTGTACTAGACTTGGTTACGTTCAAATGCCACTATTCACGTTGGTGTGAAAT
AAGTTTTTGTTAAACAATAAATATGAACGCAGATAGATGGTGAGAGGAGCAGCATCTATAATTCATTGA
AAACGCAGAAGGGTTACCAAAAAAGGGGAGTTTCCAAAAGATGGTGCTGATGAGAAACAGAGCCCATCC
CTCTCCTTTTTTCCTTTCTCATGAAAGAAATTGGATGGCCCTCCTTCAATGTCCTCCACCTACTTACCA
CTCATTTTTTTTTCCTTATTATTTCAATAATTGATTAATAATTAGTTTCTAATTTCAACTTCCAGTTCT
GTAAACAGCAAAAATTATATATACAATCTAACATCTCACTTGTATATACCTATATAAATATTCGTATCT
ATTTATATGCATGTCTAGAGGATAAAAAGTGTGAGCTTTGTTGTGTATATGTGCTTTTTGACAGTTGCT
AGATAATTGGTATGCCTGTTTTTCTTTTTCTGCTATTTATAAATACATCTCAGCTAAGAAAGAACTTGT
AACCTTCTGTTTTCTGCAAGTGGGGTCAAAGTACCTTCAGAGAAATATTCTTTCAAGTGAAACTCGTAA
ACCAAAAAAAAATTTACACAAAGAAAGAGAGATATTTTTCAAGAACATTATTATTACGAAAGCAGAACC
AAGACTTAAGTTACACTGAGATCAATAATAATTATAATATATATTATCGCTTCAAAACCAGTTTCTCAT
TAGTAACTTCTCCTTGTGTCCTGATCTCCAGGTAAGGTTGTGAATGATACAGTATATATATTAACCCTA
AAAACAAGGTTTATGATAAAATATCTGATCCTTGATTTAACAATTCGTGGGTCTGATATCGTTCTTGGT
TTATTTGTTTATAATGTATAAATTAAAGAGTTCTA
SEQ ID 101
CTGTCCCCTGCATGATGCAATTTCTTGCTTAAATTAATATGTGGATGATATTACGGCAAAACAATAAAC
CTCTAATATTCAAGATGCCGTTGGACTAACCAATTTTCCAAGGATAAGACTCTCAAACATAAGATTTCG
AAAAGACAAAACCAATTAAACTATTTATCGAGCAATTGTTCCTAAATCTTAACCCAAACCATTATTATT
TTTCTTAAGTTCTGCGTTTGATTTTACATTTTAGTCTAAGAACACTAATATTTTATGTTTTTTTTTTAA
TTTAACTTGAAGTATCTTTTTTTTTTGAATGAATGTTAAATTTATTCATGCAAAAACATATTTACATCA
TGTGCAACTGTTTATGAATCAAAGAATCAGCTCATGAAACTAAGAACAGAATTCCGAAGTTAAGGATCC
ACTCTAAATTCCTAACTTGAAATATCACACTTAGTATCCAAACGTAAACACAAATTCAAAATGTATAAA
AGGGCAATTAATTAAACCTGAATTATCTCATTCATTGGCTCTCATGATACATGATAAGTTGTAAAACTT
CATGTCAGTTGGGTTAAGTTTTGTTTAATTGGAATACAATAATTCAAAAATATAATAGCATTAATACTA
TACCAGCTTCATATTAATGTAGGAGTAGGGCAATAAAAAGAAAAGAAGAAATAAAAAAAAGGATTTACC
CAAAAAGGAGAATTTCCAGAAGTTGATTCTGATGAGAAACAGAGCCCATACCTCTCTTTTTTTCCGTAG
ACATGAAAGAAAAATTGGATGGTCCTCCTTCAATGCTCTCTCCCACCCAATCCAAACCCAACTCTCTTC
GTCTTCTTTATTTTTCTATTTTGTTATTTTCTACTCCTTAATTCCCATCAATTTTCAGATTGCGATCTA
AATGTATATATATACATAGAGAATTAAAAGAATTAGGTATGAGATTTTTGTTTTAGAGTAATGGTCCAT
TTTCTTTCTTTATTTTTCTTTTATAACATTTCAGTTTGAATAAAACTACCAAACCTTCTGTTTTCTGCA
AGTGGGTTTTTAAATACTTTCAAGGAA
SEQ ID 102
AATACATACCAATGAAAAGAACAAAAATGACTTATTTATAAATCAACAAACAATTTTAGATTGCTCCAA
CATATTTTCCAAAATTAACATTTAAATTTTAATGCAAGAAAATGCATAATTTTTTACTTGATCTTTATA
GCTTATTTTTTCAGTCTAATCAACGAATATTTGAAACTCGCAACTTGATTAAAGGGATTTACAACAAGA
TATATATAAGTAGTGACAAATCTTGATTTTAAATATTTTAATTTGGAGGTCAAAATTTTACCATAACCA
TTTGATTTATAACTAAATTTTAAATATATTATTTATACATATCTAGTAAATTTTTAAATATATGTATAT
ACAAAATATAAAATTATTGTGTTCATATATGTCGATAAATCCTTAAATAATATCTGCCTTTACCACTAG
AGAAAGTAAAAAACTCTTTACCAAAAATACATGTATTATGTATACAAAAAGTTGATTTGATAACTATTG
AAATTGTATACGAGTAAGTAATAGAAATATAAAAAACTACAAAACTAAAAAAATATATGTTTTACTTTA
ATTTCGAAACTAATAGGGTCTGAGTGAAATATTCAGAAAGTGGACTACAGAGGGTCATA
SEQ ID 103
GATATCTATGCTCAATAGTGAAATTAGAAAAAAATTAAAGTAGATTAAAAAAAGTAACATAAATTCGTA
TAATAATTTGTAGCATGTTTCGAACTATCTTTATCACTACAAAGGAATTTAAAAATTAATATATAAGAT
TTGAATAGAAAAAACATAATAACAAATATATCTCAAATTATTTAGAGATCTCATGCGTTATTTTTTCCC
TTACTATTTGTAAATGATCTTTATAATTGAAGTAATACTCGTAACAGATTTGCATAATCGTATCTCTCA
AGAGAATAATCAAAAGGCCACAATTCAAATTCGAACAAACAGTTTCACAATCAATATATTATTTAAGAA
AATAATTTTAAAATTAAAACAACATTTATAATGAATTACATAATCAAATCTCTCGAAATAATGGTCAAA
AGATCATAATTCAAATAATAATATTTAAGGATCGAAGATAGAATATATTTATTATTCCAAGCATCTTAC
TGTAGGTGAATCATTCTTCTTAAAACTTAAATATAAAATTATAAATAAAAAAATAATATGACATAAAAT
AAAATATTAGAAATGATAAAGAAATGGAGTGAA
SEQ ID 104
AGTGGAGWAGCAAAGGGCTATCCGGAACCTCTTTAATGTAAGGTTTGCATACATTCTATACTCTCTTTA
CTCAACTCATGGAATCACACTGAATGTAYTGTTGATGTACCTTACTCAGTGGCGGATCTATGAAGTGCT
GTGGGGRTGCCACGCCACCCCCGAACTTCGACGGAAACTCTATATATACATAGGTATATATGTATAATA
TTTATATACATATAAAGCGTGCCACCCACAGAACAAAATTGGCTTGTGGTGCCACGGTAGGAGGGCGAC
TTTAGAAGGTTGAGGTTGCGGGTTTGAATCCCATTTGACACCCACGGACTCTAAATCCTGGATCCGCCA
CTGACCTTACTTATTATCCTTCCCTTAATATAGTCAATTTTTTTTAACGACCTCGTTTGTTCGGAACAC
AATTTTTTCTTTTTCATTTTTTATTCTCCACAGAAACTTTTCTTTTTCATTTGATAGTATAAAAAATTC
AAAAAAATATTTTTGTCGTATTTCCCTCATTATTAATTGTTGATAATAATACTTGGAGGCTATCGCTAT
CATTGTGCTCTCAAACCAACGTGGGCACACACCTAAAGAAGATAATATATGCACAAAAAAGAGTACATT
TTATACACATTCATAAATTTAGTTAATCTACACCTTCCATTTTGTACTTATCCTTTATCAACCATTCTG
ATCTCTCCATGTCATCACTATATATCCTCTAAATTTTCCTTTTATATTTTTCCAATTTCCATCTCCATC
CTTTTCCGCTCGCCCTTTAATTGAGAGTCTTTCCATAACAACTTTTCTATTTCTCAATATATAAGAATA
AGATCTGCATATATTTCACTACATTTATTGTATTATTTCATAGATTAATTGAGATGCTCGTAAGCTCAC
CCTCCAATCGAAAGTCTTTCCGAAATAACTTTTTTATTTCTCAACAGATAAGAATGATCTGCATATATT
TCATTGCATTTGTTATATTATTTCGTAGATTAATCGAGGTGCTAGTAAGCAAAAAGTAGAAGGAAAAAG
AAAGTCAATTGAGGGCATTATTGTAAATAAGTCCAATAGTGTGCCTTATCTTTTACTATATAAACACGA
GAACGTGACTCTTATTACT
SEQ ID 105
STCGAGTATGGWGTTGCAGAATCGGTTGTCCAAATTTGGAACTCTGTTAGAAATGCTACTAACTCAAAA
CAGTAATAGACCATAAATCTTGTTGGTTAGCAATGCTGCTTGTAGTCATGGTTTTTCTACTTCTGAAGT
AGAGTTTTGTTGAACTTCTGATATGCCAAAAAATAGAAAATTGTTYTCTTAAGGCCCTTTCTTTTATGA
ACATTGTGCAACCTAGTGTCATGTATCTTTAGCATRTATCACAAATTTTGGCTGATATACAGTTGTTGT
CACTCAAGATCTATGGTCTTTATCTAGACCCGATGAAAAAAGTGGGTCACCTACGTTTGTTGGTTATAC
TTGTACCTACTTTCTTACCRATAGTATTAGCAAGGGTCTATCGGAAACCTCTTTATTTCTACCAATTCA
CTAGTGATTAGAGGAGTAGCAAAGGTCTATTGGAAACCTCTTTATTTCTTTATTTCTACCAGATGGATG
TAAGGTCTGTATACACTCTATACTCTCTCTACGCAATTTATGGAATCACACTGAATATATTGTTGATGT
ACCTTGCTTATAATTCTTTCCTTAATATAATTAAATTTCTCTATAACGACCTCGTTTGTTCGGAACACA
AGTTTTTCTTTTTCATTTTTATTCTCCACATAAACTTTTCTTTTTCATTTGATATTATAAAATATTCAA
AAAAATATTTTTGTCGTATTTCCCTCATTATTAATTGTTGATAATAACACTTGGAGGCTATCACTATCA
TTGTGCTCTCAAACCAACGTGGGCACTCACCTAAAGAAGATAATATATGCACAAAAAAGAGTACATTTT
ATACACATTCATAAATTTAGTTAATCTACACCTTCCATTTTGTACTTATCCTTTATCAACCATTCTGAT
CTCTCCATGTCATCACTATTTATCCTCCAAATTTTCCTTTTATATTTTTCCAATTTCCGTCTCTATCCT
TTTTCTGCTCGCCCTCTAATCAAGAGTCTTTCCGAAATAACTTTTCTATTTCTCAATATATAAGAATAA
GATCTGCATATATCTCATTGTATTTATTATATTATTTCATAGATTAGTTAAGATGCTCGTAAATTTGAC
CTCCTATTGAGAGTTTTCAAAATAATTTTTTTATTTTTCAATAAATAAGAATAAGATCTACGTATATTT
CACTCTATTTGCTGTATTATTTCGTAGATTAGTCGAGGTGCTCTTAAGCAAAGAGTAGCAGGAAAAAGA
AAGTCAATTGAGGGCATTATTGTAAATAAGTCCAATAGTGTGCCTTATCTTTTACTATATAAACACGAG
AACGTGACTCTAATTACT
SEQ ID 106
ACGACCTCGTTTGTTCGGAACACAATTTTTTCTTTTTCATTTTTTATTCTCCACAGAAACTTTTCTTTT
TCATTTGATAGTATAAAAAATTCAAAAAAATATTTTTGTCGTATTTCCCTCATTATTAATTGTTGATAA
TAATACTTGGAGGCTATCGCTATCATTGTGCTCTCAAACCAACGTGGGCACACACCTAAAGAAGATAAT
ATATGCACAAAAAAGAGTACATTTTATACACATTCATAAATTTAGTTAATCTACACCTTCCATTTTGTA
CTTATCCTTTATCAACCATTCTGATCTCTCCATGTCATCACTATATATCCTCTAAATTTTCCTTTTATA
TTTTTCCAATTTCCATCTCCATCCTTTTCCGCTCGCCCTTTAATTGAGAGTCTTTCCATAACAACTTTT
CTATTTCTCAATATATAAGAATAAGATCTGCATATATTTCACTACATTTATTGTATTATTTCATAGATT
AATTGAGATGCTCGTAAGCTCACCCTCCAATCGAAAGTCTTTCCGAAATAACTTTTTTATTTCTCAACA
GATAAGAATGATCTGCATATATTTCATTGCATTTGTTATATTATTTCGTAGATTAATCGAGGTGCTAGT
AAGCAAAAAGTAGAAGGAAAAAGAAAGTCAATTGAGGGC
SEQ ID 107
CTCGAGTCCATTGTGGGGCTCCCATTTCTCTTTGCATTTCAAGAGGGAGCCATAAAGGCTCTAAATGTC
ATTCATCGAGTCAATTCGTCAAAATCGGCGTATGAAGTCAAATTTCAAAGTTTAGGAGATTGAAGAAAT
TTGAAGAAGACTAACTAGAAGACTTCTTTAGTTTTTTTTTTATATTTTGTGTTTCTTTTGTAATGGCCT
AAGCCCTTATGGTTTTATTTTCTTGTACCTATTCTTGTATGTCTAGACTAGGACAGGTACAAAAGAAAG
AAATGGGTCGAAAATCCAAAAAACAGGCGGATCCAAAACTTGGTCAAGGCGAACAGAACCTGAGTTTGG
ACCCAAATCTCTCTCTCTCACTTTACTATTTGTTTACGTATTTTTGCTTAAATGTCGTTAGCTTAGGAT
TAGAAACTCCAAACCCCGTCGAACGCCTTTTAAATTTTCGTCAAACTTAAAATTAACTTTTTAACGATA
ATTTGTTTCAAATTTGCAAAGCTTGTTAGATAAAACCTTAGGAAAGTTTAACTTTGAAATAGATTCGCA
AAATTGTGAAATAAACAATAAAGATTGCAAAACTTGTCGACTTGTTTAAATGAAATAAAAGTTCAACTT
CAAATTGCAAAAGTTACAAAAAATAGTCAAATAAGTTAATCGCCGGAAAATCGTATTTAACGGAGTGTC
ACCTTCCTAAGACACTAATAGGAATCCCGAACTCTTTAACATTTTCCAAACAATTTTCCTGTTTTAAAG
TTGTTTAGAAAATAAGTTTTCTTAATTTTCTCAAAATTAAGTGGCGACTCCTAAAAAGTCGAAAATCCT
CTGAGATAAAACAAACTCTTTTCGAAAATCATTTTTTTCGATAAAACAAAATAAATTAAAATGAATAGA
AAGAAAAGTTAAAACAGTGGGAGTACTAAGAATTGTATGCGTCTATATCTTTTTTTTATATCATTTAAC
TTAGTGGTACAAGCTTTCTGCCTATTATATAGAACGAGTAAGCGCCATTTGTTGCAAGATATCTTTTTA
TAACAAAATACAAGTTAATTTTCAGATTAAAAAATATTTAAGAAGTTTTTGAAAAGGGAGTTACATGAA
TTTTATTATTTTAGGAGTTAATAACTTAGTTACACTTTAGTTTGTAATATTAAATATTTTATTAAATTT
TGGTGCCCCAAAGACGTCCAAATACATGTTACTTGAGGTCAAATTTAAGTGTAATTTGAAAAAAAAAAG
ATCGTTGTAACCAAGTGTATTAGCATATATTTAGGATACATAGTAAATCTCCTTCACCTCTTTCCCATC
TTGCTTGCCACTCTCTCGTATATCTAATATTCTAGATACATGTGAATCACTCCTGATATATGTACATAG
TTTGATTCACATAATATATGTATAGGATACATACAAATTTCACTTGTTTTTTTTTCTATTTTTTGTGTA
TCACGTAACAAAAATATATATATCTCAGTGTAGAATACATAAAAAAAATTTTAATTAGTGATAAAATAT
ATAATATGATTAAAAATATAAATAATAATAATATATATAATAATAAAGTATGTCTAATTAGGTAGTTTT
TCTTTTTGAAAACTGAAATGAGAAAAAGCAAAACATAAAATTGACTTGAATGACAGCTACATGACATTT
TCATCTTGTAGTAGGGACATATGATTTGTTTTTTTCCTTTGCCACATGTGTTCTGTTATCCTTAATCTC
CAAGTAATCCCATATTTTGGTTGATGATTCACAATATAATCTATCTAATTATGCACCTCCTTCTACTTA
AAGAAGAAAAATGTGATGGCGATTGGCAATTGGGAAGATAATTAAAATCTGTTGAGTACTCTTTCATCC
GCAATGGCATTCAGTCGATGGAACAATAGTGAAAGAGATGTTTAAAAAAATTATTTACATTTAAAATGA
TTTTAGATTTGACGCAATCCGAAAAAATTAGTCTATAAAAAAAATTATTTAAAATCATGCAAGAGCTCA
ATTAACTTCATCCGCCTTTGATGTGAGTTTTTCTACATTCATCACGCTTCCCATCCCCGAACCCCAACA
CTCTATACTCCGATCCATGACGTGAACAAATTATTCAAGCGTTCAATTTGACTCTAATATCATACTAAA
TAAACCTAATTTAATAGTAAAAATTAGCTTAACAATTTACTAATTTCACACAATTTTTTATATTGTTGT
CTTGTCATTATCTTTAGGTAATAATAGTGTAAAAATTATCTTACACGATTATACTACATAATTTATACG
ATTCGTTGATAAATTGTATACCAAAGTGCCACCTCATCACACAATAATTTAATTTGGACTAAGTTCACT
ATTAGTGAATGAATGAATTTTAATTATAAATAGAGGACTTGACAAGATCATATTTGTATCAAACACCAT
ACACTTTCTAAATTATCGATAGATTTATTGTTTCAG

What is claimed is:

1. An isolated or synthesized gene promoter polynucleotide, comprising two copies of a sequence from the promoter of at least one target gene that are positioned as inverted repeats, wherein (a) the gene promoter polynucleotide does not comprise a sequence naturally found downstream of the target gene's transcription site and (b) transcription of the gene promoter polynucleotide produces a double stranded RNA molecule.

2. The isolated or synthesized gene promoter polynucleotide of claim 1, wherein the sequence of either DNA strand of target gene promoter comprises a specific non-transcribed sequence (“SNT”) which comprises at least two copies of a CAC trinucleotide in the upper and/or lower strand of the polynucleotide.

3. The isolated or synthesized gene promoter polynucleotide of claim 1, wherein the SNT sequence comprises at least about 50-100 contiguous nucleotides of the target gene promoter sequence.

4. The isolated or synthesized gene promoter polynucleotide of claim 1, wherein either strand of the SNT sequence comprises copies of at least one of a GTG trinucleotide.

5. The isolated or synthesized gene promoter polynucleotide of claim 4, wherein at least one CAC trinucleotide is located in an A/C-rich or G/T-rich region.

6. The isolated or synthesized gene promoter polynucleotide of claim 2, wherein the SNT sequence does not comprise a TATA box motif.

7. A gene silencing construct, comprising the gene promoter polynucleotide of claim 2 operably linked to a functional promoter and regulatory elements for expressing the gene promoter polynucleotide in a cell.

8. The construct of claim 7, wherein the gene promoter polynucleotide comprises multiple copies of the SNT sequence.

9. A method for downregulating a target gene in a cell, comprising introducing the gene silencing construct of claim 7 into a cell, wherein the SNT sequence of the gene promoter polynucleotide comprises a sequence that is identical to or similar to a sequence located upstream of the transcription start site of a target gene, wherein expression of the gene promoter polynucleotide brings about downregulation of expression of the target gene in the cell.

10. The method of claim 9, wherein the cell is a plant cell.

11. The method of claim 9, wherein the functional promoter is selected from the group consisting of a potato Agp promoter, a potato Gbss promoter, a potato Ubi7 promoter, an alfalfa petE promoter, a canola Fad2 promoter, and a tomato P119 promoter.

12. The method of claim 10, wherein (a) the plant cell is in a plant, (b) the gene promoter polynucleotide is integrated into the plant genome, and (c) downregulation of expression of the target gene in the plant cell modifies a trait of the plant compared to a plant that does not have the gene promoter polynucleotide integrated into its genome.

13. The method of claim 12, wherein the modified trait of the plant containing the gene promoter polynucleotide is at least one of a modified oil content, reduced cold-sweetening, reduced starch phosphate levels, increased bruise tolerance, increased starch levels, delayed postharvest softening and senescence, prevention of anthocyanin production, and reduced processing-induced acrylamide accumulation.

14. The method of claim 9, wherein the gene promoter polynucleotide comprises inverted copies of a deoxyhypusine synthase gene promoter, which is expressed in a cell from an alfalfa or canola plant.

15. The method of claim 9, wherein the gene promoter polynucleotide comprises inverted copies of at least one of (i) a shatterproof gene 1 promoter or (ii) a a shatterproof gene 2 promoter, which is expressed in a cell of a canola plant.

16. The method of claim 9, wherein the gene promoter polynucleotide comprises inverted copies of at least one of (i) a Fad2-1 promoter, (ii) a Fad2-2 promoter, (iii) a Fad3 promoter, and (iv) a FatB promoter, which is expressed in a cell of a canola, soybean, cotton, safflower, or sunflower plant.

17. The method of claim 9, wherein the gene promoter polynucleotide comprises inverted copies of at least one of (i) a C3H promoter or (ii) a C4H promoter, which is expressed in a cell of an alfalfa plant.

18. A method for downregulating a target gene in a cell, comprising introducing into a cell a gene silencing construct that comprises the gene promoter polynucleotide of claim 1, wherein the gene promoter polynucleotide (a) is not operably linked to a functional promoter or to any other regulatory elements, and wherein the presence of the construct in the cell brings about downregulation of expression of the target gene in the cell.

19. A method for identifying a gene promoter polynucleotide, comprising
(a) isolating a promoter fragment from a target gene, wherein the promoter fragment does not contain any sequence downstream of the target gene transcription start site,
(b) introducing an expression cassette comprising a functional promoter and regulatory elements operably linked to either (i) the promoter fragment or (ii) inverted copies of the promoter fragment into a cell that contains the target gene, and
(c) determining whether expression of the target gene in the cell is down-regulated compared to a cell containing the target gene but not the expression cassette, wherein the transcription of a promoter fragment or inverted copies thereof which brings about downregulation of the target gene is a gene promoter polynucleotide.

20. An isolated or synthesized gene promoter polynucleotide, comprising (i) at least one sequence from the promoter of a target gene, wherein (a) the gene promoter polynucleotide does not comprise a sequence naturally found downstream of the target gene's transcription site and (b) the gene promoter polynucleotide is positioned between functional promoters that are operably linked to the gene promoter polynucleotide in convergent orientation.

21. The isolated or synthesized gene promoter polynucleotide of claim 20, wherein the promoter sequence comprises an SNT sequence that comprises copies of a CAC- or GTG trinucleotide, or a combination thereof.

22. The isolated or synthesized gene promoter polynucleotide of claim 20, wherein the gene promoter polynucleotide comprises promoter sequences from more than one target gene.

23. The isolated or synthesized gene promoter polynucleotide of claim 20, wherein the promoter sequences are from different target genes.

24. A method for downregulating at least one target gene in a plant cell, comprising (i) introducing the gene promoter polynucleotide of claim 1 or 20 into a plant cell or (ii) integrating the gene promoter polynucleotide of claim 1 or 20 into a plant cell genome, wherein (a) the gene promoter polynucleotide is operably linked to at least one functional promoter and (b) expression of the gene promoter polynucleotide brings about downregulation of at least one endogenous target gene in the plant cell.

25. A method for downregulating more than one target gene in a cell, comprising introducing the gene silencing construct of claim 6 into a cell, wherein SNT sequences of the gene promoter polynucleotide comprise sequences that are identical to or similar to sequences located upstream of the transcription start site of at least two target genes, wherein expression of the gene promoter polynucleotide brings about downregulation of expression of the target genes in the cell.
